(19) United States
(12) Patent Application Publication
(10) Pub. No.: US 2024/0342206 A1
(43) Pub. Date: Oct. 17, 2024

(54) mRNAS FOR TREATMENT OR PROPHYLAXIS OF LIVER DISEASES

(71) Applicant: CureVac SE, Tübingen (DE)

(72) Inventors: Tim SONNTAG, Tübingen (DE); Marion PÖNISCH, Tübingen (DE); Frédéric CHEVESSIER-TÜNNESEN, Tübingen (DE); Michael OTT, Hannover (DE); Amar DEEP SHARMA, Hannover (DE)

(73) Assignee: CureVac SE, Tübingen (DE)

(21) Appl. No.: 18/293,542

(22) PCT Filed: Jul. 29, 2022
(86) PCT No.: PCT/EP2022/071449

§ 371 Date: Jan. 30, 2024

(30) Foreign Application Priority Data
Jul. 30, 2021 (WO) PCT/EP2021/071435

(51) Int. Cl. A61K 31/7105; A61K 9/00; A61K 9/127; A61K 9/51; A61K 38/00; A61P 1/16; C07K 14/705

(52) U.S. Cl. CPC A61K 31/7105; A61K 9/0019; A61K 9/1271; A61K 9/5123; A61P 1/16; C07K 14/70567; A61K 38/00

(57) ABSTRACT

The present invention relates to mRNA medicines for use in the therapy and prevention of liver diseases like liver fibrosis, liver cirrhosis, hepatocellular carcinoma (HCC), non-alcoholic fatty liver disease (NAFLD), non-alcoholic steatohepatitis (NASH) or liver cancer, and more particularly to mRNA medicines of this kind which can exhibit excellent therapeutic and preventive effects with respect to liver diseases individually developed or to complications resulting from diseases of these organs. In detail, the present invention relates to an mRNA suitable for treatment or prophylaxis of liver diseases. In particular, the present invention provides mRNAs encoding hepatocyte nuclear factor 4 alpha (HNF4A), human wild type and engineered variants thereof), or a fragment or a variant of any of these peptides or proteins. The present invention concerns said mRNA as well as compositions and kits comprising the mRNA. Furthermore, the present invention relates to the mRNA, compositions or kits as disclosed, preferably LNP formulations or compositions, herein for use as a medicament, in particular for treatment or prophylaxis of a liver disease. The present invention also provides the use of the RNA, compositions or kits as disclosed herein for increasing the expression of said encoded protein, in particular in gene therapy.

### TECHNICAL FIELD

The present invention relates to mRNA medicines for use in the therapy and prevention of liver diseases like liver fibrosis, liver cirrhosis, hepatocellular carcinoma (HCC), non-alcoholic fatty liver disease (NAFLD), non-alcoholic steatohepatitis (NASH) or liver cancer, and more particularly to mRNA medicines of this kind which can exhibit excellent therapeutic and preventive effects with respect to liver diseases individually developed or to complications resulting from diseases of these organs.

In detail, the present invention relates to an mRNA suitable for treatment or prophylaxis of liver diseases. In particular, the present invention provides mRNAs encoding hepatocyte nuclear factor 4 alpha (HNF4A), human wild type and engineered variants thereof), or a fragment or a variant of any of these peptides or proteins. The present invention concerns said mRNA as well as compositions and kits comprising the mRNA. Furthermore, the present invention relates to the mRNA, compositions or kits as disclosed, preferably LNP formulations or compositions, herein for use as a medicament, in particular for treatment or prophylaxis of a liver disease. The present invention also provides the use of the RNA, compositions or kits as disclosed herein for increasing the expression of said encoded protein, in particular in gene therapy.

### INTRODUCTION

Fibrosis is the formation of excess fibrous connective tissue or scar tissue in an organ or tissue in a reparative or reactive process. Fibrosis can occur in many tissues within the body, typically as a result of inflammation or damage, which include the lungs, liver, heart, and brain. Every year, millions of people are hospitalized due to the damaging effects of fibrosis. The liver for example is frequently exposed to insults, including toxic chemicals and alcohol, viral infection or metabolic overload. Although it can fully regenerate after acute injury, chronic liver damage causes liver fibrosis and liver cirrhosis, which can result in complete liver failure. With regard to the liver, liver diseases represent a major health concern worldwide. The major causes of liver injury range from viral diseases and autoimmune diseases over metabolic disorders to malnutrition and alcohol drug abuse and vary greatly in different populations.

Several therapeutic approaches were designed to stop or reduce the fibrotic response (for review, see Wallace K. et al.: Liver fibrosis. Biochem. J. (2008), 411:1-18). However, at present there is no therapeutic agent indicated and licensed for the primary treatment of liver fibrosis and still, no targeted anti-fibrotic therapy exists at this time. Also for other liver diseases, such as liver cirrhosis, hepatocellular carcinoma (HCC), non-alcoholic fatty liver disease (NAFLD), non-alcoholic steatohepatitis (NASH) or liver cancer, an effective cure is urgently needed. For example for non-alcoholic steatohepatitis (NASH), when simple lifestyle adjustments are not enough, insulin sensitizers, weight loss drugs, antioxidants, liver protection and lipid-lowering drugs are clinically integrated into the treatment plan. However, these drugs usually have potential safety risks and insufficient efficacy, which can not meet the treatment expectations.

Thus, there remains a considerable need for alternative or improved treatments for liver diseases like liver fibrosis, liver cirrhosis, hepatocellular carcinoma (HCC), non-alcoholic fatty liver disease (NAFLD), non-alcoholic steatohepatitis (NASH) or liver cancer.

Surprisingly, HNF4A was identified as an important player in treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) or liver cancer. It is thus an object of the present invention to provide mRNAs encoding wild type HNF4A and pharmaceutical compositions comprising said mRNA(s) suitable for use in treatment or prophylaxis of liver diseases, such as liver fibrosis, liver cirrhosis, hepatocellular carcinoma (HCC), non-alcoholic fatty liver disease (NAFLD), non-alcoholic steatohepatitis (NASH) or liver cancer.

Inside cells, the transcription factor HNF4A is a short-lived protein, which upon delivery of mRNA encoding for the wild type protein is barely detected 24 h post-transfection. Consistently, HNF4A transcriptional activity on cellular target genes quickly declines. In addition, multiple cellular pathways exist, which further inhibit HNF4A activity via post-translational modifications (PTMs). Therefore, wild type hepatocyte nuclear factor 4 alpha (HNF4A), either on mRNA or on protein level, has properties that are not optimal for an application in patients. Thus, there is a great need for engineered HNF4A mRNA and/or especially engineered HNF4A protein variants that can be used advantageously in patients, having increased expression, activity and stability over wild type HNF4A mRNA and/or proteins.

Therefore, in the context of the present invention, it was a further object to provide mRNAs encoding engineered HNF4A protein variants and pharmaceutical compositions comprising said mRNA(s) suitable for use in treatment or prophylaxis of liver diseases, such as liver fibrosis, liver cirrhosis, hepatocellular carcinoma (HCC), non-alcoholic fatty liver disease (NAFLD), non-alcoholic steatohepatitis (NASH) or liver cancer. Therefore, in particular, it is an object to provide mRNAs encoding wild type HNF4A, mRNAs encoding engineered HNF4A protein variants and pharmaceutical compositions for expressing said proteins capable of treating, preventing, attenuating or inhibiting liver diseases as described herein. It is a further object of the invention to provide such an mRNAs encoding wild type HNF4A, mRNAs encoding engineered HNF4A protein variants and pharmaceutical compositions, which allow treatment, prevention, attenuation or inhibition of liver diseases, particularly as defined herein, in a safe and effective manner.

The objects underlying the present invention are solved by the claimed subject matter.

The inventors surprisingly found that wild type HNF4A and engineered HNF4A protein variants or respectively the mRNAs encoding said wild type HNF4A or engineered HNF4A protein variants of the invention are capable of treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) or liver cancer in a subject in need. I.e. upon administration of the inventive mRNA(s) to a mammalian subject, a liver disease as described herein, can be treated successfully. Sufficiently high expression HNF4A levels can surprisingly be obtained by using the mRNAs of the invention, being superior in comparison with the expression levels obtained by using a reference construct known in the art encoding the respective peptide or protein. Advantageously, the HNF4A encoded by the mRNA can thus exert its effects, which are preferably exploited in treatment or prophylaxis of a liver disease, in particular in the liver of a mammalian subject, preferably without or with acceptable side-effects. In preferred embodiments, the expression level of the encoded peptide or protein is sufficiently high in liver in order to exert its effect in liver and lower in other organs, in order to reduce side-effects. The inventive effect was proven in different liver fibrosis or respectively liver cirrhosis models (toxin CCl₄ model, cholestasis-induced fibrosis through feeding and a genetic model based on a surrogate mouse model of progressive familial intrahepatic cholestasis), which underline the robustness of the experimental data.

In more detail, as described herein further below, the inventors found that mRNA encoding an engineered HNF4A protein variant designated “combo 11” comprising 17 distinct amino acid exchanges (S87A K106R K108R K126R K127R S142A S143A S148A T166A S167A S313A S378A T429A T432A S436A K458R S461E), was more potent than mRNA encoding the HNF4A wild type protein with regard to the treatment, prevention, attenuation or inhibition of liver cirrhosis or respectively liver fibrosis, as measured by positive effects on hydroxyproline, sirus red staining, desmin staining, and Acta2 RNA levels and Col1a1 RNA level, even at RNA doses of 0.3 mg/kg (Acta2 and Col1a1 being fibrogenic marker genes).

The present application is filed together with a sequence listing in electronic format. The information contained in the electronic format of the sequence listing filed together with this application is part of the description of the present application and is incorporated herein by reference in its entirety. Where reference is made herein to a “SEQ ID NO:”, the corresponding nucleic acid sequence or amino acid sequence in the sequence listing having the respective identifier is referred to, unless specified otherwise.

### Definitions

For the sake of clarity and readability the following definitions are provided. Any technical feature mentioned for these definitions may be read on each and every embodiment of the invention. Additional definitions and explanations may be specifically provided in the context of these embodiments.

Artificial nucleic acid molecule and mRNA: An artificial nucleic acid molecule may typically be understood to be a nucleic acid molecule, e.g. a DNA, an RNA or mRNA that does not occur naturally. In other words, an artificial nucleic acid molecule may be understood as a non-natural nucleic acid molecule. Such nucleic acid molecule may be non-natural due to its individual sequence (which does not occur naturally) and/or due to other modifications, e.g. structural modifications of nucleotides, which do not occur naturally. An artificial nucleic acid molecule may be a DNA molecule, an RNA molecule, an mRNA molecule or a hybrid-molecule comprising DNA and RNA or mRNA portions. Typically, artificial nucleic acid molecules may be designed and/or generated by genetic engineering methods to correspond to a desired artificial sequence of nucleotides (heterologous sequence). In this context, an artificial sequence is usually a sequence that may not occur naturally, i.e. it differs from the wild type sequence by at least one nucleotide. The term “wild type” may be understood as a sequence occurring in nature or non-codon optimized. Further, the term “artificial nucleic acid molecule” is not restricted to mean “one single molecule” but is, typically, understood to comprise an ensemble of identical molecules. Accordingly, it may relate to a plurality of identical molecules contained in an aliquot. Thus, in preferred embodiments, the mRNA of the invention is an isolated mRNA or an artificial nucleic acid molecule.

Bicistronic RNA, multicistronic RNA: A bicistronic or multicistronic RNA is typically an RNA, preferably an mRNA, that typically may have two (bicistronic) or more (multicistronic) open reading frames (ORF). An open reading frame in this context is a sequence of codons that is translatable into a peptide or protein.

Carrier/polymeric carrier: A carrier in the context of the invention may typically be a compound that facilitates transport and/or complexation of another compound (cargo). A polymeric carrier is typically a carrier that is formed of a polymer. A carrier may be associated with its cargo by covalent or non-covalent interaction. A carrier may transport nucleic acids, e.g. RNA or DNA, to the target cells. The carrier may—for some embodiments—be a cationic component.

Cationic component: The term “cationic component” typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4. Accordingly, a cationic component may be any positively charged compound or polymer, preferably a cationic peptide or protein, which is positively charged under physiological conditions, particularly under physiological conditions in vivo. A “cationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the conditions given.

5′-cap: A 5′-cap is an entity, typically a modified nucleotide entity, which generally “caps” the 5′-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyi nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. Preferred 5′-ends when using mCap or e.g., CleanCap GG, are “GGGAGA” or, e.g. when using CleanCap AG “AGGAGA”.

DNA: DNA is the usual abbreviation for deoxyribonucleic acid. It is a nucleic acid molecule, i.e. a polymer consisting of nucleotides. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerise by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing.

Fragment of a sequence: A fragment of a sequence may typically be a shorter portion of a full-length sequence of e.g. a nucleic acid molecule or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 20%, preferably at least 30%, more preferably at least 40%, more preferably at least 50%, even more preferably at least 60%, even more preferably at least 70%, and most preferably at least 80% of the total (i.e. full-length) molecule, from which the fragment is derived.

G/C modified: A G/C-modified nucleic acid may typically be a nucleic acid, preferably an artificial nucleic acid molecule as defined herein, based on a modified wild type sequence comprising a preferably increased number of guanosine and/or cytosine nucleotides as compared to the wild type sequence. Such an increased number may be generated by substitution of codons containing adenosine or thymidine nucleotides by codons containing guanosine or cytosine nucleotides. If the enriched G/C content occurs in a coding region of DNA or RNA, it makes use of the degeneracy of the genetic code. Accordingly, the codon substitutions preferably do not alter the encoded amino acid residues, but exclusively increase the G/C content of the nucleic acid molecule. As used herein, the term “G/C modification” comprises, in particular, the modifications of the number of guanosine and/or cytosine nucleotides in the mRNA according to the invention, such as GC optimization of sequences, adaptation of sequences to human codon usage, codon optimization, or C-optimization of sequences.

Gene therapy: Gene therapy may typically be understood to mean a treatment of a patient's body or isolated elements of a patient's body, for example isolated tissues/cells, by nucleic acids encoding a peptide or protein. It typically may comprise at least one of the steps of a) administration of a nucleic acid, preferably an RNA as defined herein, directly to the patient—by whatever administration route—or in vitro to isolated cells/tissues of the patient, which results in transfection of the patient's cells either in vivo/ex vivo or in vitro; b) transcription and/or translation of the introduced nucleic acid molecule; and optionally c) re-administration of isolated, transfected cells to the patient, if the nucleic acid has not been administered directly to the patient. The term “gene therapy” as used herein typically comprises treatment as well as prevention or prophylaxis of a disease, preferably a liver disease.

Heterologous sequence: Two sequences are typically understood to be “heterologous” if they are not derivable from the same gene. I.e., although heterologous sequences may be derivable from the same organism, they naturally (in nature) do not occur in the same nucleic acid molecule, such as in the same mRNA.

Cloning site: A cloning site is typically understood to be a segment of a nucleic acid molecule, which is suitable for insertion of a nucleic acid sequence, e.g., a nucleic acid sequence comprising an open reading frame. Insertion may be performed by any molecular biological method known to the one skilled in the art, e.g. by restriction and ligation. A cloning site typically comprises one or more restriction enzyme recognition sites (restriction sites). These one or more restrictions sites may be recognized by restriction enzymes which cleave the DNA at these sites. A cloning site which comprises more than one restriction site may also be termed a multiple cloning site (MCS) or a polylinker.

miRNA (microRNA) (miR) binding site(s): miRNAs (miR(s) or microRNA) are small single-stranded, 19-25 nucleotide long, non-coding RNA molecules found in plants, animals and some viruses, that functions in mRNA silencing and post-transcriptional regulation of gene expression. They function via Watson-Crick base-pairing with complementary sequences within the 5′ and/or 3′-untranslated regions (3′-UTR) of target mRNA molecules. As result, these mRNA molecules are silenced. Hereby, a “seed sequence” of 2-8 nucleotides, must be perfectly complementary. Functional seeds may be located in the 5′ and/or 3′-UTRs of mRNAs; multiple binding sites for the same miRNA in 5′ and/or 3′-UTRs can strongly enhance the degree of regulation. MiRNAs are derived from longer, primary transcripts termed “pri-miRNAs”. The pri-miRNAs, which can be more than 1000 nt in length, contain an RNA hairpin in which one of the two strands includes the mature miRNA (Lee et al 2002). When two mature miRNAs originate from opposite arms of the same pri-miRNA and are found in roughly similar amounts, they are denoted with a -3p or -5p suffix.

The term “miRNA (microRNA) (miR) binding site” as used herein, refers to a miRNA (miR) target site or a miRNA (miR) recognition site, or any nucleotide sequence to which a miRNA (miR) binds or associates. In some embodiments, a miRNA (miR) binding site represents a nucleotide location or region of a polynucleotide (e.g., an mRNA) to which at least the “seed” region of a miRNA (miR) binds. It should be understood that “binding” may follow traditional Watson-Crick hybridization rules or may reflect any stable association of the miRNA with the target sequence at or adjacent to the miRNA site. When referring to a miRNA (miR) binding site, a miRNA (miR) sequence is to be understood as having complementarity (e.g., partial, substantial, or complete (or full) complementarity) with the miRNA that binds to the miRNA binding site. A miRNA (miR) binding site can be partially complementary to a miRNA (miR), e.g., to an endogenous miRNA (miR), as is the case when the miRNA (miR) may exert translational control and/or transcript stability control of its corresponding mRNA. Alternatively, a miRNA (miR) binding site can be fully complementary (complete complementarity) to a miRNA (miR), e.g., to an endogenous miRNA (miR), as is the case when the miRNA (miR) may exert post-transcriptional and/or translational control of its corresponding mRNA. In preferred aspects of the disclosure, a miRNA (miR) binding site is fully complementary to a miRNA (miR), e.g., to an endogenous miRNA (miR), and may cause cleavage of the mRNA comprising said miRNA (miR) in cells and/or tissues in vivo, where the corresponding miR is expressed, e.g., endogenously expressed.

Nucleic acid molecule: A nucleic acid molecule is a molecule comprising, preferably consisting of nucleic acid components. The term “nucleic acid molecule” preferably refers to DNA, RNA or mRNA molecules. It is preferably used synonymous with the term “polynucleotide”. Preferably, a nucleic acid molecule is a polymer comprising or consisting of nucleotide monomers, which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA, RNA or mRNA molecules.

Open reading frame: An open reading frame (ORF) in the context of the invention may typically be a sequence of several nucleotide triplets, which may be translated into a peptide or protein. An open reading frame preferably contains a start codon, i.e. a combination of three subsequent nucleotides coding usually for the amino acid methionine (ATG), at its 5′-end and a subsequent region, which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present invention is preferably a nucleotide sequence, consisting of a number of nucleotides that may be divided by three, which starts with a start codon (e.g. ATG) and which preferably terminates with a stop codon (e.g., TAA, TGA, or TAG). The open reading frame may be isolated or it may be incorporated in a longer nucleic acid sequence, for example in a vector or an mRNA. An open reading frame may also be termed “(protein) coding region” or, preferably, “coding sequence” or CDS.

Peptide: A peptide or polypeptide is typically a polymer of amino acid monomers, linked by peptide bonds. It typically contains less than 50 monomer units. Nevertheless, the term “peptide” is not a disclaimer for molecules having more than 50 monomer units. Long peptides are also called polypeptides, typically having between 50 and 600 monomeric units.

Pharmaceutically effective amount: A pharmaceutically effective amount in the context of the invention is typically understood to be an amount that is sufficient to induce a pharmaceutical effect, such as an immune response, altering a pathological level of an expressed peptide or protein, or substituting a lacking gene product, e.g., in case of a pathological situation.

Protein: A protein typically comprises one or more peptides or polypeptides. A protein is typically folded into 3-dimensional form, which may be required for the protein to exert its biological function.

Polyadenylation: Polyadenylation is typically understood to be the addition of a poly(A) sequence to a nucleic acid molecule, such as an RNA or mRNA molecule, e.g. to a premature mRNA. Enzymatic polyadenylation may be induced by a so-called polyadenylation signal. This signal is preferably located within a stretch of nucleotides at the 3′-end of a nucleic acid molecule, such as an RNA or mRNA molecule, to be polyadenylated. A polyadenylation signal for enzymatic polyadenylation typically comprises a hexamer consisting of adenine and uracil/thymine nucleotides, preferably the hexamer sequence AAUAAA. Other sequences, preferably hexamer sequences, are also conceivable. Polyadenylation typically occurs during processing of a pre-mRNA (also called premature-mRNA). Typically, RNA maturation (from pre-mRNA to mature mRNA) comprises the step of polyadenylation.

Poly(A) sequence: A poly(A) sequence, also called poly(A)tail or 3′-poly(A)tail, is typically understood to be a sequence of adenosine nucleotides, e.g., of up to about 400 adenosine nucleotides, e.g. from about 20 to about 400, preferably from about 50 to about 400, more preferably from about 50 to about 300, even more preferably from about 50 to about 250, most preferably from about 60 to about 250 adenosine nucleotides. As used herein, a poly(A) sequence may also comprise about 10 to 200 adenosine nucleotides, preferably about 10 to 100 adenosine nucleotides, more preferably about 40 to 80 adenosine nucleotides or even more preferably about 50 to 70 adenosine nucleotides. A poly(A) sequence is typically located at the 3′-end of an mRNA. In the context of the present invention, a poly(A) sequence may be located within an mRNA or any other nucleic acid molecule, such as, e.g., in a vector, for example, in a vector serving as template for the generation of an RNA, preferably an mRNA, e.g., by transcription of the vector.

Restriction site: A restriction site, also termed restriction enzyme recognition site, is a nucleotide sequence recognized by a restriction enzyme. A restriction site is typically a short, preferably palindromic nucleotide sequence, e.g. a sequence comprising 4 to 8 nucleotides. A restriction site is preferably specifically recognized by a restriction enzyme. The restriction enzyme typically cleaves a nucleotide sequence comprising a restriction site at this site. In a double-stranded nucleotide sequence, such as a double-stranded DNA sequence, the restriction enzyme typically cuts both strands of the nucleotide sequence.

RNA, mRNA: RNA is the usual abbreviation for ribonucleic-acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the mRNA-sequence. Usually RNA may be obtainable by transcription of a DNA-sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino-acid sequence of a particular peptide or protein. Typically, a mature mRNA comprises a 5′-cap, a 5′-UTR, an open reading frame, a 3′-UTR and a poly(A) sequence. Aside from messenger RNA, several non-coding types of RNA exist which may be involved in regulation of transcription and/or translation. The term mRNA is used interchangeably with RNA herein.

Sequence of a nucleic acid molecule: The sequence of a nucleic acid molecule is typically understood to be the particular and individual order, i.e. the succession of its nucleotides. The sequence of a protein or peptide is typically understood to be the order, i.e. the succession of its amino acids.

Sequence identity: Two or more sequences are identical if they exhibit the same length and order of nucleotides or amino acids. The percentage of identity typically describes the extent to which two sequences are identical, i.e. it typically describes the percentage of nucleotides that correspond in their sequence position with identical nucleotides of a reference-sequence. For determination of the degree of identity, the sequences to be compared are considered to exhibit the same length, i.e. the length of the longest sequence of the sequences to be compared. This means that a first sequence consisting of 8 nucleotides is 80% identical to a second sequence consisting of 10 nucleotides comprising the first sequence. In other words, in the context of the present invention, identity of sequences preferably relates to the percentage of nucleotides of a sequence which have the same position in two or more sequences having the same length. Gaps are usually regarded as non-identical positions, irrespective of their actual position in an alignment.

Stabilized nucleic acid molecule: A stabilized nucleic acid molecule is a nucleic acid molecule, preferably a DNA, RNA or mRNA molecule that is modified such, that it is more stable to disintegration or degradation, e.g., by environmental factors or enzymatic digest, such as by an exo- or endonuclease degradation, than the nucleic acid molecule without the modification. Preferably, a stabilized nucleic acid molecule in the context of the present invention is stabilized in a cell, such as a prokaryotic or eukaryotic cell, preferably in a mammalian cell, such as a human cell. The stabilization effect may also be exerted outside of cells, e.g. in a buffer solution etc., for example, in a manufacturing process for a pharmaceutical composition comprising the stabilized nucleic acid molecule.

Transfection: The term “transfection” refers to the introduction of nucleic acid molecules, such as DNA or RNA (e.g. mRNA) molecules, into cells, preferably into eukaryotic cells. In the context of the present invention, the term “transfection” encompasses any method known to the skilled person for introducing nucleic acid molecules into cells, preferably into eukaryotic cells, such as into mammalian cells. Such methods encompass, for example, electroporation, lipofection, e.g. based on cationic lipids and/or liposomes or LNPs, calcium phosphate precipitation, nanoparticle based transfection, virus based transfection, or transfection based on cationic polymers, such as DEAE-dextran or polyethylenimine etc. Preferably, the introduction is non-viral.

Vector: The term “vector” refers to a nucleic acid molecule, preferably to an artificial nucleic acid molecule. A vector in the context of the present invention is suitable for incorporating or harboring a desired nucleic acid sequence, such as a nucleic acid sequence comprising an open reading frame. Such vectors may be storage vectors, expression vectors, cloning vectors, transfer vectors etc. A storage vector is a vector, which allows the convenient storage of a nucleic acid molecule, for example, of an mRNA molecule. Thus, the vector may comprise a sequence corresponding, e.g., to a desired mRNA sequence or a part thereof, such as a sequence corresponding to the coding sequence and the 3′-UTR of an mRNA. An expression vector may be used for production of expression products such as RNA, e.g. mRNA, or peptides, polypeptides or proteins. For example, an expression vector may comprise sequences needed for transcription of a sequence stretch of the vector, such as a promoter sequence, e.g. an RNA polymerase promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector, which is suitable for transferring nucleic acid molecules into cells or organisms, for example, viral vectors. A vector in the context of the present invention may be, e.g., an RNA vector or a DNA vector. Preferably, a vector is a DNA molecule. Preferably, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector.

Vehicle: A vehicle is typically understood to be a material that is suitable for storing, transporting, and/or administering a compound, such as a pharmaceutically active compound. For example, it may be a physiologically acceptable liquid, which is suitable for storing, transporting, and/or administering a pharmaceutically active compound.

5′-Terminal Oligopyrimidine Tract (TOP): The 5′-terminal oligopyrimidine tract (TOP) is typically a stretch of pyrimidine nucleotides located in the 5′-untranslated/terminal region of a nucleic acid molecule, such as the 5′-untranslated region of certain mRNA molecules or the 5′-untranslated region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′TOP (5′-terminal oligopyrimidine) ends one nucleotide 5′ to the first purine nucleotide located downstream of the TOP. Messenger RNA that contains a 5′-terminal oligopyrimidine tract is often referred to as TOP mRNA. Accordingly, genes that provide such messenger RNAs are referred to as TOP genes. TOP sequences have, for example, been found in genes and mRNAs encoding peptide elongation factors and ribosomal proteins.

TOP motif: In the context of the present invention, a TOP motif is a nucleic acid sequence which corresponds to a 5′TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′-end with a cytosine nucleotide. In TOP genes and TOP mRNAs, the TOP-motif preferably starts at its 5′-end with the transcriptional start site and ends one nucleotide 5′ to the first purin residue in said gene or mRNA. A TOP motif in the sense of the present invention is preferably located at the 5′-end of a sequence, which represents a 5′-UTR, or at the 5′-end of a sequence, which codes for a 5′-UTR. Thus, preferably, a stretch of 3 or more pyrimidine nucleotides is called “TOP motif” in the sense of the present invention if this stretch is located at the 5′-end of a respective sequence, such as the artificial nucleic acid molecule, the 5′-UTR element of the artificial nucleic acid molecule, or the nucleic acid sequence which is derived from the 5′-UTR of a TOP gene as described herein. In other words, a stretch of 3 or more pyrimidine nucleotides, which is not located at the 5′-end of a 5′-UTR or a 5′-UTR element but anywhere within a 5′-UTR or a 5′-UTR element, is preferably not referred to as “TOP motif”.

TOP gene: TOP genes are typically characterised by the presence of a 5′-terminal oligopyrimidine tract. Furthermore, most TOP genes are characterized by a growth-associated translational regulation. However, also TOP genes with a tissue specific translational regulation are known. As defined above, the 5′-UTR of a TOP gene corresponds to the sequence of a 5′-UTR of a mature mRNA derived from a TOP gene, which preferably extends from the nucleotide located 3′ to the 5′-cap to the nucleotide located 5′ to the start codon. A 5′-UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′-UTRs of TOP genes are generally rather short. The lengths of 5′-UTRs of TOP genes may vary between 20 nucleotides up to 500 nucleotides, and are typically less than about 200 nucleotides, preferably less than about 150 nucleotides, more preferably less than about 100 nucleotides. Exemplary 5′-UTRs of TOP genes in the sense of the present invention are the nucleic acid sequences extending from the nucleotide at position 5 to the nucleotide located immediately 5′ to the start codon (e.g. the ATG) in the sequences according to SEQ ID NO:1-1363 of patent application WO2013143700, whose disclosure is incorporated herewith by reference. In this context, a particularly preferred fragment of a 5′-UTR of a TOP gene is a 5′-UTR of a TOP gene lacking the 5′TOP motif. The terms “5′-UTR of a TOP gene” or “5′TOP-UTR” preferably refer to the 5′-UTR of a naturally occurring TOP gene.

5′-untranslated region (5′-UTR): A 5′-UTR is typically understood to be a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites. The 5′-UTR may be post-transcriptionally modified, for example by addition of a 5′-cap. In the context of the present invention, a 5′-UTR corresponds to the sequence of a mature mRNA, which is located between the 5′-cap and the start codon. Preferably, the 5′-UTR corresponds to the sequence, which extends from a nucleotide located 3′ to the 5′-cap, preferably from the nucleotide located immediately 3′ to the 5′-cap, to a nucleotide located 5′ to the start codon of the protein coding region, preferably to the nucleotide located immediately 5′ to the start codon of the protein coding region. The nucleotide located immediately 3′ to the 5′-cap of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence, which corresponds to such RNA sequence. In the context of the present invention, the term “a 5′-UTR of a gene” is the sequence, which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the mRNA sequence of the 5′-UTR. By the inventive embodiments such a 5′-UTR may be provided 5′-terminal to the coding sequence. Its length is typically less than 500, 400, 300, 250 or less than 200 nucleotides. In other embodiments its length may be in the range of at least 10, 20, 30 or 40, preferably up to 100 or 150, nucleotides.

3′-untranslated region (3′-UTR): Generally, the term “3′-UTR” refers to a part of the artificial nucleic acid molecule, which is located 3′ (i.e. “downstream”) of an open reading frame and which is not translated into protein. Typically, a 3′-UTR is the part of an mRNA which is located between the protein coding region (open reading frame (ORF) or coding sequence (CDS)) and the poly(A) sequence of the mRNA. In the context of the invention, the term “3′-UTR” may also comprise elements, which are not encoded in the template, from which an RNA is transcribed, but which are added after transcription during maturation, e.g. a poly(A) sequence. A 3′-UTR of the mRNA is not translated into an amino acid sequence. The 3′-UTR sequence is generally encoded by the gene, which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′-capping, splicing the pre-mature mRNA to excise optional introns and modifications of the 3′-end, such as polyadenylation of the 3′-end of the pre-mature mRNA and optional endo-/or exonuclease cleavages etc. In the context of the present invention, a 3′-UTR corresponds to the sequence of a mature mRNA, which is located between the stop codon of the protein coding region, preferably immediately 3′ to the stop codon of the protein coding region, and the poly(A) sequence of the mRNA. The term “corresponds to” means that the 3′-UTR sequence may be an RNA sequence, such as in the mRNA sequence used for defining the 3′-UTR sequence, or a DNA sequence, which corresponds to such RNA sequence. In the context of the present invention, the term “a 3′-UTR of a gene”, such as “a 3′-UTR of a ribosomal protein gene”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the mRNA sequence (both sense and antisense strand and both mature and immature) of the 3′-UTR.

“Isolated”: As used herein, the term “isolated”, in regard to a nucleic acid molecule, preferably an isolated mRNA, or a polypeptide, means that the nucleic acid molecule, preferably isolated mRNA, or polypeptide is in a condition other than its native environment, such as apart from blood and/or animal tissue. In some embodiments, an isolated nucleic acid molecule, preferably isolated mRNA, or polypeptide is substantially free of other nucleic acid molecules or other polypeptides, particularly other nucleic acid molecules or polypeptides of animal origin. In some embodiments, the nucleic acid molecule, preferably isolated mRNA, or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms. Isolated substances may also have varying levels of purity in reference to the substances from which they have been associated. Isolated substances and/or entities may also be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In the context of the present invention, description and claims, the term “mRNA” preferably means an “isolated mRNA” and vice versa.

### DETAILED DESCRIPTION OF THE INVENTION

The present invention relates to novel mRNAs and to compositions and kits comprising the mRNA. Furthermore, several uses, in particular medical uses, of the mRNA according to the invention and of the pharmaceutical compositions and kits are provided. Said novel mRNAs encode novel engineered hepatocyte nuclear factor 4 alpha (HNF4A) protein variants, which are superior in terms of expression, stability, activity and/or therapeutic effect and which are in sum beneficial when administered to a patient in need.

In a first aspect, the present invention relates to an isolated mRNA encoding an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer.

In another embodiment of the first aspect, an isolated mRNA encoding an engineered HNF4A comprising a S87A mutation and/or a S461E mutation, more preferably an engineered HNF4A comprising a S461E mutation is used for treating, preventing, attenuating or inhibiting liver fibrosis or liver cirrhosis.

As liver cirrhosis is a late stage of scarring (fibrosis) of the liver caused and often liver cirrhosis has no signs or symptoms until liver damage is extensive. Accordingly, in yet another embodiment of the first aspect, an isolated mRNA encoding an engineered HNF4A comprising a S87A mutation and/or a S461E mutation, more preferably an engineered HNF4A comprising a S461E mutation is used for treating, preventing, attenuating or inhibiting liver cirrhosis.

In a second aspect, the present invention relates to an isolated mRNA encoding wild type hepatocyte nuclear factor 4 alpha (HNF4A) for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer.

In another embodiment of the second aspect, an isolated mRNA encoding wild type hepatocyte nuclear factor 4 alpha (HNF4A) is used for treating, preventing, attenuating or inhibiting liver fibrosis or liver cirrhosis.

In yet another embodiment of the second aspect, an isolated mRNA encoding wild type hepatocyte nuclear factor 4 alpha (HNF4A) is used for treating, preventing, attenuating or inhibiting liver cirrhosis.

In a third aspect, the present invention relates to an isolated mRNA encoding wild type hepatocyte nuclear factor 4 alpha (HNF4A) or preferably an isolated mRNA encoding an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation for use in treating or for use in supportive treatment of a disease which results in or leads to liver fibrosis or liver cirrhosis, said disease being selected from the group consisting of acute hepatic porphyria; Alagille syndrome; acute alcoholic hepatitis/alcoholic hepatitis; alcoholic steatohepatitis (ASH); alcoholic (fatty) liver disease (ALD); alcohol-related liver disease (ARLD); alpha-1 antitrypsin deficiency; autoimmune hepatitis (AIH); bile duct cancer (cholangiocarcinoma); biliary atresia; Brucellosis or syphilis; Budd-Chiari syndrome (BCS); chronic heart failure (HF); cystic fibrosis; galactosemia; glycogen storage disease Type 1/glycogen storage diseases; haemochromatosis; hepatitis A (HAV)/hepatitis A infection; hepatitis B (HBV)/chronic hepatitis B infection (CHB); hepatitis C (HCV)/chronic hepatitis C infection (CHC); hepatitis D (HDV)/chronic hepatitis D infection (CHD); hepatocellular carcinoma (HCC); benign liver tumors; lysosomal acid lipase deficiency (LAL-D); non-alcoholic fatty liver disease (NAFLD); non-alcoholic fatty liver (NAFL); non-alcoholic steatohepatitis (NASH); primary biliary cholangitis (PBC); primary biliary cirrhosis; primary sclerosing cholangitis (PSC); progressive familial intrahepatic cholestasis (PFIC); and Wilson disease.

In other words, the inventive mRNAs according to the invention encoding the inventive engineered hepatocyte nuclear factor 4 alpha (HNF4A) protein variants can be used for example for

As used herein, the term “hepatocyte nuclear factor-4-alpha” or “HNF4A” relates to an HNF4A mRNA or HNF4A protein having the biological activity of a HNF4A protein (OMIM entry No: 600281; HGNC: 5024, HGNC Approved Gene Symbol: HNF4A, alternative names HNF4-ALPHA, HEPATOCYTE NUCLEAR FACTOR 4, HNF4, TRANSCRIPTION FACTOR 14, HEPATIC NUCLEAR FACTOR or TCF14). Generally, a transcription factor (TF) or 2sequence-specific DNA-binding factor” is a protein controlling the rate of transcription of genetic information from DNA to messenger RNA (mRNA), by binding to a specific DNA sequence to regulate (turn on and off) genes.

As used herein, the term “HNF4A WT mRNA” or “WT HNF4A mRNA” is intended to describe mRNA encoding the unmodified wild type (human) HNF4A protein, in contrast to mRNA encoding engineered HNF4A protein variants which do not occur in nature.

The term “increased expression”, “enhanced expression” or “overexpression” as used herein means any form of expression which occurs in addition to the original wild type mRNA expression level.

As used herein, the terms “transcriptional activity or respectively DNA binding capacity”, “increased transcriptional activity”, “increased DNA binding capacity”, “greater transcriptional activity”, “greater DNA binding capacity”, “enhanced transcriptional activity” or respectively “enhanced DNA binding capacity” all refer to an improved property of the engineered HNF4A protein variants disclosed herein (either on protein level, or mRNA encoded), which can be represented by HNF4A transcriptional activity, HNF4A activity to drive gene expression, HNF4A potency, and/or HNF4A ability to recruit polymerases or additional factors of the transcriptional machinery. In the present specific case related to the transcription factor HNF4A, the terms preferably are intended to describe the “transcriptional activity” or respectively “DNA binding capacity” of the transcription factor HNF4A. The term “engineered” in the context of the present invention relates to not naturally occurring HNF4A protein variants occurring in nature and comprising one or more amino acid substitution, deletion, and/or insertion mutation, but thus being modified by the inventors of the present invention, to exhibit an increased “transcriptional activity” as compared to the human WT HNF4A transcription factor protein. Exemplary methods to determine transcriptional activity or respectively DNA binding capacity of the engineered HNF4A of the present invention are provided in the Examples section. Any property relating to transcriptional activity or respectively DNA binding capacity may be affected. For example, improvements in transcriptional activity or respectively DNA binding capacity can be from about 1.1 fold the transcriptional activity or respectively DNA binding capacity of the corresponding wild type HNF4A, to as much as 2-fold, 5-fold, 10-fold, 20-fold, 25-fold, 50-fold, 75-fold, 100-fold, 150-fold, 200-fold or more transcriptional activity or respectively DNA binding capacity than the naturally occurring HNF4A or another engineered HNF4A from which the HNF4A polypeptides or proteins were derived. An inventive advantage of an “increased transcriptional activity”, “increased DNA binding capacity”, “greater transcriptional activity”, “greater DNA binding capacity”, “enhanced transcriptional activity” or respectively “enhanced DNA binding capacity” of the encoded engineered HNF4A protein(s) is visible through the potential of being able to lower the mRNA dose need for treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer. E.g. in the inventive examples, it was possible to have a positive effect for treating, preventing, attenuating or inhibiting a liver cirrhosis with a low dose of 0.3 mg/kg mRNA encoding an engineered HNF4A protein.

In another preferred embodiment, the inventive mRNA encoding a WT or an engineered HNF4A protein can be administered at a dose of about 0.5 mg/kg. In another embodiment, the inventive mRNA encoding a WT or an engineered HNF4A protein can be administered at a dose of about 0.4 mg/kg. In another embodiment, the inventive mRNA encoding a WT or an engineered HNF4A protein can be administered at a dose of about 0.3 mg/kg. In another embodiment, the inventive mRNA encoding a WT or an engineered HNF4A protein can be administered at a dose of about 0.2 mg/kg. In another embodiment, the inventive mRNA encoding a WT or an engineered HNF4A protein can be administered at a dose of about 0.1 mg/kg or lower than about 0.1 mg/kg.

The terms “increased stability”, “greater stability”, “enhanced stability” or “longer-lasting HNF4A half-life” as used herein are used interchangeably in this application, and mean that the engineered HNF4A protein variants shows less degradation, than a WT HNF4A protein, when exposed to similar or identical conditions (either on protein level or mRNA encoded). Degradation can be determined, for example, by the difference in loss of biological activity (i.e. difference in rate and/or extent of biological activity), after a certain time period under similar or identical conditions (e.g. 1 day, 2 days, 3 days, 4 days, 5 days, 6 days, one week, two weeks, three weeks, or 1 month after administration of the mRNA of the invention. An inventive advantage of an “increased stability”, “greater stability”, “enhanced stability” or “longer-lasting HNF4A half-life” for mRNA encoding an engineered HNF4A protein or respectively the engineered HNF4A protein itself is visible through the potential of being able to lower the mRNA dose need for treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer. E.g. in the inventive examples, it was possible to have a positive effect for treating, preventing, attenuating or inhibiting a liver cirrhosis with a low dose of 0.3 mg/kg mRNA encoding an engineered HNF4A protein.

In the context of the present invention, the terms “WT HNF4A” or “human HNF4A” refer to the human unmodified HNF4A protein (or respectively an mRNA encoding said WT HNF4A).

The term “preventing” refers to decreasing the probability that an organism contracts or develops an abnormal condition, like e.g. a liver disease.

The terms “treating”, “reversing”, “attenuating” or “curing” refer to having a therapeutic effect and at least partially alleviating or abrogating an abnormal condition in the organism, like e.g. a liver disease. Reversing, treating, attenuating or curing include inhibition of the liver disease, maintenance of the liver disease, reducing, curing and induction of remission of the liver disease. The terms “inhibits a liver disease” or “inhibiting a liver disease”, “inhibits liver cirrhosis or liver fibrosis”, “curing liver cirrhosis or liver fibrosis”, “reversing liver cirrhosis or liver fibrosis”, “attenuating liver cirrhosis or liver fibrosis” or “inhibiting liver cirrhosis or liver fibrosis” are to be seen in a similar way as “treating” said disease(s); in any case, a patient suffering under a “liver disease” will experience an improvement of said liver disease, upon treatment with the mRNA sequences of the invention. In each of the aforementioned cases, the diseases “liver cirrhosis or liver fibrosis” can be exchanged with any of the herein mentioned liver diseases. Furthermore, in any of the cases in this paragraph, the term “reversing liver fibrosis” or “reversing liver cirrhosis” can be used equivalently. The term “attenuation” (attenuate: weaken, mitigate) of a disease means in principle the reduction, mitigation or lessening of the negative/disadvantageous symptoms, impacts or effects of the liver disease on the patient or subject in need.

The term “therapeutic effect” or “therapeutic activity” refers to the inhibition of an abnormal condition, like e.g. a liver disease upon administration of an mRNA of the present invention, encoding HNF4A or an engineered HNF4A. A therapeutic effect relieves to some extent one or more of the symptoms of the abnormal condition, like e.g. a liver disease. In reference to the treatment of abnormal conditions, a therapeutic effect can refer to one or more of the following: (a) a decrease in the proliferation, growth, and/or progression of a liver disease; (b) inhibition (i.e., slowing or stopping) of a liver disease in vivo; and (c) relieving to some extent one or more of the symptoms associated with the abnormal condition e.g. the liver disease. The administration of the mRNA of the invention encoding either wild type HNF4A protein or preferably engineered HNF4A protein variants as described herein effectuate the therapeutic effect. The term “therapeutic effect” as used herein, also refers to the effective provision of protection effects to prevent, inhibit, or arrest the symptoms and/or progression of a liver disease as described herein.

The term “a therapeutically effective amount” as used herein means a sufficient amount of the HNF4A protein of the invention to produce a therapeutic effect, as defined above, in a subject or patient in need of treatment.

The terms “subject” or “patient” are used herein mean any mammal, including but not limited to human beings, including a human patient or subject to which the pharmaceutical compositions of the invention can be administered. The term mammals include human patients and non-human primates, as well as experimental animals such as rabbits, rats, and mice, and other animals.

Thus, in some embodiments, the disclosure provides mRNAs encoding wild type HNF4A protein or engineered HNF4A protein variants, which have an amino acid sequence that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence corresponding to any one of SEQ ID NO:100-148, 149-197, or 198-246, and further comprises any one or more of the amino acid differences, or combinations of differences (e.g. amino acid differences selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A, preferably S87A or S461E, or a combination thereof), found in any of the exemplary engineered HNF4A protein variant amino acid sequences.

In preferred embodiments, the engineered HNF4A protein variants of the invention comprises a S461A mutation. In other preferred embodiments, the engineered HNF4A protein variants of the invention comprises a S461E mutation. This S461E mutation preferably leads to a HNF4A phospho-mimetic phenotype, by mutating a serine residue to a charged amino acid such as glutamate or asparate. In other preferred embodiments, the engineered HNF4A protein variants of the invention comprises a S87A mutation i.e. preferably leading to a phospho-incompetent HNF4A phenotype via mutation of a serine residue. In further preferred embodiments, the engineered HNF4A protein variants of the invention comprises a S87A and/or a S461E mutation. In even further preferred embodiments, the engineered HNF4A protein variants of the invention comprises a S461E mutation.

In other preferred embodiments, the engineered HNF4A protein variants of the invention are phospho-incompetent HNF4A mutants or phosphomimetic HNF4A mutants.

In some embodiments, these recombinant HNF4A polypeptides can have one or more amino acid differences at other residue positions, substitutions, insertions, deletions at other positions, and/or additional amino or carboxy terminal extensions.

In some embodiments, the HNF4A mRNA or HNF4A polypeptides of the present disclosure exhibit improved properties of increased HNF4A expression, HNF4A transcriptional activity, DNA binding capacity, HNF4A stability, longer-lasting HNF4A half-life and/or increased therapeutic effect as compared to the wild type HNF4A of SEQ ID NO:100.

In other preferred embodiments, the HNF4A mRNA encoding engineered HNF4A polypeptides of the present disclosure exhibit improved HNF4A transcriptional activity as compared to the wild type HNF4A of SEQ ID NO:100.

In other preferred embodiments, the HNF4A mRNA encoding engineered HNF4A polypeptides of the present disclosure exhibit improved HNF4A DNA binding capacity as compared to the wild type HNF4A of SEQ ID NO:100.

In other preferred embodiments, the HNF4A mRNA encoding engineered HNF4A polypeptides of the present disclosure exhibit improved properties of increased HNF4A expression as compared to the wild type HNF4A of SEQ ID NO:100.

In other preferred embodiments, the HNF4A mRNA encoding engineered HNF4A polypeptides of the present disclosure exhibit improved HNF4A stability as compared to the wild type HNF4A of SEQ ID NO:100.

In other preferred embodiments, the HNF4A mRNA encoding engineered HNF4A polypeptides of the present disclosure exhibit improved HNF4A half-life as compared to the wild type HNF4A of SEQ ID NO:100.

In other preferred embodiments, the HNF4A mRNA encoding engineered HNF4A polypeptides of the present disclosure exhibit improved therapeutic effect as compared to the wild type HNF4A of SEQ ID NO:100.

In some embodiments, the recombinant HNF4A polypeptides exhibit at least 1.2-fold, at least 1.3-fold, at least 1.5-fold, at least 2-fold, at least 3-fold, at least 4-fold, at least 5-fold, at least 10-fold, at least 15-fold, at least 20-fold, or at least 25-fold, or more, increased stability, transcriptional activity and/or respectively DNA binding capacity as compared to a reference HNF4A (e.g., wild type of SEQ ID NO:100) under suitable conditions.

The mRNA according to the invention is preferably suitable for use in the treatment or prophylaxis of a liver disease or a liver disorder as described herein, preferably selected from liver fibrosis and liver cirrhosis. More preferably, the mRNA according to the present invention is preferably suitable for use in safe and effective treatment or prophylaxis of a liver disease as described herein in mammals, preferably in human.

As used herein, the term “liver disease” or “liver disorder” typically relates to any condition, which is associated with, or which leads to structural alteration or damage of liver tissue, in particular which leads to fibrosis or cirrhosis of the liver. In particular, a “liver disease’ as used herein, such as liver fibrosis or liver cirrhosis, is a chronic disease characterized by an excess production of hepatic connective tissue, in particular by the accumulation of extracellular matrix (ECM) proteins, which is preferably caused by activated hepatic stellate cells (HSC). The process of excess production of hepatic connective tissue and/or the accumulation of extracellular matrix (ECM) proteins may also be referred to as “fibrosis”. The liver diseases addressed herein may also be termed “fibrotic liver diseases”.

In the context of the present invention, the term “liver disease” preferably also refers to a disease or disorder, which is capable of causing fibrosis of the liver tissue and which potentially leads to liver fibrosis, such as infectious diseases (e.g. Hepatitis B, Hepatitis C or Hepatitis D), autoimmune diseases (e.g. primary biliary cirrhosis or autoimmune hepatitis), genetic/inherited diseases (hereditary haematochromatosis, Wilson's disease, cystic fibrosis, diabetes), metabolic and/or diet-related diseases (obesity, diabetes, alcohol abuse, alcoholic liver disease, non-alcoholic fatty liver disease (NAFLD) and/or non-alcoholic steatohepatitis (NASH)), cancer or tumor diseases (e.g. hepatocellular carcinoma (HCC)) or other diseases such as gallstones, Budd-Chiari syndrome or primary sclerosing cholangitis. Preferably, the term “liver disease” is to be understood as being selected from the group consisting of liver fibrosis, liver cirrhosis, hepatocellular carcinoma (HCC), non-alcoholic fatty liver disease (NAFLD), non-alcoholic steatohepatitis (NASH) and liver cancer, or combinations thereof. It is to be understood, that fibrogenesis falls within the term “liver fibrosis”, and thus a reduced fibrogenesis is a preferred outcome of the treatment with the mRNA medicines of the present invention. Further, severe forms of cholestasis are frequent causes of liver cirrhosis and thus to be understood as falling under the term “liver disease”.

In the following, the mRNA according to the invention is described. The disclosure concerning the inventive mRNA as such also applies to the mRNA for (medical) use as described herein as well as to the mRNA in the context of the pharmaceutical composition or the mRNA in the context of the kit (of parts) comprising the mRNA according to the invention. In particular, when referring to an “RNA according to the invention” or “mRNA according to the invention”, the present disclosure also relates to an “RNA for use according to the invention” or “mRNA for use according to the invention” and vice versa.

HNF4A mRNA Sequences

In preferred embodiments, the present invention relates to an mRNA for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-148, 149-197, and 198-246, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140 or a fragment or variant of said sequences having the biological activity of a HNF4A protein.

In even further preferred embodiments, the present invention relates to an mRNA for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-148, 149-197, and 198-246, preferably an engineered HNF4A comprising (i) a S461E mutation, (ii) a S87A mutation, or (iii) a S87A and a S461E mutation or a fragment or variant of said sequences having the biological activity of a HNF4A protein.

In preferred embodiments, the present invention relates to an mRNA for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140,
more preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, most preferably to SEQ ID NO:140;
or a fragment or variant of said sequences having the biological activity of a HNF4A protein.

In even further preferred embodiments, the present invention relates to an mRNA for use in treating, reversing, preventing, attenuating or inhibiting a liver disease, preferably selected from the group consisting of liver fibrosis or liver cirrhosis, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising (i) a S461E mutation, (ii) a S87A mutation, or (iii) a S87A and a S461E mutation,
more preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, most preferably to SEQ ID NO:140;
or a fragment or variant of said sequences having the biological activity of a HNF4A protein.

In most preferred embodiments, the present invention relates to an mRNA for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising S87A K106R K108R K126R K127R S142A S143A S148A T166A S167A S313A S378A T429A T432A S436A K458R S461E mutations, preferably SEQ ID NO:140 or a fragment or variant of said sequences having the biological activity of a HNF4A protein.

In further most preferred embodiments, the present invention relates to an mRNA for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising S87A K106R K108R K126R K127R S142A S143A S148A T166A S167A S313A S378A T429A T432A S436A K458R S461E mutations, preferably SEQ ID NO:140 or a fragment or variant of said sequences having the biological activity of a HNF4A protein.

In even further most preferred embodiments, the present invention relates to an mRNA for use in treating, reversing, preventing, attenuating or inhibiting liver fibrosis or liver cirrhosis, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising a S461E mutation, preferably SEQ ID NO:129 or a fragment or variant of said sequences having the biological activity of a HNF4A protein.

In other preferred embodiments, the present invention relates to an mRNA comprising an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100 for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said mRNA preferably has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:250-297, 692-740, 1135-1183, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140 or a fragment or variant of said sequences, wherein the encoded protein has the biological activity of a HNF4A protein.

In further preferred embodiments, the present invention relates to an mRNA comprising an ORF encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:250-297, 692-740, 1135-1183 for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, wherein said engineered HNF4A comprises at least one substitution or substitution set at one or more positions selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, 5183E, S461A, S461E, S167A, S167E, S378A, T429A, T432A, S436A, S378E, T429E, T432E, S436E, S87A, S87E, S95A, S99A, S138A, and T139A and/or combinations thereof, and wherein the amino acid positions of said amino acid sequence are numbered with reference to the human wild-type HNF4A protein (SEQ ID NO:100), preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

As used throughout the context of the whole specification, the term “mutation” in for example “S461E mutation” is to be understood as “substitution”, i.e. is to be understood as an, for example, “S461E substitution”, i.e. a substitution of S461 to S461E.

A “substitution set” in the context of the present invention is considered to be at least one substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

Further preferably, the substitution set comprises at least a S142A and/or S143A substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In another preferred embodiment, the substitution set comprises at least a S87A substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87E, S95A, S99A, S138A, and T139A.

In yet another preferred embodiment, the substitution set comprises at least a S461A substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In yet another preferred embodiment, the substitution set comprises at least a S461E substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In yet another preferred embodiment, the substitution set comprises at least a S87A K106R K108R K126R K127R substitution and optionally at least one additional substitution selected from the group consisting of R2V, KSV, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In yet another preferred embodiment, the substitution set comprises at least a K106 K108 K126 K127 substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In yet another preferred embodiment, the substitution set comprises at least a S378A T429A T432A S436A substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In yet another preferred embodiment, the substitution set comprises at least a S87A K106R K108R K126R K127R S142A S143A S148A T166A S167A S313A S378A T429A T432A S436A K458R substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In yet another preferred embodiment, the substitution set comprises at least a S87A K106R K108R K126R K127R S142A S143A S148A T166A S167A S313A S378A T429A T432A S436A K458R S461E substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A.

In a further preferred embodiment, the substitution set comprises at least S87A K106R K108R K126R K127R S142A S143A S148A T166A S167A S313A S378A T429A T432A S436A K458R S461E and optionally at least one additional substitution, e.g. based on an exemplary conservative amino acid substitution as shown in Table (i).

More preferably, the substitution set comprises at least a S461E substitution and optionally at least one additional substitution selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S167A, S167E, S378A, T429A, T432A, S436A, S378E, T429E, T432E, S436E, S87A, S87E, S95A, S99A, S138A, and T139A.

In particularly preferred embodiments, a HNF4A substitution set of the invention for engineering HNF4A is selected from the group consisting of
(i) “S142A S143A” (i.e. present in combo 1 to 8 and 10 to 19);
(ii) “5×SP/TP to AP” (S167A S378A T429A T432A S436A, i.e. present in combo 3 to 8, 10 to 11 and 13 to 19; (iii) “5×SP/TP to EP” (S167E S378E T429E T432E S436E; i.e. present in combo 12);
(iv) “4×K to R” (K106R K108R K126R K127R; present in combo 5, 8 and 9 to 19);
(v) “S142E S143E”; and
(vi) any of the substitution sets which are shown in FIG. 1 or FIG. 2 and not mentioned herein in this list (e.g. K179R K180R or K447R K470R).

In further preferred embodiments, the present invention relates to an mRNA encoding an unmodified human wild type hepatocyte nuclear factor 4 alpha (HNF4A) according to SEQ ID NO:100, preferably wherein said mRNA has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:249, 298, 347, 396, 445, 494, 543, 592, 641, 1576, 1577, 1578, 1627, 1676, 1725, 1774, 1823, 1872, 1921, 1970, 2905, 2906, 2907, 2956, 3005, 3054, 3103, 3152, 3201, 3250, 3299, 4234, 4235, 4236, 4285, 4334, 4383, 4432, 4481, 4530, 4579, 4628, 5719, 5722, 5725, 5728, 5731, or 5734 or a fragment or variant of said sequences, wherein the encoded protein has the biological activity of a HNF4A protein, further, wherein the mRNA further comprises an UTR combination selected from the group consisting of (i) a 5′-UTR derived from a mouse solute carrier family 7 (cationic amino acid transporter, y+ system) (SLC7A3) and a 3′-UTR derived from PSMB3; (ii) a 5′-UTR derived from mouse ribosomal protein L31 (RPL31) and a 3′-UTR derived from a human ribosomal protein S9 (RPS9); (iii) a 5′-UTR derived from ubiquilin 2 (Ubqln2) and a 3′-UTR derived from Guanine nucleotide-binding protein G(s) subunit alpha isoforms short (Gnas); and (iv) a 5′-UTR derived from a hydroxysteroid (17-beta) dehydrogenase 4 gene (HSD17B4) and a 3′-UTR derived from a proteasome subunit beta type-3 (PSMB3) UTR.

In other embodiments, the
(i) G/C content of the HNF4A coding sequence is increased compared to the coding sequence of the corresponding wild type HNF4A coding sequence of SEQ ID NO:247 or 248;
(ii) C content of the HNF4A coding sequence is increased compared to the coding sequence of the corresponding wild type HNF4A coding sequence of SEQ ID NO:247 or 248; and/or
(iii) at least one codon of the HNF4A coding sequence is adapted to human codon usage, wherein the codon adaptation index (CAI) is preferably increased or maximised in the corresponding HNF4A coding sequence compared to the coding sequence of the corresponding wild type HNF4A coding sequence of SEQ ID NO:247 or 248.

In even further embodiments, the mRNA of the invention comprises a 5′-cap structure, a poly(A) sequence comprising at least 70 A nucleotides, preferably about 100 A nucleotides, a poly(C) sequence, preferably comprising 10 to 200, 10 to 100, 20 to 70, 20 to 60 or 10 to 40 cytosine nucleotides, and/or at least one histone stem-loop, preferably, wherein the mRNA comprises a 3′-terminal A nucleotide.

In other embodiments, the mRNA of the invention comprises, preferably in 5′ to 3′ direction, the following elements:
a) a 5′-cap1 structure;
b) a 5′-UTR element comprising a nucleic acid sequence, preferably derived from a 5′-UTR of a HSD17B4 gene, comprising the nucleic acid sequence according to SEQ ID NO:1 or 2, or a homolog, a fragment or a variant thereof;
c) at least one coding sequence as defined herein above or below;
d) a 3′-UTR element comprising a nucleic acid sequence, preferably derived from a 3′-UTR of a PSMB3 gene, comprising the nucleic acid sequence according to SEQ ID NO:33 or 34, or a homolog, a fragment or a variant thereof;
e) a poly(A) sequence comprising about 100 adenosine nucleotides, preferably, wherein the mRNA comprises a 3′-terminal A nucleotide;
f) an optional poly(C) tail, preferably comprising 10 to 40 cytosine nucleotides; and/or
g) an optional histone stem-loop, preferably comprising the nucleic acid sequence according to SEQ ID NO:63 or 64.

In more preferred embodiments, the nucleic acid of the invention comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to the sequences according to any one of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list] or a fragment or variant of any of these sequences, encoding wild type or engineered HNF4A protein variants as provided in Table A and Table B (reference to Table B is intended to be understood herein as being to Table B-I and/or Table B-II and/or Table B-III), and under <223> identifier of the ST25 sequence listing of respective sequence SEQ ID NOs [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]. A further preferred nucleic acid sequence is any sequence selected from the group consisting of SEQ ID NO:249-5562, and SEQ ID NO:5719-5736.

Preferred nucleic acid sequences, preferably mRNA sequences of the invention are provided in Table A. Therein, each row represents a specific suitable HNF4A construct of the invention (for column designation: see above), wherein the description of the HNF4A construct is indicated in column A of Table A and the SEQ ID NOs of the amino acid sequence of the respective HNF4A construct is provided in column B. The corresponding SEQ ID NOs of the coding sequences encoding the respective HNF4A constructs are provided in Table A. Further detailed information on the specific sequences is provided under <223> identifier of the respective SEQ ID NOs in the sequence listing [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)].

Table A: preferred mRNA sequences and constructs of the invention. The first column (“SEQ ID NO: Protein”) describes each HNF protein of the invention. The second column (“SEQ ID NO: CDS wt”) describes different SET CDS encoding HNF4A. The following columns (“CDS opt1” to “CDS opt29”) describe different inventive mRNA constructs (i.e. “i-3”, “a-1”) having different CDS optimization (i.e. “CDS opt1” to “CDS opt29”), each encoding HNF4A WT or engineered HNF4A variants. More information on the sequences is disclosed in the ST.25 sequence listing under <223> Other Information [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]. Each sequence or construct as shown in Table A or the sequence listing resembles a preferred construct of the invention.

TABLE A
preferred mRNA sequences and constructs of the invention

| | SEQ ID | SEQ ID | | | | | | | | |
|---|---|---|---|---|---|---|---|---|---|---|
| | NO: | NO: | CDS | CDS | CDS | CDS | CDS | CDS | CDS | CDS |
| | Protein | CDS wt | opt1 | opt2 | opt3 | opt4 | opt5 | opt11 | opt28 | opt29 |
| Sum of | 100- | 247, | 249- | 347- | 396- | 445- | 494- | 543- | 592- | 641- |
| All | 246 | 248, | 346, | 395, | 444, | 493, | 542, | 591, | 640, | 689, |
| HNF4A | | 690, | 692- | 790- | 839- | 888- | 937- | 986- | 1035- | 1084- |
| | | 691, | 789, | 838, | 887, | 936, | 985, | 1034, | 1083, | 1132, |
| | | 1133, | 1135- | 1233- | 1282- | 1331- | 1380- | 1429- | 1478- | 1527- |
| | | 1134 | 1232 | 1281 | 1330 | 1379 | 1428 | 1477 | 1526 | 1575 |
| Sum of | 100- | 247, | 249- | 347- | 396- | 445- | 494- | 543- | 592- | 641- |
| HNF4A | 148 | 248 | 346 | 395 | 444 | 493 | 542 | 591 | 640 | 689 |
| 1-474 | | | | | | | | | | |
| Sum of | 149- | 690, | 692- | 790- | 839- | 888- | 937- | 986- | 1035- | 1084 |
| HNF4A | 197 | 691 | 789 | 838 | 887 | 936 | 985 | 1034 | 1083 | 1132 |
| 1-375 | | | | | | | | | | |
| Sum of | 198- | 1133, | 1135- | 1233- | 1282- | 1331- | 1380- | 1429- | 1478- | 1527- |
| HNF4A | 246 | 1134 | 1232 | 1281 | 1330 | 1379 | 1428 | 1477 | 1526 | 1575 |
| 39-474 | | | | | | | | | | |
| HNF1A | 5672 | 5673, | 5675 | 5676 | 5677 | 5678 | 5679 | 5680 | 5681 | 5682 |
| 1-631 | | 5674 | | | | | | | | |
| Sum of | 100- | 247, | 249- | 347- | 396- | 445- | 494- | 543- | 592- | 641- |
| All | 246, | 248, | 346, | 395, | 444, | 493 | 542, | 591, | 640, | 689, |
| HNF4A + | 5672 | 690, | 692- | 790- | 839- | 888- | 937- | 986- | 1035- | 1084- |
| HNF1A | | 691, | 789, | 838, | 887, | 936, | 985, | 1034, | 1083, | 1132, |
| | | 1133, | 1135- | 1233- | 1282- | 1331- | 1380- | 1429- | 1478- | 1527- |
| | | 1134, | 1232, | 1281, | 1330, | 1379, | 1428, | 1477, | 1526, | 1575, |
| | | 5673, | 5675 | 5676 | 5677 | 5678 | 5679 | 5680 | 5681 | 5682 |
| | | 5674 | | | | | | | | |

| | | mRNA | mRNA | mRNA | mRNA | mRNA | mRNA | mRNA | mRNA |
|---|---|---|---|---|---|---|---|---|---|
| | mRNA | i-3 | i-3 | i-3 | i-3 | i-3 | i-3 | i-3 | i-3 |
| | i-3 wt | opt1 | opt2 | opt3 | opt4 | opt5 | opt11 | opt28 | opt29 |
| Sum of | 1576, | 1578- | 1676- | 1725- | 1774- | 1823- | 1872- | 1921- | 1970- |
| All | 1577, | 1675, | 1724, | 1773, | 1822, | 1871, | 1920, | 1969, | 2018, |
| HNF4A | 2019, | 2021- | 2119- | 2168- | 2217- | 2266- | 2315- | 2364- | 2413- |
| | 2020, | 2118, | 2167, | 2216, | 2265, | 2314, | 2363, | 2412, | 2461, |
| | 2462, | 2464- | 2562- | 2611- | 2660- | 2709- | 2758- | 2807- | 2856- |
| | 2463 | 2561 | 2610 | 2659 | 2708 | 2757 | 2806 | 2855 | 2904 |
| Sum of | 1576, | 1578- | 1676- | 1725- | 1774- | 1823- | 1872- | 1921- | 1970- |
| HNF4A | 1577 | 1675 | 1724 | 1773 | 1822 | 1871 | 1920 | 1969 | 2018 |
| 1-474 | | | | | | | | | |
| Sum of | 2019, | 2021- | 2119- | 2168- | 2217- | 2266- | 2315- | 2364- | 2413- |
| HNF4A | 2020 | 2118 | 2167 | 2216 | 2265 | 2314 | 2363 | 2412 | 2461 |
| 1-375 | | | | | | | | | |
| Sum of | 2462, | 2464- | 2562- | 2611- | 2660- | 2709- | 2758- | 2807- | 2856- |
| HNF4A | 2463 | 2561 | 2610 | 2659 | 2708 | 2757 | 2806 | 2855 | 2904 |
| 39-474 | | | | | | | | | |
| HNF1A | 5683, | 5685 | 5686 | 5687 | 5688 | 5689 | 5690 | 5691 | 5692 |
| 1-631 | 5684 | | | | | | | | |
| Sum of | 1576, | 1578- | 1676- | 1725- | 1774- | 1823- | 1872- | 1921- | 1970- |
| All | 1577, | 1675, | 1724, | 1773, | 1822, | 1871, | 1920, | 1969, | 2018, |
| HNF4A + | 2019, | 2021- | 2119- | 2168- | 2217- | 2266- | 2315- | 2364- | 2413- |
| HNF1A | 2020, | 2118, | 2167, | 2216, | 2265, | 2314, | 2363, | 2412, | 2461, |
| | 2462, | 2464- | 2562- | 2611- | 2660- | 2709- | 2758- | 2807- | 2856- |
| | 2463, | 2561, | 2610, | 2659, | 2708, | 2757, | 2806, | 2855, | 2904, |
| | 5683, | 5685 | 5686 | 5687 | 5688 | 5689 | 5690 | 5691 | 5692 |
| | 5684 | | | | | | | | |
| | mRNA | mRNA | mRNA | mRNA | mRNA | mRNA | mRNA | mRNA | mRNA |
| | a-1 | a-1 | a-1 | a-1 | a-1 | a-1 | a-1 | a-1 | a-1 |
| | hSL- | hSL- | hSL- | hSL- | hSL- | hSL- | hSL- | hSL- | hSL- |
| | A100 | A100 | A100 | A100 | A100 | A100 | A100 | A100 | A100 |
| | wt | opt1 | opt2 | opt3 | opt4 | opt5 | opt11 | opt28 | opt29 |
| Sum of | 2905, | 2907- | 3005- | 3054- | 3103- | 3152- | 3201- | 3250- | 3299- |
| All | 2906, | 3004, | 3053, | 3102, | 3151, | 3200, | 3249, | 3298, | 3347, |
| HNF4A | 3348, | 3350- | 3448- | 3497- | 3546- | 3595- | 3644- | 3693- | 3742- |
| | 3349, | 3447, | 3496, | 3545, | 3594, | 3643, | 3692, | 3741, | 3790, |
| | 3791, | 3793- | 3891- | 3940- | 3989- | 4038- | 4087- | 4136- | 4185- |
| | 3792 | 3890, | 3939 | 3988 | 4037 | 4086 | 4135 | 4184 | 4233, |
| | | 5719- | | | | | | | 5728- |
| | | 5727 | | | | | | | 5736 |
| Sum of | 2905, | 2907- | 3005- | 3054- | 3103- | 3152- | 3201- | 3250- | 3299- |
| HNF4A | 2906 | 3004, | 3053 | 3102 | 3151 | 3200 | 3249 | 3298 | 3347, |
| 1-474 | | 5719- | | | | | | | 5728- |
| | | 5727 | | | | | | | 5736 |
| Sum of | 3348, | 3350- | 3448- | 3497- | 3546- | 3595 | 3644- | 3693- | 3742- |
| HNF4A | 3349 | 3447 | 3496 | 3545 | 3594 | 3643 | 3692 | 3741 | 3790 |
| 1-375 | | | | | | | | | |
| Sum of | 3791, | 3793- | 3891- | 3940- | 3989 | 4038- | 4087- | 4136- | 4185- |
| HNF4A | 3792 | 3890 | 3939 | 3988 | 4037 | 4086 | 4135 | 4184 | 4233 |
| 39-474 | | | | | | | | | |
| HNF1A | 5693, | 5695, | 5696 | 5697 | 5698 | 5699 | 5700 | 5701 | 5702 |
| 1-631 | 5694 | 5716- | | | | | | | |
| | | 5718 | | | | | | | |
| Sum of | 2905, | 2907- | 3005- | 3054- | 3103- | 3152- | 3201- | 3250- | 3299- |
| All | 2906, | 3004, | 3053, | 3102, | 3151, | 3200, | 3249, | 3298, | 3347, |
| HNF4A + | 3348, | 3350- | 3448- | 3497- | 3546- | 3595- | 3644- | 3693- | 3742- |
| HNF1A | 3349, | 3447, | 3496, | 3545, | 3594, | 3643, | 3692, | 3741, | 3790, |
| | 3791, | 3793- | 3891- | 3940- | 3989- | 4038- | 4087- | 4136- | 4185- |
| | 3792, | 3890, | 3939, | 3988, | 4037, | 4086, | 4135, | 4184, | 4233, |
| | 5693, | 5695, | 5696 | 5697 | 5698 | 5699 | 5700 | 5701 | 5702, |
| | 5694 | 5716- | | | | | | | 5728- |
| | | 5727 | | | | | | | 5736 |
| | mRNA | mRNA | mRNA | mRNA | mRNA | mRNA | mRNA | mRNA | mRNA |
| | a-1 | a-1 | a-1 | a-1 | a-1 | a-1 | a-1 | a-1 | a-1 |
| | A100 | A100 | A100 | A100 | A100 | A100 | A100 | A100 | A100 |
| | wt | opt1 | opt2 | opt3 | opt4 | opt5 | opt11 | opt28 | opt29 |
| Sum of | 4234, | 4236- | 4334- | 4383- | 4432- | 4481- | 4530- | 4579- | 4628- |
| All | 4235, | 4333, | 4382, | 4431, | 4480, | 4529, | 4578, | 4627, | 4676, |
| HNF4A | 4677, | 4679- | 4777- | 4826- | 4875- | 4924- | 4973- | 5022- | 5071- |
| | 4678, | 4776, | 4825, | 4874, | 4923, | 4972, | 5021, | 5070, | 5119, |
| | 5120, | 5122- | 5220- | 5269- | 5318- | 5367- | 5416- | 5465- | 5514- |
| | 5121 | 5219 | 5268 | 5317 | 5366 | 5415 | 5464 | 5513 | 5562 |
| Sum of | 4234, | 4236- | 4334- | 4383- | 4432- | 4481- | 4530- | 4579- | 4628- |
| HNF4A | 4235 | 4333 | 4382 | 4431 | 4480 | 4529 | 4578 | 4627 | 4676 |
| 1-474 | | | | | | | | | |
| Sum of | 4677, | 4679- | 4777- | 4826- | 4875- | 4924- | 4973- | 5022- | 5071- |
| HNF4A | 4678 | 4776 | 4825 | 4874 | 4923 | 4972 | 5021 | 5070 | 5119 |
| 1-375 | | | | | | | | | |
| Sum of | 5120, | 5122- | 5220- | 5269- | 5318- | 5367- | 5416- | 5465- | 5514- |
| HNF4A | 5121 | 5219 | 5268 | 5317 | 5366 | 5415 | 5464 | 5513 | 5562 |
| 39-474 | | | | | | | | | |
| HNF1A | 5703, | 5705 | 5706 | 5707 | 5708 | 5709 | 5710 | 5711 | 5712 |
| 1-631 | 5704 | | | | | | | | |
| Sum of | 4234, | 4236- | 4334- | 4383- | 4432- | 4481- | 4530- | 4579- | 4628- |
| All | 4235, | 4333, | 4382, | 4431, | 4480, | 4529, | 4578, | 4627, | 4676, |
| HNF4A + | 4677, | 4679- | 4777- | 4826- | 4875- | 4924- | 4973- | 5022- | 5071- |
| HNF1A | 4678, | 4776, | 4825, | 4874, | 4923, | 4972, | 5021, | 5070, | 5119, |
| | 5120, | 5122- | 5220- | 5269- | 5318- | 5367- | 5416- | 5465- | 5514- |
| | 5121 | 5219 | 5268 | 5317 | 5366 | 5415 | 5464 | 5513 | 5562 |
| | 5703, | 5705 | 5706 | 5707 | 5708 | 5709 | 5710 | 5711 | 5712 |
| | 5704 | | | | | | | | |

Thus, SEQ ID NO:1 to SEQ ID NO:5736 are preferred sequences useful in the context of the present invention. Detailed information related to the preferred sequences of the present invention is disclosed in the ST.25 sequence listing under <223> Other Information [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)], which is easily accessible for a skilled artisan. Generally, regarding lists of sequences, if only engineered HNF4A sequences are meant to appear in a certain list of sequences, sequences related to WT HNF4A which erroneously were included are herewith excluded from said list. The same is true vice versa for lists which should comprise only WT HNF4A sequences.

In further preferred embodiments, by engineering of the protein sequence the intracellular stability was improved and/or the transcriptional activity of HNF4A—both specific features, which make use of the specific mRNA medicine feature of being active intracellularly, in contrast to classic protein replacement therapies which would not be sufficient to treat intracellular defects.

It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 or fragments or variants thereof, may be selected and may accordingly be understood as suitable coding sequence of the invention. Further information regarding said amino acid sequences is also provided in Table A and Table B-I/B-II, and under <223> identifier of the ST25 sequence listing of respective sequence SEQ ID NOs [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)].

According to preferred embodiments, the nucleic acid of the invention comprises at least one coding sequence encoding at least one HNF4A protein or engineered protein variant, preferably as defined above or below, or fragments and variants thereof. In that context, any coding sequence encoding at least one protein or engineered protein variant as defined herein, or fragments and variants thereof may be understood as suitable coding sequence and may therefore be comprised in the nucleic acid of the invention.

### HNF4A Protein Sequences (WT Protein and Engineered HNF4A Protein Variants)

Details regarding engineered HNF4A protein variants and constructs being superior in terms of expression, activity and/or therapeutic effect and which are in sum beneficial when administered to a patient in need (any one of SEQ ID NO:100-148, 149-197, or 198-246, preferably SEQ ID NO:138, more preferably SEQ ID NO:186, even more preferably SEQ ID NO:140) are provided herein below in Table B-I/B-II. Amino acid positions provided in Table B-I/B-II and also throughout the whole specification, are made with reference to SEQ ID NO:100. The column designations of the following tables are as follows (reference is made again to the <223> identifier of the ST25 sequence listing of respective sequence SEQ ID NOs, which provides more detailed information on the sequences [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]):
Column A provides a short description of suitable HNF4A variant(s) or construct(s);
Column B provides protein (amino acid) SEQ ID NOs of respective HNF4A variant(s) or construct(s);
Column C provides SEQ ID NO of the corresponding wild type nucleic acid coding sequence(s);
Column D provides SEQ ID NO of the corresponding G/C optimized nucleic acid coding sequence(s) (opt1, gc);
Column E provides SEQ ID NO of the corresponding human codon usage adapted nucleic acid coding sequence(s) (opt3, human); and
Column F provides SEQ ID NO of other corresponding codon or sequence optimized nucleic acid coding sequence(s).

For example, to prevent inhibitory ubiquitination and acetylation of HNF4A, arginine mutants were inserted. As the impact of phosphorylation on HNF4A activity is unclear, complementary phospho-incompetent (alanine) and phospho-mimetic (glutamate) mutants were generated.

Notably, the description of the invention explicitly includes the information provided under <223> identifier of the ST.25 sequence listing of the present application [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]. Preferred nucleic acid constructs comprising coding sequences of Table B-I/B-II, e.g. mRNA sequences comprising the coding sequences of Table B-I/B-II are provided in Table C1/2.

TABLE B-I
Exemplary suitable HNF4A protein designs including mRNAs encoding said proteins
(see also Table B-II for description of the specific related SEQ ID NO).

| | Set- | | Mutated amino acid positions made |
|---|---|---|---|
| Row | Number | Alternative name | with reference to SEQ ID NO: 100. |
| 1 | Set A-1 | — | Δ376-474 |
| 2 | Set A-2 | — | Δ1-38 |
| 3 | Set A-3 | — | E285Q |
| 4 | Set A-4 | — | R2V |
| 5 | Set A-5 | — | R2V K5V |
| 6 | Set A-6 | — | K179R K180R |
| 7 | Set A-7 | — | K234R |
| 8 | Set A-8 | — | K300R |
| 9 | Set A-9 | — | K300R K307R K309R |
| 10 | Set A-10 | — | K447R K470R |
| 11 | Set A-11 | — | K458R |
| 12 | Set A-12 | 4x K to R | K106R K108R K126R K127R |
| 13 | Set B-1 | — | S313A |
| 14 | Set B-2 | — | S313E |
| 15 | Set B-3 | — | S142A S143A |
| 16 | Set B-4 | — | S142E S143E |
| 17 | Set B-5 | — | S143A |
| 18 | Set B-6 | — | T166A |
| 19 | Set B-7 | — | T166E |
| 20 | Set B-8 | — | S148A |
| 21 | Set B-9 | — | S148E |
| 22 | Set B-10 | — | S183A |
| 23 | Set B-11 | — | S183E |
| 24 | Set B-12 | — | S461A |
| 25 | Set B-13 | — | S461E |
| 26 | Set B-14 | — | S167A |
| 27 | Set B-15 | — | S167E |
| 28 | Set B-16 | — | S167A S378A T429A T432A S436A |
| 29 | Set B-17 | — | S167E S378E T429E T432E S436E |
| 30 | Set B-18 | — | S87A |
| 31 | Set B-19 | — | S87E |
| 32 | Set C-1 | combo 1 | S142A S143A S313A |
| 33 | Set C-2 | combo 2 | S142A S143A S313A K458R |
| 34 | Set C-3 | combo 3 | S142A S143A S167A S313A S378A T429A T432A |
| | | | S436A K458R |
| 35 | Set C-4 | combo 4 | S142A S143A S148A S167A S313A S378A T429A |
| | | | T432A S436A K458R |
| 36 | Set C-5 | combo 5 | K106R K108R K126R K127R S142A S143A S148A |
| | | | S167A S313A S378A T429A T432A S436A K458R |
| 37 | Set C-6 | combo 6 | S142A S143A S148A T166A S167A S313A S378A |
| | | | T429A T432A S436A K458R |
| 38 | Set C-7 | combo 7 | S87A S142A S143A S148A T166A S167A S313A |
| | | | S378A T429A T432A S436A K458R |
| 39 | Set C-8 | combo 8 | S87A K106R K108R K126R K127R S142A S143A |
| | | | S148A T166A S167A S313A S378A T429A T432A |
| | | | S436A K458R |
| 39.1 | Set D-0.1 | 5x SP/TP to AP | S167A S378A T429A T432A S436A |
| 40 | Set D-1 | combo 9 (=S87A 4x K to R) | S87A K106R K108R K126R K127R |
| 41 | Set D-2 | combo 10 (=combo 8 + | S87A K106R K108R K126R K127R S142A S143A |
| | | S461A) | S148A T166A S167A S313A S378A T429A T432A |
| | | | S436A K458R S461A |
| 42 | Set D-3 | combo 11 (=combo 8 + | S87A K106R K108R K126R K127R S142A S143A |
| | | S461E) | S148A T166A S167A S313A S378A T429A T432A |
| | | | S436A K458R S461E |
| 43 | Set D-4 | combo 12 | S87A K106R K108R K126R K127R S142A S143A |
| | | | S148E T166A S167E S313A S378E T429E T432E |
| | | | S436E K458R S461E |
| 44 | Set D-5 | combo 13 | S87A K106R K108R K126R K127R S142A S143A |
| | | | S148A T166A S167A K179R K180R S313A S378A |
| | | | T429A T432A S436A K458R S461E |
| 45 | Set D-6 | combo 14 | S87A K106R K108R K126R K127R S142A S143A |
| | | | S148A T166A S167A K300R K307R K309R S313A |
| | | | S378A T429A T432A S436A K458R S461E |
| 46 | Set D-7 | combo 15 | S87A K106R K108R K126R K127R S142A S143A |
| | | | S148A T166A S167A S313A S378A T429A T432A |
| | | | S436A K447R K458R S461E K470R |
| 47 | Set D-8 | combo 16 | S87A K106R K108R K126R K127R S142A S143A |
| | | | S148A T166A S167A K179R K180R K234R K300R |
| | | | K307R K309R S313A S378A T429A T432A S436A |
| | | | K447R K458R S461E K470R |
| 48 | Set D-9 | combo 17 | S87A S95A S99A K106R K108R K126R K127R S142A |
| | | | S143A S148A T166A S167A S313A S378A T429A |
| | | | T432A S436A K458R S461E |
| 49 | Set D-10 | combo 18 | S87A K106R K108R K126R K127R S138A T139A |
| | | | S142A S143A S148A T166A S167A S313A S378A |
| | | | T429A T432A S436A K458R S461E |
| 50 | Set D-11 | combo 19 | S87A S95A S99A K106R K108R K126R K127R S138A |
| | | | T139A S142A S143A S148A T166A S167A S313A |
| | | | S378A T429A T432A S436A K458R S461E |

More information on the sequences is disclosed in the ST.25 sequence listing under <223> Other Information [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]. Each construct as shown in the sequence listing resembles a preferred construct of the invention.

### Regarding the Different Sets of Mutations

the beneficial mutation set “S142A S143A” is present in combo 1 to 8 and 10 to 19;
the beneficial mutation set “5×SP/TP to AP” (S167A S378A T429A T432A S436A) is present in combo 3-8, 10-11, 13-19
the beneficial mutation set “5×SP/TP to EP” (S167E S378E T429E T432E S436E) is present in combo 12;
the beneficial mutation set “4×K to R” (K106R K108R K126R K127R) is present in combo 5, 8 and 9 to 19.

TABLE B-II
Exemplary suitable HNF4A protein designs including mRNAs encoding the respective proteins including
SEQ ID NO-references (see also Table B-I for description of the specific HNF4A mutations).

| A (Set-Number) | B | C | D | E | F |
|---|---|---|---|---|---|
| WT and Set A-1, Set A-2, Set A-3, Set A- | 100-148, | 247, | 249-297, | 396-444, | 347-395, 790-838, |
| 4, Set A-5, Set A-6, Set A-7, Set A-8, Set | 149-197, | 248, | 692-740, | 839-887, | 1233-1281, 396-444, |
| A-9, Set A-10, Set A-11, Set A-12, Set B- | 198-246 | 690, | 1135-1183, | 1282-1330 | 839-887, 1282-1330, |
| 1, Set B-2, Set B-3, Set B-4, Set B-5, Set | | 691, | 298-346, | | 445-493, 888-936, |
| B-6, Set B-7, Set B-8, Set B-9, Set B-10, | | 1133, | 741-789, | | 1331-1379, 494-542, |
| Set B-11, Set B-12, Set B-13, Set B-14, | | 1134 | 1184-1232 | | 937-985, 1380-1428, |
| Set B-15, Set B-16, Set B-17, Set B-18, | | | | | 543-591, 986-1034, |
| Set B-19, Set C-1 − combo 1, Set C-2 − | | | | | 1429-1477, 592-640, |
| combo 2, Set C-3 − combo 3, Set C-4 − | | | | | 1035-1083, 1478-1526, |
| combo 4, Set C-5 − combo 5, Set C-6 − | | | | | 641-689, 1084-1132, |
| combo 6, Set C-7 − combo 7, Set C-8 − | | | | | 1527-1575 |
| combo 8, combo 9 (Set D-1 − S87A 4x K | | | | | |
| to R), combo 10 (Set D-2 − combo 8 + | | | | | |
| S461A), combo 11 (Set D-3), Set D-4 − | | | | | |
| combo 12, Set D-5 − combo 13, Set D-6 − | | | | | |
| combo 14, Set D-7 − combo 15, Set D-8 − | | | | | |
| combo 16, Set D-9 − combo 17, Set D-10 − | | | | | |
| combo 18, Set D-11 − combo 19 | | | | | |

More information on the sequences is disclosed in the ST.25 sequence listing under <223> Other Information [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]. Each sequence or construct as shown in Table A or the sequence listing resembles a preferred construct of the invention.
Column A provides a short description of suitable HNF4A variant(s) or construct(s), wherein
   SEQ ID NO:100, 247, 248, 249, 298, 347, 396, 445, 494, 543, 592, 641, 1576, 1577, 1578, 1627, 1676, 1725, 1774, 1823, 1872, 1921, 1970, 2905, 2906, 2907, 2956, 3005, 3054, 3103, 3152, 3201, 3250, 3299, 4234, 4235, 4236, 4285, 4334, 4383, 4432, 4481, 4530, 4579, 4628, 5719, 5722, 5725, 5728, 5731, and 5734 are related to WT sequences (HNF4A protein and encoding mRNA);
   SEQ ID NO:149, 690, 691, 692, 741, 790, 839, 888, 937, 986, 1035, 1084, 2019, 2020, 2021, 2070, 2119, 2168, 2217, 2266, 2315, 2364, 2413, 3348, 3349, 3350, 3399, 3448, 3497, 3546, 3595, 3644, 3693, 3742, 4677, 4678, 4679, 4728, 4777, 4826, 4875, 4924, 4973, 5022, 5071 are related to WT sequences (HNF4A protein and encoding mRNA); and
   SEQ ID NO:198, 1133, 1134, 1135, 1184, 1233, 1282, 1331, 1380, 1429, 1478, 1527, 2462, 2463, 2464, 2513, 2562, 2611, 2660, 2709, 2758, 2807, 2856, 3791, 3792, 3793, 3842, 3891, 3940, 3989, 4038, 4087, 4136, 4185, 5120, 5121, 5122, 5171, 5220, 5269, 5318, 5367, 5416, 5465, 5514 are related to WT sequences (HNF4A protein and encoding mRNA)
Column B provides protein (amino acid) SEQ ID NOs of respective HNF4A variant(s) or construct(s);
Column C provides SEQ ID NO of the corresponding wild type nucleic acid coding sequence(s);
Column D provides SEQ ID NO of the corresponding G/C optimized nucleic acid coding sequence(s) (opt1, gc);
Column E provides SEQ ID NO of the corresponding human codon usage adapted nucleic acid coding sequence(s) (opt3, human); and
Column F provides SEQ ID NO of other corresponding codon or sequence optimized nucleic acid coding sequence(s).

In particularly preferred aspects and embodiments of the invention, the protein of the invention comprises or consists of at least one of the amino acid sequences having at least 80%, 81%, 82%, 83%, 84%, 85%, 86%, 87%, 88% h, 89%, 90%, 91%, 92%, 93%, 94%, 95%, 96%, 97%, 98%, 99%, or 100% sequence identity to any one of SEQ ID NO:100-148, 149-197, or 198-246, or a fragment or variant of said sequences having the biological activity of a HNF4A hepatocyte nuclear factor 4 alpha (HNF4A) protein.

In preferred embodiments, the engineered HNF4A protein variant with increased HNF4A transcriptional activity, DNA binding capacity, stability, longer-lasting HNF4A half-life and/or therapeutic effect is selected from Table B-III. In preferred embodiments, any of these sequences is encoded by an mRNA of the invention, as described herein or in the sequence listing.

TABLE B-III
Preferred exemplary suitable HNF4A protein designs of the invention (see
also Table B-I or II for description of the specific related SEQ ID NO)

| SEQ ID NO: | engineered HNF4A protein sequence |
|---|---|
| 101 | HNF4A(1-474)(S313A) |
| 102 | HNF4A(1-474)(S313E) |
| 103 | HNF4A(1-474)(S142A S143A) |
| 104 | HNF4A(1-474)(S142E S143E) |
| 105 | HNF4A(1-474)(S143A) |
| 106 | HNF4A(1-474)(T166A) |
| 107 | HNF4A(1-474)(S167E) |
| 108 | HNF4A(1-474)(S167A) |
| 109 | HNF4A(1-474)(S167A S378A T429A T432A S436A) |
| 110 | HNF4A(1-474)(S167E S378E T429E T432E S436E) |
| 111 | HNF4A(1-474)(S87A) |
| 112 | HNF4A(1-474)(S87E) |
| 113 | HNF4A(1-474)(R2V) |
| 114 | HNF4A(1-474)(R2V K5V) |
| 115 | HNF4A(1-474)(K179R K180R) |
| 116 | HNF4A(1-474)(K234R) |
| 117 | HNF4A(1-474)(K300R) |
| 118 | HNF4A(1-474)(K300R K307R K309R) |
| 119 | HNF4A(1-474)(K447R K470R) |
| 120 | HNF4A(1-474)(K458R) |
| 121 | HNF4A(1-474)(K106R K108R K126R K127R) |
| 122 | HNF4A(1-474)(E285Q) |
| 123 | HNF4A(1-474)(T166E) |
| 124 | HNF4A(1-474)(S148A) |
| 125 | HNF4A(1-474)(S148E) |
| 126 | HNF4A(1-474)(S183A) |
| 127 | HNF4A(1-474)(S183E) |
| 128 | HNF4A(1-474)(S461A) |
| 129 | HNF4A(1-474)(S461E) |
| 130 | HNF4A(1-474)(S142A S143A S313A) |
| 131 | HNF4A(1-474)(S142A S143A S313A K458R) |
| 132 | HNF4A(1-474)(S142A S143A S167A S313A S378A T429A T432A S436A K458R) |
| 133 | HNF4A(1-474)(S142A S143A S148A S167A S313A S378A T429A T432A S436A K458R) |
| 134 | HNF4A(1-474)(K106R K108R K126R K127R S142A S143A S148A S167A S313A S378A T429A |
| | T432A S436A K458R) |
| 135 | HNF4A(1-474)(S142A S143A S148A T166A S167A S313A S378A T429A T432A S436A K458R) |
| 136 | HNF4A(1-474)(S87A S142A S143A S148A T166A S167A S313A S378A T429A T432A S436A |
| | K458R) |
| 137 | HNF4A(1-474)(S87A K106R K108R K126R K127R S142A S143A S148A T166A S167A S313A |
| | S378A T429A T432A S436A K458R) |
| 138 | HNF4A(1-474)(S87A K106R K108R K126R K127R) |
| 139 | HNF4A(1-474)(S87A K106R K108R K126R K127R S142A S143A S148A T166A S167A S313A |
| | S378A T429A T432A S436A K458R S461A) |
| 140 | HNF4A(1-474)(S87A K106R K108R K126R K127R S142A S143A S148A T166A S167A S313A |
| | S378A T429A T432A S436A K458R S461E) |
| 141 | HNF4A(1-474)(S87A K106R K108R K126R K127R S142A S143A S148E T166A S167E S313A |
| | S378E T429E T432E S436E K458R S461E) |
| 142 | HNF4A(1-474)(S87A K106R K108R K126R K127R S142A S143A S148A T166A S167A K179R |
| | K180R S313A S378A T429A T432A S436A K458R S461E) |
| 143 | HNF4A(1-474)(S87A K106R K108R K126R K127R S142A S143A S148A T166A S167A K300R |
| | K307R K309R S313A S378A T429A T432A S436A K458R S461E) |
| 144 | HNF4A(1-474)(S87A K106R K108R K126R K127R S142A S143A S148A T166A S167A S313A |
| | S378A T429A T432A S436A K447R K458R S461E K470R) |
| 145 | HNF4A(1-474)(S87A K106R K108R K126R K127R S142A S143A S148A T166A S167A K179R |
| | K180R K234R K300R K307R K309R S313A S378A T429A T432A S436A K447R K458R S461E |
| | K470R) |
| 146 | HNF4A(1-474)(S87A S95A S99A K106R K108R K126R K127R S142A S143A S148A T166A |
| | S167A S313A S378A T429A T432A S436A K458R S461E) |
| 147 | HNF4A(1-474)(S87A K106R K108R K126R K127R S138A T139A S142A S143A S148A T166A |
| | S167A S313A S378A T429A T432A S436A K458R S461E) |
| 148 | HNF4A(1-474)(S87A S95A S99A K106R K108R K126R K127R S138A T139A S142A S143A |
| | S148A T166A S167A S313A S378A T429A T432A S436A K458R S461E) |
| 149 | HNF4A(1-375) |
| 150 | HNF4A(1-375)(S313A) |
| 151 | HNF4A(1-375)(S313E) |
| 152 | HNF4A(1-375)(S142A S143A) |
| 153 | HNF4A(1-375)(S142E S143E) |
| 154 | HNF4A(1-375)(S143A) |
| 155 | HNF4A(1-375)(T166A) |
| 156 | HNF4A(1-375)(S167E) |
| 157 | HNF4A(1-375)(S167A) |
| 158 | HNF4A(1-375)(S167A S378A T429A T432A S436A) |
| 159 | HNF4A(1-375)(S167E S378E T429E T432E S436E) |
| 160 | HNF4A(1-375)(S87A) |
| 161 | HNF4A(1-375)(S87E) |
| 162 | HNF4A(1-375)(R2V) |
| 163 | HNF4A(1-375)(R2V K5V) |
| 164 | HNF4A(1-375)(K179R K180R) |
| 165 | HNF4A(1-375)(K234R) |
| 166 | HNF4A(1-375)(K300R) |
| 167 | HNF4A(1-375)(K300R K307R K309R) |
| 168 | HNF4A(1-375)(K447R K470R) |
| 169 | HNF4A(1-375)(K458R) |
| 170 | HNF4A(1-375)(K106R K108R K126R K127R) |
| 171 | HNF4A(1-375)(E285Q) |
| 172 | HNF4A(1-375)(T166E) |
| 173 | HNF4A(1-375)(S148A) |
| 174 | HNF4A(1-375)(S148E) |
| 175 | HNF4A(1-375)(S183A) |
| 176 | HNF4A(1-375)(S183E) |
| 177 | HNF4A(1-375)(S461A) |
| 178 | HNF4A(1-375)(S461E) |
| 179 | HNF4A(1-375)(S142A S143A S313A) |
| 180 | HNF4A(1-375)(S142A S143A S313A K458R) |
| 181 | HNF4A(1-375)(S142A S143A S167A S313A S378A T429A T432A S436A K458R) |
| 182 | HNF4A(1-375)(S142A S143A S148A S167A S313A S378A T429A T432A S436A K458R) |
| 183 | HNF4A(1-375)(K106R K108R K126R K127R S142A S143A S148A S167A S313A S378A T429A |
| | T432A S436A K458R) |
| 184 | HNF4A(1-375)(S142A S143A S148A T166A S167A S313A S378A T429A T432A S436A K458R) |
| 185 | HNF4A(1-375)(S87A S142A S143A S148A T166A S167A S313A S378A T429A T432A S436A |
| | K458R) |
| 186 | HNF4A(1-375)(S87A K106R K108R K126R K127R S142A S143A S148A T166A S167A S313A |
| | S378A T429A T432A S436A K458R) |
| 187 | HNF4A(1-375)(S87A K106R K108R K126R K127R) |
| 188 | HNF4A(1-375)(S87A K106R K108R K126R K127R S142A S143A S148A T166A S167A S313A |
| | S378A T429A T432A S436A K458R S461A) |
| 189 | HNF4A(1-375)(S87A K106R K108R K126R K127R S142A S143A S148A T166A S167A S313A |
| | S378A T429A T432A S436A K458R S461E) |
| 190 | HNF4A(1-375)(S87A K106R K108R K126R K127R S142A S143A S148E T166A S167E S313A |
| | S378E T429E T432E S436E K458R S461E) |
| 191 | HNF4A(1-375)(S87A K106R K108R K126R K127R S142A S143A S148A T166A S167A K179R |
| | K180R S313A S378A T429A T432A S436A K458R S461E) |
| 192 | HNF4A(1-375)(S87A K106R K108R K126R K127R S142A S143A S148A T166A S167A K300R |
| | K307R K309R S313A S378A T429A T432A S436A K458R S461E) |
| 193 | HNF4A(1-375)(S87A K106R K108R K126R K127R S142A S143A S148A T166A S167A S313A |
| | S378A T429A T432A S436A K447R K458R S461E K470R) |
| 194 | HNF4A(1-375)(S87A K106R K108R K126R K127R S142A S143A S148A T166A S167A K179R |
| | K180R K234R K300R K307R K309R S313A S378A T429A T432A S436A K447R K458R S461E |
| | K470R) |
| 195 | HNF4A(1-375)(S87A S95A S99A K106R K108R K126R K127R S142A S143A S148A T166A |
| | S167A S313A S378A T429A T432A S436A K458R S461E) |
| 196 | HNF4A(1-375)(S87A K106R K108R K126R K127R S138A T139A S142A S143A S148A T166A |
| | S167A S313A S378A T429A T432A S436A K458R S461E) |
| 197 | HNF4A(1-375)(S87A S95A S99A K106R K108R K126R K127R S138A T139A S142A S143A |
| | S148A T166A S167A S313A S378A T429A T432A S436A K458R S461E) |
| 198 | HNF4A(39-474) |
| 199 | HNF4A(39-474)(S313A) |
| 200 | HNF4A(39-474)(S313E) |
| 201 | HNF4A(39-474)(S142A S143A) |
| 202 | HNF4A(39-474)(S142E S143E) |
| 203 | HNF4A(39-474)(S143A) |
| 204 | HNF4A(39-474)(T166A) |
| 205 | HNF4A(39-474)(S167E) |
| 206 | HNF4A(39-474)(S167A) |
| 207 | HNF4A(39-474)(S167A S378A T429A T432A S436A) |
| 208 | HNF4A(39-474)(S167E S378E T429E T432E S436E) |
| 209 | HNF4A(39-474)(S87A) |
| 210 | HNF4A(39-474)(S87E) |
| 211 | HNF4A(39-474)(R2V) |
| 212 | HNF4A(39-474)(R2V K5V) |
| 213 | HNF4A(39-474)(K179R K180R) |
| 214 | HNF4A(39-474)(K234R) |
| 215 | HNF4A(39-474)(K300R) |
| 216 | HNF4A(39-474)(K300R K307R K309R) |
| 217 | HNF4A(39-474)(K447R K470R) |
| 218 | HNF4A(39-474)(K458R) |
| 219 | HNF4A(39-474)(K106R K108R K126R K127R) |
| 220 | HNF4A(39-474)(E285Q) |
| 221 | HNF4A(39-474)(T166E) |
| 222 | HNF4A(39-474)(S148A) |
| 223 | HNF4A(39-474)(S148E) |
| 224 | HNF4A(39-474)(S183A) |
| 225 | HNF4A(39-474)(S183E) |
| 226 | HNF4A(39-474)(S461A) |
| 227 | HNF4A(39-474)(S461E) |
| 228 | HNF4A(39-474)(S142A S143A S313A) |
| 229 | HNF4A(39-474)(S142A S143A S313A K458R) |
| 230 | HNF4A(39-474)(S142A S143A S167A S313A S378A T429A T432A S436A K458R) |
| 231 | HNF4A(39-474)(S142A S143A S148A S167A S313A S378A T429A T432A S436A K458R) |
| 232 | HNF4A(39-474)(K106R K108R K126R K127R S142A S143A S148A S167A S313A S378A T429A |
| | T432A S436A K458R) |
| 233 | HNF4A(39-474)(S142A S143A S148A T166A S167A S313A S378A T429A T432A S436A K458R) |
| 234 | HNF4A(39-474)(S87A S142A S143A S148A T166A S167A S313A S378A T429A T432A S436A |
| | K458R) |
| 235 | HNF4A(39-474)(S87A K106R K108R K126R K127R S142A S143A S148A T166A S167A S313A |
| | S378A T429A T432A S436A K458R) |
| 236 | HNF4A(39-474)(S87A K106R K108R K126R K127R) |
| 237 | HNF4A(39-474)(S87A K106R K108R K126R K127R S142A S143A S148A T166A S167A S313A |
| | S378A T429A T432A S436A K458R S461A) |
| 238 | HNF4A(39-474)(S87A K106R K108R K126R K127R S142A S143A S148A T166A S167A S313A |
| | S378A T429A T432A S436A K458R S461E) |
| 239 | HNF4A(39-474)(S87A K106R K108R K126R K127R S142A S143A S148E T166A S167E S313A |
| | S378E T429E T432E S436E K458R S461E) |
| 240 | HNF4A(39-474)(S87A K106R K108R K126R K127R S142A S143A S148A T166A S167A K179R |
| | K180R S313A S378A T429A T432A S436A K458R S461E) |
| 241 | HNF4A(39-474)(S87A K106R K108R K126R K127R S142A S143A S148A T166A S167A K300R |
| | K307R K309R S313A S378A T429A T432A S436A K458R S461E) |
| 242 | HNF4A(39-474)(S87A K106R K108R K126R K127R S142A S143A S148A T166A S167A S313A |
| | S378A T429A T432A S436A K447R K458R S461E K470R) |
| 243 | HNF4A(39-474)(S87A K106R K108R K126R K127R S142A S143A S148A T166A S167A K179R |
| | K180R K234R K300R K307R K309R S313A S378A T429A T432A S436A K447R K458R S461E |
| | K470R) |
| 244 | HNF4A(39-474)(S87A S95A S99A K106R K108R K126R K127R S142A S143A S148A T166A |
| | S167A S313A S378A T429A T432A S436A K458R S461E) |
| 245 | HNF4A(39-474)(S87A K106R K108R K126R K127R S138A T139A S142A S143A S148A T166A |
| | S167A S313A S378A T429A T432A S436A K458R S461E) |
| 246 | HNF4A(39-474)(S87A S95A S99A K106R K108R K126R K127R S138A T139A S142A S143A |
| | S148A T166A S167A S313A S378A T429A T432A S436A K458R S461E) |

In more preferred embodiments, the engineered HNF4A protein variant with increased HNF4A transcriptional activity, DNA binding capacity, stability, longer-lasting HNF4A half-life and/or therapeutic effect is
(i) engineered HNF4A protein variant S87A K106R K108R K126R K127R, also designated as combo 9 (preferably SEQ ID NO:138);
(ii) engineered HNF4A protein variant with S87A K106R K108R K126R K127R S142A S143A S148A T166A S167A S313A S378A T429A T432A S436A K458R, also designated as “Set C-3”=combo 8, (preferably SEQ ID NO: 186); and most preferred the
(iii) engineered HNF4A protein variant with S87A K106R K108R K126R K127R S142A S143A S148A T166A S167A S313A S378A T429A T432A S436A K458R S461E mutations, also designated as combo 11, “Set D-3” or “combo 8+S461E” herein (preferably SEQ ID NO: 140).

In further embodiments, mRNA encoding engineered HNF4A protein variant with increased HNF4A transcriptional activity, DNA binding capacity, stability, longer-lasting HNF4A half-life and/or therapeutic effect preferably encodes an engineered HNF4A protein variant comprising a S87A substitution and/or a S461E substitution.

In further embodiments, mRNA encoding engineered HNF4A protein variant with increased HNF4A transcriptional activity, DNA binding capacity, stability, longer-lasting HNF4A half-life and/or therapeutic effect
(i) preferably encodes engineered HNF4A protein variant S87A K106R K108R K126R K127R, also designated as combo 9 (preferably SEQ ID NO:138);
(ii) more preferably encodes engineered HNF4A protein variant with S87A K106R K108R K126R K127R S142A S143A S148A T166A S167A S313A S378A T429A T432A S436A K458R, also designated as “Set C-3”=combo 8, (preferably SEQ ID NO:186); and most preferred the
(iii) most preferably encodes engineered HNF4A protein variant with S87A K106R K108R K126R K127R S142A S143A S148A T166A S167A S313A S378A T429A T432A S436A K458R S461E mutations, also designated as combo 11, “Set D-3” or “combo 8+S461E” herein (preferably SEQ ID NO:140).

Surprisingly, the combinations of different amino acid substitutions not only led to an additive effect, which would already be a surprising result as mutating a HNF4A protein without destroying the transcriptional activity or DNA binding capacity would be a surprising result alone, but combinations of mutations partly even led to synergistic effects.

As used herein, “wild type” and “naturally-occurring” refer to the form found in nature. For example, a wild type polypeptide or polynucleotide sequence is a sequence present in an organism that can be isolated from a source in nature and which has not been intentionally modified by human manipulation.

As used herein, “recombinant, “engineered, “variant” and “non-naturally occurring” when used with reference to a cell, nucleic acid, polypeptide or protein, refers to a material, or a material corresponding to the natural or native form of the material, that has been modified in a manner that would not otherwise exist in nature. In some embodiments, the cell, nucleic acid, polypeptide or protein is identical a naturally occurring cell, nucleic acid, polypeptide or protein, but is produced or derived from synthetic materials and/or by manipulation using recombinant techniques. Non-limiting examples include, among others, recombinant cells expressing genes that are not found within the native (non recombinant) form of the cell or express native genes that are otherwise expressed at a different level.

The terms “the same biological activity”, “essentially the same biological activity”, “similar biological activity” or “increased biological activity” in connection with an engineered HNF4A protein variant all refer to a HNF4A protein having (essentially) the same, similar or increased structural, regulatory, biochemical functions or biological activity as compared to a wild type HNF4A having WT HNF4A biological activity. Accordingly, the term “biological activity” in connection with HNF4A as used herein means the biological properties characteristic for a HNF4A protein. Further, a polynucleotide comprising a fragment of any of the aforementioned nucleic acid sequences is also encompassed as a polynucleotide of the present invention. The fragment shall encode a polypeptide which still has a biological activity as specified herein.

In the context of the present invention, the at least one coding sequence of the mRNA according to the invention preferably comprises a nucleic acid sequence encoding a peptide or protein comprising or consisting of a peptide or protein as defined herein. In some embodiments, a peptide or protein substantially comprises the entire amino acid sequence of the reference peptide or protein, such as the naturally occurring peptide or protein (e.g. HNF4A). Alternatively, the at least one coding sequence of the mRNA according to the invention may also comprise a nucleic acid sequence encoding a peptide or protein comprising or consisting of a fragment of a peptide or protein or a fragment of a variant of a peptide or protein as defined herein.

In the context of the present invention, a “fragment” of a peptide or protein or of a variant thereof may comprise a sequence of a peptide or protein or of a variant thereof as defined above, which is, with regard to its amino acid sequence (or its encoded nucleic acid sequence), N-terminally, C-terminally and/or intrasequentially truncated compared to the reference amino acid sequence, such as the amino acid sequence of the naturally occurring protein or a variant thereof (or its encoded nucleic acid sequence). Such truncation may occur either on the amino acid level or on the nucleic acid level, respectively. A sequence identity with respect to such a fragment as defined herein therefore preferably refers to the entire peptide or protein or a variant thereof as defined herein or to the entire (coding) nucleic acid sequence of such a peptide or protein or of a variant thereof.

According to some embodiments of the invention, the mRNA comprises at least one coding sequence encoding a peptide or protein comprising or consisting of a variant of a peptide or protein as defined herein, or a fragment of a variant of a peptide or protein.

In certain embodiments of the present invention, a “variant” of a peptide or protein or a fragment thereof as defined herein may be encoded by the mRNA comprising at least one coding sequence as defined herein, wherein the amino acid sequence encoded by the at least one coding sequence differs in at least one amino acid residue from the reference amino acid sequence, such as a naturally occurring amino acid sequence. In this context, the “change” in at least one amino acid residue may consist, for example, in a mutation of an amino acid residue to another amino acid, a deletion or an insertion. More preferably, the term “variant” as used in the context of the amino acid sequence encoded by the at least one coding sequence of the mRNA according to the invention comprises any homolog, isoform or transcript variant of a peptide or protein or a fragment thereof as defined herein, wherein the homolog, isoform or transcript variant is preferably characterized by a degree of identity or homology, respectively, as defined herein.

Naturally, it is to be understood, that HNF4A isoforms, e.g. produced by alternative promoter usage or alternative splicing, are comprised within the scope of the current invention. I.e. known isoforms like Isoform HNF4-Alpha-1 (Uniprot identifier: P41235-1), Isoform HNF4-Alpha-2 (Uniprot identifier: P41235-2), Isoform HNF4-Alpha-3 (Uniprot identifier: P41235-3), Isoform HNF4-Alpha-4 (Uniprot identifier: P41235-4), Isoform HNF4-Alpha-7 (Uniprot identifier: P41235-5), Isoform HNF4-Alpha-8 (Uniprot identifier: P41235-6), or Isoform HNF4-Alpha-9 (Uniprot identifier: P41235-7) are considered to be valid alternatives for the HNF4A which was used as a basis for the present invention.

In the context of the present invention, a “fragment” or a “variant” of a protein or peptide may have at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity over a stretch of at least 10, at least 20, at least 30, at least 50, at least 75 or at least 100 amino acids of such protein or peptide. More preferably, a “fragment” or a “variant” of a protein or peptide as used herein is at least 40%, preferably at least 50%, more preferably at least 60%, more preferably at least 70%, even more preferably at least 80%, even more preferably at least 90%, most preferably at least 95% identical to the protein or peptide, from which the variant is derived.

Preferably, a variant of a peptide or protein or a fragment thereof may be encoded by the mRNA comprising at least one coding sequence as defined herein, or may be provided simply by provision of the peptide or protein sequence per se, wherein at least one amino acid residue of the amino acid sequence encoded by the at least one coding sequence is substituted. Substitutions, wherein amino acids, which originate from the same class, are exchanged for one another, are called conservative substitutions or respectively conservative amino acid substitutions. In particular, these are amino acids having aliphatic side chains, positively or negatively charged side chains, aromatic groups in the side chains or amino acids, the side chains of which can form hydrogen bridges, e.g. side chains which have a hydroxyl function. By conservative constitution, e.g. an amino acid having a polar side chain may be replaced by another amino acid having a corresponding polar side chain, or, for example, an amino acid characterized by a hydrophobic side chain may be substituted by another amino acid having a corresponding hydrophobic side chain (e.g. serine (threonine) by threonine (serine) or leucine (isoleucine) by isoleucine (leucine)). In a preferred embodiment, a variant of a peptide or protein or a fragment thereof may be encoded by the mRNA according to the invention, wherein at least one amino acid residue of the amino acid sequence encoded by the at least one coding sequence comprises at least one conservative substitution compared to a reference sequence, such as the respective naturally occurring sequence. These amino acid sequences as well as their encoding nucleic acid sequences in particular are comprised by the term “variant” as defined herein.

Further, as used herein, the phrase “conservative amino acid substitutions” refers to the interchangeability of residues having similar side chains, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with a hydroxyl side chain is substituted with another amino acid with a hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table (i).

TABLE i
Exemplary conservative amino acid substitutions

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar (R, K, D, E, N, Q) |
| H, Y, W, F | Other aromatic (H, Y, W, F) |
| C, P | Non-polar (A, V, L, G, I, M, W, F, P) |

Insertions, deletions and/or non-conservative substitutions are also possible, in particular, at those sequence positions, which preferably do not cause a substantial modification of the three-dimensional structure. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

Preferred embodiments also are related to e.g. an amino acid substitution S87A or S461E and other alternatives which a skilled person would naturally chose to change this position. Other preferred alternatives are phospho-mimetics and phospho-incompetent exchanges, preferably but not limited to S87A or S87G, the latter also being a preferred phospho-incompetent mutant, as well as other phospho-incompetent mutants on this basis, which are easily derivable for a person skilled in the art. A further preferred alternative related to phospho-mimetics is S461E or S461D, which are examples for a phospho-mimetic mutation. Phospho-mimetic mutations naturally are not limited to S461E or S461D and a person skilled in the art naturally would conceive other alternative phospho-mimetic mutations. The same is true for ubiquitination and acetylation-incompetent mutations of HNF4A, where a skilled person naturally would conceive alternatives which are equally active.

In order to determine the percentage, to which two sequences (nucleic acid sequences, e.g. RNA or mRNA sequences as defined herein, or amino acid sequences, preferably the amino acid sequence encoded by the mRNA according to the invention) are identical, the sequences can be aligned in order to be subsequently compared to one another. For this purpose, e.g. gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage, to which two sequences are identical, can be determined using a mathematical algorithm. A preferred, but not limiting, example of a mathematical algorithm, which can be used is the algorithm of Karlin et al. (1993), PNAS USA, 90:5873-5877 or Altschul et al. (1997), Nucleic Acids Res., 25:3389-3402. Such an algorithm is integrated, for example, in the BLAST program. Sequences, which are identical to the sequences of the present invention to a certain extent, can be identified by this program.

In the context of the present invention, a fragment of a peptide or protein or a variant thereof encoded by the at least one coding sequence of the mRNA according to the invention may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with a reference amino acid sequence, preferably with the amino acid sequence of the respective naturally occurring full-length peptide or protein or a variant thereof.

Preferably, the at least one coding sequence of the mRNA, which encodes the at least one peptide or protein as defined herein, comprises or consists of a nucleic acid sequence selected from any single element from the group consisting of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list], or a fragment or variant of any one of these nucleic acid sequences. As used herein, a nucleic acid sequence comprising or consisting of a nucleic acid sequence selected from any single element from the group consisting of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list] may also be referred to as “full-length nucleic acid (sequence)”. In this regard and throughout the specification, the term “element”, naturally, is deemed to indicate a single SEQ ID NO from the aforementioned list of SEQ ID NO.

In certain embodiments, the mRNA according to the invention, preferably the at least one coding sequence of the mRNA according to the invention, may comprise or consist of a fragment of a nucleic acid sequence encoding a peptide or protein or a fragment or variant thereof as defined herein. Preferably, the at least one coding sequence of the mRNA according to the invention comprises or consists of a fragment, preferably as defined herein, of any one of the nucleic acid sequences according to any single element from the group consisting of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list], or a variant of any one of these sequences.

In this context, a “fragment of a nucleic acid (sequence)” is preferably a nucleic acid sequence encoding a fragment of a peptide or protein or of a variant thereof as described herein. More preferably, the expression “fragment of a nucleic acid sequence” refers to a nucleic acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with a respective full-length nucleic acid sequence, preferably with a nucleic acid sequence selected from any single element from the group consisting of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list], or a variant of any of these nucleic acid sequences.

In another preferred embodiment, the mRNA according to the invention, preferably the at least one coding sequence of the mRNA according to the invention, may comprise or consist of a variant of a nucleic acid sequence as defined herein, preferably of a nucleic acid sequence encoding a peptide or protein or a fragment thereof as defined herein.

The expression “variant of a nucleic acid sequence” as used herein in the context of a nucleic acid sequence encoding a peptide or protein as described herein or a fragment thereof, typically refers to a nucleic acid sequence, which differs by at least one nucleic acid residue from the respective reference nucleic acid sequence, for example from the respective naturally occurring nucleic acid sequence or from a full-length nucleic acid sequence as defined herein, or from a fragment thereof. More preferably, the expression “variant of a nucleic acid sequence” as used in the context of the present invention refers to a nucleic acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with a nucleic acid sequence, from which it is derived.

Preferably, the mRNA according to the invention, more preferably the at least one coding sequence of the mRNA according to the invention, encodes a variant of a peptide or protein or a fragment thereof, preferably as defined herein.

In a preferred embodiment, the mRNA according to the invention, more preferably the at least one coding sequence of the mRNA according to the invention, comprises or consists of a variant of a nucleic acid sequence encoding a peptide or protein or a fragment thereof as defined herein, wherein the variant of the nucleic acid sequence encodes an amino acid sequence comprising at least one conservative substitution of an amino acid residue.

In another embodiment, the mRNA according to the invention, more preferably the at least one coding sequence of the mRNA according to the invention, comprises or consists of a variant of a nucleic acid sequence encoding a peptide or protein or a fragment thereof as defined herein, wherein the nucleic acid sequence of the variant differs from a reference nucleic acid sequence, preferably from the respective naturally occurring nucleic acid sequence in at least one nucleic acid residue, more preferably without resulting—due to the degenerated genetic code—in an alteration of the encoded amino acid sequence, i.e. the amino acid sequence encoded by the variant or at least part thereof may preferably not differ from the naturally occurring amino acid sequence in one or more mutation(s) within the above meaning.

Furthermore, a “variant” of a nucleic acid sequence encoding a peptide or protein or a “fragment or variant” thereof as defined herein, may also comprise mRNA or DNA sequences, which correspond to RNA or mRNA sequences as defined herein and may also comprise further RNA or mRNA sequences, which correspond to DNA sequences as defined herein. Those skilled in the art are familiar with the translation of an RNA or mRNA sequence into a DNA sequence (or vice versa) or with the creation of the complementary strand sequence (i.e. by substitution of U residues with T residues and/or by constructing the complementary strand with respect to a given sequence).

A “fragment” refers to a portion of the mRNA or nucleotide sequence encoding an HNF4A protein or a portion of the amino acid sequence of the HNF4A protein of the invention. A fragment of an HNF4A mRNA or nucleotide sequence of the invention may encode a biologically active portion of an HNF4A protein, or it may be a fragment that can be used as a hybridization probe or PCR primer using methods known to skilled persons in the art. A fragment of an HNF4A polypeptide may encompass a biologically active fragment of the HNF4A protein.

The term “biologically active fragments or variants” refers to fragments or variants of the exemplified nucleic acid molecules and polypeptides that comprise or encode HNF4A activity.

MRNAs or nucleic acid molecules that are “variants” of the mRNAs or nucleotide sequences disclosed herein are also encompassed by the present invention. “Variants” of the HNF4A nucleotide sequences of the invention include those sequences that encode the HNF4A proteins disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the HNF4A protein disclosed in the present invention as discussed herein above or below. Generally, nucleotide sequence variants of the invention will have at least about 75%, 80%, 85%, 90%, 91%, 92%, 93%, 94%, 95%, 96%, 97%, 98%, or 99% identity to a particular nucleotide sequence disclosed herein. A variant HNF4A mRNA or nucleotide sequence will encode a HNF4A protein, respectively, that has an amino acid sequence having at least about 75%, 80%, 85%, 90%, 91%, 92%, 93%, 94%, 95%, 96%, 97%, 98%, or 99% identity to the amino acid sequence of a HNF4A protein disclosed herein.

In another embodiment, the present invention concerns an mRNA comprising at least one coding sequence, wherein the at least one coding sequence encodes at least one HNF4A protein, or a fragment or variant of any of these peptides or proteins, having the biological activity of a wild type HNF4A protein. Moreover, an mRNA comprising at least one coding sequence, wherein the at least one coding sequence encodes at least one HNF4A protein, or a fragment or variant of any of these peptides or proteins, is provided for use in the treatment or prophylaxis of a liver disease, wherein the liver disease is preferably selected from liver fibrosis, liver cirrhosis, hepatocellular carcinoma (HCC), non-alcoholic fatty liver disease (NAFLD), non-alcoholic steatohepatitis (NASH) and liver cancer, more preferably wherein the disease is liver fibrosis or liver cirrhosis.

According to certain embodiments, the mRNA according to the invention is mono-, bi-, or multicistronic, preferably as defined herein. The coding sequences in a bi- or multicistronic RNA preferably encode a distinct peptide or protein as defined herein or a fragment or variant thereof. Preferably, the coding sequences encoding two or more peptides or proteins may be separated in the bi- or multicistronic RNA by at least one IRES (internal ribosomal entry site) sequence, as defined below. Thus, the term “encoding two or more peptides or proteins” may mean, without being limited thereto, that the bi- or even multicistronic RNA, may encode e.g. at least two, three, four, five, six or more (preferably different) peptides or proteins as described herein or their fragments or variants within the definitions provided herein. More preferably, without being limited thereto, the bi- or even multicistronic mRNA, may encode, for example, at least two, three, four, five, six or more (preferably different) peptides or proteins as defined herein or their fragments or variants as defined herein. In this context, a so-called IRES (internal ribosomal entry site) sequence as defined above can function as a sole ribosome binding site, but it can also serve to provide a bi- or even multicistronic mRNA as defined above, which encodes several peptides or proteins, which are to be translated by the ribosomes independently of one another. Examples of IRES sequences, which can be used according to the invention, are those from picornaviruses (e.g. FMDV), pestiviruses (CFFV), polioviruses (PV), encephalomyocarditis viruses (ECMV), foot and mouth disease viruses (FMDV), hepatitis C viruses (HCV), classical swine fever viruses (CSFV), mouse leukoma virus (MLV), simian immunodeficiency viruses (SIV) or cricket paralysis viruses (CrPV).

According to a further embodiment the at least one coding sequence of the mRNA according to the invention may encode at least two, three, four, five, six, seven, eight, nine and more peptides or proteins (or fragments or variants thereof) as defined herein linked with or without an amino acid linker sequence, wherein said linker sequence can comprise rigid linkers, flexible linkers, cleavable linkers (e.g., self-cleaving peptides) or a combination thereof. Therein, the peptides or proteins (or fragments or variants thereof) may be identical or different or a combination thereof.

Preferably, the at least one coding sequence of the mRNA according to the invention comprises at least two, three, four, five, six, seven, eight, nine or more nucleic acid sequences identical to or having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with a nucleic acid sequence selected from any single element from the group consisting of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list], or a fragment or variant of any one of these nucleic acid sequences, wherein each mRNA may encode a different HNF4A protein or engineered HNF4A protein variant, i.e. a cocktail of different HNF4A proteins.

Preferably, the mRNA comprising at least one coding sequence as defined herein typically comprises a length of about 50 to about 20000, or 100 to about 20000 nucleotides, preferably of about 250 to about 20000 nucleotides, more preferably of about 500 to about 10000, even more preferably of about 500 to about 5000.

The mRNA according to the invention may further be single stranded or double stranded. When provided as a double stranded RNA, the mRNA according to the invention preferably comprises a sense and a corresponding antisense strand.

In a preferred embodiment, the mRNA comprising at least one coding sequence as defined herein is an mRNA, a viral RNA or a replicon RNA. Preferably, the mRNA is an artificial nucleic acid, more preferably as described herein.

According to a further embodiment, the RNA, preferably an mRNA, according to the invention is a modified RNA, preferably a modified RNA as described herein. A modified RNA as used herein does preferably not comprise a chemically modified sugar, a chemically modified backbone or a chemically modified nucleobase. More preferably, a modified RNA as used herein does not comprise a chemically modified nucleoside or a chemically modified nucleotide. It is further preferred that a modified RNA as used herein does not comprise a chemical modification as described in international patent application WO 2014158795.

In the context of the present invention, a modification as defined herein preferably leads to a stabilization of the mRNA according to the invention. More preferably, the invention thus provides a stabilized RNA comprising at least one coding sequence as defined herein.

According to one embodiment, the mRNA of the present invention may thus be provided as a “stabilized mRNA”, that is to say as an RNA that is essentially resistant to in vivo degradation (e.g. by an exo- or endo-nuclease).

Stabilization of an RNA can be achieved, for example, by a modified phosphate backbone of the mRNA of the present invention. A backbone modification in connection with the present invention is a modification, in which phosphates of the backbone of the nucleotides contained in the mRNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized RNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)).

In the following, specific modifications are described, which are preferably capable of “stabilizing” the mRNA as defined herein.

### RNA Constructs

The mRNA according to the invention, which comprises at least one coding sequence as defined herein, may preferably comprise a 5′-UTR and/or a 3′-UTR preferably containing at least one histone stem-loop. Where, in addition to the peptide or protein as defined herein or a fragment or variant thereof, a further peptide or protein is encoded by the at least one coding sequence of the mRNA according to the invention, the encoded peptide or protein is preferably no histone protein, no reporter protein and/or no marker or selection protein, as defined herein. The 3′-UTR of the mRNA according to the invention preferably comprises also a poly(A) and/or a poly(C) sequence as defined herein. The single elements of the 3′-UTR may occur therein in any order from 5′ to 3′ along the sequence of the mRNA of the present invention. In addition, further elements as described herein, may also be contained, such as a stabilizing sequence as defined herein (e.g. derived from the UTR of a globin gene), IRES sequences, etc. Each of the elements may also be repeated in the mRNA according to the invention at least once (particularly in di- or multicistronic constructs), preferably twice or more. As an example, the single elements may be present in the mRNA according to the invention in the following order (wherein the mRNA may optionally comprise a 5′-UTR element as described herein 5′ of the coding region/CDS and/or a 3′-UTR element as described herein 3′ of the coding region/CDS):
5′-coding region-histone stem-loop-poly(A)/(C) sequence-3′; or
5′-coding region-poly(A)/(C) sequence-histone stem-loop-3′; or
5′-coding region-histone stem-loop-polyadenylation signal-3′; or
5′-coding region-polyadenylation signal-histone stem-loop-3′; or
5′-coding region-histone stem-loop-histone stem-loop-poly(A)/(C) sequence-3′; or
5′-coding region-histone stem-loop-histone stem-loop-polyadenylation signal-3′; or
5′-coding region-poly(A)/(C) sequence-histone stem-loop-3′; or
5′-coding region-poly(A)/(C) sequence-poly(A)/(C) sequence-histone stem-loop-3′; or
5′-coding region-poly(A)/(C) sequence-histone stem-loop-3′ etc.

According to a further embodiment, the mRNA of the present invention preferably comprises at least one of the following structural elements: a 5′- and/or 3′-untranslated region element (UTR element), particularly a 5′-UTR element, which preferably comprises or consists of a nucleic acid sequence which is derived from the 5′-UTR of a TOP gene or from a fragment, homolog or a variant thereof, or a 5′- and/or 3′-UTR element which may preferably be derivable from a gene that provides a stable mRNA or from a homolog, fragment or variant thereof; a histone-stem-loop structure, preferably a histone-stem-loop in its 3′-untranslated region; a 5′-cap structure; a poly(A) tail; or a poly(C) sequence. Preferably, the mRNA of the invention comprises a 3′-terminal A nucleotide.

According to some embodiments, it is particularly preferred that—if, in addition to a peptide or protein as defined herein or a fragment or variant thereof, a further peptide or protein is encoded by the at least one coding sequence as defined herein—the encoded peptide or protein is preferably no histone protein, no reporter protein (e.g. Luciferase, GFP, EGFP, β-Galactosidase, particularly EGFP) and/or no marker or selection protein (e.g. alpha-Globin, Galactokinase and Xanthine:Guanine phosphoribosyl transferase (GPT)). In a preferred embodiment, the mRNA according to the invention does not comprise a reporter gene or a marker gene. Preferably, the mRNA according to the invention does not encode, for instance, luciferase; green fluorescent protein (GFP) and its variants (such as EGFP, RFP or BFP); α-globin; hypoxanthine-guanine phosphoribosyltransferase (HGPRT); β-galactosidase; galactokinase; alkaline phosphatase; secreted embryonic alkaline phosphatase (SEAP)) or a resistance gene (such as a resistance gene against neomycin, puromycin, hygromycin and zeocin). In a preferred embodiment, the mRNA according to the invention does not encode luciferase. In another embodiment, the mRNA according to the invention does not encode GFP or a variant thereof.

In another embodiment, the mRNA according to the present invention comprises, preferably in 5′ to 3′ direction, the following elements:
a) a 5′-cap structure, preferably m7GpppN,
b) a 5′-UTR element, which comprises or consists of a nucleic acid sequence, which is derived from the 5′-UTR of a TOP gene, preferably comprising a nucleic acid sequence according to SEQ ID NO:1 or 2 (HSD17B4), or a homolog, a fragment or a variant thereof or a 5′-UTR element, which comprises or consists of a nucleic acid sequence according to SEQ ID NO:27 or 28 (Slc7a3) or SEQ ID NO:23 or 24 (Rpl31), or a homolog, a fragment or a variant thereof,
c) at least one coding sequence as defined herein,
d) a 3′-UTR element comprising a nucleic acid sequence, which is derived from a PSMB3 gene, preferably comprising a nucleic acid sequence according to SEQ ID NO:33 or 34, or a homolog, a fragment or a variant thereof; and/or a 3′-UTR element comprising a nucleic acid sequence, which is derived from an albumin gene, preferably comprising a nucleic acid sequence according to SEQ ID NO:37 or 38, or a homolog, a fragment or a variant thereof,
e) a poly(A) tail, about 10 to about 200, about 10 to about 100, about 40 to about 80 or about 50 to about 70 adenosine nucleotides, more preferably about 100 adenosine nucleotides, preferably, wherein the mRNA comprises a 3′-terminal A nucleotide,
f) a poly(C) tail, preferably consisting of 10 to 200, 10 to 100, 20 to 70, 20 to 60 or 10 to 40 cytidine nucleotides, and
g) a histone stem-loop, preferably comprising a nucleic acid sequence according to SEQ ID NO:63 or 64.

In a further embodiment, the mRNA according to the present invention comprises, preferably in 5′ to 3′ direction, the following elements:
a) a 5′-cap structure, preferably m7GpppN,
b) a 5′-UTR element, which comprises or consists of a nucleic acid sequence, which is derived from the 5′-UTR of the HSD17B4 gene, preferably comprising a nucleic acid sequence according to SEQ ID NO:1 or 2, or a homolog, a fragment or a variant thereof,
c) at least one coding sequence as defined herein,
d) a 3′-UTR element comprising a nucleic acid sequence, which is derived from an PSMB3 gene, preferably comprising a nucleic acid sequence according to SEQ ID NO:33 or 34, or a homolog, a fragment or a variant thereof;
e) optionally, a histone stem-loop selected from SEQ ID NO:63 or 64; and/or
f) a poly(A) tail, preferably consisting of about 10 to about 200, about 10 to about 100, about 40 to about 80 or about 50 to about 70 adenosine nucleotides, more preferably about 100 adenosine nucleotides.

The mRNA according to the present invention may be prepared using any method known in the art, including synthetic methods such as e.g. solid phase RNA synthesis, as well as in vitro methods, such as RNA in vitro transcription reactions.

Preferred sequences are shown herein below in Table C1/C2.

TABLE C1
Preferred sequences of the present invention:

| Sequence | SEQ ID NO: # |
|---|---|
| All CDS | 247-1575, 5673-5682 |
| All CDS optimized (opt1-5, opt11, opt28, opt29) | 249-689, 692-1132, 1135-1575, 5675-5682 |
| All CDS wt | 247, 248, 690, 691, 1133, 1134, 5673, 5674 |
| All CDS opt1/GC | 249-346, 692-789, 1135-1232, 5675 |
| All CDS opt2/c-rich | 347-395, 790-838, 1233-1281, 5676 |
| All CDS opt3/human | 396-444, 839-887, 1282-1330, 5677 |
| All CDS opt4/main_codon | 445-493, 888-936, 1331-1379, 5678 |
| All CDS opt5 | 494-542, 937-985, 1380-1428, 5679 |
| All CDS opt11 | 543-591, 986-1034, 1429-1477, 5680 |
| All CDS opt28 | 592-640, 1035-1083, 1478-1526, 5681 |
| All CDS opt29 | 641-689, 1084-1132, 1527-1575, 5682 |
| All mRNAs | 1576-5615, 5683-5712, 5716-5736 |
| All mRNAs i-3 —/muag (A64-N5-C30-hSL-N5) | 1576-2904, 5683-5692 |
| All mRNAs a-1 HSD17B4/PSMB3 | 2905-5615, 5693-5712, 5716-5736 |
| All mRNAs a-1 HSD17B4/PSMB3 (hSL-A100) | 2905-4233, 5563-5615, 5693-5702, 5716-5736 |
| All mRNAs a-1 HSD17B4/PSMB3 (A100) | 4234-5562, 5703-5712 |

as apparent, different construct designs were applied. More information on the sequences is disclosed in the ST.25 sequence listing under <223> Other Information [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]. Each construct as shown in the sequence listing resembles a preferred construct of the invention.

In another embodiment, the nucleic acid of the present invention are DNA sequences, comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to any of the sequences selected from the group consisting of SEQ ID NO:1576-5615, 5683-5712, and 5716-5736 or SEQ ID NO:2906, 2956, 3005, 3250, 3299, 5728, 5731, 5734, 5563, 5564, 5565, 5566, 5567, 5568, 5569, 5570, 5571, 5572, 5573, 5574, 5575, 5576, 5577, 5578, 5579, 5580, 5581, 5582, 5583, 5584, 5585, 5586, 5587, 5588, 5589, 5590, 5591, 5592, 5593, 5594, 5595, 5596, 5597, 5598, 5599, 5600, 5601, 5602, 5603, 5604, 5605, 5606, 5607, 5608, 5609, 5610, 5611, 5612, 5613, 5614, and 5615 or to any one of the sequences as disclosed in the Table C2 (“Constructs of the invention”) herein below, wherein all Uracils (U) in the respective sequences are substituted by Thymidines (T), or a fragment or variant of any of these sequences.

TABLE C2
“Constructs of the invention”

| | | | | 5′-UTR/ | | SEQ ID NO: | SEQ ID NO: |
|---|---|---|---|---|---|---|---|
| SEQ ID NO: | RNA | | CDS | 3′-UTR; | 3′- | Protein w/o | CDS w/o |
| RNA | ID | Name | opt. | UTR Design | end | HA-tag | HA-tag |
| 1576-5615 | see respective information under <223> in the sequence listing of the invention [here, and throughout | | | | | | |
| | the whole specification, it has to be noted that the priority application was filed with a sequence listing | | | | | | |
| | in accordance with the WIPO Standard ST.25, which then was converted into a sequence listing | | | | | | |
| | according to WIPO Standard ST.26 - information which was comprised within line <223> in ST.25 now | | | | | | |
| | was added to the respective SEQ ID NO: as a note under “feature key”, i.e. “misc_feature” (for nucleic | | | | | | |
| | acids) or “REGION” (for proteins)] | | | | | | |
| 5683-5712 | see respective information under <223> in the sequence listing of the invention [here, and throughout | | | | | | |
| | the whole specification, it has to be noted that the priority application was filed with a sequence listing | | | | | | |
| | in accordance with the WIPO Standard ST.25, which then was converted into a sequence listing | | | | | | |
| | according to WIPO Standard ST.26 - information which was comprised within line <223> in ST.25 now | | | | | | |
| | was added to the respective SEQ ID NO: as a note under “feature key”, i.e. “misc_feature” (for nucleic | | | | | | |
| | acids) or “REGION” (for proteins)] | | | | | | |
| 2906 | R9821 | HNF4A(1-474) | wt | HSD17B4/ | hSL- | 100 | 248 |
| | | | | PSMB3; a-1 | A100 | | |
| 2956 | R9823, | HNF4A(1-474) | opt1 | HSD17B4/ | hSL- | 100 | 298 |
| | R10103 | | | PSMB3; a-1 | A100 | | |
| 3005 | R9824 | HNF4A(1-474) | opt2 | HSD17B4/ | hSL- | 100 | 347 |
| | | | | PSMB3; a-1 | A100 | | |
| 3250 | R9828 | HNF4A(1-474) | opt28 | HSD17B4/ | hSL- | 100 | 592 |
| | | | | PSMB3; a-1 | A100 | | |
| 3299 | R9822, | HNF4A(1-474) | opt29 | HSD17B4/ | hSL- | 100 | 641 |
| | R10595 | | | PSMB3; a-1 | A100 | | |
| 5563 | R9455 | HNF4A(1-474) HA-tag | wt | HSD17B4/ | hSL- | 100 | 248 |
| | | | | PSMB3; a-1 | A100 | | |
| 5564 | R9456 | HNF4A(1-474) HA-tag | opt1 | HSD17B4/ | hSL- | 100 | 249 |
| | | | | PSMB3; a-1 | A100 | | |
| 5565 | R9457 | HNF4A(1-474)(S313A) HA-tag | opt1 | HSD17B4/ | hSL- | 101 | 250 |
| | | | | PSMB3; a-1 | A100 | | |
| 5566 | R9458 | HNF4A(1-474)(S313E) HA-tag | opt1 | HSD17B4/ | hSL- | 102 | 251 |
| | | | | PSMB3; a-1 | A100 | | |
| 5567 | R9459 | HNF4A(1-474)(S142A S143A) | opt1 | HSD17B4/ | hSL- | 103 | 252 |
| | | HA-tag | | PSMB3; a-1 | A100 | | |
| 5568 | R9460 | HNF4A(1-474)(S142E S143E) | opt1 | HSD17B4/ | hSL- | 104 | 253 |
| | | HA-tag | | PSMB3; a-1 | A100 | | |
| 5569 | R9461 | HNF4A(1-474)(S143A) HA-tag | opt1 | HSD17B4/ | hSL- | 105 | 254 |
| | | | | PSMB3; a-1 | A100 | | |
| 5570 | R9462 | HNF4A(1-474)(T166A) HA-tag | opt1 | HSD17B4/ | hSL- | 106 | 255 |
| | | | | PSMB3; a-1 | A100 | | |
| 5571 | R9463 | HNF4A(1-474)(S167E) HA-tag | opt1 | HSD17B4/ | hSL- | 107 | 256 |
| | | | | PSMB3; a-1 | A100 | | |
| 5572 | R9464 | HNF4A(1-474)(S167A) HA-tag | opt1 | HSD17B4/ | hSL- | 108 | 257 |
| | | | | PSMB3; a-1 | A100 | | |
| 5573 | R9465 | HNF4A(1-474)(S167A S378A | opt1 | HSD17B4/ | hSL- | 109 | 258 |
| | | T429A T432A S436A) HA-tag | | PSMB3; a-1 | A100 | | |
| 5574 | R9466 | HNF4A(1-474)(S167E S378E | opti | HSD17B4/ | hSL- | 110 | 259 |
| | | T429E T432E S436E) HA-tag | | PSMB3; a-1 | A100 | | |
| 5575 | R9467 | HNF4A(1-474)(S87A) HA-tag | opt1 | HSD17B4/ | hSL- | 111 | 260 |
| | | | | PSMB3; a-1 | A100 | | |
| 5576 | R9468 | HNF4A(1-474)(S87E) HA-tag | opt1 | HSD17B4/ | hSL- | 112 | 261 |
| | | | | PSMB3; a-1 | A100 | | |
| 5577 | R9469 | HNF4A(1-474)(R2V) HA-tag | opt1 | HSD17B4/ | hSL- | 113 | 262 |
| | | | | PSMB3; a-1 | A100 | | |
| 5578 | R9470 | HNF4A(1-474)(R2V K5V) | opt1 | HSD17B4/ | hSL- | 114 | 263 |
| | | HA-tag | | PSMB3; a-1 | A100 | | |
| 5579 | R9471 | HNF4A(1-474)(K179R K180R) | opt1 | HSD17B4/ | hSL- | 115 | 264 |
| | | HA-tag | | PSMB3; a-1 | A100 | | |
| 5580 | R9472 | HNF4A(1-474)(K234R) HA-tag | opt1 | HSD17B4/ | hSL- | 116 | 265 |
| | | | | PSMB3; a-1 | A100 | | |
| 5581 | R9473 | HNF4A(1-474)(K300R) HA-tag | opt1 | HSD17B4/ | hSL- | 117 | 266 |
| | | | | PSMB3; a-1 | A100 | | |
| 5582 | R9474 | HNF4A(1-474)(K300R K307R | opt1 | HSD17B4/ | hSL- | 118 | 267 |
| | | K309R) HA-tag | | PSMB3; a-1 | A100 | | |
| 5583 | R9475 | HNF4A(1-474)(K447R K470R) | opt1 | HSD17B4/ | hSL- | 119 | 268 |
| | | HA-tag | | PSMB3; a-1 | A100 | | |
| 5584 | R9476 | HNF4A(1-474)(K458R) HA-tag | opt1 | HSD17B4/ | hSL- | 120 | 269 |
| | | | | PSMB3; a-1 | A100 | | |
| 5585 | R9477 | HNF4A(1-474)(K106R K108R | opt1 | HSD17B4/ | hSL- | 121 | 270 |
| | | K126R K127R) HA-tag | | PSMB3; a-1 | A100 | | |
| 5586 | R9480 | HNF4A(1-474)(E285Q) HA-tag | opt1 | HSD17B4/ | hSL- | 122 | 271 |
| | | | | PSMB3; a-1 | A100 | | |
| 5587 | R9481 | HNF4A(1-474)(T166E) HA-tag | opt1 | HSD17B4/ | hSL- | 123 | 272 |
| | | | | PSMB3; a-1 | A100 | | |
| 5588 | R9532 | HNF4A(1-474)(S148A) HA-tag | opt1 | HSD17B4/ | hSL- | 124 | 273 |
| | | | | PSMB3; a-1 | A100 | | |
| 5589 | R9533 | HNF4A(1-474)(S148E) HA-tag | opt1 | HSD17B4/ | hSL- | 125 | 274 |
| | | | | PSMB3; a-1 | A100 | | |
| 5590 | R9534 | HNF4A(1-474)(S183A) HA-tag | opt1 | HSD17B4/ | hSL- | 126 | 275 |
| | | | | PSMB3; a-1 | A100 | | |
| 5591 | R9535 | HNF4A(1-474)(S183E) HA-tag | opt1 | HSD17B4/ | hSL- | 127 | 276 |
| | | | | PSMB3; a-1 | A100 | | |
| 5592 | R9536 | HNF4A(1-474)(S461A) HA-tag | opt1 | HSD17B4/ | hSL- | 128 | 277 |
| | | | | PSMB3; a-1 | A100 | | |
| 5593 | R9537 | HNF4A(1-474)(S461E) HA-tag | opt1 | HSD17B4/ | hSL- | 129 | 278 |
| | | | | PSMB3; a-1 | A100 | | |
| 5594 | R9849 | HNF4A(1-474)(S142A S143A | opt1 | HSD17B4/ | hSL- | 130 | 279 |
| | | S313A) HA-tag | | PSMB3; a-1 | A100 | | |
| 5595 | R9850 | HNF4A(1-474)(S142A S143A | opt1 | HSD17B4/ | hSL- | 131 | 280 |
| | | S313A K458R) HA-tag | | PSMB3; a-1 | A100 | | |
| 5596 | R9851 | HNF4A(1-474)(S142A S143A | opt1 | HSD17B4/ | hSL- | 132 | 281 |
| | | S167A S313A S378A T429A | | PSMB3; a-1 | A100 | | |
| | | T432A S436A K458R) HA-tag | | | | | |
| 5597 | R9852 | HNF4A(1-474)(S142A S143A | opt1 | HSD17B4/ | hSL- | 133 | 282 |
| | | S148A S167A S313A S378A | | PSMB3; a-1 | A100 | | |
| | | T429A T432A S436A K458R) | | | | | |
| | | HA-tag | | | | | |
| 5598 | R9853 | HNF4A(1-474)(K106R K108R | opt1 | HSD17B4/ | hSL- | 134 | 283 |
| | | K126R K127R S142A S143A | | PSMB3; a-1 | A100 | | |
| | | S148A S167A S313A S378A | | | | | |
| | | T429A T432A S436A K458R) | | | | | |
| | | HA-tag | | | | | |
| 5599 | R9873 | HNF4A(1-474)(S142A S143A | opt1 | HSD17B4/ | hSL- | 135 | 284 |
| | | S148A T166A S167A S313A | | PSMB3; a-1 | A100 | | |
| | | S378A T429A T432A S436A | | | | | |
| | | K458R) HA-tag | | | | | |
| 5600 | R9874 | HNF4A(1-474)(S87A S142A | opt1 | HSD17B4/ | hSL- | 136 | 285 |
| | | S143A S148A T166A S167A | | PSMB3; a-1 | A100 | | |
| | | S313A S378A T429A T432A | | | | | |
| | | S436A K458R) HA-tag | | | | | |
| 5601 | R9875 | HNF4A(1-474)(S87A K106R | opt1 | HSD17B4/ | hSL- | 137 | 286 |
| | | K108R K126R K127R S142A | | PSMB3; a-1 | A100 | | |
| | | S143A S148A T166A S167A | | | | | |
| | | S313A S378A T429A T432A | | | | | |
| | | S436A K458R) HA-tag | | | | | |
| 5602 | R10313 | HNF4A(1-474)(S87A K106R | opt1 | HSD17B4/ | hSL- | 138 | 287 |
| | | K108R K126R K127R) HA-tag | | PSMB3; a-1 | A100 | | |
| 5603 | R10314 | HNF4A(1-474)(S87A K106R | opt1 | HSD17B4/ | hSL- | 139 | 288 |
| | | K108R K126R K127R S142A | | PSMB3; a-1 | A100 | | |
| | | S143A S148A T166A S167A | | | | | |
| | | S313A S378A T429A T432A | | | | | |
| | | S436A K458R S461A) HA-tag | | | | | |
| 5604 | R10315 | HNF4A(1-474)(S87A K106R | opt1 | HSD17B4/ | hSL- | 140 | 289 |
| | | K108R K126R K127R S142A | | PSMB3; a-1 | A100 | | |
| | | S143A S148A T166A S167A | | | | | |
| | | S313A S378A T429A T432A | | | | | |
| | | S436A K458R S461E) HA-tag | | | | | |
| 5605 | R10316 | HNF4A(1-474)(S87A K106R | opt1 | HSD17B4/ | hSL- | 141 | 290 |
| | | K108R K126R K127R S142A | | PSMB3; a-1 | A100 | | |
| | | S143A S148E T166A S167E | | | | | |
| | | S313A S378E T429E T432E | | | | | |
| | | S436E K458R S461E) HA-tag | | | | | |
| 5606 | R10317 | HNF4A(1-474)(S87A K106R | opt1 | HSD17B4/ | hSL- | 142 | 291 |
| | | K108R K126R K127R S142A | | PSMB3; a-1 | A100 | | |
| | | S143A S148A T166A S167A | | | | | |
| | | K179R K180R S313A S378A | | | | | |
| | | T429A T432A S436A K458R | | | | | |
| | | S461E) HA-tag | | | | | |
| 5607 | R10318 | HNF4A(1-474)(S87A K106R | opt1 | HSD17B4/ | hSL- | 143 | 292 |
| | | K108R K126R K127R S142A | | PSMB3; a-1 | A100 | | |
| | | S143A S148A T166A S167A | | | | | |
| | | K300R K307R K309R S313A | | | | | |
| | | S378A T429A T432A S436A | | | | | |
| | | K458R S461E) HA-tag | | | | | |
| 5608 | R10319 | HNF4A(1-474)(S87A K106R | opt1 | HSD17B4/ | hSL- | 144 | 293 |
| | | K108R K126R K127R S142A | | PSMB3; a-1 | A100 | | |
| | | S143A S148A T166A S167A | | | | | |
| | | S313A S378A T429A T432A | | | | | |
| | | S436A K447R K458R S461E | | | | | |
| | | K470R) HA-tag | | | | | |
| 5609 | R10320 | HNF4A(1-474)(S87A K106R | opt1 | HSD17B4/ | hSL- | 145 | 294 |
| | | K108R K126R K127R S142A | | PSMB3; a-1 | A100 | | |
| | | S143A S148A T166A S167A | | | | | |
| | | K179R K180R K234R K300R | | | | | |
| | | K307R K309R S313A S378A | | | | | |
| | | T429A T432A S436A K447R | | | | | |
| | | K458R S461E K470R) HA-tag | | | | | |
| 5610 | R10321 | HNF4A(1-474)(S87A S95A | opt1 | HSD17B4/ | hSL- | 146 | 295 |
| | | S99A K106R K108R K126R | | PSMB3; a-1 | A100 | | |
| | | K127R S142A S143A S148A | | | | | |
| | | T166A S167A S313A S378A | | | | | |
| | | T429A T432A S436A K458R | | | | | |
| | | S461E) HA-tag | | | | | |
| 5611 | R10322 | HNF4A(1-474)(S87A K106R | opt1 | HSD17B4/ | hSL- | 147 | 296 |
| | | K108R K126R K127R S138A | | PSMB3; a-1 | A100 | | |
| | | T139A S142A S143A S148A | | | | | |
| | | T166A S167A S313A S378A | | | | | |
| | | T429A T432A S436A K458R | | | | | |
| | | S461E) HA-tag | | | | | |
| 5612 | R10323 | HNF4A(1-474)(S87A S95A | opt1 | HSD17B4/ | hSL- | 148 | 297 |
| | | S99A K106R K108R K126R | | PSMB3; a-1 | A100 | | |
| | | K127R S138A T139A S142A | | | | | |
| | | S143A S148A T166A S167A | | | | | |
| | | S313A S378A T429A T432A | | | | | |
| | | S436A K458R S461E) HA-tag | | | | | |
| 5613 | R9478 | HNF4A(1-375) HA-tag | opt1 | HSD17B4/ | hSL- | 149 | 692 |
| | | | | PSMB3; a-1 | A100 | | |
| 5614 | R9479 | HNF4A(39-474) HA-tag | opt1 | HSD17B4/ | hSL- | 198 | 1135 |
| | | | | PSMB3; a-1 | A100 | | |
| 5615 | R9697 | HNF4A(1-474) HA-tag | opt29 | HSD17B4/ | hSL- | 100 | 641 |
| | | | | PSMB3; a-1 | A100 | | |
| 5713 | R9011 | EGFP-3xHA | | HSD17B4/ | hSL- | | |
| | | | | PSMB3; a-1 | A100 | | |
| 5714 | R7797 | ZsGreen | | HSD17B4/ | A64- | | |
| | | | | PSMB3; a-1 | N5 | | |
| 5715 | R7805 | HNF4A(1-474) | opt29 | HSD17B4/ | A64- | 100 | 641 |
| | | | | PSMB3; a-1 | N5 | | |
| 2907 | R10692 | HNF4A(1-474) | opt1 | HSD17B4/ | hSL- | 100 | 249 |
| | | | | PSMB3; a-1 | A100 | | |
| 2936, | R11470 | HNF4A(1-474)(S461E) | opt1 | HSD17B4/ | hSL- | 129 | 278, 327 |
| 2985 | | | | PSMB3; a-1 | A100 | | |
| 2944 | R10596 | HNF4A(1-474) | opt1 | HSD17B4/ | hSL- | 137 | 286 |
| | | (S87A_K106R_K108R_K126R_{—} | | PSMB3; a-1 | A100 | | |
| | | K127R_S142A_S143A_S148A_{—} | | | | | |
| | | T166A_S167A_S313A_S378A_{—} | | | | | |
| | | T429A_T432A_S436A_K458R) | | | | | |
| 2945 | R10664 | HNF4A(1-474) | opt1 | HSD17B4/ | hSL- | 138 | 287 |
| | | (S87A_K106R_K108R_K126R_{—} | | PSMB3; a-1 | A100 | | |
| | | K127R) | | | | | |
| 2947 | R10665 | HNF4A(1-474) | opt1 | HSD17B4/ | hSL- | 140 | 289 |
| | | (S87A_K106R_K108R_K126R_{—} | | PSMB3; a-1 | A100 | | |
| | | K127R_S142A_S143A_S148A_{—} | | | | | |
| | | T166A_S167A_S313A_S378A_{—} | | | | | |
| | | T429A_T432A_S436A_K458R_{—} | | | | | |
| | | S461E) | | | | | |
| 5719 | R11540 | HNF4A(1-474) | opt1 | HSD17B4/ | hSL- | 100 | 249 |
| | | | | PSMB3; a-1 | A100 | | |
| 5720 | R11542 | HNF4A(1-474)(S461E) | opt1 | HSD17B4/ | hSL- | 129 | 278, 327 |
| | | | | PSMB3; a-1 | A100 | | |
| 5721 | R11541 | HNF4A(1-474) | opt1 | HSD17B4/ | hSL- | 140 | 289 |
| | | (S87A_K106R_K108R_K126R_{—} | | PSMB3; a-1 | A100 | | |
| | | K127R_S142A_S143A_S148A_{—} | | | | | |
| | | T166A_S167A_S313A_S378A_{—} | | | | | |
| | | T429A_T432A_S436A_K458R_{—} | | | | | |
| | | S461E) | | | | | |
| 5722 | R11554 | HNF4A(1-474) | opt1 | HSD17B4/ | hSL- | 100 | 249 |
| | * | | | PSMB3; a-1 | A100 | | |
| 5723 | R11556 | HNF4A(1-474)(S461E) | opt1 | HSD17B4/ | hSL- | 129 | 278, 327 |
| | * | | | PSMB3; a-1 | A100 | | |
| 5724 | R11555 | HNF4A(1-474) | opt1 | HSD17B4/ | hSL- | 140 | 289 |
| | * | (S87A_K106R_K108R_K126R_{—} | | PSMB3; a-1 | A100 | | |
| | | K127R_S142A_S143A_S148A_{—} | | | | | |
| | | T166A_S167A_S313A_S378A_{—} | | | | | |
| | | T429A_T432A_S436A_K458R_{—} | | | | | |
| | | S461E) | | | | | |
| 5695 | R11128 | HNF1A(1-631) | opt1 | HSD17B4/ | hSL- | 5672 | 5675 |
| | | | | PSMB3; a-1 | A100 | | |

* mRNA with N1-methylpseudouridine (N1MPU, m1f)

Constructs as shown in Table C2 “Constructs of the invention” which comprised a HA-tag for antibody detection in the experimental section naturally will not comprise a HA-tag when they are used in a therapeutic mRNA applicable for humans. Thus, those sequences without HA-tag are understood to be preferably comprised within the disclosure of the current invention.

In another embodiment, the constructs as described above may comprise the CDS as disclosed in patent application WO2018104538 under SEQ ID NO:44, 45, 85, 113, 141, 169, 197, 225, 253, 281, 309, 2011, 337, 365, 393, 420, 421, 458, 483, 511, 536, 561, 586, 611, 636, 661, 686, 711, 2025, 2067, 2089, 2090, 2127, 2152, 2177, 2202, 2227, 2252, 2277, 2302, 2327, 2352, 2377, or 2402; WO2018104538 which is herein incorporated by reference in its entirety, also especially SEQ ID NO:44, 45, 85, 113, 141, 169, 197, 225, 253, 281, 309, 2011, 337, 365, 393, 420, 421, 458, 483, 511, 536, 561, 586, 611, 636, 661, 686, 711, 2025, 2067, 2089, 2090, 2127, 2152, 2177, 2202, 2227, 2252, 2277, 2302, 2327, 2352, 2377, or 2402.

Further preferred constructs of the invention are disclosed herein in Table C3. As apparent, each line as shown in Table C3 resembles specific preferred mRNA(s) encoding a specific preferred HNF4A protein(s).

TABLE C3
“Further preferred constructs of the invention”: designation of combinations of amino acid substitutions
or deletions (for further information Table B-I shows further exemplary suitable HNF4A protein designs
including mRNAs encoding said proteins including their SEQ ID NOs) - “*” indicates an mRNA
with N1-methylpseudouridine (N1MPU, mlf), “**” indicates an mRNA with pseudouridine (psi-uridine, p)

| | | | Exemplary | Exemplary |
|---|---|---|---|---|
| | | | preferred | preferred |
| | amino acid substitutions or deletions in | PRT SEQ | CDS SEQ | mRNA SEQ |
| Designation | comparison to HNF4A WT protein | ID NO: | ID NO: | ID NO: |
| WT | — | 100 | 249 | 2907, 5719, |
| | | | | 5722*, 5725** |
| S87A | S87A | 111 | 260 | 2918 |
| S461E | S461E | 129 | 278 | 2936, 5720, |
| | | | | 5723*, 5726** |
| combo 1 | S142A S143A S313A | 130 | 279 | 2937 |
| combo 2 | S142A S143A S313A K458R | 131 | 280 | 2938 |
| combo 3 | S142A S143A S167A S313A S378A T429A | 132 | 281 | 2939 |
| | T432A S436A K458R | | | |
| combo 4 | S142A S143A S148A S167A S313A S378A | 133 | 282 | 2940 |
| | T429A T432A S436A K458R | | | |
| combo 5 | K106R K108R K126R K127R S142A S143A | 134 | 283 | 2941 |
| | S148A S167A S313A S378A T429A T432A | | | |
| | S436A K458R | | | |
| combo 6 | S142A S143A S148A T166A S167A S313A | 135 | 284 | 2942 |
| | S378A T429A T432A S436A K458R | | | |
| combo 7 | S87A S142A S143A S148A T166A S167A S313A | 136 | 285 | 2943 |
| | S378A T429A T432A S436A K458R | | | |
| combo 8 | S87A K106R K108R K126R K127R S142A S143A | 137 | 286 | 2944 |
| | S148A T166A S167A S313A S378A T429A | | | |
| | T432A S436A K458R | | | |
| combo 9 (S87A | S87A K106R K108R K126R K127R | 138 | 287 | 2945 |
| 4x K to R) | | | | |
| combo 10 (combo | S87A K106R K108R K126R K127R S142A S143A | 139 | 288 | 2946 |
| 8 + S461A) | S148A T166A S167A S313A S378A T429A | | | |
| | T432A S436A K458R S461A | | | |
| combo 11 (combo | S87A K106R K108R K126R K127R S142A S143A | 140 | 289 | 2947, 5721, |
| 8 + S461E) | S148A T166A S167A S313A S378A T429A | | | 5724*, 5727** |
| | T432A S436A K458R S461E | | | |
| combo 12 | S87A K106R K108R K126R K127R S142A S143A | 141 | 290 | 2948 |
| | S148E T166A S167E S313A S378E T429E T432E | | | |
| | S436E K458R S461E | | | |
| combo 13 | S87A K106R K108R K126R K127R S142A S143A | 142 | 291 | 2949 |
| | S148A T166A S167A K179R K180R S313A | | | |
| | S378A T429A T432A S436A K458R S461E | | | |
| combo 14 | S87A K106R K108R K126R K127R S142A S143A | 143 | 292 | 2950 |
| | S148A T166A S167A K300R K307R K309R | | | |
| | S313A S378A T429A T432A S436A K458R | | | |
| | S461E | | | |
| combo 15 | S87A K106R K108R K126R K127R S142A S143A | 144 | 293 | 2951 |
| | S148A T166A S167A S313A S378A T429A | | | |
| | T432A S436A K447R K458R S461E K470R | | | |
| combo 16 | S87A K106R K108R K126R K127R S142A S143A | 145 | 294 | 2952 |
| | S148A T166A S167A K179R K180R K234R | | | |
| | K300R K307R K309R S313A S378A T429A | | | |
| | T432A S436A K447R K458R S461E K470R | | | |
| combo 17 | S87A S95A S99A K106R K108R K126R K127R | 146 | 295 | 2953 |
| | S142A S143A S148A T166A S167A S313A | | | |
| | S378A T429A T432A S436A K458R S461E | | | |
| combo 18 | S87A K106R K108R K126R K127R S138A T139A | 147 | 296 | 2954 |
| | S142A S143A S148A T166A S167A S313A | | | |
| | S378A T429A T432A S436A K458R S461E | | | |
| combo 19 | S87A S95A S99A K106R K108R K126R K127R | 148 | 297 | 2955 |
| | S138A T139A S142A S143A S148A T166A | | | |
| | S167A S313A S378A T429A T432A S436A | | | |
| | K458R S461E | | | |

Accordingly, a preferred mRNA of the invention is SEQ ID NO:2907 (WNT HNF4A protein). Another preferred mRNA of the invention is SEQ ID NO:5719 (WT HNF4A protein). Another preferred mRNA of the invention is SEQ ID NO:5722 (WT HNF4A protein). Another preferred mRNA of the invention is SEQ ID NO:5725 (WT HNF4A protein).

In a preferred embodiment, the mRNA encoding “combo 11” is SEQ ID NO:2947. In another preferred embodiment, the mRNA encoding “combo 11” is SEQ ID NO:5721. In another preferred embodiment, the mRNA encoding “combo 11” is SEQ ID NO:5724. In another preferred embodiment, the mRNA encoding “combo 11” is SEQ ID NO:5727.

In another preferred embodiment, the mRNA encoding “S461E” is SEQ ID NO:2936. In another preferred embodiment, the mRNA encoding “S461E” is SEQ ID NO:5720. In another preferred embodiment, the mRNA encoding “S461E” is SEQ ID NO:5723. In another preferred embodiment, the mRNA encoding “S461E” is SEQ ID NO:5726.

### Modifications

### Chemical Modifications

The term “RNA modification” as used herein may refer to chemical modifications comprising backbone modifications as well as sugar modifications or base modifications.

In this context, a modified RNA as defined herein may contain nucleotide analogues/modifications, e.g. backbone modifications, sugar modifications or base modifications. A backbone modification in connection with the present invention is a modification, in which phosphates of the backbone of the nucleotides contained in an RNA as defined herein are chemically modified. A sugar modification in connection with the present invention is a chemical modification of the sugar of the nucleotides of the mRNA as defined herein. Furthermore, a base modification in connection with the present invention is a chemical modification of the base moiety of the nucleotides of the mRNA. In this context, nucleotide analogues or modifications are preferably selected from nucleotide analogues, which are applicable for transcription and/or translation.

### Base Modifications

In certain embodiments, the open reading frame of the mRNA of the invention does not comprise any chemically modified uracil or cytosine nucleotides.

In other embodiments, the mRNA of the invention is chemically modified, preferably wherein the mRNA comprises pseudouridine (psi-uridine), N1-methylpseudouridine (N1MPU), 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosme, 7-deazaguanosine, 8-oxoadenosine, 8-oxoguanosine, O(6)-methylguanine, and/or 2-thiocytidine, more preferably wherein all uridine bases of the mRNA are fully chemically modified, even more preferably wherein all uridine bases of the mRNA are pseudouridine or N1-methylpseudouridine (N1MPU) bases, most preferably wherein all uridine bases of the mRNA are N1-methylpseudouridine (N1MPU) bases.

In some embodiments of the invention, essentially all, e.g. essentially 100% of the uracil in the coding sequence (or the full nucleic acid sequence) have a chemical modification, preferably a chemical modification in the 5-position of the uracil. Incorporating modified nucleotides such as e.g. pseudouridine (ψ) or N1-methylpseudouridine (m1ψ or N1MPU) into the coding sequence (or the full nucleic acid sequence) may be advantageous as unwanted innate immune responses (upon administration of the RNA) may be adjusted or reduced (if required). In other preferred embodiments of the invention, essentially all uracil in the coding sequence or the full nucleic acid sequence are exchanged with pseudouridine (ψ). In some preferred embodiments of the invention, essentially all, e.g. essentially 100% of the uracil in the coding sequence or the full nucleic acid sequence are exchanged with N1-methylpseudourdine (m1P).

In other preferred embodiments, the artificial nucleic add, preferably the RNA, does not comprise chemically modified nucleotides. Notably, a 5′-cap structure as defined below is typically not considered to be a chemically modified nucleotide. Accordingly, the artificial nucleic add, preferably the RNA, comprises a sequence that consists only of G, C, A and U nucleotides and therefore does not comprise modified nucleotides, and optionally comprises a 5′-cap structure. In further preferred embodiments, the artificial nucleic acid, preferably the RNA of the invention does not comprise N1-methylpseudouridine (m1ψ) substituted positions or pseudouridine (ψ) substituted positions.

In some preferred embodiments, the nucleotide mixture comprises at least one modified nucleotide and/or at least one nucleotide analogue or nucleotide derivative. The modified nucleosides and nucleotides, which may be incorporated into a modified RNA as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group. In preferred embodiments the nucleotide mixture comprises least one modified nucleotide and/or at least one nucleotide analogues is selected from a backbone modified nucleotide, a sugar modified nucleotide and/or a base modified nucleotide, or any combination thereof.

In particularly preferred embodiments of the present invention, the nucleotide analogues/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further specific embodiments, a modified RNA may comprise nucleoside modifications selected from N1-methyl-pseudouridine (N1MPU), 6-aza-cytidine, 2-thio-cytidine, α-thio-cytidine, Pseudo-iso-cytidine, 5-aminoallyl-uridine, 5-iodo-uridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine.

In other preferred embodiments, the open reading frame from any mRNA of the invention does not comprise any chemically modified nucleotides, more preferably does not comprise any chemically modified uracil or cytosine nucleotides.

### Sugar Modifications:

The modified nucleosides and nucleotides, which may be incorporated into a modified RNA as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R=H, alkyl, cycloalkyl, aryl, aralkyl, heteroaryl or sugar); polyethyleneglycols (PEG), —O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar group can also contain one or more carbons that possess the opposite stereochemical configuration than that of the corresponding carbon in ribose. Thus, a modified RNA can include nucleotides containing, for instance, arabinose as the sugar.

### Backbone Modifications:

The phosphate backbone may further be modified in the modified nucleosides and nucleotides, which may be incorporated into a modified RNA as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

### Lipid Modification

According to a further embodiment, a modified RNA as defined herein can contain a lipid modification. Such a lipid-modified RNA typically comprises an RNA as defined herein. Such a lipid-modified RNA as defined herein typically further comprises at least one linker covalently linked with that RNA, and at least one lipid covalently linked with the respective linker. Alternatively, the lipid-modified RNA comprises at least one RNA as defined herein and at least one (bifunctional) lipid covalently linked (without a linker) with that RNA. According to a third alternative, the lipid-modified RNA comprises an RNA molecule as defined herein, at least one linker covalently linked with that RNA, and at least one lipid covalently linked with the respective linker, and also at least one (bifunctional) lipid covalently linked (without a linker) with that RNA. In this context, it is particularly preferred that the lipid modification is present at the terminal ends of a linear RNA sequence.

### G/C Content Modification

According to another embodiment, the mRNA of the present invention, may be modified, and thus stabilized, by modifying the guanosine/cytosine (G/C) content of the mRNA, preferably of the at least one coding sequence of the mRNA of the present invention.

In a particularly preferred embodiment of the present invention, the G/C content of the coding sequence (coding region or CDS) of the mRNA of the present invention is modified, particularly increased, compared to the G/C content of the coding region of the respective wild type RNA, i.e. the unmodified RNA. The amino acid sequence encoded by the mRNA is preferably not modified as compared to the amino acid sequence encoded by the respective wild type RNA. This modification of the mRNA of the present invention is based on the fact that the sequence of any RNA region to be translated is important for efficient translation of that RNA. Thus, the pharmaceutical composition of the mRNA and the sequence of various nucleotides are important. In particular, sequences having an increased G (guanosine)/C (cytosine) content are more stable than sequences having an increased A (adenosine)/U (uracil) content. According to the invention, the codons of the mRNA are therefore varied compared to the respective wild type RNA, while retaining the translated amino acid sequence, such that they include an increased amount of G/C nucleotides. In respect to the fact that several codons code for one and the same amino acid (so-called degeneration of the genetic code), the most favourable codons for the stability can be determined (so-called alternative codon usage). Depending on the amino acid to be encoded by the mRNA, there are various possibilities for modification of the mRNA sequence, compared to its wild type sequence. In the case of amino acids, which are encoded by codons, which contain exclusively G or C nucleotides, no modification of the codon is necessary. Thus, the codons for Pro (CCC or CCG), Arg (CGC or CGG), Ala (GCC or GCG) and Gly (GGC or GGG) require no modification, since no A or U is present. In contrast, codons which contain A and/or U nucleotides can be modified by substitution of other codons, which code for the same amino acids but contain no A and/or U. Examples of these are: the codons for Pro can be modified from CCU or CCA to CCC or CCG; the codons for Arg can be modified from CGU or CGA or AGA or AGG to CGC or CGG; the codons for Ala can be modified from GCU or GCA to GCC or GCG; the codons for Gly can be modified from GGU or GGA to GGC or GGG. In other cases, although A or U nucleotides cannot be eliminated from the codons, it is however possible to decrease the A and U content by using codons which contain a lower content of A and/or U nucleotides. Examples of these are: the codons for Phe can be modified from UUU to UUC; the codons for Leu can be modified from UUA, UUG, CUU or CUA to CUC or CUG; the codons for Ser can be modified from UCU or UCA or AGU to UCC, UCG or AGC; the codon for Tyr can be modified from UAU to UAC; the codon for Cys can be modified from UGU to UGC; the codon for His can be modified from CAU to CAC; the codon for GIn can be modified from CAA to CAG; the codons for Ile can be modified from AUU or AUA to AUC; the codons for Thr can be modified from ACU or ACA to ACC or ACG; the codon for Asn can be modified from AAU to AAC; the codon for Lys can be modified from AAA to AAG; the codons for Val can be modified from GUU or GUA to GUC or GUG; the codon for Asp can be modified from GAU to GAC; the codon for Glu can be modified from GAA to GAG; the stop codon UAA can be modified to UAG or UGA. In the case of the codons for Met (AUG) and Trp (UGG), on the other hand, there is no possibility of sequence modification. The substitutions listed above can be used either individually or in all possible combinations to increase the G/C content of the at least one mRNA of the pharmaceutical composition of the present invention compared to its particular wild type mRNA (i.e. the original sequence). Thus, for example, all codons for Thr occurring in the wild type sequence can be modified to ACC (or ACG). Preferably, however, for example, combinations of the above substitution possibilities are used:
substitution of all codons coding for Thr in the original sequence (wild type mRNA) to ACC (or ACG) and/or
substitution of all codons originally coding for Ser to UCC (or UCG or AGC); and/or
substitution of all codons coding for Ile in the original sequence to AUC; and/or
substitution of all codons originally coding for Lys to AAG and/or
substitution of all codons originally coding for Tyr to UAC; and/or
substitution of all codons coding for Val in the original sequence to GUC (or GUG) and/or
substitution of all codons originally coding for Glu to GAG and/or
substitution of all codons originally coding for Ala to GCC (or GCG) and/or
substitution of all codons originally coding for Arg to CGC (or CGG); and/or
substitution of all codons coding for Val in the original sequence to GUC (or GUG) and/or
substitution of all codons originally coding for Glu to GAG and/or
substitution of all codons originally coding for Ala to GCC (or GCG); and/or
substitution of all codons originally coding for Gly to GGC (or GGG); and/or
substitution of all codons originally coding for Asn to AAC; and/or
substitution of all codons coding for Val in the original sequence to GUC (or GUG) and/or
substitution of all codons originally coding for Phe to UUC and/or
substitution of all codons originally coding for Cys to UGC and/or
substitution of all codons originally coding for Leu to CUG (or CUC) and/or
substitution of all codons originally coding for Gin to CAG and/or
substitution of all codons originally coding for Pro to CCC (or CCG); etc.

Preferably, the G/C content of the coding region of the mRNA of the present invention is increased by at least 7%, more preferably by at least 15%, particularly preferably by at least 20%, compared to the G/C content of the coding region of the wild type RNA, which codes for a peptide or protein as defined herein or a fragment or variant thereof. According to a specific embodiment at least 5%, 10%, 20%, 30%, 40%, 50%, 60%, more preferably at least 70%, even more preferably at least 80% and most preferably at least 90%, 95% or even 100% of the substitutable codons in the region coding for a peptide or protein as defined herein or a fragment or variant thereof or the whole sequence of the wild type RNA sequence are substituted, thereby increasing the GC/content of said sequence. In this context, it is particularly preferable to increase the G/C content of the mRNA of the present invention, preferably of the at least one coding region of the mRNA according to the invention, to the maximum (i.e. 100% of the substitutable codons) as compared to the wild type sequence. According to the invention, a further preferred modification of the mRNA of the present invention is based on the finding that the translation efficiency is also determined by a different frequency in the occurrence of tRNAs in cells. Thus, if so-called “rare codons” are present in the mRNA of the present invention to an increased extent, the corresponding modified RNA sequence is translated to a significantly poorer degree than in the case where codons coding for relatively “frequent” tRNAs are present. According to the invention, in the modified RNA of the present invention, the region which codes for a peptide or protein as defined herein or a fragment or variant thereof is modified compared to the corresponding region of the wild type RNA such that at least one codon of the wild type sequence, which codes for a tRNA which is relatively rare in the cell, is exchanged for a codon, which codes for a tRNA which is relatively frequent in the cell and carries the same amino acid as the relatively rare tRNA. By this modification, the sequences of the mRNA of the present invention is modified such that codons for which frequently occurring tRNAs are available are inserted. In other words, according to the invention, by this modification all codons of the wild type sequence, which code for a tRNA which is relatively rare in the cell, can in each case be exchanged for a codon, which codes for a tRNA which is relatively frequent in the cell and which, in each case, carries the same amino acid as the relatively rare tRNA. Which tRNAs occur relatively frequently in the cell and which, in contrast, occur relatively rarely is known to a person skilled in the art; cf. e.g. Akashi, Curr. Opin. Genet. Dev. 2001, 11(6): 660-666. The codons, which use for the particular amino acid the tRNA which occurs the most frequently, e.g. the Gly codon, which uses the tRNA, which occurs the most frequently in the (human) cell, are particularly preferred. According to the invention, it is particularly preferable to link the sequential G/C content which is increased, in particular maximized, in the modified RNA of the present invention, with the “frequent” codons without modifying the amino acid sequence of the peptide or protein encoded by the coding region of the mRNA. This preferred embodiment allows provision of a particularly efficiently translated and stabilized (modified) RNA of the present invention. The determination of a modified RNA of the present invention as described above (increased G/C content; exchange of tRNAs) can be carried out using the computer program explained in WO2002098443—the disclosure content of which is included in its full scope in the present invention. Using this computer program, the nucleotide sequence of any desired RNA can be modified with the aid of the genetic code or the degenerative nature thereof such that a maximum G/C content results, in combination with the use of codons which code for tRNAs occurring as frequently as possible in the cell, the amino acid sequence coded by the modified RNA preferably not being modified compared to the non-modified sequence. Alternatively, it is also possible to modify only the G/C content or only the codon usage compared to the original sequence. The source code in Visual Basic 6.0 (development environment used: Microsoft Visual Studio Enterprise 6.0 with Servicepack 3) is also described in WO2002098443. In a further preferred embodiment of the present invention, the A/U content in the environment of the ribosome binding site of the mRNA of the present invention is increased compared to the A/U content in the environment of the ribosome binding site of its respective wild type mRNA. This modification (an increased A/U content around the ribosome binding site) increases the efficiency of ribosome binding to the mRNA. An effective binding of the ribosomes to the ribosome binding site (Kozak sequence=ACC; the AUG forms the start codon) in turn has the effect of an efficient translation of the mRNA. Accordingly, in one embodiment, the coding RNA comprises a ribosome binding site, also referred to as “Kozak sequence” identical to or at least 80%, 85%, 90%, 95% identical to SEQ ID NO:59, SEQ ID NO:60 or the sequence “ACC”. According to a further embodiment of the present invention, the mRNA of the present invention may be modified with respect to potentially destabilizing sequence elements. Particularly, the coding region and/or the 5′- and/or 3′-untranslated region of this RNA may be modified compared to the respective wild type RNA such that it contains no destabilizing sequence elements, the encoded amino acid sequence of the modified RNA preferably not being modified compared to its respective wild type RNA. It is known that, for example in sequences of eukaryotic RNAs, destabilizing sequence elements (DSE) occur, to which signal proteins bind and regulate enzymatic degradation of RNA in vivo. For further stabilization of the modified RNA, optionally in the region which encodes a peptide or protein as defined herein or a fragment or variant thereof, one or more such modifications compared to the corresponding region of the wild type RNA can therefore be carried out, so that no or substantially no destabilizing sequence elements are contained there. According to the invention, DSE present in the untranslated regions (3′- and/or 5′-UTR) can also be eliminated from the mRNA of the present invention by such modifications. Such destabilizing sequences are e.g. AU-rich sequences (AURES), which occur in 3′-UTR sections of numerous unstable RNAs (Caput et al., Proc. Natl. Acad. Sci. USA 1986, 83: 1670 to 1674). The mRNA of the present invention is therefore preferably modified compared to the respective wild type RNA such that the mRNA of the present invention contains no such destabilizing sequences. This also applies to those sequence motifs which are recognized by possible endonucleases, e.g. the sequence GAACAAG, which is contained in the 3′-UTR segment of the gene encoding the transferrin receptor (Binder et al., EMBO J. 1994, 13: 1969-1980). These sequence motifs are also preferably removed in the mRNA of the present invention.

In preferred embodiments, the nucleic acid may be modified, wherein the G/C content of the at least one coding sequence may be optimized compared to the G/C content of the corresponding wild type or reference coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the nucleic acid is preferably not modified as compared to the amino acid sequence encoded by the respective wild type or reference coding sequence. The generation of a G/C content optimized nucleic acid sequence (RNA or DNA) may be carried out using a method according to WO2002098443. In this context, the disclosure of WO2002/098443 is included in its full scope in the present invention. Throughout the description, including the <223> identifier of the sequence listing, G/C optimized coding sequences are indicated by the abbreviations “opt1” or “gc” [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)].

### Sequences Adapted to Human Codon Usage

According to the invention, a further preferred modification of the mRNA of the present invention is based on the finding that codons encoding the same amino acid typically occur at different frequencies. According to the invention, in the modified RNA of the present invention, the coding sequence (coding region) as defined herein is preferably modified compared to the corresponding region of the respective wild type RNA such that the frequency of the codons encoding the same amino acid corresponds to the naturally occurring frequency of that codon according to the human codon usage as e.g. shown in Table D.

For example, in the case of the amino acid alanine (Ala) present in an amino acid sequence encoded by the at least one coding sequence of the mRNA according to the invention, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table D).

TABLE D
Human codon usage table

| Amino acid | codon | fraction | /1000 |
|---|---|---|---|
| Ala | GCG | 0.10 | 7.4 |
| Ala | GCA | 0.22 | 15.8 |
| Ala | GCT | 0.28 | 18.5 |
| Ala | GCC* | 0.40 | 27.7 |
| Cys | TGT | 0.42 | 10.6 |
| Cys | TGC* | 0.58 | 12.6 |
| Asp | GAT | 0.44 | 21.8 |
| Asp | GAC* | 0.56 | 25.1 |
| Glu | GAG* | 0.59 | 39.6 |
| Glu | GAA | 0.41 | 29.0 |
| Phe | TTT | 0.43 | 17.6 |
| Phe | TTC* | 0.57 | 20.3 |
| Gly | GGG | 0.23 | 16.5 |
| Gly | GGA | 0.26 | 16.5 |
| Gly | GGT | 0.18 | 10.8 |
| Gly | GGC* | 0.33 | 22.2 |
| His | CAT | 0.41 | 10.9 |
| His | CAC* | 0.59 | 15.1 |
| Ile | ATA | 0.14 | 7.5 |
| Ile | ATT | 0.35 | 16.0 |
| Ile | ATC* | 0.52 | 20.8 |
| Lys | AAG* | 0.60 | 31.9 |
| Lys | AAA | 0.40 | 24.4 |
| Leu | TTG | 0.12 | 12.9 |
| Leu | TTA | 0.06 | 7.7 |
| Leu | CTG* | 0.43 | 39.6 |
| Leu | CTA | 0.07 | 7.2 |
| Leu | CTT | 0.12 | 13.2 |
| Leu | CTC | 0.20 | 19.6 |
| Met | ATG* | 1 | 22.0 |
| Asn | AAT | 0.44 | 17.0 |
| Asn | AAC* | 0.56 | 19.1 |
| Pro | CCG | 0.11 | 6.9 |
| Pro | CCA | 0.27 | 16.9 |
| Pro | CCT | 0.29 | 17.5 |
| Pro | CCC* | 0.33 | 19.8 |
| Gln | CAG* | 0.73 | 34.2 |
| Gln | CAA | 0.27 | 12.3 |
| Arg | AGG | 0.22 | 12.0 |
| Arg | AGA* | 0.21 | 12.1 |
| Arg | CGG | 0.19 | 11.4 |
| Arg | CGA | 0.10 | 6.2 |
| Arg | CGT | 0.09 | 4.5 |
| Arg | CGC | 0.19 | 10.4 |
| Ser | AGT | 0.14 | 12.1 |
| Ser | AGC* | 0.25 | 19.5 |
| Ser | TCG | 0.06 | 4.4 |
| Ser | TCA | 0.15 | 12.2 |
| Ser | TCT | 0.18 | 15.2 |
| Ser | TCC | 0.23 | 17.7 |
| Thr | ACG | 0.12 | 6.1 |
| Thr | ACA | 0.27 | 15.1 |
| Thr | ACT | 0.23 | 13.1 |
| Thr | ACC* | 0.38 | 18.9 |
| Val | GTG* | 0.48 | 28.1 |
| Val | GTA | 0.10 | 7.1 |
| Val | GTT | 0.17 | 11.0 |
| Val | GTC | 0.25 | 14.5 |
| Trp | TGG* | 1 | 13.2 |
| Tyr | TAT | 0.42 | 12.2 |
| Tyr | TAC* | 0.58 | 15.3 |
| Stop | TGA* | 0.61 | 1.6 |
| Stop | TAG | 0.17 | 0.8 |
| Stop | TAA | 0.22 | 1.0 |

*: most frequent codon

In preferred embodiments, the nucleic acid may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the nucleic acid is preferably modified such that the frequency of the codons encoding the same amino acid corresponds to the naturally occurring frequency of that codon according to the human codon usage. For example, in the case of the amino acid Ala, the wild type or reference coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table D). Accordingly, such a procedure (as exemplified for Ala) is applied for each amino acid encoded by the coding sequence of the nucleic acid to obtain sequences adapted to human codon usage. Throughout the description, including the <223> identifier of the sequence listing, human codon usage adapted coding sequences are indicated by the abbreviation “opt3” or “human” [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)].

### Codon-Optimized Sequences

As described above it is preferred according to the invention, that all codons of the wild type sequence which code for a tRNA, which is relatively rare in the cell, are exchanged for a codon which codes for a tRNA, which is relatively frequent in the cell and which, in each case, carries the same amino acid as the relatively rare tRNA. Therefore it is particularly preferred that the most frequent codons are used for each encoded amino acid (see Table D, most frequent codons are marked with asterisks). Such an optimization procedure increases the codon adaptation index (CAI) and ultimately maximises the CAI. In the context of the invention, sequences with increased or maximized CAI are typically referred to as “codon-optimized” sequences and/or CAI increased and/or maximized sequences. According to a preferred embodiment, the mRNA of the present invention comprises at least one coding sequence, wherein the coding sequence is codon-optimized as described herein. More preferably, the codon adaptation index (CAI) of the at least one coding sequence is at least 0.5, at least 0.8, at least 0.9 or at least 0.95. Most preferably, the codon adaptation index (CAI) of the at least one coding sequence is 1.

For example, in the case of the amino acid alanine (Ala) present in the amino acid sequence encoded by the at least one coding sequence of the mRNA according to the invention, the wild type coding sequence is adapted in a way that the most frequent human codon “GCC” is always used for said amino acid, or for the amino acid Cysteine (Cys), the wild type sequence is adapted in a way that the most frequent human codon “TGC” is always used for said amino acid etc.

### C-Optimized Sequences

According to another embodiment, the mRNA of the present invention may be modified by modifying, preferably increasing, the cytosine (C) content of the mRNA, preferably of the coding region of the mRNA.

In a particularly preferred embodiment of the present invention, the C content of the coding region of the mRNA of the present invention is modified, preferably increased, compared to the C content of the coding region of the respective wild type RNA, i.e. the unmodified RNA. The amino acid sequence encoded by the at least one coding sequence of the mRNA of the present invention is preferably not modified as compared to the amino acid sequence encoded by the respective wild type mRNA.

In a preferred embodiment of the present invention, the modified RNA is modified such that at least 10%, 20%, 30%, 40%, 50%, 60%, 70% or 80%, or at least 90% of the theoretically possible maximum cytosine-content or even a maximum cytosine-content is achieved.

In further preferred embodiments, at least 10%, 20%, 30%, 40%, 50%, 60%, 70%, 80%, 90% or even 100% of the codons of the target RNA wild type sequence, which are “cytosine content optimizable” are replaced by codons having a higher cytosine-content than the ones present in the wild type sequence.

In a further preferred embodiment, some of the codons of the wild type coding sequence may additionally be modified such that a codon for a relatively rare tRNA in the cell is exchanged by a codon for a relatively frequent tRNA in the cell, provided that the substituted codon for a relatively frequent tRNA carries the same amino acid as the relatively rare tRNA of the original wild type codon. Preferably, all of the codons for a relatively rare tRNA are replaced by a codon for a relatively frequent tRNA in the cell, except codons encoding amino acids, which are exclusively encoded by codons not containing any cytosine, or except for glutamine (Gln), which is encoded by two codons each containing the same number of cytosines.

In a further preferred embodiment of the present invention, the modified target RNA is modified such that at least 80%, or at least 90% of the theoretically possible maximum cytosine-content or even a maximum cytosine-content is achieved by means of codons, which code for relatively frequent tRNAs in the cell, wherein the amino acid sequence remains unchanged.

Due to the naturally occurring degeneracy of the genetic code, more than one codon may encode a particular amino acid. Accordingly, 18 out of 20 naturally occurring amino acids are encoded by more than one codon (with Tryp and Met being an exception), e.g. by 2 codons (e.g. Cys, Asp, Glu), by three codons (e.g. Ile), by 4 codons (e.g. Al, Gly, Pro) or by 6 codons (e.g. Leu, Arg, Ser). However, not all codons encoding the same amino acid are utilized with the same frequency under in vivo conditions. Depending on each single organism, a typical codon usage profile is established.

The term “cytosine content-optimizable codon” as used within the context of the present invention refers to codons, which exhibit a lower content of cytosines than other codons encoding the same amino acid. Accordingly, any wild type codon, which may be replaced by another codon encoding the same amino acid and exhibiting a higher number of cytosines within that codon, is considered to be cytosine-optimizable (C-optimizable). Any such substitution of a C-optimizable wild type codon by the specific C-optimized codon within a wild type coding region increases its overall C-content and reflects a C-enriched modified mRNA sequence. According to a preferred embodiment, the mRNA of the present invention, preferably the at least one coding sequence of the mRNA of the present invention, comprises or consists of a C-maximized RNA sequence containing C-optimized codons for all potentially C-optimizable codons. Accordingly, 100% or all of the theoretically replaceable C-optimizable codons are preferably replaced by C-optimized codons over the entire length of the coding region.

In this context, cytosine-content optimizable codons are codons, which contain a lower number of cytosines than other codons coding for the same amino acid.

Any of the codons GCG, GCA, GCU codes for the amino acid Ala, which may be exchanged by the codon GCC encoding the same amino acid, and/or
the codon UGU that codes for Cys may be exchanged by the codon UGC encoding the same amino acid, and/or
the codon GAU which codes for Asp may be exchanged by the codon GAC encoding the same amino acid, and/or
the codon that UUU that codes for Phe may be exchanged for the codon UUC encoding the same amino acid, and/or
any of the codons GGG, GGA, GGU that code Gly may be exchanged by the codon GGC encoding the same amino acid, and/or
the codon CAU that codes for His may be exchanged by the codon CAC encoding the same amino acid, and/or
any of the codons AUA, AUU that code for Ile may be exchanged by the codon AUC, and/or
any of the codons UUG, UUA, CUG, CUA, CUU coding for Leu may be exchanged by the codon CUC encoding the same amino acid, and/or
the codon AAU that codes for Asn may be exchanged by the codon AAC encoding the same amino acid, and/or
any of the codons CCG, CCA, CCU coding for Pro may be exchanged by the codon CCC encoding the same amino acid, and/or
any of the codons AGG, AGA, CGG, CGA, CGU coding for Arg may be exchanged by the codon CGC encoding the same amino acid, and/or
any of the codons AGU, AGC, UCG, UCA, UCU coding for Ser may be exchanged by the codon UCC encoding the same amino acid, and/or
any of the codons ACG, ACA, ACU coding for Thr may be exchanged by the codon ACC encoding the same amino acid, and/or
any of the codons GUG, GUA, GUU coding for Val may be exchanged by the codon GUC encoding the same amino acid, and/or
the codon UAU coding for Tyr may be exchanged by the codon UAC encoding the same amino acid.

In any of the above instances, the number of cytosines is increased by 1 per exchanged codon. Exchange of all non C-optimized codons (corresponding to C-optimizable codons) of the coding region results in a C-maximized coding sequence. In the context of the invention, at least 70%, preferably at least 80%, more preferably at least 90%, of the non C-optimized codons within the at least one coding region of the mRNA according to the invention are replaced by C-optimized codons.

It may be preferred that for some amino acids the percentage of C-optimizable codons replaced by C-optimized codons is less than 70%, while for other amino acids the percentage of replaced codons is higher than 70% to meet the overall percentage of C-optimization of at least 70% of all C-optimizable wild type codons of the coding region.

Preferably, in a C-optimized RNA of the invention, at least 50% of the C-optimizable wild type codons for any given amino acid are replaced by C-optimized codons, e.g. any modified C-enriched RNA preferably contains at least 50% C-optimized codons at C-optimizable wild type codon positions encoding any one of the above mentioned amino acids Ala, Cys, Asp, Phe, Gly, His, Ile, Leu, Asn, Pro, Arg, Ser, Thr, Val and Tyr, preferably at least 60%.

In this context codons encoding amino acids, which are not cytosine content-optimizable and which are, however, encoded by at least two codons, may be used without any further selection process. However, the codon of the wild type sequence that codes for a relatively rare tRNA in the cell, e.g. a human cell, may be exchanged for a codon that codes for a relatively frequent tRNA in the cell, wherein both code for the same amino acid. Accordingly, the relatively rare codon GAA coding for Glu may be exchanged by the relative frequent codon GAG coding for the same amino acid, and/or
the relatively rare codon AAA coding for Lys may be exchanged by the relative frequent codon AAG coding for the same amino acid, and/or
the relatively rare codon CAA coding for Gln may be exchanged for the relative frequent codon CAG encoding the same amino acid.

In this context, the amino acids Met (AUG) and Trp (UGG), which are encoded by only one codon each, remain unchanged. Stop codons are not cytosine-content optimized, however, the relatively rare stop codons amber, ochre (UAA, UAG) may be exchanged by the relatively frequent stop codon opal (UGA).

The single substitutions listed above may be used individually as well as in all possible combinations in order to optimize the cytosine-content of the modified RNA compared to the wild type mRNA sequence.

Accordingly, the at least one coding sequence as defined herein may be changed compared to the coding region of the respective wild type RNA in such a way that an amino acid encoded by at least two or more codons, of which one comprises one additional cytosine, such a codon may be exchanged by the C-optimized codon comprising one additional cytosine, wherein the amino acid is preferably unaltered compared to the wild type sequence.

According to a particularly preferred embodiment, the invention provides an mRNA, comprising at least one coding sequence as defined herein, wherein the G/C content of the at least one coding sequence of the mRNA is increased compared to the G/C content of the corresponding coding sequence of the corresponding wild type RNA, and/or wherein the C content of the at least one coding sequence of the mRNA is increased compared to the C content of the corresponding coding sequence of the corresponding wild type RNA, and/or
wherein the codons in the at least one coding sequence of the mRNA are adapted to human codon usage, wherein the codon adaptation index (CAI) is preferably increased or maximised in the at least one coding sequence of the mRNA,
and wherein the amino acid sequence encoded by the mRNA is preferably not being modified compared to the amino acid sequence encoded by the corresponding wild type RNA.

### 5′-Cap

According to another preferred embodiment of the invention, a modified RNA as defined herein, can be modified by the addition of a so-called “5′-cap” structure, which preferably stabilizes the mRNA as described herein. A 5′-cap is an entity, typically a modified nucleotide entity, which generally “caps” the 5′-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an mRNA. m7GpppN is the 5′-cap structure, which naturally occurs in mRNA transcribed by polymerase II and is therefore preferably not considered as modification comprised in a modified mRNA in this context. Accordingly, a modified RNA of the present invention may comprise a m7GpppN as 5′-cap, but additionally the modified RNA typically comprises at least one further modification as defined herein.

Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-cap structures are regarded as at least one modification in this context.

Particularly preferred modified 5′-cap structures are cap1 (methylation of the ribose of the adjacent nucleotide of m7G), cap2 (additional methylation of the ribose of the 2nd nucleotide downstream of the m7G), cap3 (additional methylation of the ribose of the 3rd nucleotide downstream of the m7G), cap4 (methylation of the ribose of the 4th nucleotide downstream of the m7G), ARCA (anti-reverse cap analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine. Accordingly, the mRNA according to the invention preferably comprises a 5′-cap structure.

In a preferred embodiment, the mRNA according to the invention comprises at least one 5′- or 3′-UTR element. In this context, an UTR element comprises or consists of a nucleic acid sequence, which is derived from the 5′- or 3′-UTR of any naturally occurring gene or which is derived from a fragment, a homolog or a variant of the 5′- or 3′-UTR of a gene. Preferably, the 5′- or 3′-UTR element used according to the present invention is heterologous to the at least one coding sequence of the mRNA of the invention. Even if 5′- or 3′-UTR elements derived from naturally occurring genes are preferred, also synthetically engineered UTR elements may be used in the context of the present invention.

The term “3′-UTR element” typically refers to a nucleic acid sequence, which comprises or consists of a nucleic acid sequence that is derived from a 3′-UTR or from a variant of a 3′-UTR. A 3′-UTR element in the sense of the present invention may represent the 3′-UTR of an RNA, preferably an mRNA. Thus, in the sense of the present invention, preferably, a 3′-UTR element may be the 3′-UTR of an RNA, preferably of an mRNA, or it may be the transcription template for a 3′-UTR of an RNA. Thus, a 3′-UTR element preferably is a nucleic acid sequence which corresponds to the 3′-UTR of an RNA, preferably to the 3′-UTR of an mRNA, such as an mRNA obtained by transcription of a genetically engineered vector construct. Preferably, the 3′-UTR element fulfils the function of a 3′-UTR or encodes a sequence which fulfils the function of a 3′-UTR.

According to a preferred embodiment, CleanCap© Reagent AG from TriLink is used as co-transcriptional capping reagent for in vitro transcription of 5′-capped mRNA resulting in a cap1 structure. CleanCap AG requires an AG initiator and use yields in a naturally occurring cap1 structure.

In preferred embodiments, the artificial nucleic acid, preferably the RNA, comprises a 5′-cap structure.

Such a 5′-cap structure suitably stabilizes the nucleic acid and/or enhances expression of the encoded transcription factor inhibitor and/or reduces the stimulation of the innate immune system after administration.

Accordingly, in preferred embodiments, the artificial nucleic acid, preferably the RNA, comprises a 5′-cap structure, preferably m7G, cap0, cap1, cap2, a modified cap0 or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. Preferably, the 5′-cap structure is connected via a 5′-5′-triphosphate linkage to the RNA.

5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Suitably, a 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of an RNA molecule when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG; m7,2′dGpppG; m7,3′OmeGpppG; m7,3′dGpppG and their tetraphosphate derivatives). Further suitable cap analogues are described in WO2008016473, WO2008157688, WO2009149253, WO2011015347, WO2013059475, WO2017066793, WO2017066781, WO2017066791, WO2017066789, WO2017053297, WO2017066782, WO2018075827 and WO2017066797, the disclosures referring to cap analogues herewith incorporated by reference.

In embodiments, a cap1 structure is generated using tri-nucleotide cap analogue as disclosed in WO2017053297, WO2017066793, WO2017066781, WO2017066791, WO2017066789, WO2017066782, WO2018075827 and WO2017066797. Preferably, cap structures derivable from the structure disclosed in claim 1-5 of WO2017053297 may be suitably used to co-transcriptionally generate a cap1 structure. Further, any cap structures derivable from the structure defined in claim 1 or claim 21 of WO2018075827 may be suitably used to generate a cap1 structure.

In preferred embodiments, the 5′-cap structure may suitably be added co-transcriptionally using tri-nucleotide cap analogue as defined herein, preferably in an RNA in vitro transcription reaction as defined herein.

In particularly preferred embodiments, the artificial nucleic acid, preferably the RNA of the invention comprises a cap1 structure or a modified cap1 structure.

In preferred embodiments, the cap1 structure is formed via co-transcriptional capping using tri-nucleotide cap analogues m7G(5)ppp(5′)(2′OMeA)pG or m7G(5′ppp(5′)(2′OMeG)pG. A particularly preferred cap1 analog in that context is m7G(5)ppp(5)(2′OMeA)pG.

In other preferred embodiments, the cap1 structure is a modified cap1 structure and is formed using co-transcriptional capping using tri-nucleotide cap analogue 3′OMe-m7G(5)ppp(5′)(2′OMeA)pG.

In other embodiments, the 5′-cap structure is formed via enzymatic capping using capping enzymes (e.g. vaccinia virus capping enzymes and/or cap-dependent 2′-O methyltransferases) to generate cap0 or cap1 or cap2 structures. In that context, the 5′-cap structure (cap0 or cap1) may be added using immobilized capping enzymes and/or cap-dependent 2′-O methyltransferases using methods and means disclosed in published PCT patent application WO2016/193226.

In preferred embodiments, about 70%, 75%, 80%, 85%, 90%, 95% of the RNA (species) comprises a cap structure, preferably a cap1 structure as determined by a capping assay.

For determining the presence or absence of a cap structure, capping assays as described in published PCT application WO2015101416, in particular, as described in claims 27 to 46 of published PCT application WO2015101416 can be used. Other capping assays that may be used to determine the presence or absence of a cap structure of an RNA are described in published PCT application WO2020127959.

According to a preferred embodiment, the mRNA according to the invention comprises a 5′-cap structure and/or at least one 3′-untranslated region element (3′-UTR element), preferably as defined herein. More preferably, the mRNA further comprises a 5′-UTR element as defined herein.

### UTRs

In a preferred embodiment, the pharmaceutical composition comprises an mRNA compound comprising at least one 5′- or 3′-UTR element. In this context, an UTR element comprises or consists of a nucleic acid sequence, which is derived from the 5′- or 3′-UTR of any naturally occurring gene or which is derived from a fragment, a homolog or a variant of the 5′- or 3′-UTR of a gene. Preferably, the 5′- or 3′-UTR element used according to the present invention is heterologous to the at least one coding region of the mRNA sequence of the invention. Even if 5′- or 3′-UTR elements derived from naturally occurring genes are preferred, also synthetically engineered UTR elements may be used in the context of the present invention.

The term “3′-UTR element” typically refers to a nucleic acid sequence, which comprises or consists of a nucleic acid sequence that is derived from a 3′-UTR or from a variant of a 3′-UTR. A 3′-UTR element in the sense of the present invention may represent the 3′-UTR of an RNA, preferably an mRNA. Thus, in the sense of the present invention, preferably, a 3′-UTR element may be the 3′-UTR of an RNA, preferably of an mRNA, or it may be the transcription template for a 3′-UTR of an RNA. Thus, a 3′-UTR element preferably is a nucleic acid sequence which corresponds to the 3′-UTR of an RNA, preferably to the 3′-UTR of an mRNA, such as an mRNA obtained by transcription of a genetically engineered vector construct. Preferably, the 3′-UTR element fulfils the function of a 3′-UTR or encodes a sequence which fulfils the function of a 3′-UTR.

Preferably, the at least one 3′-UTR element comprises or consists of a nucleic acid sequence derived from the 3′-UTR of a chordate gene, preferably a vertebrate gene, more preferably a mammalian gene, most preferably a human gene, or from a variant of the 3′-UTR of a chordate gene, preferably a vertebrate gene, more preferably a mammalian gene, most preferably a human gene.

Preferably, the pharmaceutical composition comprises an mRNA compound that comprises a 3′-UTR element, which may be derivable from a gene that relates to an mRNA with an enhanced half-life (that provides a stable mRNA), for example a 3′-UTR element as defined and described below. Preferably, the 3′-UTR element comprises or consists of a nucleic acid sequence derived from a 3′-UTR of a gene, which preferably encodes a stable mRNA, or from a homolog, a fragment or a variant of said gene.

In one preferred embodiment, the UTR-combinations which are disclosed in Table 1, claims 1 and claim 4, claims 6-8 and claim 9 of WO2019077001 are preferred UTR-combinations for mRNA compounds of the present invention. Further, preferably, the UTR-combinations as disclosed on page 24, second full paragraph after Table 1 and page 24, last paragraph to page 29, second paragraph of WO2019077001 are preferred UTR-combinations for mRNA compounds of the present invention. WO2019077001 is incorporated herein by reference in its entirety.

In a further preferred embodiment, that 3′-UTR element comprises or consists of a nucleic acid sequence which is derived from a 3′-UTR of a gene selected from the group consisting of a 3′-UTR of a gene selected from PSMB3 (see Table 1-5′-UTRs and 3′-UTRs herein below), ALB/albumin (see Table 1-5′-UTRs and 3′-UTRs herein below), alpha-globin (referred to as “muag” i.e. a mutated alpha-globin 3′-UTR; see Table 1-5′-UTRs and 3′-UTRs herein below), CASP1 (see Table 1-5′-UTRs and 3′-UTRs herein below), COX6B1 (see Table 1-5′-UTRs and 3′-UTRs herein below), GNAS (see Table 1-5′-UTRs and 3′-UTRs herein below), NDUFA1 (see Table 1-5′-UTRs and 3′-UTRs herein below) and RPS9 (see Table 1-5′-UTRs and 3′-UTRs herein below), or from a homolog, a fragment or a variant of any one of these genes (for example, human albumin/alb 3′-UTR as disclosed in SEQ ID NO:1369 of WO2013143700, which is incorporated herein by reference), or from a homolog, a fragment or a variant thereof. In a further preferred embodiment, the 3′-UTR element comprises the nucleic acid sequence derived from a fragment of the human albumin gene according to SEQ ID NO:1376 of WO2013143700 (albumin/alb 3′-UTR). In a further preferred embodiment, the 3′-UTR element comprises or consists of a nucleic acid sequence which is derived from a 3′-UTR of an albumin gene, preferably a vertebrate albumin gene, more preferably a mammalian albumin gene, most preferably a human albumin gene such as from the 3′-UTR of the human albumin gene according to GenBank Accession number NM_000477.5, or a fragment or variant thereof. In another preferred embodiment, the 3′-UTR element comprises or consists of the center, α-complex-binding portion of the 3′-UTR of an α-globin gene, such as of a human α-globin gene, or an α-complex-binding portion of the 3′-UTR of an α-globin gene (also named herein as “muag”), corresponding to SEQ ID NO:1393 of patent application WO2013143700.

TABLE 1
5′-UTRs and 3′-UTRs

| UTR | Name | Type | SEQ ID NO: | Type | SEQ ID NO: | Type | SEQ ID NO: |
|---|---|---|---|---|---|---|---|
| 5′-UTR | HSD17B4 | DNA | 1 | RNA | 2 | DNA, RNA | 1, 2 |
| 5′-UTR | RPL32_(32L4, 32L3) | DNA | 3 | RNA | 4 | DNA, RNA | 3, 4 |
| 5′-UTR | AIG1 | DNA | 5 | RNA | 6 | DNA, RNA | 5, 6 |
| 5′-UTR | ASAH1 | DNA | 7 | RNA | 8 | DNA, RNA | 7, 8 |
| 5′-UTR | ATP5A1 | DNA | 9 | RNA | 10 | DNA, RNA | 9, 10 |
| 5′-UTR | COX6C | DNA | 11 | RNA | 12 | DNA, RNA | 11, 12 |
| 5′-UTR | Dpysl2 | DNA | 13 | RNA | 14 | DNA, RNA | 13, 14 |
| 5′-UTR | MDR (ABCB7) | DNA | 15 | RNA | 16 | DNA, RNA | 15, 16 |
| 5′-UTR | Mp68_(2010107E04Rik) | DNA | 17 | RNA | 18 | DNA, RNA | 17, 18 |
| 5′-UTR | Ndufa4 | DNA | 19 | RNA | 20 | DNA, RNA | 19, 20 |
| 5′-UTR | Nosip | DNA | 21 | RNA | 22 | DNA, RNA | 21, 22 |
| 5′-UTR | Rpl31 | DNA | 23 | RNA | 24 | DNA, RNA | 23, 24 |
| 5′-UTR | RPL35A | DNA | 25 | RNA | 26 | DNA, RNA | 25, 26 |
| 5′-UTR | Slc7a3 | DNA | 27 | RNA | 28 | DNA, RNA | 27, 28 |
| 5′-UTR | TUBB4B | DNA | 29 | RNA | 30 | DNA, RNA | 29, 30 |
| 5′-UTR | Ubqln2 | DNA | 31 | RNA | 32 | DNA, RNA | 31, 32 |
| 3′-UTR | PSMB3 | DNA | 33 | RNA | 34 | DNA, RNA | 33, 34 |
| 3′-UTR | muag | DNA | 35 | RNA | 36 | DNA, RNA | 35, 36 |
| 3′-UTR | ALB7 | DNA | 37 | RNA | 38 | DNA, RNA | 37, 38 |
| 3′-UTR | ANXA4 | DNA | 39 | RNA | 40 | DNA, RNA | 39, 40 |
| 3′-UTR | CASP1 | DNA | 41 | RNA | 42 | DNA, RNA | 41, 42 |
| 3′-UTR | COX6B1 | DNA | 43 | RNA | 44 | DNA, RNA | 43, 44 |
| 3′-UTR | FIG4 | DNA | 45 | RNA | 46 | DNA, RNA | 45, 46 |
| 3′-UTR | Gnas | DNA | 47 | RNA | 48 | DNA, RNA | 47, 48 |
| 3′-UTR | Ndufa1 | DNA | 49 | RNA | 50 | DNA, RNA | 49, 50 |
| 3′-UTR | RPS9 | DNA | 51 | RNA | 52 | DNA, RNA | 51, 52 |
| 3′-UTR | Slc7a3 | DNA | 53 | RNA | 54 | DNA, RNA | 53, 54 |
| 3′-UTR | TUBB4B | DNA | 55 | RNA | 56 | DNA, RNA | 55, 56 |

In this context it is very preferred that the 3′-UTR element of the mRNA sequence according to the invention comprises or consists of a corresponding RNA sequence of the nucleic acid sequence according to SEQ ID NO:33 or 34, or a homolog, a fragment or variant thereof.

UTR-combination SLC7A3 (5′-UTR of mouse solute carrier family 7 (cationic amino acid transporter, y+ system), member 3)/PSMB3: in another preferred embodiment, the mRNA compound comprises a 5′-UTR element, which comprises or consists of a nucleic acid sequence which is derived from a cationic amino acid transporter 3 (solute carrier family 7 member 3, SLC7A3) gene, wherein said 5′-UTR element comprises or consists of a DNA sequence according to SEQ ID NO:15 as disclosed in WO2019077001 or respectively a RNA sequence according to SEQ ID 10 NO:16 as disclosed in WO2019077001. In another preferred embodiment, the mRNA compound comprises a 3′-UTR element, which comprises or consists of a nucleic acid sequence which is derived from a proteasome subunit beta type-3 (PSMB3) gene, wherein said 3′-UTR element comprises or consists of a DNA sequence according to SEQ ID NO:23 as disclosed in WO2019077001 or respectively a RNA sequence according to SEQ ID NO:24 as disclosed in WO2019077001. In further preferred embodiments, the mRNA compound comprises an UTR-combination as disclosed in WO2019077001, i.e. both a 5′-UTR element, which comprises or consists of a nucleic acid sequence which is derived from a SLC7A3 gene and a 3′-UTR element, which comprises or consists of a nucleic acid sequence which is derived from a PSMB3 gene. For direct reference to the sequence listing of the present invention, see Table 1-5′-UTRs and 3′-UTRs.

UTR-combination RPL31 (5′-UTR of mouse ribosomal protein L31)/RPS9 (3-UTR of human ribosomal protein S9 (RPS9): In another preferred embodiment, the mRNA compound comprises a 5′-UTR element, which comprises or consists of a nucleic acid sequence which is derived from a 60S ribosomal protein L31 (RPL31) gene, wherein said 5′-UTR element comprises or consists of a DNA sequence according to SEQ ID NO:13 as disclosed in WO2019077001 or respectively a RNA sequence according to SEQ ID NO:14 as disclosed in WO2019077001. In another preferred embodiment, the mRNA compound comprises a 3′-UTR element, which comprises or consists of a nucleic acid sequence which is derived from a 40S ribosomal protein S9 (RPS9) gene, wherein said 3′-UTR element comprises or consists of a DNA sequence according to SEQ ID NO:33 as disclosed in WO2019077001 or respectively a RNA sequence according to SEQ ID NO:34 as disclosed in WO2019077001. In further preferred embodiments, the mRNA compound comprises an UTR-combination as disclosed in WO2019077001, i.e. both a 5′-UTR element, which comprises or consists of a nucleic acid sequence which is derived from a RPL31 gene and a 3′-UTR element, which comprises or consists of a nucleic acid sequence which is derived from a RPS9 gene (preferably SEQ ID NO:51/52). For direct reference to the sequence listing of the present invention, see Table 1-5′-UTRs and 3′-UTRs.

In another preferred embodiment, the UTR-combination 5′-UTR Ubqln2 (ubiquilin 2, see Table 1-5′-UTRs and 3′-UTRs) and 3′-UTR Gnas (Guanine nucleotide-binding protein G(s) subunit alpha isoforms short, see Table 1-5′-UTRs and 3′-UTRs) is used.

In a very preferred embodiment, the 5′-UTR element of the mRNA sequence according to the invention comprises or consists of a corresponding RNA sequence of the nucleic acid sequence according to SEQ ID NO:1 or SEQ ID NO:2, i.e. HSD17B4. Also, in a very preferred embodiment, the 3′-UTR element of the mRNA sequence according to the invention comprises or consists of a corresponding RNA sequence of the nucleic acid sequence according to SEQ ID NO:33 or SEQ ID NO:34, i.e. PSMB3. In also a very preferred embodiment, the 5′-UTR element of the mRNA sequence and the 3′-UTR-element according to the invention comprises or consists of a combination of aforementioned HSD17B4 and PSMB3-UTRs.

The term “a nucleic acid sequence which is derived from a variant of the 3′-UTR of a [ . . . ] gene” preferably refers to a nucleic acid sequence, which is based on a variant of the 3′-UTR sequence of a gene, such as on a variant of the 3′-UTR of an albumin gene, an α-globin gene, a β-globin gene, a tyrosine hydroxylase gene, a lipoxygenase gene, or a collagen alpha gene, such as a collagen alpha 1(I) gene, or on a part thereof as described above. This term includes sequences corresponding to the entire sequence of the variant of the 3′-UTR of a gene, i.e. the full length variant 3′-UTR sequence of a gene, and sequences corresponding to a fragment of the variant 3′-UTR sequence of a gene. A fragment in this context preferably consists of a continuous stretch of nucleotides corresponding to a continuous stretch of nucleotides in the full-length variant 3′-UTR, which represents at least 20%, preferably at least 30%, more preferably at least 40%, more preferably at least 50%, even more preferably at least 60%, even more preferably at least 70%, even more preferably at least 80%, and most preferably at least 90% of the full-length variant 3′-UTR. Such a fragment of a variant, in the sense of the present invention, is preferably a functional fragment of a variant as described herein.

According to a preferred embodiment, the mRNA compound comprising an mRNA sequence according to the invention comprises a 5′-cap structure and/or at least one 3′-untranslated region element (3′-UTR element), preferably as defined herein. More preferably, the RNA further comprises a 5′-UTR element as defined herein.

In a further preferred embodiment, the pharmaceutical composition comprises an mRNA compound comprising at least one 5′-untranslated region element (5′-UTR element). Preferably, the at least one 5′-UTR element comprises or consists of a nucleic acid sequence, which is derived from the 5′-UTR of a TOP gene or which is derived from a fragment, homolog or variant of the 5′-UTR of a TOP gene. It is preferred that the 5′-UTR element does not comprise a TOP motif or a 5′-TOP, as defined above.

In some embodiments, the nucleic acid sequence of the 5′-UTR element, which is derived from a 5′-UTR of a TOP gene, terminates at its 3′-end with a nucleotide located at position 1, 2, 3, 4, 5, 6, 7, 8, 9 or 10 upstream of the start codon (e.g. A(U/T)G) of the gene or mRNA it is derived from. Thus, the 5′-UTR element does not comprise any part of the protein coding region. Thus, preferably, the only protein coding part of the at least one mRNA sequence is provided by the coding region.

The nucleic acid sequence derived from the 5′-UTR of a TOP gene is preferably derived from a eukaryotic TOP gene, preferably a plant or animal TOP gene, more preferably a chordate TOP gene, even more preferably a vertebrate TOP gene, most preferably a mammalian TOP gene, such as a human TOP gene.

For example, the 5′-UTR element may be selected from 5′-UTR elements comprising or consisting of a nucleic acid sequence, which is derived from a nucleic acid sequence selected from the group consisting of SEQ ID NO:1-1363, SEQ ID NO:1395, SEQ ID NO:1421 and SEQ ID NO:1422 of patent application WO2013143700, whose disclosure is incorporated herein by reference, from the homologs of SEQ ID NO:1-1363, SEQ ID NO:1395, SEQ ID NO:1421 and SEQ ID NO:1422 of patent application WO2013143700, from a variant thereof, or preferably from a corresponding RNA sequence. The term “homologs of SEQ ID NO:1-1363, SEQ ID NO:1395, SEQ ID NO:1421 and SEQ ID NO:1422 of patent application WO2013143700” refers to sequences of other species than Homo sapiens, which are homologous to the sequences according to SEQ ID NO:1-1363, SEQ ID NO:1395, SEQ ID NO:1421 and SEQ ID NO:1422 of patent application WO2013143700. For direct reference to the sequence listing of the present invention, see Table 1-5′-UTRs and 3′-UTRs.

In a preferred embodiment, the 5′-UTR element of the mRNA compound comprises or consists of a nucleic acid sequence, which is derived from a nucleic acid sequence extending from nucleotide position 5 (i.e. the nucleotide that is located at position 5 in the sequence) to the nucleotide position immediately 5′ to the start codon (located at the 3′-end of the sequences), e.g. the nucleotide position immediately 5′ to the ATG sequence, of a nucleic acid sequence selected from SEQ ID NO:1-1363, SEQ ID NO:1395, SEQ ID NO:1421 and SEQ ID NO:1422 of patent application WO2013143700, from the homologs of SEQ ID NO:1-1363, SEQ ID NO:1395, SEQ ID NO:1421 and SEQ ID NO:1422 of patent application WO2013143700 from a variant thereof, or a corresponding RNA sequence. It is particularly preferred that the 5′-UTR element is derived from a nucleic acid sequence extending from the nucleotide position immediately 3′ to the 5′-TOP to the nucleotide position immediately 5′ to the start codon (located at the 3′-end of the sequences), e.g. the nucleotide position immediately 5′ to the ATG sequence, of a nucleic acid sequence selected from SEQ ID NO:1-1363, SEQ ID NO:1395, SEQ ID NO:1421 and SEQ ID NO:1422 of patent application WO2013143700, from the homologs of SEQ ID NO:1-1363, SEQ ID NO:1395, SEQ ID NO:1421 and SEQ ID NO:1422 of patent application WO2013143700, from a variant thereof, or a corresponding RNA sequence. For direct reference to the sequence listing of the present invention, see Table 1-5′-UTRs and 3′-UTRs.

In a further preferred embodiment, the 5′-UTR element comprises or consists of a nucleic acid sequence, which is derived from a 5′-UTR of a TOP gene encoding a ribosomal protein or from a variant of a 5′-UTR of a TOP gene encoding a ribosomal protein. For example, the 5′-UTR element comprises or consists of a nucleic acid sequence, which is derived from a 5′-UTR of a nucleic acid sequence according to any of SEQ ID NO:67, 170, 193, 244, 259, 554, 650, 675, 700, 721, 913, 1016, 1063, 1120, 1138, and 1284-1360 of patent application WO2013143700, a corresponding RNA sequence, a homolog thereof, or a variant thereof as described herein, preferably lacking the 5′-TOP motif. As described above, the sequence extending from position 5 to the nucleotide immediately 5′ to the ATG (which is located at the 3′-end of the sequences) corresponds to the 5′-UTR of said sequences. For direct reference to the sequence listing of the present invention, see Table 1-5′-UTRs and 3′-UTRs.

In further preferred embodiments, the preferred 5′-UTR or 3′-UTR element comprises or consists of a nucleic acid sequence, which is disclosed in Table 1-5′-UTRs and 3′-UTRs.

Accordingly, in a preferred embodiment, the 5′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO:1368, or SEQ ID NO:1412-1420 of patent application WO2013143700, or a corresponding RNA sequence, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO:1368, or SEQ ID NO:1412-1420 of patent application WO2013143700, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

Preferably, the at least one 5′-UTR element and the at least one 3′-UTR element act synergistically to increase protein production from the at least one mRNA sequence as described above.

According to a preferred embodiment, the pharmaceutical composition of the invention comprises an mRNA compound that comprises, preferably in 5′- to 3′-direction:
a) a 5′-cap structure, preferably m7GpppN, more preferably cap1 or m7G(5′)ppp(5)(2′OMeA)pG;
b) optionally, a 5′-UTR element which preferably comprises or consists of a nucleic acid sequence which is optionally derived from the 5′-UTR of a TOP gene, more preferably comprising or consisting of the corresponding RNA sequence of a nucleic acid sequence according to any of the 5′-UTRs as disclosed in Table 1-5′-UTRs and 3′-UTRs, a homolog, a fragment or a variant thereof, most preferably according to SEQ ID NO:1 or 2 (HSD17B4), or a 5′-UTR element which preferably comprises or consists of a nucleic acid sequence which is derived from a solute carrier family 7 cationic amino acid transporter, y+ system), member 3 (SEQ ID NO:15 as disclosed in WO2019077001 or respectively a RNA sequence according to SEQ ID NO:16 as disclosed in WO2019077001) or a 60S ribosomal protein L31 (RPL31) gene (SEQ ID NO:13 as disclosed in WO2019077001 or respectively a RNA sequence according to SEQ ID NO:14 as disclosed in WO2019077001);
c) (i) at least one coding region encoding at least one engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100 as described herein above or below or as disclosed in the sequence listing of the present invention or (ii) at least one coding region encoding a wild type hepatocyte nuclear factor 4 alpha (HNF4A) or a fragment or variant thereof, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140;
d) optionally, a 3′-UTR element which preferably comprises or consists of a nucleic acid sequence which is derived from a gene providing a stable mRNA, preferably comprising or consisting of the corresponding RNA sequence of a nucleic acid sequence according to any of the 3′-UTRs as disclosed in Table 1-5′-UTRs and 3′-UTRs, most preferably according to SEQ ID NO:33 or 34 (PSMB3) or a 3′-UTR element which preferably comprises or consists of a nucleic acid sequence which is derived from a 40S ribosomal protein S9 (RPS9) gene (SEQ ID NO:33 as disclosed in WO2019077001 or respectively a RNA sequence according to SEQ ID NO:34 as disclosed in WO2019077001);
e) optionally, a histone stem-loop selected from SEQ ID NO:63 or 64; and/or
f) optionally, a poly(A) sequence preferably comprising 64 adenosines or 100 adenosines; and
g) optionally, a poly(C) sequence, preferably comprising 30 cytosines;
h) optionally, a histone stem-loop selected from SEQ ID NO:63 or 64; and/or
i) optionally, a 3′-terminal sequence element selected from SEQ ID NO:65-99.

According to one embodiment, the mRNA compound comprises an miRNA binding site located in the 5′ or 3′ UTR. A miRNA (microRNA) is typically a small, non-coding single stranded RNA molecules of about 20 to 25 nucleotides in length which may function in gene regulation, for example, but not limited to, by mRNA degradation or translation inhibition or repression. miRNAs are typically produced from hairpin precursor RNAs (pre-miRNAs), and they may form functional complexes with proteins. Furthermore, miRNAs may bind to 5′ and/or 3′-UTR regions of target mRNAs. Preferably, the microRNA binding site is for a microRNA selected from the group consisting of miR-126, miR-142, miR-144, miR-146, miR-150, miR-155, miR-16, miR-21, miR-223, miR-24, miR-27, miR-26a binding site, preferably a miR-122 or miR-142 binding site, or any combination of the aforementioned miRNA binding sites.

In one embodiment, the miRNA binding site is a naturally occurring miRNA binding site. In another embodiment, the miRNA binding site may be a mimetic, or a modification of a naturally-occurring miRNA binding site.

According to one preferred embodiment, the mRNA compound comprising an mRNA sequence according to the invention may further comprise, as defined herein:
a) a 5′-cap structure, preferably m7GpppN, more preferably cap1 or m7G(5)ppp(5′)(2′OMeA)pG;
b) at least one miRNA binding site, preferably wherein the microRNA binding site is selected from the group consisting of a miR-126, miR-142, miR-144, miR-146, miR-150, miR-155, miR-16, miR-21, miR-223, miR-24, miR-27, miR-26a binding site, preferably a miR-122 or miR-142 binding site, or any combination of the aforementioned miRNA binding sites;
c) at least one 5′-UTR element;
d) at least one 3′-UTR element;
e) at least one poly(A) sequence;
f) optionally at least one poly(C) sequence;
g) optionally, a histone stem-loop selected from SEQ ID NO:63 or 64;
h) optionally, a 3′-terminal sequence element selected from SEQ ID NO:65-99; or any combinations of these.

In some embodiments, the artificial nucleic acid molecule according to the invention may comprise UTR elements according to a-2 (NDUFA4/PSMB3); a-5 (MP68/PSMB3); c-1 (NDUFA4/RPS9); a-1 (HSD17B4/PSMB3); e-3 (MP68/RPS9); e-4 (NOSIP/RPS9); a-4 (NOSIP/PSMB3); e-2 (RPL31/RPS9); e-5 (ATP5A1/RPS9); d-4 (HSD17B4/NUDFA1); b-5 (NOSIP/COX6B1); a-3 (SLC7A3/PSMB3); b-1 (UBQLN2/RPS9); b-2 (ASAH1/RPS9); b-4 (HSD17B4/CASP1); e-6 (ATPSA1/COX6B1); b-3 (HSD17B4/RPS9); g-5 (RPL31/CASP1); h-1 (RPL31/COX6B1); and/or c-5 (ATP5A1/PSMB3) in accordance with the disclosure of WO2019077001, the aforementioned UTR combinations and the subject-matter of claim 4 and subject-matter related thereto in WO2019077001 is incorporated herein by reference.

### UTR-Combination HSD17B4/PSMB3:

In a very preferred embodiment, the 5′-UTR element of the mRNA sequence according to the invention comprises or consists of a corresponding RNA sequence of the nucleic acid sequence according to SEQ ID NO:1 or SEQ ID NO:2, i.e. HSD17B4. Also, in a very preferred embodiment, the 3′-UTR element of the mRNA sequence according to the invention comprises or consists of a corresponding RNA sequence of the nucleic acid sequence according to SEQ ID NO:33 or SEQ ID NO:34), i.e. PSMB3. In also a very preferred embodiment, the 5′-UTR element of the mRNA sequence and the 3′-UTR-element according to the invention comprises or consists of a combination of aforementioned HSD17B4 and PSMB3-UTRs.

### UTR-Combination Rpl31/RPS9:

In another preferred embodiment, the mRNA compound of the present invention comprises a 5′-UTR element, which comprises or consists of a nucleic acid sequence which is derived from a 60S ribosomal protein L31 (RPL31) gene, wherein said 5′-UTR element comprises or consists of a DNA sequence according to SEQ ID NO:13 as disclosed in WO2019077001A1 or respectively a RNA sequence according to SEQ ID NO:14 as disclosed in WO2019077001A1. In another preferred embodiment, the mRNA compound comprises a 3′-UTR element, which comprises or consists of a nucleic acid sequence which is derived from a 40S ribosomal protein S9 (RPS9) gene, wherein said 3′-UTR element comprises or consists of a DNA sequence according to SEQ ID NO:33 as disclosed in WO2019077001A1 or respectively a RNA sequence according to SEQ ID NO:34 as disclosed in WO2019077001A1. In further preferred embodiments, the mRNA compound comprises an UTR-combination as disclosed in WO2019077001A1, i.e. both a 5′-UTR element, which comprises or consists of a nucleic acid sequence which is derived from a RPL31 gene and a 3′-UTR element, which comprises or consists of a nucleic acid sequence which is derived from a RPS9 gene.

### UTR-Combination Slc7a3/PSMB3:

In another preferred embodiment, the mRNA compound of the present invention comprises a 5′-UTR element, which comprises or consists of a nucleic acid sequence which is derived from a cationic amino acid transporter 3 (solute carrier family 7 member 3, SLC7A3) gene, wherein said 5′-UTR element comprises or consists of a DNA sequence according to SEQ ID NO:15 as disclosed in WO2019077001A1 or respectively a RNA sequence according to SEQ ID NO:16 as disclosed in WO2019077001A1. In another preferred embodiment, the mRNA compound comprises a 3′-UTR element, which comprises or consists of a nucleic acid sequence which is derived from a proteasome subunit beta type-3 (PSMB3) gene, wherein said 3′-UTR element comprises or consists of a DNA sequence according to SEQ ID NO:23 as disclosed in WO2019077001A1 or respectively a RNA sequence according to SEQ ID NO:24 as disclosed in WO2019077001A1. In further preferred embodiments, the mRNA compound comprises an UTR-combination as disclosed in WO2019077001A1, i.e. both a 5′-UTR element, which comprises or consists of a nucleic acid sequence which is derived from a SLc7a3 gene and a 3′-UTR element, which comprises or consists of a nucleic acid sequence which is derived from a PSMB3 gene.

### Poly(A) Tail

According to a further preferred embodiment, the mRNA of the present invention may contain a poly(A) tail on the 3′-terminus of typically about 10 to 200 adenosine nucleotides, preferably about 10 to 100 adenosine nucleotides, more preferably about 40 to 80 adenosine nucleotides or even more preferably about 50 to 70 adenosine nucleotides, more preferably about 100 adenosine nucleotides. Preferably, the mRNA of the invention comprises a 3′-terminal A nucleotide.

Preferably, the poly(A) sequence in the mRNA of the present invention is derived from a DNA template by RNA in vitro transcription. Alternatively, the poly(A) sequence may also be obtained in vitro by common methods of chemical-synthesis without being necessarily transcribed from a DNA-progenitor. Moreover, poly(A) sequences, or poly(A) tails may be generated by enzymatic polyadenylation of the mRNA according to the present invention using commercially available polyadenylation kits and corresponding protocols known in the art.

Alternatively, the mRNA as described herein optionally comprises a polyadenylation signal for enzymatic polyadenylation, which is defined herein as a signal, which conveys polyadenylation to a (transcribed) RNA by specific protein factors (e.g. cleavage and polyadenylation specificity factor (CPSF), cleavage stimulation factor (CstF), cleavage factors I and II (CF I and CF II), poly(A) polymerase (PAP)). In this context, a consensus polyadenylation signal for enzymatic polyadenylation is preferred comprising the NN(U/T)ANA consensus sequence. In a particularly preferred aspect, the polyadenylation signal comprises one of the following sequences: AA(U/T)AAA or A(U/T)(U/T)AAA (wherein uridine is usually present in RNA and thymidine is usually present in DNA). It is clear for a skilled artisan, that said consensus sequence is not mandatory for enzymatic or non-enzymatic polyadenylation.

### Poly(C) Tail

According to a further preferred embodiment, the mRNA of the present invention may contain a poly(C) tail on the 3′-terminus of typically about 10 to 200 cytidine nucleotides, preferably about 10 to 100 cytidine nucleotides, more preferably about 20 to 70 cytidine nucleotides or even more preferably about 20 to 60 or even 10 to 40 cytidine nucleotides.

### Histone Stem-Loop

In a further preferred embodiment, the pharmaceutical composition comprises an mRNA compound comprising a histone stem-loop sequence/structure (HSL, hSL, histoneSL, preferably according to SEQ ID NO:3 or SEQ ID NO:4). In said embodiment, the mRNA sequence may comprise at least one (or more) histone stem loop sequence or structure. Such histone stem-loop sequences are preferably selected from histone stem-loop sequences as disclosed in WO2012019780, the disclosure of which is incorporated herewith by reference. A histone stem-loop sequence that may be used within the present invention may preferably be derived from formulae (I) or (II) of WO2012019780. According to a further preferred embodiment the coding RNA may comprise at least one histone stem-loop sequence derived from at least one of the specific formulae (Ia) or (IIa) of patent application WO2012019780. According to a further preferred embodiment the coding RNA may comprise at least one histone stem-loop sequence derived from a Histone stem-loop as disclosed in patent application WO2018104538 under formula (I), formula (II), formula (Ia) or on pages 49-52 under section “Histone stem-loop” and WO2018104538—SEQ ID NO:1451-1452 as disclosed in WO2018104538; WO2018104538A1 which is herein incorporated by reference in its entirety, also especially SEQ ID NO:1451-1452 (herein SEQ ID NO:63 or 64).

In particularly preferred embodiment, the RNA of the invention comprises at least one histone stem-loop sequence, wherein said histone stem-loop sequence comprises a nucleic acid sequence being identical or at least 70%, 80%, 90%, 91%, 92%, 93%, 94%, 95%, 96%, 97%, 98%, or 99% identical to SEQ ID NO:63 or 64, or fragments or variants thereof.

### Signal Peptide

According to another particularly preferred embodiment, the mRNA according to the invention may additionally or alternatively encode a secretory signal peptide. Such signal peptides are sequences, which typically exhibit a length of about 15 to 30 amino acids and are preferably located at the N-terminus of the encoded peptide, without being limited thereto. Signal peptides as defined herein preferably allow the transport of the peptide or protein as encoded by the at least one mRNA of the pharmaceutical composition into a defined cellular compartment, preferably the cell surface, the endoplasmic reticulum (ER) or the endosomal-lysosomal compartment. Examples of secretory signal peptide sequences as defined herein include, without being limited thereto, signal sequences of classical or non-classical MHC-molecules (e.g. signal sequences of MHC I and II molecules, e.g. of the MHC class I molecule HLA-A*0201), signal sequences of cytokines or immunoglobulins as defined herein, signal sequences of the invariant chain of immunoglobulins or antibodies as defined herein, signal sequences of Lamp1, Tapasin, Erp57, Calretikulin, Calnexin, and further membrane associated proteins or of proteins associated with the endoplasmic reticulum (ER) or the endosomal-lysosomal compartment. Most preferably, signal sequences of MHC class I molecule HLA-A*0201 may be used according to the present invention. For example, a signal peptide derived from HLA-A is preferably used in order to promote secretion of the encoded peptide or protein as defined herein or a fragment or variant thereof. More preferably, an HLA-A signal peptide is fused to an encoded peptide or protein as defined herein or to a fragment or variant thereof:

Any of the above modifications may be applied to the mRNA of the present invention, and further to any RNA as used in the context of the present invention and may be, if suitable or necessary, be combined with each other in any combination, provided, these combinations of modifications do not interfere with each other in the respective at least one mRNA. A person skilled in the art will be able to take his choice accordingly.

### Lipid-Based Carriers

In particularly preferred embodiments, the at least one artificial nucleic acid, preferably the at least one RNA of the pharmaceutical composition is formulated in lipid-based carriers.

In the context of the invention, the term “lipid-based carriers” encompass lipid-based delivery systems for nucleic acid (e.g. RNA) that comprise a lipid component. A lipid-based carrier may additionally comprise other components suitable for encapsulating/incorporating/complexing a nucleic acid (e.g. RNA) including a cationic or polycationic polymer, a cationic or polycationic polysaccharide, a cationic or polycationic protein, a cationic or polycationic peptide, or any combinations thereof.

In the context of the invention, a typical “lipid-based carrier” is selected from liposomes, lipid nanoparticles (LNPs), lipoplexes, solid lipid nanoparticles, and/or nanoliposomes. The nucleic acid, preferably the RNA of the pharmaceutical composition may completely or partially incorporated or encapsulated in a lipid-based carrier, wherein the nucleic acid (e.g. RNA) may be located in the interior space of the lipid-based carrier, within the lipid layer/membrane of the lipid-based carrier, or associated with the exterior surface of the lipid-based carrier. The incorporation of nucleic acid, preferably the RNA into lipid-based carriers may be referred to as “encapsulation”. A “lipid-based carrier” is not restricted to any particular morphology, and include any morphology generated when e.g. an aggregation reducing lipid and at least one further lipid are combined, e.g. in an aqueous environment in the presence of nucleic acid (e.g. RNA). For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the at least one nucleic acid (e.g. RNA) is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanoparticles (LNPs), a specific type of lipid-based carrier, are characterized as microscopic lipid particles having a solid core or partially solid core. Typically, an LNP does not comprise an interior aqua space sequestered from an outer medium by a bilayer. In an LNP, the at least one nucleic acid (e.g. RNA) may be encapsulated or incorporated in the lipid portion of the LNP enveloped by some or the entire lipid portion of the LNP. An LNP may comprise any lipid capable of forming a particle to which the nucleic acid (e.g. RNA) may be attached, or in which the nucleic acid may be encapsulated. Preferably, said lipid-based carriers are particularly suitable for ocular administration.

In preferred embodiments, the lipid-based carriers of the pharmaceutical composition are selected from liposomes, lipid nanoparticles, lipoplexes, solid lipid nanoparticles, lipo-polylexes, and/or nanoliposomes.

In preferred embodiments, the lipid-based carriers of the pharmaceutical composition are lipid nanoparticles (LNPs). In particularly preferred embodiments, the lipid nanoparticles of the pharmaceutical composition encapsulate the at least one nucleic acid, preferably the at least one RNA of the invention.

The term “encapsulated”, e.g. incorporated, complexed, encapsulated, partially encapsulated, associated, partially associated, refers to the essentially stable combination of nucleic acid, preferably RNA with one or more lipids into lipid-based carriers (e.g. larger complexes or assemblies) preferably without covalent binding of the nucleic acid. The lipid-based carriers—encapsulated nucleic acid (e.g. RNA) may be completely or partially located in the interior of the lipid-based carrier (e.g. the lipid portion and/or an interior space) and/or within the lipid layer/membrane of the lipid-based carriers. The encapsulation of an nucleic acid (e.g. RNA) into lipid-based carriers is also referred to herein as “incorporation” as the nucleic acid (e.g. RNA) is preferably contained within the interior of the lipid-based carriers. Without wishing to be bound to theory, the purpose of incorporating or encapsulating nucleic acid into lipid-based carriers may be to protect the nucleic acid from an environment which may contain enzymes, chemicals, or conditions that degrade the nucleic acid (e.g. RNA). Moreover, incorporating nucleic acid into lipid-based carriers may promote the uptake of the nucleic acid and their release from the endosomal compartment, and hence, may enhance the therapeutic effect of the nucleic acid (e.g. RNA) when administered to a cell or a subject.

In preferred embodiments, the lipid-based carriers of the pharmaceutical composition comprise at least one or more lipids selected from at least one aggregation-reducing lipid, at least one cationic lipid, at least one neutral lipid or phospholipid, or at least one steroid or steroid analog, or any combinations thereof.

In preferred embodiments, the lipid-based carriers of the pharmaceutical composition comprise (i) an aggregation-reducing lipid, (ii) a cationic lipid or ionizable lipid, and (iii) a neutral lipid/phospholipid or a steroid/steroid analog.

In particularly preferred embodiments, the lipid-based carriers of the pharmaceutical composition comprise an (i) aggregation-reducing lipid, (ii) a cationic lipid or ionizable lipid, (iii) a neutral lipid or phospholipid, (iv) and a steroid or steroid analog.

### Lipid Nanoparticles (LNPs)

Lipid nanoparticles as used herein preferably have the structure of a liposome. A liposome is a structure having lipid-containing membranes enclosing an aqueous interior. Liposomes preferably have one or more lipid membranes. Liposomes are preferably single-layered, referred to as unilamellar, or multi-layered, referred to as multilamellar. When complexed with nucleic acids, lipid particles may also be lipoplexes, which are composed of cationic lipid bilayers sandwiched between DNA layers. Liposomes can further be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposome design preferably includes, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety. The nucleic acid may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, the mRNA is preferably formulated in a cationic oil-in-water emulsion where the emulsion particle comprises an oil core and a cationic lipid which can interact with the polynucleotide anchoring the molecule to the emulsion particle (see International Pub. No. WO2012006380; herein incorporated by reference in its entirety). In one embodiment, the mRNA may be formulated in a water-in-oil emulsion comprising a continuous hydrophobic phase in which the hydrophilic phase is dispersed.

In one embodiment, the mRNA pharmaceutical compositions is formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In another embodiment, the lipid nanoparticles have a median diameter size of from about 50 nm to about 300 nm, such as from about 50 nm to about 250 nm, for example, from about 50 nm to about 200 nm.

In some embodiments, the mRNA is delivered using smaller LNPs. Such particles may comprise a diameter from below 0.1 μm up to 100 nm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm, In another embodiment, RNA is delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 nm to about 50 nm, from about 20 nm to about 50 nm, from about 30 nm to about 50 nm, from about 40 nm to about 50 nm, from about 20 nm to about 60 nm, from about 30 nm to about 60 nm, from about 40 nm to about 60 nm, from about 20 nm to about 70 nm, from about 30 nm to about 70 nm, from about 40 nm to about 70 nm, from about 50 nm to about 70 nm, from about 60 nm to about 70 nm, from about 20 nm to about 80 nm, from about 30 nm to about 80 nm, from about 40 nm to about 80 nm, from about 50 nm to about 80 nm, from about 60 nm to about 80 nm, from about 20 nm to about 90 nm, from about 30 nm to about 90 nm, from about 40 nm to about 90 nm, from about 50 nm to about 90 nm, from about 60 nm to about 90 nm and/or from about 70 nm to about 90 nm.

In one embodiment, the lipid nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In yet another embodiment, the lipid nanoparticles in the formulation of the present invention have a single mode particle size distribution (i.e., they are not bi- or poly-modal).

The lipid nanoparticles preferably further comprise one or more lipids and/or other components in addition to those mentioned above. Other lipids may be included in the liposome compositions for a variety of purposes, such as to prevent lipid oxidation or to attach ligands onto the liposome surface. Any of a number of lipids may be present in lipid particles, including amphipathic, neutral, cationic, and anionic lipids. Such lipids can be used alone or in combination.

Additional components that may be present in a lipid particle include bilayer stabilizing components such as polyamide oligomers (see, e.g., U.S. Pat. No. 6,320,017, which is incorporated by reference in its entirety), peptides, proteins, and detergents.

Different lipid nanoparticles having varying molar ratios of cationic lipid, non-cationic (or neutral) lipid, sterol (e.g., cholesterol), and aggregation reducing agent (such as a PEG-modified lipid) on a molar basis (based upon the total moles of lipid in the lipid nanoparticles) are provided in Table D herein below.

TABLE D
Exemplary lipid nanoparticle compositions

| | Molar Ratio of Lipids | | | |
|---|---|---|---|---|
| | (Based upon 100% total moles of lipid in the lipid nanoparticle) | | | |
| | | Non-Cationic | | |
| Formulation | Cationic | (or neutral) | | Aggregation Reducing Agent (e.g., |
| No. | Lipid | lipid | Sterol | conjugated lipid) |
| 1 | from about 35 | from about 3 to | from about 15 | from about 0.1 to about 10% (preferably |
| | to about 65% | about 12 or 15% | to about 45% | from about 0.5 to about 2 or 3%) |
| 2 | from about 20 | from about 5 | from about 20 | from about 0.1 to about 10% (preferably |
| | to about 70% | to about 45% | to about 55% | from about 0.5 to about 2 or 3%) |
| 3 | from about 45 | from about 5 | from about 25 | from about 0.1 to about 3% |
| | to about 65% | to about 10% | to about 40% | |
| 4 | from about 20 | from about 5 | from about 25 | from about 0.1 to about 5% (preferably |
| | to about 60% | to about 25% | to about 55% | from about 0.1 to about 3%) |
| 5 | about 40% | about 10% | about 40% | about 10% |
| 6 | about 35% | about 15% | about 40% | about 10% |
| 7 | about 52% | about 13% | about 30% | about 5% |
| 8 | about 50% | about 10% | about 38.5% | about 1.5% |

In one embodiment, the weight ratio of lipid to RNA is at least about 0.5:1, at least about 1:1, at least about 2:1, at least about 3:1, at least about 4:1, at least about 5:1, at least about 6:1, at least about 7:1, at least about 11:1, at least about 20:1, at least about 25:1, at least about 27:1, at least about 30:1, or at least about 33:1. In one embodiment, the weight ratio of lipid to RNA is from about 1:1 to about 35:1, about 3:1 to about 15:1, about 4:1 to about 15:1, or about 5:1 to about 13:1 or about 25:1 to about 33:1. In one embodiment, the weight ratio of lipid to RNA is from about 0.5:1 to about 12:1.

In one embodiment, the mRNA of the present invention may be encapsulated in a therapeutic nanoparticle, referred to herein as “therapeutic nanoparticle nucleic acids”. Therapeutic nanoparticles may be formulated by methods described herein and known in the art such as, but not limited to, International Pub Nos. WO2010/005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, the contents of which is herein incorporated by reference in its entirety.

In one embodiment, the mRNA according to the invention may be encapsulated in, linked to and/or associated with synthetic nanocarriers. Synthetic nanocarriers include, but are not limited to, those described in International Pub. Nos. WO2010005740, WO2010030763, WO2012135010, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US2012244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, and WO2010030763 and WO2012135010 and US Pub. Nos. US20110262491, US20100104645, US20100087337 and US2012244222, each of which is herein incorporated by reference in their entirety. In another embodiment, the synthetic nanocarrier formulations may be lyophilized by methods described in International Pub. No. WO2011072218 and U.S. Pat. No. 8,211,473; the content of each of which is herein incorporated by reference in their entirety. In yet another embodiment, formulations of the present invention, including, but not limited to, synthetic nanocarriers, may be lyophilized or reconstituted by the methods described in US Patent Publication No. US20130230568, the contents of which are herein incorporated by reference in its entirety.

In one embodiment, the mRNA of the invention is formulated for delivery using the drug encapsulating microspheres described in International Patent Publication No. WO2013063468 or U.S. Pat. No. 8,440,614, each of which is herein incorporated by reference in its entirety.

According to preferred embodiments, the mRNA according to the invention may be formulated in order to target a specific tissue or organ. In particular, the mRNA according to the invention or a pharmaceutical carrier formulated together with the mRNA preferably forms a conjugate with a targeting group. Said targeting group preferably targets the conjugate, preferably the conjugate comprising the mRNA, to a specific tissue or organ. In other words, by conjugating the mRNA according to the invention with a target group (either directly by forming an RNA-target group conjugate or indirectly by forming a conjugate of a target group of a pharmaceutical carrier that is present in a complex with the mRNA according to the invention), the mRNA is delivered to a specific tissue or organ due to the targeting of said target group to that specific tissue or organ. Most preferably, the targeting group provides for delivery to liver tissue, preferably to liver macrophages, hepatocytes and or liver sinusoidal endothelial cells (LSEC). In this context, a targeting group is preferably selected from the group consisting of folate, GalNAc, galactose, mannose, mannose-6P, aptamers, integrin receptor ligands, chemokine receptor ligands, transferrin, biotin, serotonin receptor ligands, PSMA, endothelin, GCPII, somatostatin, LDL ligands and HDL ligands. Suitable approaches for targeted delivery to the liver, which may be applied to the mRNA of the invention, are also described in Bartneck et al. (Bartneck et al.: Therapeutic targeting of liver inflammation and fibrosis by nanomedicine. Hepatobiliary Surgery and Nutrition 2014; 3(6):364-376), the disclosure of which is incorporated herein in its entirety.

In another embodiment, liposomes or LNPs may be formulated for targeted delivery. Preferably, the liposome or LNP is formulated for targeted delivery of the mRNA according to the invention to the liver, preferably to liver macrophages, hepatocytes and/or liver sinusoidal endothelial cells (LSEC). The liposome or LNP used for targeted delivery may include, but is not limited to, the liposomes or LNPs described herein.

The mRNA of the invention preferably forms conjugates, such as RNA covalently linked to a carrier or targeting group, preferably as described herein, in order to provide for targeted delivery. Alternatively, the mRNA according to the invention may encode a fusion protein, e.g. bearing a targeting group fused to the peptide or protein as described herein, which is then targeted to a specific tissue or organ, preferably to the liver.

The conjugates preferably include targeting groups, e.g., a cell or tissue targeting agent, e.g., a lectin, glycoprotein, lipid or protein, e.g., an antibody, that binds to a specified cell type such as a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups may further be proteins, e.g., glycoproteins, or peptides, e.g., molecules having a specific affinity for a co-ligand, or antibodies e.g., an antibody, that binds to a specified cell type such as a liver cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, or an activator of p38 MAP kinase.

The targeting group is preferably a ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. Preferably, the aptamer is unmodified or has any combination of modifications disclosed herein.

In a preferred embodiment, the pharmaceutical composition according to the invention comprises the mRNA according to the invention that is formulated together with a cationic or polycationic compound and/or with a polymeric carrier. Accordingly, in a further embodiment of the invention, it is preferred that the mRNA as defined herein or any other nucleic acid comprised in the inventive pharmaceutical composition is associated with or complexed with a cationic or polycationic compound or a polymeric carrier, optionally in a weight ratio selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w) of mRNA or nucleic acid to cationic or polycationic compound and/or with a polymeric carrier; or optionally in a nitrogen/phosphate (N/P) ratio of mRNA or nucleic acid to cationic or polycationic compound and/or polymeric carrier in the range of about 0.1-10, preferably in a range of about 0.3-4 or 0.3-1, and most preferably in a range of about 0.5-1 or 0.7-1, and even most preferably in a range of about 0.3-0.9 or 0.5-0.9. More preferably, the N/P ratio of the mRNA to the one or more polycations is in the range of about 0.1 to 10, including a range of about 0.3 to 4, of about 0.5 to 2, of about 0.7 to 2 and of about 0.7 to 1.5.

Therein, the mRNA as defined herein or any other nucleic acid comprised in the pharmaceutical composition according to the invention can also be associated with a vehicle, transfection or complexation agent for increasing the transfection efficiency and/or the expression of the mRNA according to the invention or of optionally comprised further included nucleic acids.

Cationic or polycationic compounds, being particularly preferred agents in this context include protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, or histones. More preferably, the mRNA according to the invention is complexed with one or more polycations, preferably with protamine or oligofectamine, most preferably with protamine. In this context protamine is particularly preferred.

Additionally, preferred cationic or polycationic proteins or peptides may be selected from the following proteins or peptides having the following total formula (III):

(Arg)ₗ;(Lys)ₘ;(His)ₙ;(Orn)ₒ;(Xaa)ₓ(Gly)_{y}, (formula (III))

wherein l+m+n+o+x=8-15, and l, m, n, o, or y independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide. Particularly preferred cationic peptides in this context are e.g. Arg₇, Arg₈, Arg₉, H₃R₉, R₉H₃, H₃R₉H₃, YSSR₉SSY, (RKH)₄, Y(RKH)₂R, etc. In this context the disclosure of WO 2009/030481 is incorporated herewith by reference. According to another embodiment, the pharmaceutical composition of the present invention comprises the mRNA as defined herein and a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO2012/013326 is incorporated herewith by reference. Also in this context, the disclosure of WO2011/026641 is incorporated herewith by reference.

Further preferred cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

Preferably, the inventive composition comprises at least one RNA as defined herein, which is complexed with one or more polycations, and at least one free RNA, wherein the at least one complexed RNA is preferably identical to the at least one free RNA. In this context, it is particularly preferred that the pharmaceutical composition of the present invention comprises the mRNA according to the invention that is complexed at least partially with a cationic or polycationic compound and/or a polymeric carrier, preferably cationic proteins or peptides. In this context, the disclosure of WO2010037539 and WO2012113513 is incorporated herewith by reference. Partially means that only a part of the mRNA as defined herein is complexed in the pharmaceutical composition according to the invention with a cationic compound and that the rest of the mRNA as defined herein is (comprised in the inventive pharmaceutical composition) in uncomplexed form (“free”). Preferably, the molar ratio of the complexed RNA to the free RNA is selected from a molar ratio of about 0.001:1 to about 1:0.001, including a ratio of about 1:1. More preferably the ratio of complexed RNA to free RNA (in the pharmaceutical composition of the present invention) is selected from a range of about 5:1 (w/w) to about 1:10 (w/w), more preferably from a range of about 4:1 (w/w) to about 1:8 (w/w), even more preferably from a range of about 3:1 (w/w) to about 1:5 (w/w) or 1:3 (w/w), and most preferably the ratio of complexed mRNA to free mRNA in the inventive pharmaceutical composition is selected from a ratio of about 1:1 (w/w).

The complexed RNA in the pharmaceutical composition according to the present invention, is preferably prepared according to a first step by complexing the mRNA according to the invention with a cationic or polycationic compound and/or with a polymeric carrier, preferably as defined herein, in a specific ratio to form a stable complex. In this context, it is highly preferable, that no free cationic or polycationic compound or polymeric carrier or only a negligibly small amount thereof remains in the component of the complexed RNA after complexing the mRNA. Accordingly, the ratio of the mRNA and the cationic or polycationic compound and/or the polymeric carrier in the component of the complexed RNA is typically selected in a range so that the mRNA is entirely complexed and no free cationic or polycationic compound or polymeric carrier or only a negligibly small amount thereof remains in the pharmaceutical composition.

Preferably the ratio of the mRNA as defined herein to the cationic or polycationic compound and/or the polymeric carrier, preferably as defined herein, is selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w). Alternatively, the ratio of the mRNA as defined herein to the cationic or polycationic compound and/or the polymeric carrier, preferably as defined herein, in the component of the complexed mRNA, may also be calculated on the basis of the nitrogen/phosphate ratio (N/P-ratio) of the entire complex. In the context of the present invention, an N/P-ratio is preferably in the range of about 0.1 to 10, preferably in a range of about 0.3 to 4 and most preferably in a range of about 0.5 to 2 or 0.7 to 2 regarding the ratio of RNA:cationic or polycationic compound and/or polymeric carrier, preferably as defined herein, in the complex, and most preferably in a range of about 0.7 to 1.5, 0.5 to 1 or 0.7 to 1, and even most preferably in a range of about 0.3 to 0.9 or 0.5 to 0.9, preferably provided that the cationic or polycationic compound in the complex is a cationic or polycationic cationic or polycationic protein or peptide and/or the polymeric carrier as defined above.

In other embodiments, the pharmaceutical composition according to the invention comprising the mRNA as defined herein may be administered naked without being associated with any further vehicle, transfection or complexation agent.

It has to be understood and recognized, that according to the present invention, the inventive composition may comprise at least one mRNA, and/or at least one formulated/complexed mRNA as defined herein, wherein every formulation and/or complexation as disclosed above may be used.

In embodiments, wherein the pharmaceutical composition comprises more than one RNA species, these RNA species may be provided such that, for example, two, three, four, five, six, seven, eight, nine or more separate compositions, which may contain at least one RNA species each (e.g. three distinct mRNA species), each encoding a distinct peptide or protein as defined herein or a fragment or variant thereof, are provided, which may or may not be combined. Also, the pharmaceutical composition may be a combination of at least two distinct compositions, each composition comprising at least one mRNA encoding at least one of the peptides or proteins defined herein. Alternatively, the pharmaceutical composition may be provided as a combination of at least one mRNA, preferably at least two, three, four, five, six, seven, eight, nine or more mRNAs, each encoding one of the peptides or proteins defined herein. The pharmaceutical composition may be combined to provide one single composition prior to its use or it may be used such that more than one administration is required to administer the distinct mRNA species encoding a certain combination of the proteins as defined herein. If the pharmaceutical composition contains at least one mRNA molecule, typically at least two mRNA molecules, encoding of a combination of peptides or proteins as defined herein, it may e.g. be administered by one single administration (combining all mRNA species), by at least two separate administrations. Accordingly, any combination of mono-, bi- or multicistronic mRNAs encoding a peptide or protein or any combination of peptides or proteins as defined herein (and optionally further proteins), provided as separate entities (containing one mRNA species) or as combined entity (containing more than one mRNA species), is understood as a pharmaceutical composition according to the present invention. According to a particularly preferred embodiment of the pharmaceutical composition, the at least one peptide or protein, preferably a combination of at least two, three, four, five, six, seven, eight, nine or more peptides or proteins encoded by the pharmaceutical composition as a whole, is provided as an individual (monocistronic) mRNA, which is administered separately.

The pharmaceutical composition according to the present invention may be provided in liquid and or in dry (e.g. lyophilized) form.

The pharmaceutical composition typically comprises a safe and effective amount of the mRNA according to the invention as defined herein, encoding a peptide or protein as defined herein or a fragment or variant thereof or a combination of peptides or proteins, preferably as defined herein. As used herein, “safe and effective amount” means an amount of the mRNA that is sufficient to significantly induce a positive modification of a disease or disorder as defined herein. At the same time, however, a “safe and effective amount” is small enough to avoid serious side-effects, that is to say to permit a sensible relationship between advantage and risk. The determination of these limits typically lies within the scope of sensible medical judgment. In relation to the pharmaceutical composition of the present invention, the expression “safe and effective amount” preferably means an amount of the mRNA (and thus of the encoded peptide or protein) that is suitable for obtaining an appropriate expression level of the encoded protein(s). Such a “safe and effective amount” of the mRNA of the pharmaceutical composition as defined herein may furthermore be selected in dependence of the type of RNA, e.g. monocistronic, bi- or even multicistronic RNA, since a bi- or even multicistronic RNA may lead to a significantly higher expression of the encoded protein(s) than the use of an equal amount of a monocistronic RNA. A “safe and effective amount” of the mRNA of the pharmaceutical composition as defined above may furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The pharmaceutical composition according to the invention can be used according to the invention for human and also for veterinary medical purposes.

In a preferred embodiment, the mRNA of the pharmaceutical composition or kit of parts according to the invention is provided in lyophilized form. Preferably, the lyophilized RNA is reconstituted in a suitable buffer, advantageously based on an aqueous carrier, prior to administration, e.g. Ringer-Lactate solution, which is preferred, Ringer solution, a phosphate buffer solution. In a preferred embodiment, the pharmaceutical composition or the kit of parts according to the invention contains at least two, three, four, five, six, seven, eight, nine or more RNAs, preferably mRNAs, which are provided separately in lyophilized form (optionally together with at least one further additive) and which are preferably reconstituted separately in a suitable buffer (such as Ringer-Lactate solution) prior to their use so as to allow individual administration of each of the (monocistronic) RNAs.

The pharmaceutical composition according to the invention may typically contain a pharmaceutically acceptable carrier. The expression “pharmaceutically acceptable carrier” as used herein preferably includes the liquid or non-liquid basis of the pharmaceutical composition. If the pharmaceutical composition is provided in liquid form, the carrier will be water, typically pyrogen-free water; isotonic saline or buffered (aqueous) solutions, e.g phosphate, citrate etc. buffered solutions. Particularly for injection of the pharmaceutical composition, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, Cal₂, CaBr₂, CaCO₃, CaSO₄, Ca(OH)₂. Furthermore, organic anions of the aforementioned cations may be contained in the buffer. According to a more preferred embodiment, the buffer suitable for injection purposes as defined above, may contain salts selected from sodium chloride (NaCl), calcium chloride (CaCl₂) and optionally potassium chloride (KCl), wherein further anions may be present additional to the chlorides. CaCl₂ can also be replaced by another salt like KCl. Typically, the salts in the injection buffer are present in a concentration of at least 50 mM sodium chloride (NaCl), at least 3 mM potassium chloride (KCl) and at least 0.01 mM calcium chloride (CaCl₂). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. in “in vivo” methods occurring liquids such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds may be used as well, which are suitable for administration to a person. The term “compatible” as used herein means that the constituents of the pharmaceutical composition according to the invention are capable of being mixed with the mRNA according to the invention as defined herein, in such a manner that no interaction occurs, which would substantially reduce the pharmaceutical effectiveness of the pharmaceutical composition according to the invention under typical use conditions. Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from Theobroma; polyols, such as, for example, polypropylene glycol, glycerol, sorbitol, mannitol and polyethylene glycol; alginic acid.

The choice of a pharmaceutically acceptable carrier is determined, in principle, by the manner, in which the pharmaceutical composition according to the invention is administered. The pharmaceutical composition can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections. More preferably, the pharmaceutical composition according to the present invention may be administered by an intradermal, subcutaneous, or intramuscular route, preferably by injection, which may be needle-free and/or needle injection. The pharmaceutical composition is therefore preferably formulated in liquid or solid form. The suitable amount of the pharmaceutical composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

Further additives which may be included in the pharmaceutical composition are emulsifiers, such as, for example, Tween; wetting agents, such as, for example, sodium lauryl sulfate; colouring agents; taste-imparting agents, pharmaceutical carriers; tablet-forming agents; stabilizers; antioxidants; preservatives.

In preferred embodiments, the pharmaceutical composition according to the invention comprises a further pharmaceutically active ingredient in addition to the mRNA according to the invention. Preferably, the further pharmaceutically active ingredient is selected from compounds suitable for use in the treatment or prophylaxis of a liver disease or disorder as defined herein.

The pharmaceutical composition as defined herein may also be administered orally in any orally acceptable dosage form including, but not limited to, capsules, tablets, aqueous suspensions or solutions.

The pharmaceutical composition may also be administered topically. Suitable topical formulations are readily prepared for each of these areas or organs. For topical applications, the pharmaceutical composition may be formulated in a suitable ointment, containing the mRNA according to the invention suspended or dissolved in one or more carriers.

According to a preferred embodiment of this aspect of the invention, the pharmaceutical composition according to the invention is administered via a parenteral route, preferably by injection. Preferably, the inventive composition is administered by intradermal, subcutaneous, intramuscular or intravenous injection, most preferably by intravenous injection. Any suitable injection technique known in the art may be employed, for example conventional needle injection or needle-less injection techniques, such as jet-injection, or intravenous infusion or respectively intravenous therapy (IV therapy).

In one embodiment, the pharmaceutical composition comprises at least two, three, four, five, six, seven, eight, nine or more RNAs as defined herein, each of which is preferably injected separately, preferably by needle-less injection. Alternatively, the pharmaceutical composition comprises at least two, three, four, five, six, seven, eight, nine or more RNAs, wherein the at least two, three, four, five, six, seven, eight, nine or more RNAs are administered, preferably by injection as defined herein, as a mixture.

Administration of the mRNA as defined herein or the pharmaceutical composition according to the invention may be carried out in a time staggered treatment. A time staggered treatment may be e.g. administration of the mRNA or the pharmaceutical composition prior, concurrent and/or subsequent to a conventional therapy of a disease or disorder, preferably as described herein, e.g. by administration of the mRNA or the pharmaceutical composition prior, concurrent and/or subsequent to a therapy or an administration of a therapeutic agent suitable for the treatment or prophylaxis of a disease or disorder as described herein, preferably a liver disease. Such time staggered treatment may be carried out using e.g. a kit, preferably a kit of parts as defined herein. The term disease and disorder are used interchangeably herein.

Time staggered treatment may additionally or alternatively also comprise an administration of the mRNA as defined herein or the pharmaceutical composition according to the invention in a form, wherein the mRNA encoding a peptide or protein as defined herein or a fragment or variant thereof, preferably forming part of the pharmaceutical composition, is administered parallel, prior or subsequent to another RNA encoding a peptide or protein as defined above, preferably forming part of the same inventive composition. Preferably, the administration (of all RNAs) occurs within an hour, more preferably within 30 minutes, even more preferably within 15, 10, 5, 4, 3, or 2 minutes or even within 1 minute. Such time staggered treatment may be carried out using e.g. a kit, preferably a kit of parts as defined herein.

According to a further aspect, the present invention also provides kits, particularly kits of parts for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, more preferably treating, preventing, attenuating or inhibiting liver fibrosis or liver cirrhosis. Such kits, particularly kits of parts, typically comprise as components alone or in combination with further components as defined herein at least one inventive RNA species as defined herein, or the inventive pharmaceutical composition comprising the mRNA according to the invention. The at least one RNA as defined herein, is optionally in combination with further components as defined herein, whereby the at least one RNA is provided separately (first part of the kit) from at least one other part of the kit comprising one or more other components. The pharmaceutical composition may occur in one or different parts of the kit. As an example, e.g. at least one part of the kit may comprise at least one RNA as defined herein, and at least one further part of the kit at least one other component as defined herein, e.g. at least one other part of the kit may comprise at least one pharmaceutical composition or a part thereof, e.g. at least one part of the kit may comprise the mRNA as defined herein, at least one further part of the kit at least one other component as defined herein, at least one further part of the kit at least one component of the pharmaceutical composition or the pharmaceutical composition as a whole, and at least one further part of the kit e.g. at least one pharmaceutical carrier or vehicle, etc. In case the kit or kit of parts comprises a plurality of RNAs as described herein, one component of the kit can comprise only one, several or all RNAs comprised in the kit. In an alternative embodiment every/each RNA species may be comprised in a different/separate component of the kit such that each component forms a part of the kit. Also, more than one RNA as defined herein may be comprised in a first component as part of the kit, whereas one or more other (second, third etc.) components (providing one or more other parts of the kit) may either contain one or more than one RNA as defined herein, which may be identical or partially identical or different from the first component. The kit or kit of parts may furthermore contain technical instructions with information on the administration and dosage of the mRNA according to the invention, the pharmaceutical composition of the invention or of any of its components or parts, e.g. if the kit is prepared as a kit of parts.

In a further aspect, the present invention furthermore provides several applications and uses of the mRNA, of the pharmaceutical composition or the kit of parts according to the invention. In particular, the present invention provides medical uses of the mRNA according to the invention. Moreover, the use of the mRNA according to the invention, of the pharmaceutical composition or the kit of parts according to the invention is envisaged in gene therapy.

In a preferred embodiment of the present invention, the mRNA, the pharmaceutical composition or the kit or kit of parts as described herein is provided for use in the treatment or prophylaxis of a liver disease. Accordingly, the present invention concerns an mRNA comprising at least one coding sequence, wherein the coding sequence encodes at least one peptide or protein as described herein, preferably comprising or consisting of a HNF4A protein, or a fragment or a variant of any of these peptides or proteins having the biological activity of a wild type HNF4A protein, or a pharmaceutical composition or kit or kit of parts comprising the mRNA according to the invention, for use in the treatment or prophylaxis of a liver disease. In other preferred embodiments of the present invention, the mRNA, the pharmaceutical composition or the kit or kit of parts as described herein is provided for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, more preferably treating, preventing, attenuating or inhibiting liver fibrosis or liver cirrhosis.

In a preferred embodiment, the mRNA according to the invention is provided for use in the treatment or prophylaxis of a liver disease, which is preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer. In a preferred embodiment, the mRNA according to the invention is provided for use in the treatment or prophylaxis of hepatocellular carcinoma (HCC). Most preferably, the mRNA according to the invention is provided for use in the treatment or prophylaxis of liver fibrosis or liver cirrhosis.

In a preferred embodiment, the mRNA as described herein or the pharmaceutical composition is provided for treatment or prophylaxis of a liver disease, which comprises targeted delivery of the mRNA. Preferably, the mRNA is targeted to the liver upon administration to a mammalian subject. Targeted delivery of the mRNA according to the invention is preferably achieved by formulating the mRNA in a suitable manner (e.g. as a liposome or lipid nanoparticle as described herein) and/or by administering the mRNA or the pharmaceutical composition, respectively, according to the invention via a suitable route.

Preferably, the treatment or prophylaxis of a liver disease as described herein comprises administration of the mRNA or the pharmaceutical composition according to the invention in any suitable manner, preferably as described herein with respect to the pharmaceutical composition. The description of the pharmaceutical composition, where appropriate, also applies to the medical use of the mRNA according to the invention.

In preferred embodiments, the treatment or prophylaxis comprises administration of a further pharmaceutically active ingredient in combination with the mRNA according to the invention or the pharmaceutical composition according to the invention. Preferably, the further pharmaceutically active ingredient is selected from compounds suitable for use in the treatment or prophylaxis of a liver disease or disorder as defined herein.

Also comprised by the present invention are methods of treating or preventing a disease or disorder, preferably a liver disease or disorder as defined herein, by administering to a subject in need thereof a pharmaceutically effective amount of the mRNA or the pharmaceutical composition according to the invention. Such a method typically comprises an optional first step of preparing the mRNA or the pharmaceutical composition of the present invention, and a second step, comprising administering (a pharmaceutically effective amount of) said composition to a patient/subject in need thereof. A subject in need thereof will typically be a mammal. In the context of the present invention, the mammal is preferably selected from the group comprising, without being limited thereto, e.g. goat, cattle, swine, dog, cat, donkey, monkey, ape, a rodent such as a mouse, hamster, rabbit and, particularly, human, wherein the mammal typically suffers from a disease or disorder, preferably from a liver disease or disorder as defined herein.

According to a further aspect, the present invention also provides a method for increasing the expression of a peptide or protein as described herein comprising the steps, e.g. a) providing the mRNA as defined herein or the pharmaceutical composition as defined herein, b) applying or administering the mRNA or the pharmaceutical composition to an expression system, e.g. to a cell-free expression system, a cell (e.g. an expression host cell or a somatic cell), a tissue or an organism. The method may be applied for laboratory, for research, for diagnostic, for commercial production of peptides or proteins and/or for therapeutic purposes. In this context, typically after preparing the mRNA or the pharmaceutical composition, it is typically applied or administered to a cell-free expression system, a cell (e.g. an expression host cell or a somatic cell), a tissue or an organism, e.g. in naked or complexed form or as a pharmaceutical composition as described herein, preferably via transfection or by using any of the administration modes as described herein. The method may be carried out in vitro, in vivo or ex vivo. The method may furthermore be carried out in the context of the treatment of a specific disease, preferably as defined herein.

In this context in vitro is defined herein as transfection or transduction of the mRNA or the pharmaceutical composition according to the invention into cells in culture outside of an organism; in vivo is defined herein as transfection or transduction of the mRNA or the pharmaceutical composition according to the invention into cells by application of the mRNA or the pharmaceutical composition to the whole organism or individual and ex vivo is defined herein as transfection or transduction of the mRNA or the pharmaceutical composition according to the invention into cells outside of an organism or individual and subsequent application of the transfected cells to the organism or individual.

Likewise, according to another aspect, the present invention also provides the use of the mRNA or the pharmaceutical composition according to the invention, preferably for diagnostic or therapeutic purposes, for increasing the expression of a peptide or protein as described herein, particularly in gene therapy e.g. by applying or administering the mRNA or the pharmaceutical composition, e.g. to a cell-free expression system, a cell (e.g. an expression host cell or a somatic cell), a tissue or an organism. The use may be applied for laboratory, for research, for diagnostic for commercial production of peptides or proteins and/or for therapeutic purposes, preferably for gene therapy. In this context, typically after preparing the mRNA or the pharmaceutical composition according to the invention, it is typically applied or administered to a cell-free expression system, a cell (e.g. an expression host cell or a somatic cell), a tissue or an organism, preferably in naked form or complexed form, or as a pharmaceutical composition as described herein, preferably via transfection or by using any of the administration modes as described herein. The use may be carried out in vitro, in vivo or ex vivo. The use may furthermore be carried out in the context of the treatment of a specific disease, preferably a liver disease or disorder as defined herein.

In yet another aspect the present invention also relates to an inventive expression system comprising the mRNA according to the invention or an expression vector or plasmid comprising a corresponding nucleic acid sequence according to the first aspect of the present invention. In this context the expression system may be a cell-free expression system (e.g. an in vitro transcription/translation system), a cellular expression system (e.g. mammalian cells like CHO cells, insect cells, yeast cells, bacterial cells like E. coli) or organisms used for expression of peptides or proteins (e.g. plants or animals like cows).

In a third aspect, the present invention relates to a lipid nanoparticle (LNP), comprising the mRNA of the invention, wherein the LNP comprises an ionizable or cationic lipid, a phospholipid, a structural lipid, and a polymer conjugated lipid.

In a preferred embodiment, the lipids comprised in the LNP of the invention have a molar ratio of about 20-60% cationic or ionizable lipid, about 5-25% non-cationic lipid, about 25-55% sterol and about 0.5-15% polymer conjugated lipid.

In other embodiments, the LNP of the invention does not comprise polyethylene glycol (PEG) or a PEG-modified lipid.

In another aspect, the present invention relates to a pharmaceutical composition, comprising the mRNA of the invention or the LNP of the invention.

In yet another aspect, the present invention related to a kit, preferably kit of parts, comprising at least one mRNA of the invention, the LNP of the invention, or the pharmaceutical composition of the invention, and optionally a liquid vehicle for solubilising and optionally technical instructions with information on the administration and dosage of the pharmaceutical composition.

Accordingly, in a third aspect, the present invention the present invention provides the mRNA as described herein for use in the treatment, prevention, attenuation, inhibition, or prophylaxis of a the liver disease. Moreover, the mRNA as described herein is preferably provided for gene therapy.

### Cationic Lipids

The lipid nanoparticle may include any cationic lipid suitable for forming a lipid nanoparticle. Preferably, the cationic lipid carries a net positive charge at about physiological pH.

The cationic lipid is preferably an amino lipid. As used herein, the term “amino lipid” is meant to include those lipids having one or two fatty acid or fatty alkyl chains and an amino head group (including an aikylamino or dialkylamino group) that may be protonated to form a cationic lipid at physiological pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)-didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination of any of the foregoing.

Other cationic lipids include, but are not limited to, N,N-distearyl-N,N-dimethylammonium bromide (DDAB), 3P—(N—(N′,N′-dimethylaminoethane)-carbamoyl)cholesterol (DC-Chol), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL).

Other suitable cationic lipids are disclosed in International Publication Nos. WO09/086558, WO09/127060, WO10/048536, WO10/054406, WO10/088537, WO10/129709, and WO2011/153493; U.S. Patent Publication Nos. 2011/0256175, 2012/0128760, and 2012/0027803; U.S. Pat. No. 8,158,601; and Love et al, PNAS, 107(5), 1864-69, 2010. [51] Other suitable amino lipids include those having alternative fatty acid groups and other dialkylamino groups, including those in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In certain embodiments, amino or cationic lipids of the invention have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention.

In certain embodiments, the protonatable lipids have a pKa of the protonatable group in the range of about 4 to about 11, e.g., a pKa of about 5 to about 7.

Lipid particles preferably include two or more cationic lipids. The cationic lipids are preferably selected to contribute different advantageous properties. For example, cationic lipids that differ in properties such as amine pKa, chemical stability, half-life in circulation, half-life in tissue, net accumulation in tissue, or toxicity can be used in the lipid nanoparticle. In particular, the cationic lipids can be chosen so that the properties of the mixed-lipid particle are more desirable than the properties of a single-lipid particle of individual lipids.

The cationic lipid preferably comprises from about 20 mol % to about 70 mol % or 75 mol % or from about 45 mol % to about 65 mol % or about 20, 25, 30, 35, 40, 45, 50, 55, 60, 65, or about 70 mol % of the total lipid present in the particle. In another embodiment, the lipid nanoparticles include from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In one embodiment, the ratio of cationic lipid to nucleic acid is from about 3 to about 15, such as from about 5 to about 13 or from about 7 to about 11.

### Non-Cationic Lipids

The non-cationic lipid is preferably a neutral lipid, an anionic lipid, or an amphipathic lipid. Neutral lipids, when present, can be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., lipid particle size and stability of the lipid particle in the bloodstream. Preferably, the neutral lipid is a lipid having two acyl groups (e.g., diacylphosphatidylcholine and diacylphosphatidylethanolamine). In one embodiment, the neutral lipids contain saturated fatty acids with carbon chain lengths in the range of C10 to C20. In another embodiment, neutral lipids with mono or diunsaturated fatty acids with carbon chain lengths in the range of C10 to C2o are used. Additionally, neutral lipids having mixtures of saturated and unsaturated fatty acid chains can be used.

Suitable neutral lipids include, but are not limited to, distearoylphosphatidylcholine or 1,2-distearoyl-sn-glycero-3-phosphocholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-0-monomethyl PE, 16-O-dimethyl PE, 18-1-trans PE, 1-stearoyl-2-oleoyl-phosphatidyethanolamine (SOPE), cholesterol, or a mixture thereof. Anionic lipids suitable for use in lipid particles of the invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In various embodiments, the molar ratio of the cationic lipid to the neutral lipid in the lipid-based carriers ranges from about 2:1 to about 8:1.

The neutral lipid is preferably from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the lipid-based carrier. In one embodiment, the lipid-based carrier include from about 0% to about 15% or 45% on a molar basis of neutral lipid, e.g., from about 3% to about 12% or from about 5% to about 10%. For instance, the lipid-based carrier may include about 15%, about 10%, about 7.5%, or about 7.1% of neutral lipid on a molar basis (based upon 100% total moles of lipid in the lipid-based carrier).

The term “amphipathic lipid(s)” refers to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and b-acyloxyacids, can also be used.

The non-cationic lipid is preferably from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the particle. In one embodiment, the lipid nanoparticles include from about 0% to about 15% or 45% on a molar basis of neutral lipid, e.g., from about 3% to about 12% or from about 5% to about 10%. For instance, the lipid nanoparticles may include about 15%, about 10%, about 7.5%, or about 7.1% of neutral lipid on a molar basis (based upon 100% total moles of lipid in the lipid nanoparticle).

### Steroids, Steroid Analogs or Sterols

A preferred sterol is cholesterol. Further sterols as known in the art are further envisaged for use in the context of the present invention.

The sterol preferably constitutes about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the lipid nanoparticles include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

The molar ratio of the cationic lipid to cholesterol in the lipid-based carriers may be in the range from about 2:1 to about 1:1. In some embodiments, the cholesterol may be PEGylated.

In some embodiments, the lipid-based carrier comprises about 10 mol % to about 60 mol % or about 25 mol % to about 40 mol % sterol (based on 100% total moles of lipids in the lipid-based carrier). In one embodiment, the sterol is about 10, 15, 20, 25, 30, 35, 40, 45, 50, 55, or about 60 mol % of the total lipid present in the lipid-based carrier. In another embodiment, the lipid-based carriers include from about 5% to about 50% on a molar basis of the sterol, e.g., about 15% to about 45%, about 20% to about 40%, about 48%, about 40%, about 38.5%, about 35%, about 34.4%, about 31.5% or about 30% on a molar basis (based upon 100% total moles of lipid in the lipid-based carrier). In preferred embodiments, the lipid-based carrier comprises about 28%, about 29% or about 30% sterol (based on 100% total moles of lipids in the lipid-based carrier). In most preferred embodiments, the lipid-based carrier comprises about 40.9% sterol (based on 100% total moles of lipids in the lipid-based carrier).

### Polymer Conjugated Lipid—Aggregation Reducing Agent

The aggregation reducing agent is preferably a lipid capable of reducing aggregation. Examples of such lipids include, but are not limited to, polyethylene glycol (PEG)-modified lipids, monosialoganglioside Gml, and polyamide oligomers (PAO) such as those described in U.S. Pat. No. 6,320,017, which is incorporated by reference in its entirety. Other compounds with uncharged, hydrophilic, steric-barrier moieties, which prevent aggregation during formulation, like PEG, Gml or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499 and 5,885,613, each of which is incorporated by reference in its entirety.

The aggregation reducing agent may be, for example, a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof (such as PEG-Cerl4 or PEG-Cer20). The PEG-DAA conjugate may be, for example, a PEG-dilauryloxypropyl (C12), a PEG-dimyristyloxypropyl (C14), a PEG-dipalmityloxypropyl (C16), or a PEG-distearyloxypropyl (C18). Other pegylated-lipids include, but are not limited to, polyethylene glycol-didimyristoyl glycerol (C14-PEG or PEG-C14, where PEG has an average molecular weight of 2000 Da) (PEG-DMG); (R)-2,3-bis(octadecyloxy)propyl-1-(methoxy poly(ethylene glycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis(octadecyloxy)propyl-1-(methoxypoly(ethylene glycol)2000)propylcarbamate)) (GalNAc-PEG-DSG); mPEG (mw2000)-diastearoylphosphatidyl-ethanolamine (PEG-DSPE); and polyethylene glycol dipalmitoylglycerol (PEG-DPG). In one embodiment, the aggregation reducing agent is PEG-DMG. In another embodiment, the aggregation reducing agent is PEG-c-DMA.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28: 172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the pharmaceutical composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1.

The average molecular weight of the PEG moiety in the PEG-modified lipids preferably ranges from about 500 to about 8,000 Daltons (e.g., from about 1,000 to about 4,000 Daltons). In one preferred embodiment, the average molecular weight of the PEG moiety is about 2,000 Daltons.

The concentration of the aggregation reducing agent preferably ranges from about 0.1 mol % to about 15 mol %, based upon the 100% total moles of lipid in the lipid particle. In one embodiment, the formulation includes less than about 3, 2, or 1 mole percent of PEG or PEG-modified lipid, based upon the total moles of lipid in the lipid particle.

In another embodiment, the lipid nanoparticles include from about 0.1% to about 20% on a molar basis of the PEG-modified lipid, e.g., about 0.5 to about 10%, about 0.5 to about 5%, about 10%, about 5%, about 3.5%, about 1.5%, about 0.5%, or about 0.3% on a molar basis (based on 100% total moles of lipids in the lipid nanoparticle).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

A polymer conjugated lipid as defined herein, e.g. a PEG-lipid, may serve as an aggregation reducing lipid.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments, the PEGylated lipid is preferably derived from formula (IV) of published PCT patent application WO2018078053. Accordingly, PEGylated lipids derived from formula (IV) of published PCT patent application WO2018078053, and the respective disclosure relating thereto, are herewith incorporated by reference.

In a particularly preferred embodiments, the at least one nucleic acid (e.g. RNA or DNA) of the composition is complexed with one or more lipids thereby forming LNPs, wherein the LNP comprises a PEGylated lipid, wherein the PEG lipid is preferably derived from formula (IVa) of published PCT patent application WO2018078053. Accordingly, PEGylated lipid derived from formula (IVa) of published PCT patent application WO2018078053, and the respective disclosure relating thereto, is herewith incorporated by reference.

In a particularly preferred embodiment, the at least one nucleic acid, preferably the at least one RNA is complexed with one or more lipids thereby forming lipid nanoparticles (LNP), wherein the LNP comprises a PEGylated lipid/PEG lipid. Preferably, said PEG lipid is of formula (IVa):

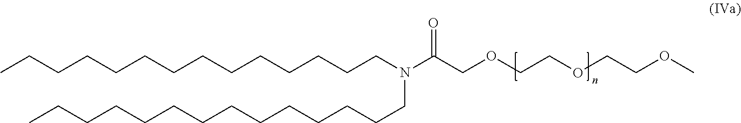

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49. In further preferred aspects said PEG lipid is of formula (IVa) wherein n is an integer selected such that the average molecular weight of the PEG lipid is about 2000 g/mol to about 3000 g/mol or about 2300 g/mol to about 2700 g/mol, even more preferably about 2500 g/mol.

The lipid of formula IVa as suitably used herein has the chemical term 2[(polyethylene glycol)-2000]-N,N-ditetradecylacetamide, also referred to as ALC-0159.

Further examples of PEG-lipids suitable in that context are provided in US20150376115A1 and WO2015199952, each of which is incorporated by reference in its entirety.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

### Encapsulation/Complexation in LNPs:

In preferred embodiments of the second aspect, the at least one nucleic acid (e.g. DNA or RNA), preferably the at least one RNA, and optionally the at least one further nucleic acid, is complexed, encapsulated, partially encapsulated, or associated with one or more lipids (e.g. cationic lipids and/or neutral lipids), thereby forming liposomes, lipid nanoparticles (LNPs), lipoplexes, and/or nanoliposomes.

The liposomes, lipid nanoparticles (LNPs), lipoplexes, and/or nanoliposomes—incorporated nucleic acid (e.g. DNA or RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the RNA is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA from an environment which may contain enzymes or chemicals or conditions that degrade nucleic acid and/or systems or receptors that cause the rapid excretion of the nucleic acid. Moreover, incorporating nucleic acid, preferably RNA into liposomes, lipid nanoparticles (LNPs), lipoplexes, and/or nanoliposomes may promote the uptake of the nucleic acid, and hence, may enhance the therapeutic effect of the nucleic acid, e.g. the mRNA medicine(s) for use in the therapy and prevention of liver diseases like liver fibrosis, liver cirrhosis, hepatocellular carcinoma (HCC), non-alcoholic fatty liver disease (NAFLD), non-alcoholic steatohepatitis (NASH) or liver cancer, and more particularly to mRNA medicines of this kind which can exhibit excellent therapeutic and preventive effects with respect to liver diseases individually developed or to complications resulting from diseases of these organs. Accordingly, incorporating a nucleic acid, e.g. RNA or DNA, into liposomes, lipid nanoparticles (LNPs), lipoplexes, and/or nanoliposomes may be particularly suitable for mRNA medicines for use in the therapy and prevention of liver diseases like liver fibrosis, liver cirrhosis, hepatocellular carcinoma (HCC), non-alcoholic fatty liver disease (NAFLD), non-alcoholic steatohepatitis (NASH) or liver cancer, and more particularly to mRNA medicines of this kind which can exhibit excellent therapeutic and preventive effects with respect to liver diseases individually developed or to complications resulting from diseases of these organs, e.g. for intravenous administration.

In this context, the terms “complexed” or “associated” refer to the essentially stable combination of nucleic acid with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the at least one nucleic acid, preferably the at least one RNA to a target tissue.

Accordingly, in preferred embodiments of all aspects of the invention, the at least one nucleic acid, preferably the at least one RNA is complexed with one or more lipids thereby forming lipid nanoparticles (LNP). Preferably, said LNP is particularly suitable for intramuscular, intradermal administration, subcutaneous, or intravenous injection, most preferably for intravenous injection i.e. intravenous infusion or respectively intravenous therapy (as IV therapy).

### Pharmaceutical Composition

In a further aspect, the present invention concerns a composition or a pharmaceutical composition comprising the mRNA according to the invention as described herein. The pharmaceutical composition according to the invention thus comprises an RNA comprising at least one coding sequence, wherein the coding sequence encodes at least one peptide or protein as described herein, preferably a HNF4A protein selected, or a fragment or a variant of any of a HNF4A protein, having the biological activity of a wild type HNF4A protein, as defined herein, and a pharmaceutically acceptable carrier. The pharmaceutical composition according to the invention is preferably provided as a pharmaceutical composition.

With respect to the mRNA comprised in the pharmaceutical composition, reference is made to the description of the mRNA according to the invention, which applies to the pharmaceutical composition.

The pharmaceutical composition according to the invention preferably comprises at least one RNA according to the invention as described herein. In alternative embodiments, the pharmaceutical composition comprises at least two species of the mRNA according to the invention.

In the context of the present invention, the pharmaceutical composition may comprise more than a single mRNA encoding a HNF4A protein as defined herein. In other words, according to a preferred embodiment, the pharmaceutical composition comprises a combination of different mRNAs encoding different HNF4A proteins, for example an mRNA(s) encoding WT HNF4A and additional mRNA(s) encoding one or more engineered HNF4A protein variants.

In a preferred embodiment, the pharmaceutical composition of the present invention may comprise at least one RNA according to the invention, wherein the at least one RNA encodes at least two, three, four, five, six, seven, eight, nine or more distinct peptides or proteins as defined herein or a fragment or variant thereof. Preferably, the pharmaceutical composition comprises several species, more preferably at least two, three, four, five, six, seven, eight, nine or more species, of the mRNA according to the invention, wherein each RNA species encodes one of the peptides or proteins or a fragment or variant thereof as defined herein. In another embodiment, the mRNA comprised in the pharmaceutical composition is a bi- or multicistronic RNA as defined herein, which encodes the at least two, three, four, five, six, seven, eight, nine or more distinct peptides or proteins. Mixtures between these embodiments are also envisaged, such as compositions comprising more than one RNA species, wherein at least one RNA species may be monocistronic, while at least one other RNA species may be bi- or multicistronic.

The pharmaceutical composition according to the present invention, preferably the at least one coding sequence of the mRNA comprised therein, may thus comprise any combination of the nucleic acid sequences as defined herein.

In a preferred embodiment of the pharmaceutical composition according to the invention, the mRNA as described herein is complexed with one or more cationic or polycationic compounds, preferably with cationic or polycationic polymers, cationic or polycationic peptides or proteins, e.g. protamine, cationic or polycationic polysaccharides and/or cationic or polycationic lipids.

In some embodiments, the mRNA may be formulated as saline or lipid formulation. According to a preferred embodiment, the mRNA according to the present invention may be complexed with lipids to form one or more liposomes, lipoplexes, or lipid nanoparticles. Therefore, in one embodiment, the inventive composition comprises liposomes, lipoplexes, and/or lipid nanoparticles comprising the mRNA according to the invention. In one embodiment the mRNA according to the present invention is complexed with cationic lipids and/or neutral lipids and thereby forms liposomes, lipid nanoparticles, lipoplexes or neutral lipid-based nanoliposomes.

In a preferred embodiment, the lipid formulation is thus selected from the group consisting of liposomes, lipoplexes, copolymers such as PLGA and lipid nanoparticles.

In one preferred embodiment, a lipid nanoparticle (LNP) comprises:
a) an RNA comprising at least one coding sequence as defined herein,
b) a cationic lipid,
c) an aggregation reducing agent (such as polyethylene glycol (PEG) lipid or PEG-modified lipid),
d) optionally a non-cationic lipid (such as a neutral lipid), and
e) optionally, a sterol.

In one embodiment, the lipid nanoparticle formulation consists of (i) at least one cationic lipid; (ii) a neutral lipid; (iii) a sterol, e.g., cholesterol; and (iv) a PEG-lipid, in a molar ratio of about 20-60% cationic lipid:5-25% neutral lipid:25-55% sterol; 0.5-15% PEG-lipid.

In one embodiment, the nucleic acids may be formulated in an aminoalcohol lipidoid. Aminoalcohol lipidoids which may be used in the present invention may be prepared by the methods described in U.S. Pat. No. 8,450,298, herein incorporated by reference in its entirety.

In another aspect, the present invention relates to a method of treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) or liver cancer, comprising administering to a human subject in need the mRNA of the invention, the LNP of the invention, the pharmaceutical composition of the invention, or the kit or kit of parts of the invention, wherein the administration results in treatment, prevention, attenuation, inhibition, or prophylaxis of the disease.

In yet another embodiment, the method of the invention is related to the administration of the mRNA of the invention to a human subject in need, resulting in
(i) improved hepatocyte metabolic activity; and/or
(ii) revived function of hepatocytes; and/or
(iii) increased mRNA levels of hepatocyte markers such as albumin (ALB), alpha-1 antitrypsin (A1AT), transferrin (TF) and/or transthyretin (TTR) in the liver or hepatocytes; and/or
(iv) reduced expression of fibrogenic marker genes Col1a1, Col2a1 Ck19, Sox9, Epcam, and/or Acta2 in the liver or hepatocytes; and/or
(v) decreased levels of bilirubin, hydroxyproline content in hepatocytes in the liver or hepatocytes; and/or
(vi) reduced liver injury as measured by histology, desmin or Sirius red staining;
(vii) increased expression of transporters of drug metabolism in the liver or hepatocytes;
(viii) increased serum paraoxonase and arylesterase 1 (PON1) expression or activity;
(ix) increased endogenous HNF4A or endogenous HNF1A levels or induction of the endogenous HNF1A-HNF4A transcriptional feedback loop; and/or
(x) increased function and/or fitness of hepatocytes;
as when compared to a non-treated human subject in need.

In yet another embodiment, the invention provides a method of treating or preventing a liver disease, wherein the method comprises administration of the inventive mRNA encoding WNT HNF4A or preferably encoding an engineered hepatocyte nuclear factor 4 alpha (HNF4A) protein variant resulting in improved hepatocyte metabolic activity as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in improved revived function of hepatocytes as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in increased mRNA levels of hepatocyte markers such as albumin (ALB), alpha-1 antitrypsin (A1AT), transferrin (TF) and/or transthyretin (TTR) in the liver or hepatocytes as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in reduced expression of fibrogenic marker genes Col1a1, Col2a1 Ck19, Sox9, Epcam, and/or Acta2 in the liver or hepatocytes as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in decreased levels of bilirubin, hydroxyproline content in hepatocytes in the liver or hepatocytes as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in reduced liver injury as measured by histology, desmin or Sirius red staining as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in increased expression of transporters of drug metabolism in the liver or hepatocytes as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in increased serum paraoxonase and arylesterase 1 (PON1) expression or activity as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in increased endogenous HNF4A or endogenous HNF1A levels or induction of the endogenous HNF1A-HNF4A transcriptional feedback loop as when compared to a non-treated human subject in need.

In yet another embodiment, the invention provides a method of treating or preventing a liver disease, wherein the method comprises administration of the inventive mRNA encoding WT HNF4A or preferably encoding an engineered hepatocyte nuclear factor 4 alpha (HNF4A) protein variant resulting in increased function and/or fitness of hepatocytes as when compared to a non-treated human subject in need.

Surprisingly, the inventors found that the transient HNF4A expression leads to a prolonged therapeutic effect by triggering the endogenous HNF4A-HNF1A feedback loop or respectively increasing endogenous HNF4A or endogenous HNF1A levels. Accordingly, the terms “increased endogenous HNF4A or endogenous HNF1A levels” or “induction of the endogenous HNF1A-HNF4A transcriptional feedback loop” are related to the upregulation or induction of the endogenous “HNF1A-HNF4A feedback loop” upon which has been observed during the studies of the present invention, i.e. the ability of HNF4A to induce endogenous HNF4A and endogenous HNF1A (itself then inducing again endogenous HNF4A), thereby prolonging the therapeutic effect. Together with HNF4A, forms a critical transcriptional feedback loop, which mutually promote their own expression (PMID: 29175243). Therefore, in yet another aspect, the present invention relates to an isolated mRNA encoding an hepatocyte nuclear factor 1 alpha (HNF1A, HNF-1A, HNF1, IDDM20, LFB1, MODY3, TCF-1, TCF1, HNF1 homeobox A, HNF4A, HNF1alpha, OMIM: 142410), for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer. In a preferred embodiment, an mRNA, preferably codon-optimized and/or GC-optimized, encoding the 631 amino acid isoform of Uniprot isoform A: P20823-1, NCBI isoform 2: NP_000536.6, or respectively REFSEQ: accession NM_000545.8 is used (SEQ ID NO:5672), more preferably a sequence encoding HNF1A selected from the group consisting of SEQ ID NO:5673-5712, 5716-5718.

Advantageously, in comparison with AAV-related approaches, the mRNA-approach of the present invention leads to a transient transcription factor (HNF4A) expression, which is beneficial compared to a more constant AAV expression of a transcription factor.

The terms “hepatocyte metabolic activity” and “revived function of hepatocytes” are used to describe the actions of a hepatocyte. In this regard, the term “metabolic activity” or the “function” includes fundamental intermediary cellular metabolism that promotes and maintains hepatocyte viability as well as the normal synthesis of various plasma proteins, e.g., albumin, fibrinogen, various globulins and blood coagulation proteins, e.g., prothrombin and factor VII. Hepatocyte metabolic activity also includes the action on compounds (toxins, drugs) that are abnormally elevated in subjects with liver failure or contribute to symptoms of liver failure. These compounds are typically elevated in samples of blood, plasma, serum, or other body fluids. The metabolic activity of the hepatocytes therefore results in detoxification of compounds that are abnormally elevated in such subjects. Such metabolic activity should include the conversion of ammonia into urea, and preferably also the metabolism of drugs or endogenously produced toxic compounds by one or more enzymes from the cytochrome P450 family of enzymes present in the hepatocytes. Hepatocyte metabolic activity includes the elimination of aromatic amino acids, the conjugation of bilirubin, glucose metabolism, and heme metabolism.

The term “function and fitness of hepatocytes” is related to the property of hepatocytes, hepatocytes self, replicate, which potentially contributes to the effect of HNF4A over- or respectively reexpression.

In other aspects and embodiments of the invention, any pharmaceutical composition which (i) increases HNF1A levels or which (ii) increases serum paraoxonase and arylesterase 1 (PON1) expression or activity is comprised within the scope of the present application. Preferably, the HNF1A sequences as disclosed in Table A (preferred mRNA sequences and constructs of the invention), will be sufficient for a use in treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer.

### Antagonists of RNA Sensing Pattern Recognition Receptors:

In preferred embodiments, in particular in embodiments where the nucleic acid of the composition is an RNA, the pharmaceutical composition may comprise at least one antagonist of at least one RNA sensing pattern recognition receptor.

In preferred embodiments in that context, the pharmaceutical composition comprises at least one antagonist of at least one RNA sensing pattern recognition receptor selected from a Toll-like receptor, preferably a TLR7 antagonist and/or a TLR8 antagonist.

Suitable antagonist of at least one RNA sensing pattern recognition receptor are disclosed in published PCT patent application WO2021028439, the full disclosure herewith incorporated by reference. In particular, the disclosure relating to suitable antagonist of at least one RNA sensing pattern recognition receptors as defined in any one of the claims 1 to 94 of WO2021028439 are incorporated by reference.

In preferred embodiments, the at least one antagonist of at least one RNA sensing pattern recognition receptor is a single stranded oligonucleotide that comprises or consists of a nucleic acid sequence being identical or at least 90%, 91%, 92%, 93%, 94%, 95%, 96%, 97%, 98%, 99%, or 100% identical to a nucleic acid sequence selected from the group consisting of SEQ ID NO:85-212 of WO2021028439, or fragments of any of these sequences. A particularly preferred antagonist in that context is 5′-GAG CGmG CCA-3′ (SEQ ID NO:85 of WO2021028439), or a fragment or variant thereof.

In preferred embodiments, the molar ratio of the at least one antagonist of at least one RNA sensing pattern recognition receptor to the at least one RNA suitably ranges from about 20:1 to about 80:1.

In preferred embodiments, the weight to weight ratio of the at least one antagonist of at least one RNA sensing pattern recognition receptor to the at least one RNA suitably ranges from about 1:2 to about 1:10.

In embodiments, the at least one antagonist of at least one RNA sensing pattern recognition receptor and the at least one RNA encoding are separately formulated in the lipid-based carriers as defined herein or co-formulated in the lipid-based carriers as defined herein.

### Routes of Administration

In a further embodiment, the method of the invention relates to the mRNA of the invention, or the LNP of the invention, or the pharmaceutical composition of the invention or the kit or kit of parts of the invention, being administered to the subject by subcutaneous, intramuscular or intravenous administration, preferably intravenous administration.

The choice of a pharmaceutically acceptable carrier is determined, in principle, by the manner, in which the pharmaceutical composition according to the invention is administered. The pharmaceutical composition of the invention can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes.

Routes for local administration in general include, for example, topical administration routes but also intradermal, transdermal, subcutaneous, or intramuscular injections or intralesional, intracranial, intrapulmonal, intracardial, intratumoral and sublingual injections. Administration to the respiratory system can be performed by spray administration or inhalation may in particular be performed by aerosol administration to the lungs, bronchi, bronchioli, alveoli, or paranasal sinuses.

In further preferred embodiments, the route of administration is selected from the group consisting of extravascular administration to a subject, such as by extravascular injection, infusion or implantation; topical administration to the skin or a mucosa; inhalation such as to deliver the pharmaceutical composition to the respiratory system; or by transdermal or percutaneous administration. In even further preferred embodiments, the pharmaceutical composition of the invention can be administered via local or locoregional injection, infusion or implantation, in particular intradermal, subcutaneous, intramuscular, intracameral, subconjunctival, suprachoroidal injection, subretinal, subtenon, retrobulbar, topical, posterior juxtascleral administration, or intrapulmonal inhalation, interstitial, locoregional, intravitreal, intratumoral, intralymphatic, intranodal, intra-articular, intrasynovial, periarticular, intraperitoneal, intra-abdominal, intracardial, intralesional, intrapericardial, intraventricular, intrapleural, perineural, intrathoracic, epidural, intradural, peridural, intrathecal, intramedullary, intracerebral, intracavernous, intracorporus cavernosum, intraprostatic, intratesticular, intracartilaginous, intraosseous, intradiscal, intraspinal, intracaudal, intrabursal, intragingival, intraovarian, intrauterine, periocular, periodontal, retrobulbar, subarachnoid, subconjunctival or suprachoroidal injection, infusion or implantation.

Preferably, compositions according to the present invention may be administered by an intradermal, subcutaneous, intramuscular or intravenous route, preferably by injection, which may be needle-free and/or needle injection. Compositions according to the present invention are therefore preferably formulated in liquid or solid form. The suitable amount of the composition according to the invention to be administered can be determined by routine experiments, e.g. by using animal models. Such models include, without implying any limitation, rabbit, sheep, mouse, rat, dog and non-human primate models.

Preferred unit dose forms for injection include sterile solutions of water, physiological saline or mixtures thereof. The pH of such solutions should be adjusted to a physiologically tolerable pH, such as about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

miRNA Binding Sites

In further preferred embodiments, the mRNA comprises a 5′- or 3′-untranslated region (UTR) comprising at least one microRNA-binding site, preferably not being a microRNA-122 (miR-122) binding site, more preferably being miR-16, miR-21, miR-24, miR-27, miR-30c, miR-132, miR-133, miR-149, miR-192, miR-194, miR-204, miR-206, miR-208, or miR-223, most preferably being miRNA-148a, miRNA-101, miRNA-192 or miRNA-194, miR-126, miR-142-3p, or miR-142-5p.

In other preferred embodiments, the nucleic acid sequences of the invention comprise at least one miRNA binding site, which is substantially complementary to miRNA sequences selected from at least one or more of the group of Table I consisting of miRNA-148a, miRNA-101, miRNA-192 or miRNA-194. In further embodiments wherein a preferred expression in immune cells has to be avoided such as for protein replacement therapy the miRNA binding site sequence according to the invention preferably comprises at least one miRNA-148a, miRNA-101, and/or optionally a miRNA-192 binding site (depending on the target tissue), preferably at least one miRNA-148a binding site.

TABLE I
miRNA binding sites

| Tissue | Implicated miRNA | Expression profile |
|---|---|---|
| Immune cells | miRNA-148a | Expressed in immune cells |
| Endothelial cells, | miRNA-101 | Expresssed in endothelial cells, |
| epithelial cells | | epithelial cells and adipocytes |
| and adipocytes | | |
| Kidney | miRNA-192 | Kidney-specific expression |
| Kidney | miRNA-194 | Kidney-specific expression |

In another embodiment, treating or preventing the liver disease or liver disorder involves at least a single administration of the mRNA, the LNP, the pharmaceutical composition or the kit or kit of parts. It is clear that still further embodiments are possible, i.e. in yet another embodiment, treating or preventing the liver disease or liver disorder involves administration of a DNA encoding an engineered HNF4A protein variant or administration of the engineered HNF4A protein variant itself. A further embodiment is the use of circular RNA encoding the WT HNF4A protein or the engineered HNF4A protein variant(s) for treating or preventing the liver disease or liver disorder.

In other embodiments, the mRNA, the LNP, the pharmaceutical composition or the kit or kit of parts of the invention are the being administered in the method of the invention
(a) once, preferably more than once, more preferably wherein administration is repeated for a period of at least 1 month, at least 2 months, at least 3 months, at least 4 months, at least 5 months, at least 6 months, at least one year, or lifelong; or
(b) about once a day, about once a week, about twice a week, about three times a week, about four times a week, about six or seven times a week, about once every two weeks, about once every three weeks, about once a month, about twice a month, about three times a month, or about four times a month.

In a next aspect, the present invention relates to an isolated mRNA of the invention, the LNP of the invention or the pharmaceutical composition of the invention, or the kit or kit of parts of the invention, for use as a medicament.

In yet another aspect, the present invention relates to an engineered HNF4A protein variant, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

In yet a further aspect, the present invention relates to the engineered HNF4A protein variant of the invention, for use as a medicament.

According to one specific aspect, the present invention is directed to the first medical use of the mRNA according to the invention, of the pharmaceutical composition or of the kit or kit of parts comprising the mRNA according to the invention or a plurality of inventive RNAs as defined herein as a medicament, particularly in gene therapy, preferably for the treatment or prophylaxis of a liver disease or disorder as defined herein.

According to another aspect, the present invention is directed to the second medical use of the mRNA according to the invention, of the pharmaceutical composition, or of the kit or kit of parts comprising the mRNA according to the invention or a plurality of inventive RNAs as defined herein, for the treatment or prophylaxis of a liver disease or disorder as defined herein, preferably to the use of the mRNA as defined herein, of the pharmaceutical composition, or the kit or kit of parts comprising the mRNA according to the invention as defined herein, for the preparation of a medicament for the prophylaxis, treatment and/or amelioration of a liver disease or disorder as defined herein. Preferably, the pharmaceutical composition is used on or to be administered to a patient in need thereof for this purpose.

According to a further aspect, the mRNA according to the invention or the pharmaceutical composition comprising the mRNA according to the invention is used in the manufacture of a medicament, wherein the medicament is preferably for treatment or prophylaxis of a liver disease or disorder as defined herein.

In yet a further aspect, the present invention relates to an isolated mRNA comprising an open reading frame (ORF) encoding an engineered HNF4A, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

In a further aspect, the present invention relates to an isolated mRNA having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any single element from the group consisting of SEQ ID NO:249-297, 692-740, 1135-1183, 298-346, 741-789, 1184-1232, 347-395, 790-838, 1233-1281, 396-444, 839-887, 1282-1330, 445-493, 888-936, 1331-1379, 494-542, 937-985, 1380-1428, 543-591, 986-1034, 1429-1477, 592-640, 1035-1083, 1478-1526, 641-689, 1084-1132, 1527-1575, 1576, 1577, 2019, 2020, 2462, 2463, 1578-1626, 2021-2069, 2464-2512, 1627-1675, 2070-2118, 2513-2561, 1676-1724, 2119-2167, 2562-2610, 1725-1773, 2168-2216, 2611-2659, 1774-1822, 2217-2265, 2660-2708, 1823-1871, 2266-2314, 2709-2757, 1872-1920, 2315-2363, 2758-2806, 1921-1969, 2364-2412, 2807-2855, 1970-2018, 2413-2461, 2856-2904, 2905, 2906, 3348, 3349, 3791, 3792, 2907-2955, 3350-3398, 3793-3841, 2956-3004, 3399-3447, 3842-3890, 3005-3053, 3448-3496, 3891-3939, 3054-3102, 3497-3545, 3940-3988, 3103-3151, 3546-3594, 3989-4037, 3152-3200, 3595-3643, 4038-4086, 3201-3249, 3644-3692, 4087-4135, 3250-3298, 3693-3741, 4136-4184, 3299-3347, 3742-3790, 4185-4233, 4234, 4235, 4677, 4678, 5120, 5121, 4236-4284, 4679-4727, 5122-5170, 4285-4333, 4728-4776, 5171-5219, 4334-4382, 4777-4825, 5220-5268, 4383-4431, 4826-4874, 5269-5317, 4432-4480, 4875-4923, 5318-5366, 4481-4529, 4924-4972, 5367-5415, 4530-4578, 4973-5021, 5416-5464, 4579-4627, 5022-5070, 5465-5513, 4628-4676, 5071-5119, 5514-5562, and 5719-5736 [if only engineered sequences are meant to appear in this list of sequences, sequences related to WT HNF4A are herewith excluded from the aforementioned list].

In yet a further aspect, the present invention relates to an isolated mRNA of the invention, LNP of the invention, composition of the invention, or kit or kit of parts of the invention, for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer in a human subject in need, comprising administering to a human subject in need the wherein the administration results in treatment, prevention, attenuation, inhibition, or prophylaxis of the liver fibrosis, liver cirrhosis, hepatocellular carcinoma (HCC), non-alcoholic fatty liver disease (NAFLD), non-alcoholic steatohepatitis (NASH) or liver cancer.

In a further aspect, the present invention relates to an isolated nucleic acid construct comprising a nucleic acid sequence encoding the mRNA of the invention, preferably an isolated nucleic acid construct having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NO:1576-5615, 5683-5712, and 5716-5736 or to any one of the sequences as disclosed in the Table C2 “Constructs of the invention”.

In a further aspect, the present invention relates an engineered HNF4A protein variant, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138 or SEQ ID NO:140, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

In a further aspect, the present invention relates an engineered HNF4A protein variant for use as a medicament.

In even a further aspect, the present invention relates an engineered hepatocyte nuclear factor 4 alpha (HNF4A) protein variant, comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

In another embodiment, the present invention relates to the engineered hepatocyte nuclear factor 4 alpha (HNF4A) protein variant, wherein the engineered protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138 or SEQ ID NO:140, or a fragment or variant of said sequences having the biological activity of a HNF4A protein, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

In another aspect, the present invention relates to a fusion protein comprising the engineered HNF4A protein variant of the invention or an mRNA encoding a fusion protein comprising the engineered HNF4A protein variant of the invention.

In the context of the present invention, a “fusion protein” contains at least one additional, heterologous amino acid sequence in addition to the amino acid sequence of the polypeptide of the present invention (which has the activity of a hepatocyte nuclear factor 4 alpha (HNF4A) protein). Often, but not necessarily, these additional sequences will be located at the N- or C-terminal end of the polypeptide. It may e.g. be convenient to initially express the polypeptide as a fusion protein from which the additional amino acid residues can be removed, e.g. by a proteinase capable of specifically trimming the polypeptide of the present invention.

In yet another aspect, the present invention relates to a vector comprising any one of the isolated mRNAs of the invention or to a host cell carrying said vector.

While the various aspects of this invention are susceptible of embodiment in many different forms, this specification and the accompanying drawings disclose only one specific form of each aspect of the invention as an example of the invention. Further, each aspect of the invention is not intended to be limited to the particular embodiment so described, however. The scope of the invention is pointed out in the appended claims. The disclosure in the present application makes available each and every combination of embodiments disclosed herein. Preferred or optional features of each aspect or embodiment of the invention are as for each of the other aspects or embodiments mutatis mutandis.

### Items

The present invention may further be characterized by the following items (for clarification purposes, back-references to e.g., item 1 comprises a back reference to any item 1 i.e. also sub-items designated “Item 1A”, “Item 1B”, “Item 1C” etc. or “Item 1.1”, “Item 1.2”, “Item 1.3” etc.):

Item 1. An isolated mRNA encoding
(i) an engineered hepatocyte nuclear factor 4 alpha (HNF4A) protein variant comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation; or
(ii) wild type hepatocyte nuclear factor 4 alpha (HNF4A);
for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, more preferably treating, preventing, attenuating or inhibiting liver fibrosis or liver cirrhosis.

Item 1B. An isolated mRNA encoding
(i) an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation; or
(ii) wild type hepatocyte nuclear factor 4 alpha (HNF4A);
for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, more preferably treating, preventing, attenuating or inhibiting liver fibrosis or liver cirrhosis.

Item 2.1 The mRNA of item 1(i) for use according to item 1, wherein said mRNA comprises an open reading frame (ORF) encoding an engineered HNF4A comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, more preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140, or a fragment or variant of said sequences having the biological activity of a HNF4A protein.

Item 2.2 The mRNA of item 1(i) for use according to item 1, wherein said mRNA comprises an open reading frame (ORF) encoding an engineered HNF4A comprising one or more amino acid substitution, deletion, and/or insertion mutation leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100,
preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140,
more preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, most preferably to SEQ ID NO:140; or a fragment or variant of said sequences having the biological activity of a HNF4A protein.

Item 2.3 The mRNA of item 1(i) for use according to item 1, wherein said mRNA comprises an open reading frame (ORF) encoding an engineered HNF4A comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100,
preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140,
more preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, most preferably to SEQ ID NO:140 having the biological activity of a HNF4A protein;
or a fragment or variant of said sequences having the biological activity of a HNF4A protein.

Item 3. The mRNA according to any one of item 1 to item 2, wherein said mRNA preferably has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any single SEQ ID NO-element of SEQ ID NO:250-297, 299-346, 348-395, 397-444, 446-493, 495-542, 544-591, 593-640, 642-689, 1579-1626, 1628-1675, 1677-1724, 1726-1773, 1775-1822, 1824-1871, 1873-1920, 1922-1969, 1971-2018, 2908-2955, 2957-3004, 3006-3053, 3055-3102, 3104-3151, 3153-3200, 3202-3249, 3251-3298, 3300-3347, 4237-4284, 4286-4333, 4335-4382, 4384-4431, 4433-4480, 4482-4529, 4531-4578, 4580-4627, 4629-4676, 5720, 5721, 5723, 5724, 5726, 5727, 5729, 5730, 5732, 5733, 5735, and 5736, or a fragment or variant of said sequences, wherein the encoded protein has the biological activity of a HNF4A protein.

Item 4. The mRNA according to any one of item 2 to item 3, wherein said engineered HNF4A comprises at least one substitution or substitution set at one or more positions selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A and/or combinations thereof, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140 and wherein the amino acid positions of said amino acid sequence are numbered with reference to the human wild-type HNF4A protein (SEQ ID NO:100).

Item 4B. The mRNA according to any one of item 2 to item 3, wherein said engineered HNF4A comprises at least one substitution or substitution set at one or more positions selected from the group consisting of R2V, K5V, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A and/or combinations thereof, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140 and wherein the amino acid positions of said amino acid sequence are numbered with reference to the human wild-type HNF4A protein (SEQ ID NO:100), more preferably an mRNA selected from the group consisting of SEQ ID NO:2947, SEQ ID NO:5721, SEQ ID NO:5724 and SEQ ID NO:5727.

Item 5. The mRNA of item 1(ii) or item 2 to item 4 or item 4B for use according to item 1, wherein said mRNA comprises an open reading frame (ORF) encoding an unmodified human wild type hepatocyte nuclear factor 4 alpha (HNF4A) according to SEQ ID NO:100, preferably wherein said mRNA has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:247, 248, 249, 298, 347, 396, 445, 494, 543, 592, 641, 1576, 1577, 1578, 1627, 1676, 1725, 1774, 1823, 1872, 1921, 1970, 2905, 2906, 2907, 2956, 3005, 3054, 3103, 3152, 3201, 3250, 3299, 4234, 4235, 4236, 4285, 4334, 4383, 4432, 4481, 4530, 4579, 4628, 5719, 5722, 5725, 5728, 5731, and 5734 or a fragment or variant of said sequences, wherein the encoded protein has the biological activity of a HNF4A protein, optionally wherein the mRNA further comprises an UTR combination selected from the group consisting of (i) a 5′-UTR derived from a mouse solute carrier family 7 (cationic amino acid transporter, y+ system) (SLC7A3) and a 3′-UTR derived from PSMB3; (ii) a 5′-UTR derived from mouse ribosomal protein L31 (RPL31) and a 3′-UTR derived from a human ribosomal protein S9 (RPS9); (iii) a 5′-UTR derived from ubiquilin 2 (Ubqln2) and a 3′-UTR derived from Guanine nucleotide-binding protein G(s) subunit alpha isoforms short (Gnas); and (iv) a 5′-UTR derived from a hydroxysteroid (17-beta) dehydrogenase 4 gene (HSD17B4) and a 3′-UTR derived from a proteasome subunit beta type-3 (PSMB3) UTR.

Item 6. The mRNA according to any one of item 1 to item 5, wherein the
(i) G/C content of the HNF4A coding sequence in said mRNA is increased compared to the coding sequence of the corresponding wild type HNF4A coding sequence of SEQ ID NO:247 or 248;
(ii) C content of the HNF4A coding sequence in said mRNA is increased compared to the coding sequence of the corresponding wild type HNF4A coding sequence of SEQ ID NO:247 or 248; and/or wherein
(iii) at least one codon of the HNF4A coding sequence in said mRNA is adapted to human codon usage, wherein the codon adaptation index (CAI) is preferably increased or maximised in the corresponding HNF4A coding sequence compared to the coding sequence of the corresponding wild type HNF4A coding sequence of SEQ ID NO:247 or 248.

Item 7. The mRNA according to any one of item 1 to item 6, wherein the mRNA comprises a 5′-cap structure, a poly(A) sequence comprising at least 70 A nucleotides, preferably about 100 A nucleotides, a poly(C) sequence, preferably comprising 10 to 200, 10 to 100, 20 to 70, 20 to 60 or 10 to 40 cytosine nucleotides, and/or at least one histone stem-loop, preferably, wherein the mRNA comprises a 3′-terminal A nucleotide.

Item 8. The mRNA according to any one of item 1 to item 7, wherein the mRNA comprises, preferably in 5′ to 3′ direction, the following elements:
a) a 5′-cap1 structure;
b) a 5′-UTR element comprising a nucleic acid sequence, preferably derived from a 5′-UTR of a HSD17B4 gene, comprising the nucleic acid sequence according to SEQ ID NO:1 or 2, or a homolog, a fragment or a variant thereof;
c) at least one coding sequence as defined in any one of item 1 to item 10;
d) a 3′-UTR element comprising a nucleic acid sequence, preferably derived from a 3′-UTR of a PSMB3 gene, comprising the nucleic acid sequence according to SEQ ID NO:33 or 34, or a homolog, a fragment or a variant thereof;
e) a poly(A) sequence comprising about 100 adenosine nucleotides, preferably, wherein the mRNA comprises a 3′-terminal A nucleotide;
f) an optional poly(C) tail, preferably comprising 10 to 40 cytosine nucleotides; and/or
g) an optional histone stem-loop, preferably comprising the nucleic acid sequence according to SEQ ID NO:63 or 64.

Item 9. The mRNA according to any one of item 1 to item 8, wherein the open reading frame does not comprise any chemically modified uracil or cytosine nucleotides.

Item 10. The mRNA according to any one of item 1 to item 8, wherein the mRNA is chemically modified, preferably wherein the mRNA comprises pseudouridine (psi-uridine), N1-methylpseudouridine (N1MPU), 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosme, 7-deazaguanosine, 8-oxoadenosine, 8-oxoguanosine, O(6)-methylguanine, and/or 2-thiocytidine, more preferably wherein all uridine bases of the mRNA are fully chemically modified, even more preferably wherein all uridine bases of the mRNA are pseudouridine or N1-methylpseudouridine (N1MPU) bases, most preferably wherein all uridine bases of the mRNA are N1-methylpseudouridine (N1MPU) bases.

Item 11. A lipid nanoparticle (LNP) comprising the mRNA according to any one of item 1 to item 10, wherein the LNP comprises an ionizable or cationic lipid, a phospholipid, a structural lipid, and a polymer conjugated lipid.

Item 12. The LNP according to item 11, wherein the lipids comprised in the LNP have a molar ratio of about 20-60% cationic or ionizable lipid, about 5-25% non-cationic lipid, about 25-55% sterol and about 0.5-15% polymer conjugated lipid.

Item 13. The LNP according to anyone of item 11 to item 12, wherein the LNP does not comprise polyethylene glycol (PEG) or a PEG-modified lipid.

Item 14. A pharmaceutical composition, comprising the mRNA according to any one of item 1 to item 10 or the LNP according to any one of item 11 to item 13.

Item 15. A kit, preferably kit of parts, comprising at least one mRNA according to any one of item 1 to item 10, the LNP according to any one of item 11 to item 13, or the pharmaceutical composition according to item 14, and optionally a liquid vehicle for solubilising and optionally technical instructions with information on the administration and dosage of the pharmaceutical composition.

Item 16. A method of treating, preventing, attenuating or inhibiting, preferably selected from the group consisting of liver fibrosis, liver cirrhosis, hepatocellular carcinoma (HCC), non-alcoholic fatty liver disease (NAFLD), non-alcoholic steatohepatitis (NASH) or liver cancer, comprising administering to a human subject in need the mRNA according to any one of item 1 to item 10, the LNP according to any one of item 11 to item 13, the pharmaceutical composition according to item 14, or the kit or kit of parts according to item 15, wherein the administration results in treatment, prevention, attenuation, inhibition, or prophylaxis of the disease.

Item 17. The method according to item 16, wherein administration of the mRNA to a human subject in need results in
(i) improved hepatocyte metabolic activity; and/or
(ii) revived function of hepatocytes; and/or
(iii) increased mRNA levels of hepatocyte markers such as albumin (ALB), alpha-1 antitrypsin (A1AT), transferrin (TF) and/or transthyretin (TTR) in the liver or hepatocytes; and/or
(iv) reduced expression of fibrogenic marker genes Col1a1, Col2a1 Ck19, Sox9, Epcam, and/or Acta2 in the liver or hepatocytes; and/or
(v) decreased levels of bilirubin, hydroxyproline content in hepatocytes in the liver or hepatocytes; and/or
(vi) reduced liver injury as measured by histology, desmin or Sirius red staining;
(vii) increased expression of transporters of drug metabolism in the liver or hepatocytes;
(viii) increased serum paraoxonase and arylesterase 1 (PON1) expression or activity;
(ix) increased endogenous HNF4A or endogenous HNF1A levels or induction of the endogenous HNF1A-HNF4A transcriptional feedback loop; and/or
(x) function and fitness of hepatocytes;
as when compared to a non-treated human subject in need.

Item 18. The method according to any one of item 16 to item 17, wherein the mRNA according to any one of item 1 to item 9, or the LNP according to any one of item 11 to item 13, or the pharmaceutical composition according to item 14 or the kit or kit of parts according to item 15 is administered to the subject by subcutaneous, intramuscular or intravenous administration, preferably intravenous administration.

Item 19. The method according to any one of item 16 to item 18, wherein the mRNA comprises a 5′- or 3′-untranslated region (UTR) comprising at least one microRNA-binding site, preferably not being a microRNA-122 (miR-122) binding site, more preferably being miR-16, miR-21, miR-24, miR-27, miR-30c, miR-132, miR-133, miR-149, miR-192, miR-194, miR-204, miR-206, miR-208, or miR-223, most preferably being miRNA-148a, miRNA-101, miRNA-192 or miRNA-194, miR-126, miR-142-3p, or miR-142-5p.

Item 20. The method according to any one of item 16 to item 19, wherein the method of treating the liver disease or liver disorder, preferably liver fibrosis, liver cirrhosis, hepatocellular carcinoma (HCC), non-alcoholic fatty liver disease (NAFLD), non-alcoholic steatohepatitis (NASH) or liver cancer, involves a single administration of the mRNA, the LNP, the pharmaceutical composition or the kit or kit of parts.

Item 21. The method according to any one of item 16 to item 20, wherein the mRNA, the LNP, the pharmaceutical composition or the kit or kit of parts is administered
(a) once, preferably more than once, more preferably wherein administration is repeated for a period of at least 1 month, at least 2 months, at least 3 months, at least 4 months, at least 5 months, at least 6 months, at least one year, or lifelong; or
(b) about once a day, about once a week, about twice a week, about three times a week, about four times a week, about six or seven times a week, about once every two weeks, about once every three weeks, about once a month, about twice a month, about three times a month, or about four times a month.

Item 22. An isolated mRNA according to any one of item 1 to item 10, or LNP according to any one of item 11 to item 13 or pharmaceutical composition according to item 14 or kit or kit of parts according to item 15, for use as a medicament.

Item 23. An engineered HNF4A protein variant, comprising one or more amino acid exchange(s), leading to an increased HNF4A transcriptional activity or respectively DNA binding capacity, stability, longer-lasting HNF4A half-life and/or therapeutic effect as compared to the unmodified human wild type HNF4A protein according to SEQ ID NO:100, preferably an engineered HNF4A protein variant comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138 or SEQ ID NO:140, more preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

Item 23B. An engineered HNF4A protein variant, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A protein variant comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138 or SEQ ID NO:140, more preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

Item 24. The engineered HNF4A protein variant according to item 23, for use as a medicament.

Item 25. An isolated mRNA comprising an open reading frame (ORF) encoding an engineered HNF4A protein variant, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A protein variant comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138, SEQ ID NO:186 or SEQ ID NO:140, more preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

Item 26. An isolated mRNA having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:250, 299, 348, 397, 446, 495, 544, 593, 642, 1579, 1628, 1677, 1726, 1775, 1824, 1873, 1922, 1971, 2908, 2957, 3006, 3055, 3104, 3153, 3202, 3251, 3300, 4237, 4286, 4335, 4384, 4433, 4482, 4531, 4580, 4629, 251, 300, 349, 398, 447, 496, 545, 594, 643, 1580, 1629, 1678, 1727, 1776, 1825, 1874, 1923, 1972, 2909, 2958, 3007, 3056, 3105, 3154, 3203, 3252, 3301, 4238, 4287, 4336, 4385, 4434, 4483, 4532, 4581, 4630, 252, 301, 350, 399, 448, 497, 546, 595, 644, 1581, 1630, 1679, 1728, 1777, 1826, 1875, 1924, 1973, 2910, 2959, 3008, 3057, 3106, 3155, 3204, 3253, 3302, 4239, 4288, 4337, 4386, 4435, 4484, 4533, 4582, 4631, 253, 302, 351, 400, 449, 498, 547, 596, 645, 1582, 1631, 1680, 1729, 1778, 1827, 1876, 1925, 1974, 2911, 2960, 3009, 3058, 3107, 3156, 3205, 3254, 3303, 4240, 4289, 4338, 4387, 4436, 4485, 4534, 4583, 4632, 254, 303, 352, 401, 450, 499, 548, 597, 646, 1583, 1632, 1681, 1730, 1779, 1828, 1877, 1926, 1975, 2912, 2961, 3010, 3059, 3108, 3157, 3206, 3255, 3304, 4241, 4290, 4339, 4388, 4437, 4486, 4535, 4584, 4633, 255, 304, 353, 402, 451, 500, 549, 598, 647, 1584, 1633, 1682, 1731, 1780, 1829, 1878, 1927, 1976, 2913, 2962, 3011, 3060, 3109, 3158, 3207, 3256, 3305, 4242, 4291, 4340, 4389, 4438, 4487, 4536, 4585, 4634, 256, 305, 354, 403, 452, 501, 550, 599, 648, 1585, 1634, 1683, 1732, 1781, 1830, 1879, 1928, 1977, 2914, 2963, 3012, 3061, 3110, 3159, 3208, 3257, 3306, 4243, 4292, 4341, 4390, 4439, 4488, 4537, 4586, 4635, 257, 306, 355, 404, 453, 502, 551, 600, 649, 1586, 1635, 1684, 1733, 1782, 1831, 1880, 1929, 1978, 2915, 2964, 3013, 3062, 3111, 3160, 3209, 3258, 3307, 4244, 4293, 4342, 4391, 4440, 4489, 4538, 4587, 4636, 258, 307, 356, 405, 454, 503, 552, 601, 650, 1587, 1636, 1685, 1734, 1783, 1832, 1881, 1930, 1979, 2916, 2965, 3014, 3063, 3112, 3161, 3210, 3259, 3308, 4245, 4294, 4343, 4392, 4441, 4490, 4539, 4588, 4637, 259, 308, 357, 406, 455, 504, 553, 602, 651, 1588, 1637, 1686, 1735, 1784, 1833, 1882, 1931, 1980, 2917, 2966, 3015, 3064, 3113, 3162, 3211, 3260, 3309, 4246, 4295, 4344, 4393, 4442, 4491, 4540, 4589, 4638, 260, 309, 358, 407, 456, 505, 554, 603, 652, 1589, 1638, 1687, 1736, 1785, 1834, 1883, 1932, 1981, 2918, 2967, 3016, 3065, 3114, 3163, 3212, 3261, 3310, 4247, 4296, 4345, 4394, 4443, 4492, 4541, 4590, 4639, 261, 310, 359, 408, 457, 506, 555, 604, 653, 1590, 1639, 1688, 1737, 1786, 1835, 1884, 1933, 1982, 2919, 2968, 3017, 3066, 3115, 3164, 3213, 3262, 3311, 4248, 4297, 4346, 4395, 4444, 4493, 4542, 4591, 4640, 262, 311, 360, 409, 458, 507, 556, 605, 654, 1591, 1640, 1689, 1738, 1787, 1836, 1885, 1934, 1983, 2920, 2969, 3018, 3067, 3116, 3165, 3214, 3263, 3312, 4249, 4298, 4347, 4396, 4445, 4494, 4543, 4592, 4641, 263, 312, 361, 410, 459, 508, 557, 606, 655, 1592, 1641, 1690, 1739, 1788, 1837, 1886, 1935, 1984, 2921, 2970, 3019, 3068, 3117, 3166, 3215, 3264, 3313, 4250, 4299, 4348, 4397, 4446, 4495, 4544, 4593, 4642, 264, 313, 362, 411, 460, 509, 558, 607, 656, 1593, 1642, 1691, 1740, 1789, 1838, 1887, 1936, 1985, 2922, 2971, 3020, 3069, 3118, 3167, 3216, 3265, 3314, 4251, 4300, 4349, 4398, 4447, 4496, 4545, 4594, 4643, 265, 314, 363, 412, 461, 510, 559, 608, 657, 1594, 1643, 1692, 1741, 1790, 1839, 1888, 1937, 1986, 2923, 2972, 3021, 3070, 3119, 3168, 3217, 3266, 3315, 4252, 4301, 4350, 4399, 4448, 4497, 4546, 4595, 4644, 266, 315, 364, 413, 462, 511, 560, 609, 658, 1595, 1644, 1693, 1742, 1791, 1840, 1889, 1938, 1987, 2924, 2973, 3022, 3071, 3120, 3169, 3218, 3267, 3316, 4253, 4302, 4351, 4400, 4449, 4498, 4547, 4596, 4645, 267, 316, 365, 414, 463, 512, 561, 610, 659, 1596, 1645, 1694, 1743, 1792, 1841, 1890, 1939, 1988, 2925, 2974, 3023, 3072, 3121, 3170, 3219, 3268, 3317, 4254, 4303, 4352, 4401, 4450, 4499, 4548, 4597, 4646, 268, 317, 366, 415, 464, 513, 562, 611, 660, 1597, 1646, 1695, 1744, 1793, 1842, 1891, 1940, 1989, 2926, 2975, 3024, 3073, 3122, 3171, 3220, 3269, 3318, 4255, 4304, 4353, 4402, 4451, 4500, 4549, 4598, 4647, 269, 318, 367, 416, 465, 514, 563, 612, 661, 1598, 1647, 1696, 1745, 1794, 1843, 1892, 1941, 1990, 2927, 2976, 3025, 3074, 3123, 3172, 3221, 3270, 3319, 4256, 4305, 4354, 4403, 4452, 4501, 4550, 4599, 4648, 270, 319, 368, 417, 466, 515, 564, 613, 662, 1599, 1648, 1697, 1746, 1795, 1844, 1893, 1942, 1991, 2928, 2977, 3026, 3075, 3124, 3173, 3222, 3271, 3320, 4257, 4306, 4355, 4404, 4453, 4502, 4551, 4600, 4649, 271, 320, 369, 418, 467, 516, 565, 614, 663, 1600, 1649, 1698, 1747, 1796, 1845, 1894, 1943, 1992, 2929, 2978, 3027, 3076, 3125, 3174, 3223, 3272, 3321, 4258, 4307, 4356, 4405, 4454, 4503, 4552, 4601, 4650, 272, 321, 370, 419, 468, 517, 566, 615, 664, 1601, 1650, 1699, 1748, 1797, 1846, 1895, 1944, 1993, 2930, 2979, 3028, 3077, 3126, 3175, 3224, 3273, 3322, 4259, 4308, 4357, 4406, 4455, 4504, 4553, 4602, 4651, 273, 322, 371, 420, 469, 518, 567, 616, 665, 1602, 1651, 1700, 1749, 1798, 1847, 1896, 1945, 1994, 2931, 2980, 3029, 3078, 3127, 3176, 3225, 3274, 3323, 4260, 4309, 4358, 4407, 4456, 4505, 4554, 4603, 4652, 274, 323, 372, 421, 470, 519, 568, 617, 666, 1603, 1652, 1701, 1750, 1799, 1848, 1897, 1946, 1995, 2932, 2981, 3030, 3079, 3128, 3177, 3226, 3275, 3324, 4261, 4310, 4359, 4408, 4457, 4506, 4555, 4604, 4653, 275, 324, 373, 422, 471, 520, 569, 618, 667, 1604, 1653, 1702, 1751, 1800, 1849, 1898, 1947, 1996, 2933, 2982, 3031, 3080, 3129, 3178, 3227, 3276, 3325, 4262, 4311, 4360, 4409, 4458, 4507, 4556, 4605, 4654, 276, 325, 374, 423, 472, 521, 570, 619, 668, 1605, 1654, 1703, 1752, 1801, 1850, 1899, 1948, 1997, 2934, 2983, 3032, 3081, 3130, 3179, 3228, 3277, 3326, 4263, 4312, 4361, 4410, 4459, 4508, 4557, 4606, 4655, 277, 326, 375, 424, 473, 522, 571, 620, 669, 1606, 1655, 1704, 1753, 1802, 1851, 1900, 1949, 1998, 2935, 2984, 3033, 3082, 3131, 3180, 3229, 3278, 3327, 4264, 4313, 4362, 4411, 4460, 4509, 4558, 4607, 4656, 278, 327, 376, 425, 474, 523, 572, 621, 670, 1607, 1656, 1705, 1754, 1803, 1852, 1901, 1950, 1999, 2936, 2985, 3034, 3083, 3132, 3181, 3230, 3279, 3328, 4265, 4314, 4363, 4412, 4461, 4510, 4559, 4608, 4657, 279, 328, 377, 426, 475, 524, 573, 622, 671, 1608, 1657, 1706, 1755, 1804, 1853, 1902, 1951, 2000, 2937, 2986, 3035, 3084, 3133, 3182, 3231, 3280, 3329, 4266, 4315, 4364, 4413, 4462, 4511, 4560, 4609, 4658, 280, 329, 378, 427, 476, 525, 574, 623, 672, 1609, 1658, 1707, 1756, 1805, 1854, 1903, 1952, 2001, 2938, 2987, 3036, 3085, 3134, 3183, 3232, 3281, 3330, 4267, 4316, 4365, 4414, 4463, 4512, 4561, 4610, 4659, 281, 330, 379, 428, 477, 526, 575, 624, 673, 1610, 1659, 1708, 1757, 1806, 1855, 1904, 1953, 2002, 2939, 2988, 3037, 3086, 3135, 3184, 3233, 3282, 3331, 4268, 4317, 4366, 4415, 4464, 4513, 4562, 4611, 4660, 282, 331, 380, 429, 478, 527, 576, 625, 674, 1611, 1660, 1709, 1758, 1807, 1856, 1905, 1954, 2003, 2940, 2989, 3038, 3087, 3136, 3185, 3234, 3283, 3332, 4269, 4318, 4367, 4416, 4465, 4514, 4563, 4612, 4661, 283, 332, 381, 430, 479, 528, 577, 626, 675, 1612, 1661, 1710, 1759, 1808, 1857, 1906, 1955, 2004, 2941, 2990, 3039, 3088, 3137, 3186, 3235, 3284, 3333, 4270, 4319, 4368, 4417, 4466, 4515, 4564, 4613, 4662, 284, 333, 382, 431, 480, 529, 578, 627, 676, 1613, 1662, 1711, 1760, 1809, 1858, 1907, 1956, 2005, 2942, 2991, 3040, 3089, 3138, 3187, 3236, 3285, 3334, 4271, 4320, 4369, 4418, 4467, 4516, 4565, 4614, 4663, 285, 334, 383, 432, 481, 530, 579, 628, 677, 1614, 1663, 1712, 1761, 1810, 1859, 1908, 1957, 2006, 2943, 2992, 3041, 3090, 3139, 3188, 3237, 3286, 3335, 4272, 4321, 4370, 4419, 4468, 4517, 4566, 4615, 4664, 286, 335, 384, 433, 482, 531, 580, 629, 678, 1615, 1664, 1713, 1762, 1811, 1860, 1909, 1958, 2007, 2944, 2993, 3042, 3091, 3140, 3189, 3238, 3287, 3336, 4273, 4322, 4371, 4420, 4469, 4518, 4567, 4616, 4665, 287, 336, 385, 434, 483, 532, 581, 630, 679, 1616, 1665, 1714, 1763, 1812, 1861, 1910, 1959, 2008, 2945, 2994, 3043, 3092, 3141, 3190, 3239, 3288, 3337, 4274, 4323, 4372, 4421, 4470, 4519, 4568, 4617, 4666, 288, 337, 386, 435, 484, 533, 582, 631, 680, 1617, 1666, 1715, 1764, 1813, 1862, 1911, 1960, 2009, 2946, 2995, 3044, 3093, 3142, 3191, 3240, 3289, 3338, 4275, 4324, 4373, 4422, 4471, 4520, 4569, 4618, 4667, 289, 338, 387, 436, 485, 534, 583, 632, 681, 1618, 1667, 1716, 1765, 1814, 1863, 1912, 1961, 2010, 2947, 2996, 3045, 3094, 3143, 3192, 3241, 3290, 3339, 4276, 4325, 4374, 4423, 4472, 4521, 4570, 4619, 4668, 290, 339, 388, 437, 486, 535, 584, 633, 682, 1619, 1668, 1717, 1766, 1815, 1864, 1913, 1962, 2011, 2948, 2997, 3046, 3095, 3144, 3193, 3242, 3291, 3340, 4277, 4326, 4375, 4424, 4473, 4522, 4571, 4620, 4669, 291, 340, 389, 438, 487, 536, 585, 634, 683, 1620, 1669, 1718, 1767, 1816, 1865, 1914, 1963, 2012, 2949, 2998, 3047, 3096, 3145, 3194, 3243, 3292, 3341, 4278, 4327, 4376, 4425, 4474, 4523, 4572, 4621, 4670, 292, 341, 390, 439, 488, 537, 586, 635, 684, 1621, 1670, 1719, 1768, 1817, 1866, 1915, 1964, 2013, 2950, 2999, 3048, 3097, 3146, 3195, 3244, 3293, 3342, 4279, 4328, 4377, 4426, 4475, 4524, 4573, 4622, 4671, 293, 342, 391, 440, 489, 538, 587, 636, 685, 1622, 1671, 1720, 1769, 1818, 1867, 1916, 1965, 2014, 2951, 3000, 3049, 3098, 3147, 3196, 3245, 3294, 3343, 4280, 4329, 4378, 4427, 4476, 4525, 4574, 4623, 4672, 294, 343, 392, 441, 490, 539, 588, 637, 686, 1623, 1672, 1721, 1770, 1819, 1868, 1917, 1966, 2015, 2952, 3001, 3050, 3099, 3148, 3197, 3246, 3295, 3344, 4281, 4330, 4379, 4428, 4477, 4526, 4575, 4624, 4673, 295, 344, 393, 442, 491, 540, 589, 638, 687, 1624, 1673, 1722, 1771, 1820, 1869, 1918, 1967, 2016, 2953, 3002, 3051, 3100, 3149, 3198, 3247, 3296, 3345, 4282, 4331, 4380, 4429, 4478, 4527, 4576, 4625, 4674, 296, 345, 394, 443, 492, 541, 590, 639, 688, 1625, 1674, 1723, 1772, 1821, 1870, 1919, 1968, 2017, 2954, 3003, 3052, 3101, 3150, 3199, 3248, 3297, 3346, 4283, 4332, 4381, 4430, 4479, 4528, 4577, 4626, 4675, 297, 346, 395, 444, 493, 542, 591, 640, 689, 1626, 1675, 1724, 1773, 1822, 1871, 1920, 1969, 2018, 2955, 3004, 3053, 3102, 3151, 3200, 3249, 3298, 3347, 4284, 4333, 4382, 4431, 4480, 4529, 4578, 4627, 4676, 5720, 5721, 5723, 5724, 5726, 5727, 5729, 5730, 5732, 5733, 5735, and 5736.

Item 27. An isolated mRNA according to any one of item 1 to item 10 and item 25 to item 26, LNP according to any one of item 11 to item 13, pharmaceutical composition according to item 14, or kit or kit of parts according to item 15, for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer in a human subject in need, comprising administering to a human subject in need the wherein the administration results in treatment, prevention, attenuation, inhibition, or prophylaxis of the liver fibrosis, liver cirrhosis, hepatocellular carcinoma (HCC), non-alcoholic fatty liver disease (NAFLD), non-alcoholic steatohepatitis (NASH) or liver cancer.

Item 28. An isolated nucleic acid construct comprising a nucleic acid sequence encoding the mRNA according to item 8, preferably an isolated nucleic acid construct having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NO:1576-5615, 5683-5712, and 5716-5736 or SEQ ID NO:2906, 2956, 3005, 3250, 3299, 5728, 5731, 5734, 5563, 5564, 5565, 5566, 5567, 5568, 5569, 5570, 5571, 5572, 5573, 5574, 5575, 5576, 5577, 5578, 5579, 5580, 5581, 5582, 5583, 5584, 5585, 5586, 5587, 5588, 5589, 5590, 5591, 5592, 5593, 5594, 5595, 5596, 5597, 5598, 5599, 5600, 5601, 5602, 5603, 5604, 5605, 5606, 5607, 5608, 5609, 5610, 5611, 5612, 5613, 5614, and 5615 or to any one of the sequences as disclosed in Table C2 “Constructs of the invention”.

Item 28B. An isolated nucleic acid construct comprising a nucleic acid sequence encoding the mRNA according to item 8, preferably an isolated nucleic acid construct having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NO:1576-5615, 5683-5712, and 5716-5736 or to any one of the sequences as disclosed in Table C2 “Constructs of the invention”.

Item 29. A fusion protein comprising the engineered HNF4A protein variant of any one of item 23 or item 24 or an mRNA encoding a fusion protein comprising the engineered HNF4A protein variant of any one of item 23 or item 24.

Item 30. A vector comprising the isolated mRNA according to any one of item 1 to item 10.

Item 31. A host cell carrying the vector of item 30.

Item 32. An isolated mRNA encoding wild type hepatocyte nuclear factor 4 alpha (HNF4A) or preferably an isolated mRNA encoding an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation for use in treating or for use in supportive treatment of a disease which results in or leads to liver fibrosis or liver cirrhosis, said disease being selected from the group consisting of acute hepatic porphyria; Alagille syndrome; acute alcoholic hepatitis/alcoholic hepatitis; alcoholic steatohepatitis (ASH); alcoholic (fatty) liver disease (ALD); alcohol-related liver disease (ARLD); alpha-1 antitrypsin deficiency; autoimmune hepatitis (AIH); bile duct cancer (cholangiocarcinoma); biliary atresia; Brucellosis or syphilis; Budd-Chiari syndrome (BCS); chronic heart failure (HF); cystic fibrosis; galactosemia; glycogen storage disease Type 1/glycogen storage diseases; haemochromatosis; hepatitis A (HAV)/hepatitis A infection; hepatitis B (HBV)/chronic hepatitis B infection (CHB); hepatitis C (HCV)/chronic hepatitis C infection (CHC); hepatitis D (HDV)/chronic hepatitis D infection (CHD); hepatocellular carcinoma (HCC); benign liver tumors; lysosomal acid lipase deficiency (LAL-D); non-alcoholic fatty liver disease (NAFLD); non-alcoholic fatty liver (NAFL); non-alcoholic steatohepatitis (NASH); primary biliary cholangitis (PBC); primary biliary cirrhosis; primary sclerosing cholangitis (PSC); progressive familial intrahepatic cholestasis (PFIC); and Wilson disease.

Item 33. An isolated mRNA encoding
(i) an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation; or
(ii) wild type hepatocyte nuclear factor 4 alpha (HNF4A);
for use in treating or for use in supportive treatment of a disease which results in or leads to liver fibrosis or liver cirrhosis, said disease being selected from the group consisting of acute hepatic porphyria; Alagille syndrome; acute alcoholic hepatitis/alcoholic hepatitis; alcoholic steatohepatitis (ASH); alcoholic (fatty) liver disease (ALD); alcohol-related liver disease (ARLD); alpha-1 antitrypsin deficiency; autoimmune hepatitis (AIH); bile duct cancer (cholangiocarcinoma); biliary atresia; Brucellosis or syphilis; Budd-Chiari syndrome (BCS); chronic heart failure (HF); cystic fibrosis; galactosemia; glycogen storage disease Type 1/glycogen storage diseases; haemochromatosis; hepatitis A (HAV)/hepatitis A infection; hepatitis B (HBV)/chronic hepatitis B infection (CHB); hepatitis C (HCV)/chronic hepatitis C infection (CHC); hepatitis D (HDV)/chronic hepatitis D infection (CHD); hepatocellular carcinoma (HCC); benign liver tumors; lysosomal acid lipase deficiency (LAL-D); non-alcoholic fatty liver disease (NAFLD); non-alcoholic fatty liver (NAFL); non-alcoholic steatohepatitis (NASH); primary biliary cholangitis (PBC); primary biliary cirrhosis; primary sclerosing cholangitis (PSC); progressive familial intrahepatic cholestasis (PFIC); and Wilson disease.

Item 34. An isolated mRNA according to any one of the previous items, wherein all of the uracil in the coding sequence or the full nucleic acid sequence are exchanged with N1-methylpseudouridine (m14J).

Item 35. An isolated mRNA encoding
(i) an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising (a) one or (b) more than one or (c) all substitutions selected from the group consisting of S87A, K106R, K108R K126R K127R S142A S143A S148A T166A S167A S313A S378A T429A T432A S436A K458R and S461E;
for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, more preferably treating, preventing, attenuating or inhibiting liver fibrosis or liver cirrhosis.

Item 36. The mRNA of item 35(i) for use according to item 35, wherein said mRNA comprises an open reading frame (ORF) encoding an engineered HNF4A comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100,
preferably an engineered HNF4A comprising (a) one or (b) more than one or (c) all substitutions selected from the group consisting of S87A, K106R, K108R K126R K127R S142A S143A S148A T166A S167A S313A S378A T429A T432A S436A K458R and S461E, preferably SEQ ID NO:140,
more preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, or a fragment or variant of said sequences having the biological activity of a HNF4A protein.

### FIGURES—BRIEF DESCRIPTION OF THE DRAWINGS

FIG. 1: expression level of engineered HNF4A protein variants in HepG2 cells compared to WT HNF4A.

FIG. 2: Fold change of APOA4 protein levels in HepG2 cells upon expression of engineered HNF4A protein variants compared to WT HNF4A. Signals were normalized to unmutated codon-optimized WT HNF4A (GC; shown in black), error bars represent standard deviation (SD) of two independent biological experiments.

FIG. 3: HNF4A protein abundance in HepG2 cells upon expression of engineered HNF4A protein variants compared to unmutated HNF4A protein. Error bars represent standard deviation (SD) of two independent biological experiments followed by Western Blot analysis. Black line indicates height of unmutated HNF4A.

FIG. 4: HNF4A-dependent induction of endogenous APOA4 protein levels in HepG2 cells upon expression of engineered HNF4A protein variants compared to unmutated HNF4A protein. Error bars represent standard deviation (SD) of two independent biological experiments followed by Western Blot analysis. Black line indicates height of unmutated HNF4A.

FIG. 5: HNF4A protein abundance in HepG2 cells upon expression of engineered HNF4A protein variants compared to unmutated HNF4A protein. Error bars represent standard deviation (SD) of two independent biological experiments followed by Western Blot analysis. Black line indicates height of unmutated HNF4A.

FIG. 6: HNF4A-dependent induction of endogenous APOA4 protein levels in HepG2 cells upon expression of engineered HNF4A protein variants compared to unmutated HNF4A protein. Error bars represent standard deviation (SD) of two independent biological experiments followed by Western Blot analysis. Black line indicates height of unmutated HNF4A.

FIG. 7: qPCR analyses of human HNF4A mRNA in patients with fibrosis; 0 (n=9), 1 (n=3), 2 (n=3), 3 (n=3) and 4 (n=3) from Hannover Medical School (MHH), Germany and S0 (n=6), 51 (n=3), S2 (n=8), and S3 (n=5) and S4 (n=5) from Zhongshan Hospital, China.

FIG. 8: qPCRs for HNF4A target genes APOA2, APOB, F7 (y-axis: respective relative mRNA levels, x-axis: first column ZsGreen, 2^{nd} to 4^{th} column: HNF4A 3H, 6H, 12H)

FIG. 9: qPCRs for ALB, A1AT, TF and TTR in the following order of the probes:
1^{st} column control PHH control w/o mRNA;
2^{nd} column control PHH+ZsGreen mRNA;
3^{rd} column control PHH+HNF4A mRNA;
4^{th} column fibrotic PHH control w/o mRNA;
5^{th} column fibrotic PHH+ZsGreen mRNA;
6^{th} column fibrotic PHH+HNF4A mRNA.

FIG. 10: qPCRs for multiple CYPs, other enzymes, and transporters in phase I, phase II and phase III in the following order of the probes:
1^{st} column control PHH control w/o mRNA;
2^{nd} column control PHH+ZsGreen mRNA;
3^{rd} column control PHH+HNF4A mRNA;
4^{th} column fibrotic PHH control w/o mRNA;
5^{th} column fibrotic PHH+ZsGreen mRNA;
6^{th} column fibrotic PHH+HNF4A mRNA.

FIG. 11: ELISA for secreted ALB and A1AT in the following order of the probes:
1^{st} column control PHH control w/o mRNA;
2^{nd} column control PHH+ZsGreen mRNA;
3^{rd} column control PHH+HNF4A mRNA;
4^{h} column fibrotic PHH control w/o mRNA;
5^{th} column fibrotic PHH+ZsGreen mRNA;
6^{th} column fibrotic PHH+HNF4A mRNA.

FIG. 12: Representative immunofluorescence images at 3 hours (h), 6h, 12h, and 24h after ZsGreen mRNA transfection of PMH cells.

FIG. 13: the qPCR analyses of representative HNF4A target genes (Apoa2, Apob, and F7) expression after HNF4A mRNA transfection or ZsGreen mRNA.

FIG. 14: In this figures, the order of the probes is as follows:
1^{st} column=PMH isolated from untreated WT mice without CCl₄ (Carbon tetrachloride) treatment+ZsGreen mRNA;
2^{nd} column=PMH isolated from WT mice without CCl₄ treatment+HNF4A mRNA;
3^{rd} column=PMH isolated from fibrotic mice with CCl₄ treatment+ZsGreen mRNA;
4^{th} column=PMH isolated from fibrotic mice with CCl₄ treatment+HNF4A mRNA.

The first and second diagram show the qPCR analyses of relative mRNA levels of A/b and Ttr. The third and fourth diagram shows the hepatocyte function analyzed by measuring secreted levels of ALB (third diagram) and urea (fourth diagram). The fifth and sixth diagram show improved drug responsiveness of fibrotic hepatocytes after transfection with HNF4A mRNA as shown by elevated levels of Cyp1a2 (fifth diagram) and Cyp3a4 (sixth diagram). Data are mean+s.e.m; two-tailed Student's t-test. *: P<0.05; **: P<0.01.

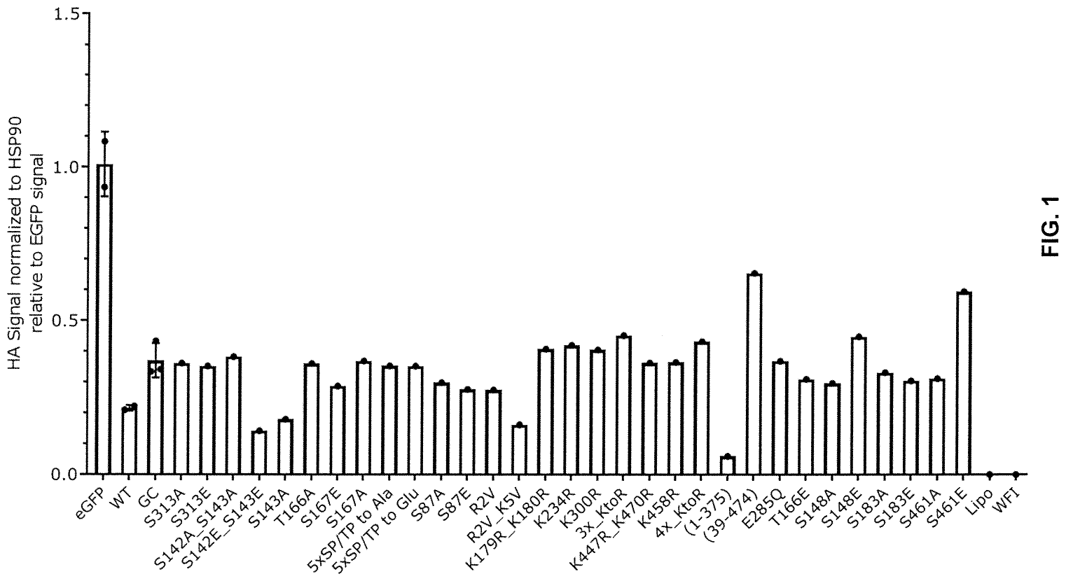

FIG. 15-1 (A to H):
Column 1: WT control
Column 2: CCl₄ control
Column 3: AAV control
Column 4: ZsGreen LNP
Column 5 AAV HNF4A
Column 6 HNF4A mRNA LNP
(A) Schematic of experiments analysing effect of human HNF4A mRNA delivery in CCl4-(A-H)-induced mouse models of liver fibrosis (n=6 mice per CCl4 model—arrows in 1^{st} row=16×PBS injections, 2^{nd} to 6^{th} row=16×CCl4 injections, 3^{rd} row=plus 6 additional HNF4A mRNA/LNP injections (bigger arrows), 4^{th} row=plus 6 additional ZsGreen mRNA/LNP injections (bigger arrows), 5^{th} row=1 AAV-HNF4A injection (big arrow), 6^{th} row=1 AAV-control injection (big arrow)).
(B) Hnf4a qPCRs after CCl4 injection.
(C) Western blots showing HNF4A protein expression.
(D) Liver function test for ALT and bilirubin show reduced injury upon HNF4A/LNP administration.
(E) Hydroxyproline assay.
(F) Immunohistochemical images of H&E, desmin, and Sirius red stainings. Scale bars, 100 μm.
(G) Quantification of Sirius red and desmin stainings.
(H) qPCRs for Col1a1, Col2a1 and Acta2.
*: P<0.05; **: P<0.01; ***: P<0.001; ****: P<0.0001.

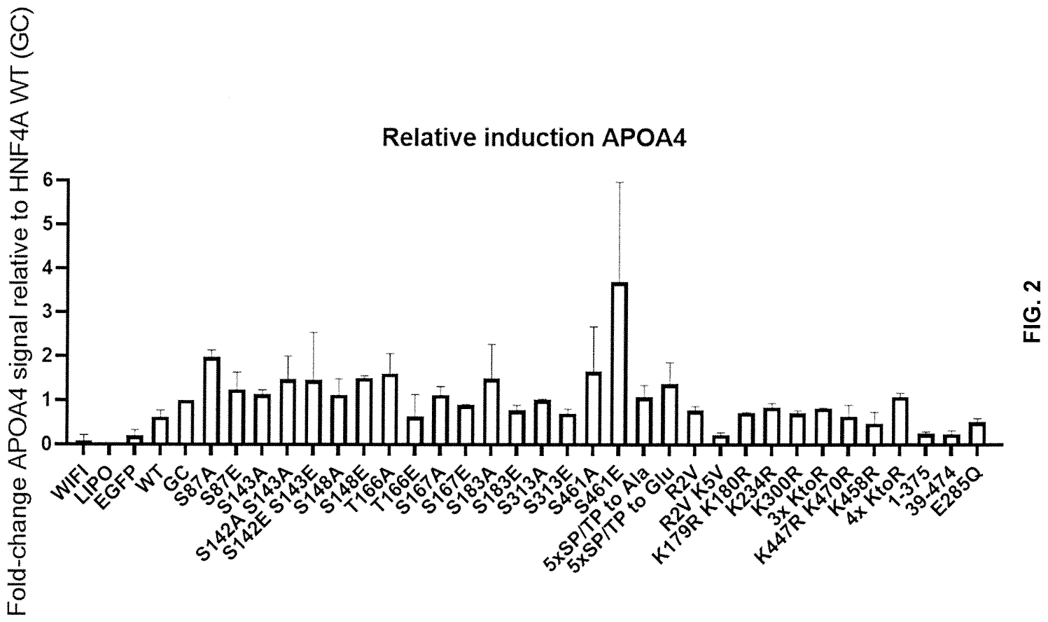

FIG. 15-2 (I to P):
Column 1: WT control
Column 2: CCl₄ control
Column 3: AAV control
Column 4: ZsGreen LNP
Column 5 AAV HNF4A
Column 6 HNF4A mRNA LNP
(I) Schematic of experiments analysing effect of human HNF4A mRNA delivery in DDC-(I-P)-induced mouse models of liver fibrosis (n=4 mice per DDC model—1^{st} row=normal diet, 2^{nd} to 6^{th} row=DDC diet, 3^{rd} row=plus 6 additional HNF4A mRNA/LNP injections (arrows), 4^{th} row=plus 6 additional ZsGreen mRNA/LNP injections (arrows), 5^{th} row=1 AAV-HNF4A injection (arrow), 6^{th} row=1 AAV-control injection (arrow)).
(J) Hnf4a qPCRs after DDC diet.
(K) Western blots showing HNF4A protein expression.
(L) Liver function test for ALT and bilirubin show reduced injury upon HNF4A/LNP administration.
(M) Hydroxyproline assay.
(N) Immunohistochemical images of H&E, desmin, and Sirius red stainings. Scale bars, 100 μm.
(O) Quantification of Sirius red and desmin stainings.
(P) qPCRs for Col1a1, Col2a1 and Acta2.
*: P<0.05; **: P<0.01; ***: P<0.001; ****: P<0.0001.

FIG. 16: Mitigation of liver injury in cirrhotic mice by delivery of HNF4A/LNP.
(A) Schematic of experimental design (small arrows in 1^{st} to 3^{rd} row=32×CCl₄ injections, 2^{nd} row=plus 6 additional HNF4A mRNA/LNP injections (bigger arrows), 3^{rd} row=plus 6 additional ZsGreen mRNA/LNP injections (bigger arrows)).
(B) qPCR for endogenous Hnf4a mRNA.
(C) Western blot revealing HNF4A protein expression in livers after HNF4A/LNP injection. Graphs showing
(D) Serum ALT levels and
(E) Hydroxyproline assay.
(F, G) Body weight and activity score for cirrhotic mice.
(H, I) Images of H&E, desmin, and Sirius red staining and quantification. Scale bars, 100 μm.
(J) qPCRs for Col1a1, Col2a1, and Acta2 in cirrhotic mice livers.
*: P<0.05; **: P<0.01; ***: P<0.001.

FIG. 17:
(A) Schematic of experimental design in Mdr2′ mice (1^{st} row=control, 2^{nd} row=plus 6 additional HNF4A mRNA/LNP injections (arrows), 3^{rd} row=plus 6 additional ZsGreen mRNA/LNP injections (arrows)).
(B) qPCR for endogenous Hnf4a mRNA in FVB control and Mdr2^{-/-} mice.
(C) Western blot revealed HNF4A protein expression after HNF4A/LNP injection. Graphs showing (D) Serum bilirubin levels and
(E) Hydroxyproline assay for collagen content.
(F) qPCRs for Ck19, Sox9, and Epcam.
(G) Images of H&E, desmin, and Sirius red staining
(H) and quantification. Scale bars, 100 μm.
(I) qPCRs for Col1a1, Col2a1, and Acta2 in livers from Mdr2^{-/-} mice.
*: P<0.05; **: P<0.01; ***: P<0.001.

FIG. 18: in vivo tolerability of repeated administration of LNP-formulated HNF4A mRNA (order starting top left, first row: IL-1b, IL-2, IL-3, IL-4, IL-10, 2^{nd} row: IL-12(p70), IL-13, IL-17, TNA-alpha, IFN-gamma). 1^{st} column=single ZsGreen/LNP injection, 2^{nd} column=single HNF4A/LNP injection, 3^{rd} column=repeated ZsGreen/LNP injection, 4^{th} column=repeated HNF4A/LNP injection. Data are mean±s.e.m; two-tailed Student's t-test for single injection groups.*: P<0.05.

FIG. 19: (A) qPCR analyses show that the endogenous Hnf4a was downregulated after 8 weeks of CCl₄ treatment, however its expression increased significantly in mice injected with multiple doses of HNF4A/LNP. n=6 for each group except WT control mice (n=4). (B) Similarly, the endogenous Hnf4a was downregulated in livers of mice fed with DDC diet, however the expression of endogenous Hnf4a increased significantly after repeated delivery of HNF4A/LNP. Data are mean±s.e.m; two-tailed Student's t-test. **: P<0.01; ***: P<0.001; ****: P<0.0001.

FIG. 20: Schematic experimental overview of Example 10. Mice were treated for 12 weeks with CCl₄ to induce liver cirrhosis. Thereafter, treatment phase began with mRNA encoding WT HNF4A protein or respectively engineered HNF4A protein variant combo 11, meanwhile CCl₄ treatment was maintained during treatment phase.

FIG. 21: Hydroxyproline assay (see Example 10). Deposition of collagen is a cornerstone of the progression of fibrotic pathology and its modulation is a promising target for a therapeutic strategy. Collagen can be quantified indirectly through hydroxyproline content since this amino acid is present almost exclusively in collagen. As apparent, hydroxyproline content is high in untreated disease model mice, while hydroxyproline content is reduced in disease model mice which received mRNA encoding WT HNF4A and reduced even stronger in mice which received engineered HNF4A protein variant combo 11. P value for CCl4+WT HNF4A was 0.02 while p value was significantly more potent for CCl4+engineered HNF4A combo 11 was 0.0015.

FIG. 22: Sirius red staining and quantification (see Example 10). Images from liver tissue stained with sirius red, staining collagen, shows, that collagen content is high in untreated disease model mice, while collagen content is reduced in disease model mice which received mRNA encoding WT HNF4A and reduced even stronger in mice which received engineered HNF4A protein variant combo 11. P value for CCl4+WT HNF4A was 0.035 while p value was significantly more potent for CCl4+engineered HNF4A combo 11 was 0.002.

FIG. 23: Col1a1 qPCR (see Example 10). Expression of fibrogenic marker gene Col1a1 was analyzed via qPCR. The detected Col1a1 RNA levels were comparable for the CCl₄ treated control and mRNA encoding WT HNF4A at 0.3 mg/kg, indicating that the mRNA encoding WT HNF4A was less efficient at this low dose and behaving as loss-of-function. In contrast, mRNA encoding combo 11 still decreased Col1a1 RNA levels at 0.3 mg/kg, confirming the conclusion that mRNA encoding engineered HNF4A protein variant combo 11 was more potent than mRNA encoding WT HNF4A. P value for CCl₄+WT HNF4A could not be assessed while p value was significantly more potent for CCl₄+engineered HNF4A combo 11 was 0.0014.

FIG. 24: Desmin staining and quantification (see Example 10). Desmin is a well-known marker of hepatic stellate cells in the normal liver. For further analysis in Example 10, images from liver tissue stained with desmin showed, that desmin positive area is high in untreated disease model mice, while desmin positive content is reduced in disease model mice which received mRNA encoding WT HNF4A and reduced even stronger in mice which received engineered HNF4A protein variant combo 11. P value for CCl₄+WT HNF4A was 0.039 while p value was significantly more potent for CCl₄+engineered HNF4A combo 11 was 0.008.

FIG. 25: Acta2 qPCR (see Example 10). Expression of fibrogenic marker gene Acta2 was analyzed via qPCR. The detected Acta2 RNA levels were high in untreated disease model mice and could be reduced through administration of mRNA encoding WT HNF4A and could be reduced even stronger through administration of engineered HNF4A protein variant combo 11. P value for CCl₄+WT HNF4A was 0.001404 while p value was significantly more potent for CCl₄+engineered HNF4A combo 11 was 0.000121.

FIG. 26: ALT (alanine aminotransferase) measurements (see Example 10). Strongly enhanced serum ALT levels were detected in untreated disease model mice. Detected ALT levels were reduced strongly in disease model mice treated with mRNA encoding WT HNF4A and engineered HNF4A protein variant combo 11, indicating improved liver function, suggesting decreased disease state. P value for CCl₄+WT HNF4A was 0.065 while p value was significantly more potent for CCl₄+engineered HNF4A combo 11 was 0.054.

### EXAMPLES

In the following section, particular examples illustrating various embodiments and aspects of the invention are presented. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the claims as disclosed herein.

Table of contents for Examples

| | |
|---|---|
| Example 1 | Preparation of DNA and RNA constructs, compositions, and mRNA/LNP formulations |
| Example 1B | Routine methods used in the working examples |
| Example 2 | Evaluation of HNF4A expression, stability and activity |
| Example 3 | In vitro WT HNF4A expression leads to improved function of fibrotic and non-fibrotic hepatocytes |
| | (PHH) |
| Example 4 | In vitro WT HNF4A expression leads to improved function of fibrotic and non-fibrotic hepatocytes |
| | (PMH) |
| Example 5 | In vivo verification of liver fibrosis inhibition in mouse model upon HNF4A mRNA delivery |
| Example 6 | In vivo verification of liver cirrhosis inhibition in mouse model upon HNF4A mRNA delivery |
| Example 7 | In vivo verification of suppression of cholestasis and fibrosis upon HNF4A mRNA delivery |
| Example 8 | In vivo tolerability of repeated administration of LNP-formulated HNF4A mRNA |
| Example 9 | Restoration of endogenous Hnf4a expression levels after repeated delivery of exogenous HNF4A |
| | mRNA encoding HNF4A WT protein in vivo |
| Example 10 | Suppression and reversal of liver cirrhosis in an In vivo disease mouse model upon administration |
| | of mRNA encoding engineered HNF4A protein variants |
| Example 11 | In vivo verification of liver cirrhosis inhibition in mouse model upon HNF4A mRNA delivery |
| [prophetic] | encoding engineered HNF4A protein variant S461E |

### Example 1: Preparation of DNA and RNA Constructs. Compositions, and mRNA/LNP Formulations

The present Example provides methods of obtaining the mRNA of the invention as well as methods of generating a pharmaceutical composition of the invention.

### 1.1. Preparation of DNA and RNA Constructs:

DNA sequences encoding different HNF4A protein designs, i.e. WT protein (SEQ ID NO:100) and engineered HNF4A protein variants (SEQ ID NO:101-246), were prepared and used for subsequent RNA in vitro transcription reactions. On DNA/RNA level, said DNA sequences were prepared by modifying the wild type or reference encoding DNA sequences by introducing a G/C optimized or modified coding sequence (e.g., “cds opt1”) for stabilization and expression optimization.

To further improve the intracellular stability as well as transcriptional activity of HNF4A, engineering of the protein sequence was performed in the context of “cds opt1”. For those engineered HNF4A protein variants, the amino acid sequence was modified on DNA/RNA level in accordance with the above and below disclosure of HNF4A variants and constructs of the invention. Molecular cloning techniques as well as gene synthesis was employed to generate engineered HNF4A protein variants/mutants. These mutants included deletions of N- and C-terminal regions, which vary across the 12 HNF4A isoforms, as well as mutants covering various post-translational modification sites (putative ubiquitination, acetylation, and phosphorylation sites). To prevent inhibitory ubiquitination and acetylation of HNF4A, arginine mutants were inserted. As the impact of phosphorylation on HNF4A activity is unclear, complementary phospho-incompetent (alanine) and phospho-mimetic (glutamate) mutants were generated.

Subsequently, sequences were introduced into a pUC derived DNA vector to comprise stabilizing 5′-UTR and 3′-UTR sequences, additionally comprising a stretch of adenosines (e.g. A64 or A100), optionally a histone-stem-loop (hSL) structure, and optionally a stretch of 30 cytosines (e.g. C30) (see Table Ex-1), also in accordance with the above and below disclosure of HNF4A variants and constructs of the invention. In sum, preferably, mRNA encoding engineered HNF4A protein variants were codon optimized (opt1), beared a CleanCap™ cap1 structure and a 3′-UTR tail ending with A100. For an overview of different designs see Table A to Table C1/2, Table Ex-1, the whole disclosure of the specification text and also the disclosure in the sequence listing, which comprises detailed information which is incorporated herein by reference in its entirety.

The obtained plasmid DNA constructs were transformed and propagated in bacteria using common protocols known in the art. Eventually, the plasmid DNA constructs were extracted, purified, and used for subsequent RNA in vitro transcription (see section 1.2.).

1.2. RNA In Vitro Transcription from Plasmid DNA Templates:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) and cap analog (e.g. m7GpppG, m7G(5′ppp(5)(2′OMeA)pG, m7G(5′)ppp(5)(2′OMeG)pG), 3′OMe-m7G(5)ppp(5′)(2′OMeA)pG, or respectively CleanCap™ purchased from TriLink) under suitable buffer conditions. The obtained RNA constructs were purified using RP-HPLC (PureMessenger®, CureVac AG, Thbingen, Germany; WO2008077592) and used for in vitro and in vivo experiments. DNA templates may also be generated using PCR. Such PCR templates can be used for DNA dependent RNA in vitro transcription using an RNA polymerase as outlined herein.

The resulting mRNA constructs of the examples or respectively RNA sequences/constructs are provided in Table Ex-1 with the encoded HNF4A protein or respectively engineered protein variant and the respective UTR elements indicated therein. If not indicated otherwise, the mRNA sequences/constructs of Table Ex-1 have been produced using RNA in vitro transcription in the presence of a m7GpppG or m7G(5)ppp(5)(2′OMeA)pG/CleanCap® respectively. Also here, for an overview of different designs see Table A to Table C1/2, Table Ex-1, the whole disclosure of the specification text and also the disclosure in the sequence listing, which comprises detailed information which is incorporated herein by reference in its entirety. Accordingly, the mRNA sequences/constructs comprise a 5′-cap1 structure. If not indicated otherwise, the mRNA sequences/constructs have been produced in the absence of chemically modified nucleotides like e.g. pseudouridine (4) or N(1)-methylpseudouridine (m14W or N1MPU).

TABLE EX-1
RNA constructs encoding different HNF4A protein or engineered HNF4A protein variants (more detailed information
can be found under <223> in the sequence listing of the invention [here, and throughout the whole
specification, it has to be noted that the priority application was filed with a sequence listing in accordance
with the WIPO Standard ST.25, which then was converted into a sequence listing according to WIPO Standard
ST.26 - information which was comprised within line <223> in ST.25 now was added to the respective SEQ ID NO:
as a note under “feature key”, i.e. “misc_feature” (for nucleic acids) or “REGION” (for proteins)])

| | 5′-UTR / | | | | | SEQ ID NO: | SEQ ID NO: |
|---|---|---|---|---|---|---|---|
| SEQ ID NO: | 3′-UTR; | RNA | | CDS | 3′- | Protein w/o | CDS w/o |
| RNA | UTR Design | ID | Name | opt. | end | HA-tag | HA-tag |
| 5713 | HSD17B4/ | R9011 | EGFP-3xHA | | hSL- | | |
| | PSMB3; a-1 | | | | A100 | | |
| 5714 | HSD17B4/ | R7797 | ZsGreen | | A64- | | |
| | PSMB3; a-1 | | | | N5 | | |
| 5715 | HSD17B4/ | R7805 | HNF4A(1-474) | opt29 | A64- | 100 | 641 |
| | PSMB3; a-1 | | | | N5 | | |
| 2906 | HSD17B4/ | R9821 | HNF4A(1-474) | wt | hSL- | 100 | 248 |
| | PSMB3; a-1 | | | | A100 | | |
| 2956 | HSD17B4/ | R9823, | HNF4A(1-474) | opt1 | hSL- | 100 | 298 |
| | PSMB3; a-1 | R10103 | | | A100 | | |
| 3005 | HSD17B4/ | R9824 | HNF4A(1-474) | opt2 | hSL- | 100 | 347 |
| | PSMB3; a-1 | | | | A100 | | |
| 3250 | HSD17B4/ | R9828 | HNF4A(1-474) | opt28 | hSL- | 100 | 592 |
| | PSMB3; a-1 | | | | A100 | | |
| 3299 | HSD17B4/ | R9822 | HNF4A(1-474) | opt29 | hSL- | 100 | 641 |
| | PSMB3; a-1 | | | | A100 | | |
| 5563 | HSD17B4/ | R9455 | HNF4A(1-474) HA-tag | wt | hSL- | 100 | 248 |
| | PSMB3; a-1 | | | | A100 | | |
| 5564 | HSD17B4/ | R9456 | HNF4A(1-474) HA-tag | opt1 | hSL- | 100 | 249 |
| | PSMB3; a-1 | | | | A100 | | |
| 5565 | HSD17B4/ | R9457 | HNF4A(1-474)(S313A) HA-tag | opt1 | hSL- | 101 | 250 |
| | PSMB3; a-1 | | | | A100 | | |
| 5566 | HSD17B4/ | R9458 | HNF4A(1-474)(S313E) HA-tag | opt1 | hSL- | 102 | 251 |
| | PSMB3; a-1 | | | | A100 | | |
| 5567 | HSD17B4/ | R9459 | HNF4A(1-474)(S142A S143A) | opt1 | hSL- | 103 | 252 |
| | PSMB3; a-1 | | HA-tag | | A100 | | |
| 5568 | HSD17B4/ | R9460 | HNF4A(1-474)(S142E S143E) | opt1 | hSL- | 104 | 253 |
| | PSMB3; a-1 | | HA-tag | | A100 | | |
| 5569 | HSD17B4/ | R9461 | HNF4A(1-474)(S143A) HA-tag | opt1 | hSL- | 105 | 254 |
| | PSMB3; a-1 | | | | A100 | | |
| 5570 | HSD17B4/ | R9462 | HNF4A(1-474)(T166A) HA-tag | opt1 | hSL- | 106 | 255 |
| | PSMB3; a-1 | | | | A100 | | |
| 5571 | HSD17B4/ | R9463 | HNF4A(1-474)(S167E) HA-tag | opt1 | hSL- | 107 | 256 |
| | PSMB3; a-1 | | | | A100 | | |
| 5572 | HSD17B4/ | R9464 | HNF4A(1-474)(S167A) HA-tag | opt1 | hSL- | 108 | 257 |
| | PSMB3; a-1 | | | | A100 | | |
| 5573 | HSD17B4/ | R9465 | HNF4A(1-474)(S167A S378A | opt1 | hSL- | 109 | 258 |
| | PSMB3; a-1 | | T429A T432A S436A) HA-tag | | A100 | | |
| 5574 | HSD17B4/ | R9466 | HNF4A(1-474)(S167E S378E | opt1 | hSL- | 110 | 259 |
| | PSMB3; a-1 | | T429E T432E S436E) HA-tag | | A100 | | |
| 5575 | HSD17B4/ | R9467 | HNF4A(1-474)(S87A) HA-tag | opt1 | hSL- | 111 | 260 |
| | PSMB3; a-1 | | | | A100 | | |
| 5576 | HSD17B4/ | R9468 | HNF4A(1-474)(S87E) HA-tag | opt1 | hSL- | 112 | 261 |
| | PSMB3; a-1 | | | | A100 | | |
| 5577 | HSD17B4/ | R9469 | HNF4A(1-474)(R2V) HA-tag | opt1 | hSL- | 113 | 262 |
| | PSMB3; a-1 | | | | A100 | | |
| 5578 | HSD17B4/ | R9470 | HNF4A(1-474)(R2V K5V) | opt1 | hSL- | 114 | 263 |
| | PSMB3; a-1 | | HA-tag | | A100 | | |
| 5579 | HSD17B4/ | R9471 | HNF4A(1-474)(K179R K180R) | opt1 | hSL- | 115 | 264 |
| | PSMB3; a-1 | | HA-tag | | A100 | | |
| 5580 | HSD17B4/ | R9472 | HNF4A(1-474)(K234R) HA-tag | opt1 | hSL- | 116 | 265 |
| | PSMB3; a-1 | | | | A100 | | |
| 5581 | HSD17B4/ | R9473 | HNF4A(1-474)(K300R) HA-tag | opt1 | hSL- | 117 | 266 |
| | PSMB3; a-1 | | | | A100 | | |
| 5582 | HSD17B4/ | R9474 | HNF4A(1-474)(K300R K307R | opt1 | hSL- | 118 | 267 |
| | PSMB3; a-1 | | K309R) HA-tag | | A100 | | |
| 5583 | HSD17B4/ | R9475 | HNF4A(1-474)(K447R K470R) | opt1 | hSL- | 119 | 268 |
| | PSMB3; a-1 | | HA-tag | | A100 | | |
| 5584 | HSD17B4/ | R9476 | HNF4A(1-474)(K458R) HA-tag | opt1 | hSL- | 120 | 269 |
| | PSMB3; a-1 | | | | A100 | | |
| 5585 | HSD17B4/ | R9477 | HNF4A(1-474)(K106R K108R | opt1 | hSL- | 121 | 270 |
| | PSMB3; a-1 | | K126R K127R) HA-tag | | A100 | | |
| 5586 | HSD17B4/ | R9480 | HNF4A(1-474)(E285Q) HA-tag | opt1 | hSL- | 122 | 271 |
| | PSMB3; a-1 | | | | A100 | | |
| 5587 | HSD17B4/ | R9481 | HNF4A(1-474)(T166E) HA-tag | opt1 | hSL- | 123 | 272 |
| | PSMB3; a-1 | | | | A100 | | |
| 5588 | HSD17B4/ | R9532 | HNF4A(1-474)(S148A) HA-tag | opt1 | hSL- | 124 | 273 |
| | PSMB3; a-1 | | | | A100 | | |
| 5589 | HSD17B4/ | R9533 | HNF4A(1-474)(S148E) HA-tag | opt1 | hSL- | 125 | 274 |
| | PSMB3; a-1 | | | | A100 | | |
| 5590 | HSD17B4/ | R9534 | HNF4A(1-474)(S183A) HA-tag | opt1 | hSL- | 126 | 275 |
| | PSMB3; a-1 | | | | A100 | | |
| 5591 | HSD17B4/ | R9535 | HNF4A(1-474)(S183E) HA-tag | opt1 | hSL- | 127 | 276 |
| | PSMB3; a-1 | | | | A100 | | |
| 5592 | HSD17B4/ | R9536 | HNF4A(1-474)(S461A) HA-tag | opt1 | hSL- | 128 | 277 |
| | PSMB3; a-1 | | | | A100 | | |
| 5593 | HSD17B4/ | R9537 | HNF4A(1-474)(S461E) HA-tag | opt1 | hSL- | 129 | 278 |
| | PSMB3; a-1 | | | | A100 | | |
| 5594 | HSD17B4/ | R9849 | HNF4A(1-474)(S142A S143A | opt1 | hSL- | 130 | 279 |
| | PSMB3; a-1 | | S313A) HA-tag | | A100 | | |
| 5595 | HSD17B4/ | R9850 | HNF4A(1-474)(S142A S143A | opt1 | hSL- | 131 | 280 |
| | PSMB3; a-1 | | S313A K458R) HA-tag | | A100 | | |
| 5596 | HSD17B4/ | R9851 | HNF4A(1-474)(S142A S143A | opt1 | hSL- | 132 | 281 |
| | PSMB3; a-1 | | S167A S313A S378A T429A T432A | | A100 | | |
| | | | S436A K458R) HA-tag | | | | |
| 5597 | HSD17B4/ | R9852 | HNF4A(1-474)(S142A S143A | opt1 | hSL- | 133 | 282 |
| | PSMB3; a-1 | | S148A S167A S313A S378A T429A | | A100 | | |
| | | | T432A S436A K458R) HA-tag | | | | |
| 5598 | HSD17B4/ | R9853 | HNF4A(1-474)(K106R K108R | opt1 | hSL- | 134 | 283 |
| | PSMB3; a-1 | | K126R K127R S142A S143A S148A | | A100 | | |
| | | | S167A S313A S378A T429A T432A | | | | |
| | | | S436A K458R) HA-tag | | | | |
| 5599 | HSD17B4/ | R9873 | HNF4A(1-474)(S142A S143A | opt1 | hSL- | 135 | 284 |
| | PSMB3; a-1 | | S148A T166A S167A S313A S378A | | A100 | | |
| | | | T429A T432A S436A K458R) | | | | |
| | | | HA-tag | | | | |
| 5600 | HSD17B4/ | R9874 | HNF4A(1-474)(S87A S142A S143A | opt1 | hSL- | 136 | 285 |
| | PSMB3; a-1 | | S148A T166A S167A S313A S378A | | A100 | | |
| | | | T429A T432A S436A K458R) | | | | |
| | | | HA-tag | | | | |
| 5601 | HSD17B4/ | R9875 | HNF4A(1-474)(S87A K106R K108R | opt1 | hSL- | 137 | 286 |
| | PSMB3; a-1 | | K126R K127R S142A S143A S148A | | A100 | | |
| | | | T166A S167A S313A S378A T429A | | | | |
| | | | T432A S436A K458R) HA-tag | | | | |
| 5602 | HSD17B4/ | R10313 | HNF4A(1-474)(S87A K106R K108R | opt1 | hSL- | 138 | 287 |
| | PSMB3; a-1 | | K126R K127R) HA-tag | | A100 | | |
| 5603 | HSD17B4/ | R10314 | HNF4A(1-474)(S87A K106R K108R | opt1 | hSL- | 139 | 288 |
| | PSMB3; a-1 | | K126R K127R S142A S143A S148A | | A100 | | |
| | | | T166A S167A S313A S378A T429A | | | | |
| | | | T432A S436A K458R S461A) | | | | |
| | | | HA-tag | | | | |
| 5604 | HSD17B4/ | R10315 | HNF4A(1-474)(S87A K106R K108R | opt1 | hSL- | 140 | 289 |
| | PSMB3; a-1 | | K126R K127R S142A S143A S148A | | A100 | | |
| | | | T166A S167A S313A S378A T429A | | | | |
| | | | T432A S436A K458R S461E) | | | | |
| | | | HA-tag | | | | |
| 5605 | HSD17B4/ | R10316 | HNF4A(1-474)(S87A K106R K108R | opt1 | hSL- | 141 | 290 |
| | PSMB3; a-1 | | K126R K127R S142A S143A S148E | | A100 | | |
| | | | T166A S167E S313A S378E T429E | | | | |
| | | | T432E S436E K458R S461E) | | | | |
| | | | HA-tag | | | | |
| 5606 | HSD17B4/ | R10317 | HNF4A(1-474)(S87A K106R K108R | opt1 | hSL- | 142 | 291 |
| | PSMB3; a-1 | | K126R K127R S142A S143A S148A | | A100 | | |
| | | | T166A S167A K179R K180R S313A | | | | |
| | | | S378A T429A T432A S436A K458R | | | | |
| | | | S461E) HA-tag | | | | |
| 5607 | HSD17B4/ | R10318 | HNF4A(1-474)(S87A K106R K108R | opt1 | hSL- | 143 | 292 |
| | PSMB3; a-1 | | K126R K127R S142A S143A S148A | | A100 | | |
| | | | T166A S167A K300R K307R K309R | | | | |
| | | | S313A S378A T429A T432A S436A | | | | |
| | | | K458R S461E) HA-tag | | | | |
| 5608 | HSD17B4/ | R10319 | HNF4A(1-474)(S87A K106R K108R | opt1 | hSL- | 144 | 293 |
| | PSMB3; a-1 | | K126R K127R S142A S143A S148A | | A100 | | |
| | | | T166A S167A S313A S378A T429A | | | | |
| | | | T432A S436A K447R K458R S461E | | | | |
| | | | K470R) HA-tag | | | | |
| 5609 | HSD17B4/ | R10320 | HNF4A(1-474)(S87A K106R K108R | opt1 | hSL- | 145 | 294 |
| | PSMB3; a-1 | | K126R K127R S142A S143A S148A | | A100 | | |
| | | | T166A S167A K179R K180R K234R | | | | |
| | | | K300R K307R K309R S313A S378A | | | | |
| | | | T429A T432A S436A K447R K458R | | | | |
| | | | S461E K470R) HA-tag | | | | |
| 5610 | HSD17B4/ | R10321 | HNF4A(1-474)(S87A S95A S99A | opt1 | hSL- | 146 | 295 |
| | PSMB3; a-1 | | K106R K108R K126R K127R S142A | | A100 | | |
| | | | S143A S148A T166A S167A S313A | | | | |
| | | | S378A T429A T432A S436A K458R | | | | |
| | | | S461E) HA-tag | | | | |
| 5611 | HSD17B4/ | R10322 | HNF4A(1-474)(S87A K106R K108R | opt1 | hSL- | 147 | 296 |
| | PSMB3; a-1 | | K126R K127R S138A T139A S142A | | A100 | | |
| | | | S143A S148A T166A S167A S313A | | | | |
| | | | S378A T429A T432A S436A K458R | | | | |
| | | | S461E) HA-tag | | | | |
| 5612 | HSD17B4/ | R10323 | HNF4A(1-474)(S87A S95A S99A | opt1 | hSL- | 148 | 297 |
| | PSMB3; a-1 | | K106R K108R K126R K127R S138A | | A100 | | |
| | | | T139A S142A S143A S148A T166A | | | | |
| | | | S167A S313A S378A T429A T432A | | | | |
| | | | S436A K458R S461E) HA-tag | | | | |
| 5613 | HSD17B4/ | R9478 | HNF4A(1-375) HA-tag | opt1 | hSL- | 149 | 692 |
| | PSMB3; a-1 | | | | A100 | | |
| 5614 | HSD17B4/ | R9479 | HNF4A(39-474) HA-tag | opt1 | hSL- | 198 | 1135 |
| | PSMB3; a-1 | | | | A100 | | |
| 5615 | HSD17B4/ | R9697 | HNF4A(1-474) HA-tag | opt29 | hSL- | 100 | 641 |
| | PSMB3; a-1 | | | | A100 | | |
| 2907 | HSD17B4/ | R10692 | HNF4A(1-474) | opt1 | hSL- | 100 | 249 |
| | PSMB3; a-1 | | | | A100 | | |
| 2947 | HSD17B4/ | R10665 | HNF4A(1-474) | opt1 | hSL- | 140 | 289 |
| | PSMB3; a-1 | | (S87A_K106R_K108R_K126R_{—} | | A100 | | |
| | | | K127R_S142A_S143A_S148A_{—} | | | | |
| | | | T166A_S167A_S313A_S378A_{—} | | | | |
| | | | T429A_T432A_S436A_K458R_S461E) | | | | |

In certain examples, e.g. Example 2, the mRNA from the basic enhanced fluorescent green fluorescent protein (EGFP) served as control SEQ ID NO: 5713). To enable detection and direct comparison of protein abundance, both HNF4A variants and EGFP were fused to a triple hemagglutinin tag (3×HA tag).

In further examples, mRNA from the basic constitutively fluorescent green fluorescent protein ZsGreen (derived from Zoanthus sp., SEQ ID NO:5714) were used as control and were produced and formulated analogously as described above or below.

For in vivo studies, mRNAs were further polyadenylated using A-Plus Poly (A) Polymerase Tailing Kit (Biozym) and carried out according to the manufacturer's recommendations.

1.3. Preparation of LNP Formulated mRNA Pharmaceutical Composition:

If for experimentation mRNAs of the invention encoding engineered HNF4A (see Table Ex-1) were formulated in LNPs, those LNPs were prepared and tested according to the general procedures described in Thess et al. (PMID 26050989), PCT Publication Nos WO2015199952, WO2017004143, WO2017075531 and most preferably WO2018078053, the full disclosures of which are incorporated herein by reference. For designation in the experimental part, the respective amino acid substitutions or deletions or combinations thereof were used, also referring to the designations provided above in Table B-I (combo 1 etc.), as can be seen here for easier reference (see Table Ex-1b).

TABLE Ex-1b
designation of combinations of amino acid substitutions or deletions (for further information
Table B-I shows further exemplary suitable HNF4A protein designs including mRNAs encoding
said proteins including their SEQ ID NOs) - “*” indicates an mRNA with N1-methylpseudouridine
(N1MPU, m1f), “**” indicates an mRNA with pseudouridine (psi-uridine, p)

| | | | Exemplary | Exemplary |
|---|---|---|---|---|
| | | | preferred | preferred |
| | amino acid substitutions or deletions in | PRT SEQ | CDS SEQ | mRNA SEQ |
| Designation | comparison to HNF4A WT protein | ID NO: | ID NO: | ID NO: |
| WT | | 100 | 249 | 2907, 5719, |
| | | | | 5722*, 5725** |
| S87A | S87A | 111 | 260 | 2918 |
| S461E | S461E | 129 | 278 | 2936, 5720, |
| | | | | 5723*, 5726** |
| combo 1 | S142A S143A S313A | 130 | 279 | 2937 |
| combo 2 | S142A S143A S313A K458R | 131 | 280 | 2938 |
| combo 3 | S142A S143A S167A S313A S378A T429A | 132 | 281 | 2939 |
| | T432A S436A K458R | | | |
| combo 4 | S142A S143A S148A S167A S313A S378A | 133 | 282 | 2940 |
| | T429A T432A S436A K458R | | | |
| combo 5 | K106R K108R K126R K127R S142A S143A | 134 | 283 | 2941 |
| | S148A S167A S313A S378A T429A T432A | | | |
| | S436A K458R | | | |
| combo 6 | S142A S143A S148A T166A S167A S313A | 135 | 284 | 2942 |
| | S378A T429A T432A S436A K458R | | | |
| combo 7 | S87A S142A S143A S148A T166A S167A S313A | 136 | 285 | 2943 |
| | S378A T429A T432A S436A K458R | | | |
| combo 8 | S87A K106R K108R K126R K127R S142A S143A | 137 | 286 | 2944 |
| | S148A T166A S167A S313A S378A T429A | | | |
| | T432A S436A K458R | | | |
| combo 9 (S87A | S87A K106R K108R K126R K127R | 138 | 287 | 2945 |
| 4x K to R) | | | | |
| combo 10 (combo | S87A K106R K108R K126R K127R S142A S143A | 139 | 288 | 2946 |
| 8 + S461A) | S148A T166A S167A S313A S378A T429A | | | |
| | T432A S436A K458R S461A | | | |
| combo 11 (combo | S87A K106R K108R K126R K127R S142A S143A | 140 | 289 | 2947, 5721, |
| 8 + S461E) | S148A T166A S167A S313A S378A T429A | | | 5724*, 5727** |
| | T432A S436A K458R S461E | | | |
| combo 12 | S87A K106R K108R K126R K127R S142A S143A | 141 | 290 | 2948 |
| | S148E T166A S167E S313A S378E T429E T432E | | | |
| | S436E K458R S461E | | | |
| combo 13 | S87A K106R K108R K126R K127R S142A S143A | 142 | 291 | 2949 |
| | S148A T166A S167A K179R K180R S313A | | | |
| | S378A T429A T432A S436A K458R S461E | | | |
| combo 14 | S87A K106R K108R K126R K127R S142A S143A | 143 | 292 | 2950 |
| | S148A T166A S167A K300R K307R K309R | | | |
| | S313A S378A T429A T432A S436A K458R | | | |
| | S461E | | | |
| combo 15 | S87A K106R K108R K126R K127R S142A S143A | 144 | 293 | 2951 |
| | S148A T166A S167A S313A S378A T429A | | | |
| | T432A S436A K447R K458R S461E K470R | | | |
| combo 16 | S87A K106R K108R K126R K127R S142A S143A | 145 | 294 | 2952 |
| | S148A T166A S167A K179R K180R K234R | | | |
| | K300R K307R K309R S313A S378A T429A | | | |
| | T432A S436A K447R K458R S461E K470R | | | |
| combo 17 | S87A S95A S99A K106R K108R K126R K127R | 146 | 29 | 2953 |
| | S142A S143A S148A T166A S167A S313A | | | |
| | S378A T429A T432A S436A K458R S461E | | | |
| combo 18 | S87A K106R K108R K126R K127R S138A T139A | 147 | 296 | 2954 |
| | S142A S143A S148A T166A S167A S313A | | | |
| | S378A T429A T432A S436A K458R S461E | | | |
| combo 19 | S87A S95A S99A K106R K108R K126R K127R | 148 | 297 | 2955 |
| | S138A T139A S142A S143A S148A T166A | | | |
| | S167A S313A S378A T429A T432A S436A | | | |
| | K458R S461E | | | |

Summarized, LNP-formulated mRNAs were prepared using an ionizable amino lipid (cationic lipid), phospholipid, cholesterol and a PEGylated lipid. LNPs were prepared as follows. Cationic lipid according to formula III-3 (ALC-0315; GAS 2036272-55-4), DSPC, cholesterol and PEG-lipid according to formula IVa (ALC-0159) were solubilized in ethanol at a molar ratio of approximately 47.5:10:40.8:1.7 (see Table Ex-2). LNPs comprising compound III-3 were prepared at a ratio of mRNA (sequences see Table Ex-1) to total lipid of 0.03-0.04 weight/weight. Briefly, the mRNA was diluted to 0.05 to 0.2 mg/mL in 10 to 50 mM citrate buffer, pH 4. Syringe pumps were used to mix the ethanolic lipid solution with the mRNA aqueous solution at a ratio of about 1:5 to 1:3 (vol/vol) with total flow rates above 15 ml/min. For injections, the ethanol was then removed and mRNA-LNPs and the external buffer was replaced with phosphate-buffered saline pH 7.4 (PBS) by dialysis. Finally, the LNPs were filtered through a 0.2 μm pore sterile filter. Lipid nanoparticle particle diameter size was 60-90 nm as determined by quasi-elastic light scattering using a Malvern Zetasizer Nano (Malvern, UK).

TABLE EX-2
Lipid-based carrier composition of the examples

| | LNP | Proportion | |
|---|---|---|---|
| | excipients | (mol %) | Structure |
| 1 | Cationic lipid ALC- 0315 CAS 2036272- 55-4 | 47.4 | 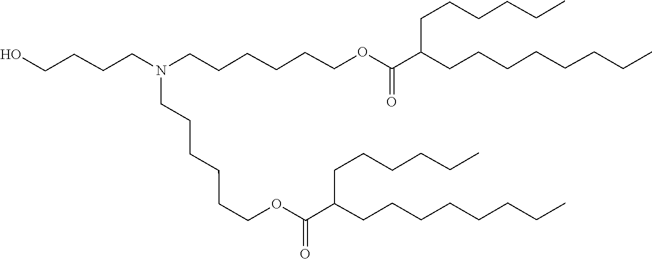 [SMILES: CCCCCCCCC(CCCCCC)C(=O)OCCCCCCN(CCCCO)CCCCCCOC(=O)C(CCCCCC)CCCCCCCC] |
| 2 | Cholesterol | 40.9 | 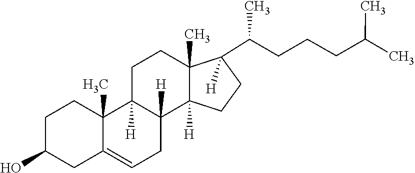 |
| 3 | 1,2- distearoyl- sn-glycero-3- phosphocholine (DSPC) | 10 | 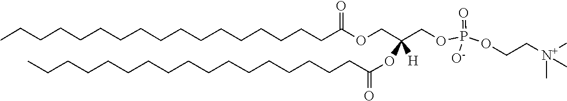 [SMILES: CCCCCCCCCCCCCCCCCC(=O)OC[C@@H](COP(=O)([O-])OCC[N+](C)(C)C)OC(=O)CCCCCCCCCCCCCCCCC] |
| 4 | PEG lipid ALC- 0159 CAS 1849616- 42-7 | 1.7 | 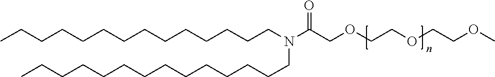 Average n = ~49 |

### Example 1B—Routine Methods Used in the Working Examples

### Reverse Transcription and Quantitative PCR (RT-qPCR)

Livers were taken at indicated time points after injection with mRNA/LNP. For in vitro studies, primary mouse hepatocytes and primary human hepatocytes were cultured according to routine methods. Total RNAs were extracted by Trizol reagent (Qiagen, Hilden, Germany) from cells or tissues according to manufacturer's instructions. cDNA was generated using TaqMan Reverse Transcription Reagents (ThermoFisher Scientific, Waltham, USA). RT-qPCR was performed according to the TaqMan (Invitrogen, Waltham, US) or SYBR Green (ThermoFisher Scientific, Waltham, USA) Gene Expression Assay protocols using a 96-well ABI StepOne Plus Real-Time PCR System (ThermoFisher Scientific, Waltham, USA). Samples were run in triplicate. Relative mRNA expression was determined after normalizing the expression with respect to GAPDH, which was used as a house-keeping gene.

### Blood Chemistry Analyses and Multiple Cytokines and Chemokines Analyses

200 μl blood from mice with different treatments and sera were prepared and stored in −80° C. Bilirubin and alanine aminotransferase (ALT) were assayed as indicators of liver function. Plasma multiple cytokines and chemokine concentrations were quantified by multiplex protein arrays kit, according to the manufacturer's protocols (BioRad Laboratories, Hercules, USA). Following cytokines and chemokines were measured according to routine methods: IL-1 beta, IL-2, IL-3, IL-4, IL-10, IL-12p70, IL-13, IL-17, tumor necrosis factor-alpha (TNF-α), and Interferon-gamma (IFN-γ). To normalize the hemodilution at the end of the procedure, all biochemistry and cytokine values were corrected by using the following formula: Value (calculated)=(Value (measured)×Hematocrit (measured))/Hematocrit (baseline)

### Histology, Immunohistochemistry, and Immunofluorescence

Liver tissues were fixed with 4% formalin, embedded in paraffin, and cut into 5 μm-thick sections for histological and immunohistochemical analyses. For Sirius red staining, following deparaffinization, the sections were stained with Picro-Sirius Red Stain Kit according to manufacturer's instructions (Abcam, ab150681). The sections were hydrated in distilled water and incubated in a picro-Sirius red solution for 60 minutes. After washing slides with acetic acid solution and absolute alcohol, the slides were then dehydrated and subsequently mounted. Immunofluorescence stainings for desmin (Thermo Scientific, RB-9140), CD45 (BioLegend, 103106), F4/80 (Abcam, 6640), SOX9 (Millipore, AB5535) and ALB (Abcam, ab19196) were performed on frozen sections following a standardized protocol. Quantification of immunofluorescence or immunohistochemical staining was performed using ImageJ software in a blinded manner.

### Histological Grading and Staging

Sections of 2 μm thickness from liver allograft biopsies with a 17 gauge needle were stained with haematoxylin and eosin, elastic van gieson stain, periodic acid-Schiff stain, silver stain, Berlin blue stain and rhodanine stain. Histological examination and scoring for fibrosis stage (Ishak score or Scheuer score) was performed by experienced liver pathologists in blinded fashion.

### Example 2—Evaluation of HNF4A Expression, Stability and Activity

Expression, stability and activity of the engineered HNF4A protein variants was evaluated in this working example. Therefore, (i) expression was analyzed in HEK293T cells and a HepG2 liver cancer cell line according to standard procedures; for analysis of expression, EGFP was used as reference control, i.e. HepG2 cells were transfected with 2 μg EGFP-3×HA RNA (SEQ ID NO:5713) and (ii) HNF4A-dependent APOA4 target gene induction (APOA4 is an endogenous HNF4A-target) was analyzed further by Western Blot analysis in a quantitative manner.

Experimental details for APOA4 target gene induction Western Blot analysis are as follows (i.e. reverse transfection of HepG2 cells): on the day of transfection, cells were trypsinized and resuspended in cell line medium+20% FCS (antibiotics-free). mRNAs were complexed with Lipofectamine2000 at a ratio of 1/1.5 (w/v) in Opti-MEM medium (Thermo Fisher) for 20 minutes. 2 μg mRNA of lipocomplexed mRNAs in Opti-MEM (in 1 ml) were then added to 1 million cells in DMEM or RPMI plus 20% FCS (in 1 ml) per 6-well, resulting in a total volume of 2 ml. Cells were maintained at 37° C., 5% CO2.

14-16 h post transfection cells were harvested and cell lysis with urea buffer was performed. For cell lysis, 400 μl lysis buffer was added to each 6-well and cells were incubated while rocking in lysis buffer at room temperature for 15 minutes (100 ml Lysis buffer contained as follows: 2M Urea (12.01 g of Urea M=60.06 g/mol BioChemica (A1360)), 4% sucrose (4.02 g of 99.5% sucrose from Sigma (84097)), 5% SDS (25 ml of 20% SDS from Applichem (A0675)) and 1 mM EDTA (37.22 mg EDTA M=372.24 g/mol from Applichem (A2937)), then filled up with H₂O to 100 ml). Resulting lysates were run over a QIAshredder column to remove the precipitated DNA. Finally, protein concentration was measured according to standard protocols in a BCA Assay Kit (Sigma Aldrich, B9643-1L/SLCB6552, C2284-25 mL/SLCB6519, 23208/UG288327D) and Licor Loading Buffer (4×) was added before loading (SDSPage and Western Blot analysis with cell lysates).

Consequently for detection, a Western Blot was performed according to standard procedures, using HA-tag or ApoA4 antibodies with HSP90 serving as common loading control. Thus, in detail for detection in Western Blot, primary antibodies anti-HA (12CA5) mouse mAb (Roche Cat.: 11583816001, 0.4 mg/ml, dilution 1:1000) and HSP90 (C45G5) rabbit mAb [Cell Signaling Technology (CST) Cat. #4877, dilution 1:2000] as well as ApoA4 (1D6B6) mouse mAb (Cell Signaling Technology (CST) Cat. #5700S, dilution 1:500) and HSP90 (C45G5) rabbit mAb (Cell Signaling Technology (CST) Cat. #4877, dilution 1:2000) were used. As secondary antibody, for antibody detection, for the HA-tag or respectively ApoA4, IRDye 800CW Goat anti-mouse IgG (H+L, LI-COR Biosciences GmbH Cat: 926-32210, dilution 1:10000) and for the HSP90 loading control, IRDye 680RD Goat anti-rabbit IgG (H+L, LI-COR Biosciences GmbH Cat: 926-68071, dilution 1:10000) were used.

Membranes were incubated with 10 ml of primary antibody dilutions (HA supplemented with milk powder, APOA4 with BSA fraction V) for 4h at room temperature and both membranes were incubated with 10 ml of secondary antibody dilutions for 30 min at room temperature, washed and detection was carried out using an Odyssey CLX image system.

### Results:

As described above, codon-optimized (GC) mRNA-encoded engineered HNF4A protein variants were compared with equally codon-optimized (GC) HNF4A mRNA encoding the amino acid sequence of unmodified HNF4A WT protein (SEQ ID NO:5564), and analysed for either (i) expression levels in HEK293T and HepG2 cells or (ii) HNF4A-dependent APOA4 target gene induction by Western Blot analysis. Experimental results are displayed in FIG. 1 and FIG. 2.

Re (i), in vitro expression of HNF4A constructs in HEK293T (data not shown), HepG2 cells (FIG. 1) clearly showed stable and higher expression of codon-optimized (GC) HNF4A in transfected cells as compared to WT HNF4A. Further, nuclear localization of HNF4A proteins was shown (image data from transfected cells and subsequent fluorescent staining with DAPI not shown).

Re (ii), upregulation of APOA4 protein in HepG2 cells was evaluated upon expression of HNF4A mRNA constructs. As clearly apparent, engineered HNF4A protein variants induced higher APOA4 gene expression i.e. have a higher activity (>2-fold) in HepG2 cells as compared to WT HNF4A (FIG. 2). In particular, S87A and S461E have higher activity (>2-fold).

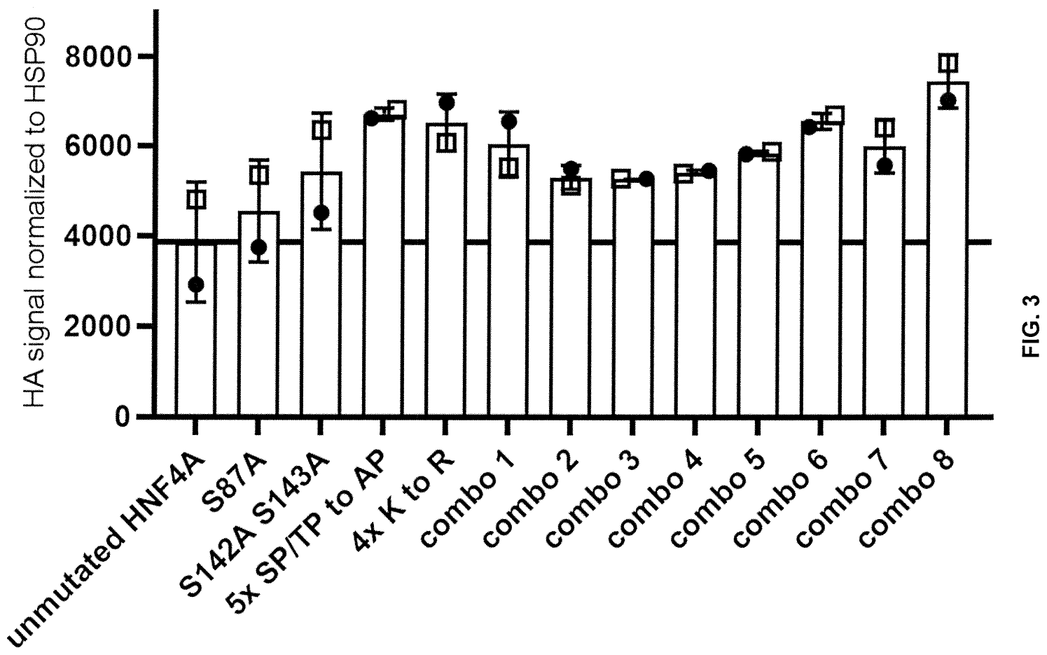

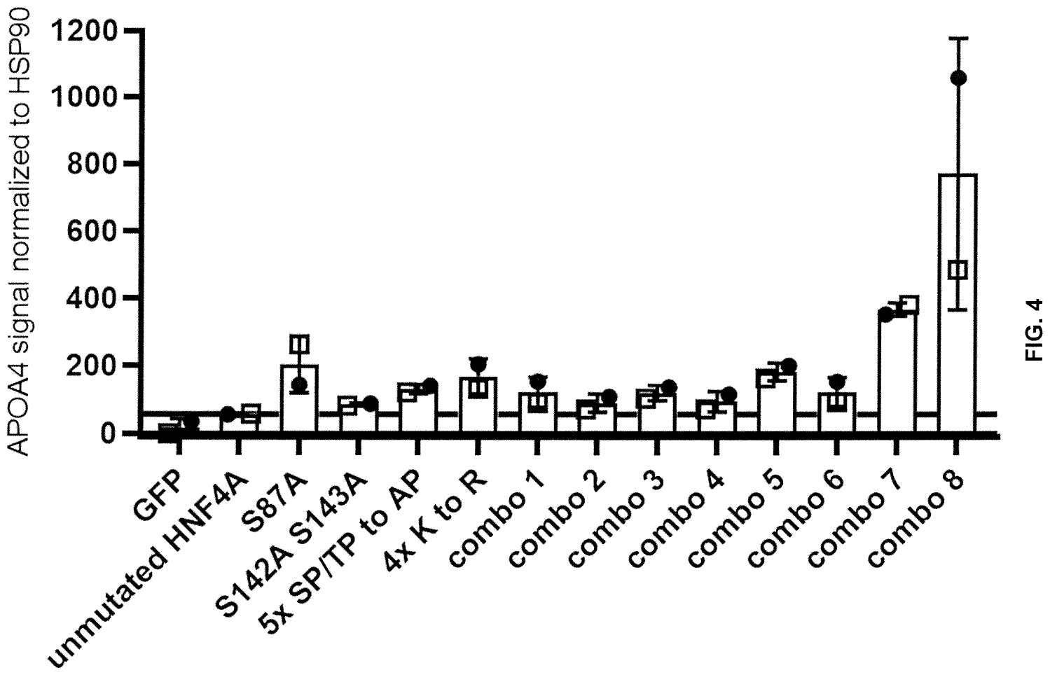

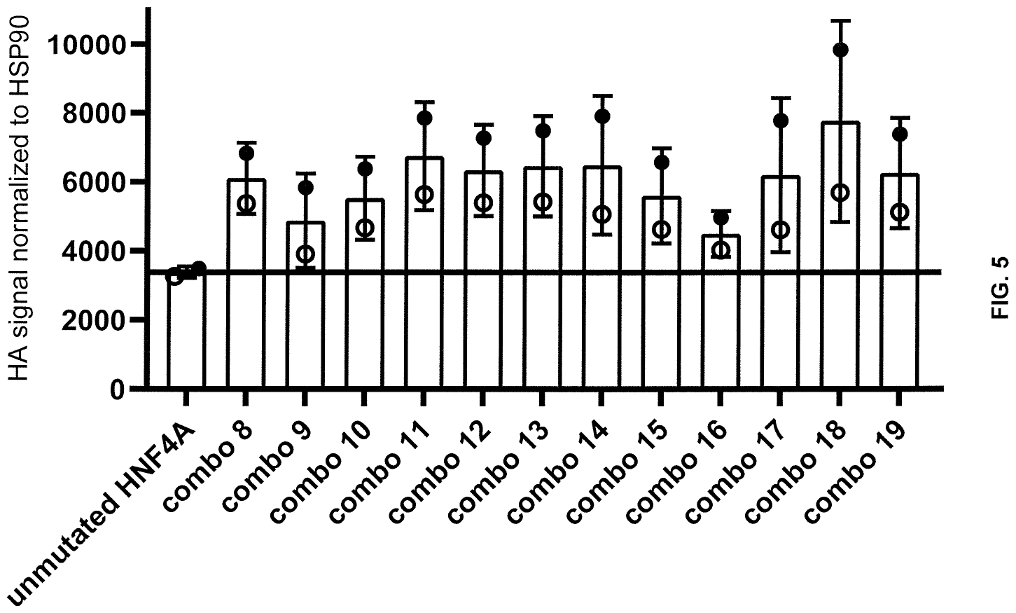

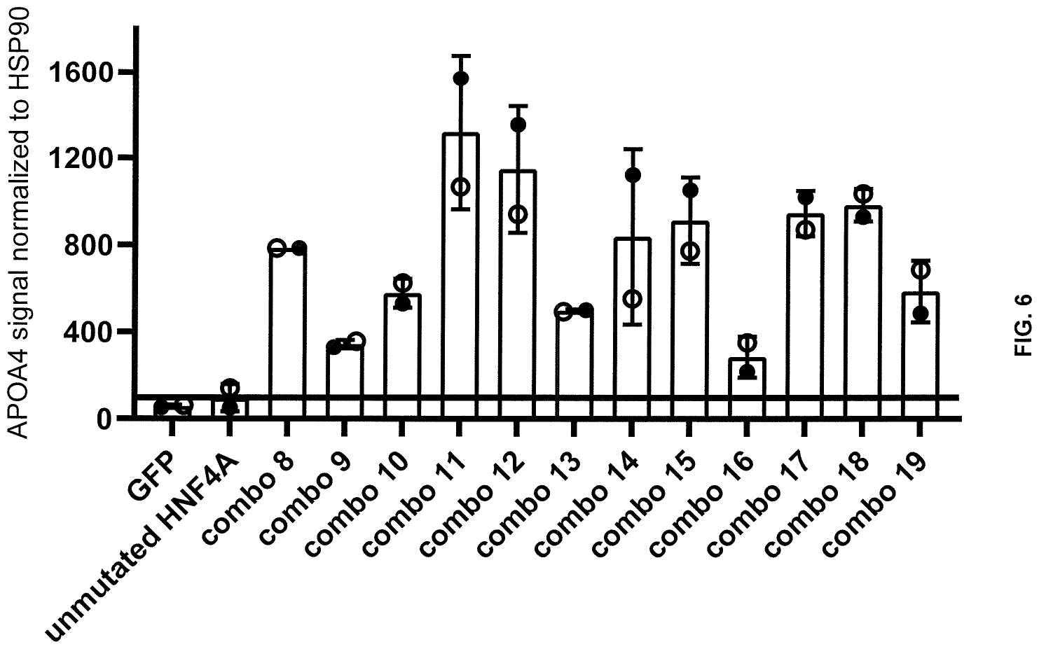

Continuing the screening of single and combinatorial mutants regarding increased (i) HNF4A expression and stability and (ii) HNF4A activity, it was found that expression/stability could be increased up to factor ˜2 and activity up to factor ˜14 (see FIG. 3 to FIG. 4). In detail, a synergistic effect of mutant S87A with K106R K108R K126R K127R was revealed and also mutations S87A K106R K108R K126R K127R S142A S143A S148A T166A S167A S313A S378A T429A T432A S436A K458R S461E (combo 11) was identified as being the most potent mutant, having ˜2-fold increased stability and ˜14-fold higher activity relative to wild type HNF4A.

Thus, as apparent, it was possible to significantly increase HNF4A expression, activity and/or stability in the inventive engineered HNF4A protein variants.

### Example 3—In Vitro WT HNF4A Expression Leads to Improved Function of Fibrotic and Non-Fibrotic Hepatocytes (PHH)

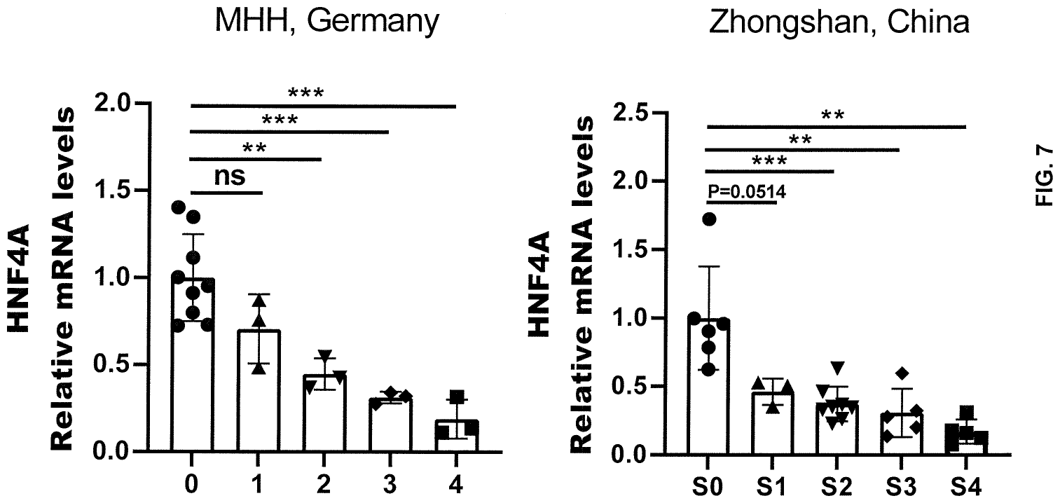

Liver tissues from fibrotic human livers were isolated by Hannover Medical School (MHH), Germany, and Shanghai Zhongshan Hospital, China and examined by MHH for stage-dependent expression of HNF4A mRNA. The analysis showed, that endogenous HNF4A mRNA levels in patients with liver fibrosis stages 0-4, graded by the Ishak score (Ishak et al., 1995; 22(6):696-9) or S1-S4 stages, graded according to the Scheuer system (Scheuer, J Hepatol. 1991; 13(3):372-4) showed reduced endogenous HNF4A mRNA levels (see FIG. 7). Similarly, HNF4A protein expression was reduced in primary human hepatocytes (PHH) from the fibrotic human livers (data not shown).

Experimental details regarding the process of RNA extraction and following quantitative PCR (qPCR) analysis to detect the endogenous Hnf4a mRNA expression are as follows. Livers were taken at indicated time points after injection with mRNA/LNP. For in vitro studies, primary mouse hepatocytes and primary human hepatocytes were cultured and treated as mentioned above. Total RNAs were extracted by Trizol reagent (Qiagen) from cells or tissues according to the manufacturer's instructions. cDNA was generated using TaqMan Reverse Transcription Reagents (ThermoFisher). qPCR was performed according to the TaqMan (Invitrogen) or SYBR Green (ThermoFisher) Gene Expression Assay protocols using a 96-well ABI StepOne Plus Real-Time PCR System (ThermoFisher). Samples were run in triplicate. The primer sequences are provided in SEQ ID NO:5616 to SEQ ID NO:5671 (more information on the primers is disclosed in the ST.25 sequence listing under <223> Other Information [here, and throughout the whole specification, it has to be noted that the priority application was filed with a sequence listing in accordance with the WIPO Standard ST.25, which then was converted into a sequence listing according to WIPO Standard ST.26—information which was comprised within line <223> in ST.25 now was added to the respective SEQ ID NO: as a note under “feature key”, i.e. “misc_feature” (for nucleic acids) or “REGION” (for proteins)]) and Table Ex-3. Relative mRNA expression was determined after normalizing the expression with respect to glyceraldehyde-3-phosphate dehydrogenase (GAPDH), which was used as a house-keeping gene.

TABLE Ex-3
primers used for Taqman qPCR

| Probes | ThermoFisher Catalog number |
|---|---|
| GAPDH (Human) | Hs02758991_g1 |
| HNF4A (Human) | Hs00604438_m1 |
| APOA2 (Human) | Hs00155788_m1 |
| APOB (Human) | Hs00181142_m1 |
| F7 (Human) | Hs01551994_m1 |
| Alb (Mouse) | Mm00802090_m1 |
| Apoa2 (Mouse) | Mm00442687_m1 |
| Apob (Mouse) | Mm01545154_g1 |
| Ck19(Krt19, Mouse) | Mm00492980_m1 |
| F7 (Mouse) | Mm00487329_m1 |
| Gapdh (Mouse) | Mm99999915_g1 |
| Hnf4a (Mouse) | Mm00433964_m1 |
| Sox9 (Mouse) | Mm00448840_m1 |
| Ttr (Mouse) | Mm00443267_m1 |

For analysing the ability of WT HNF4A expression leading to improved function of fibrotic hepatocytes, codon optimized HNF4A mRNA (SEQ ID NO:5715) was transfected into PHH according to routine methods. As control, ZsGreen (SEQ ID NO:5714) was used.

mRNA Transfection

Primary mouse hepatocytes (PMH) and primary human hepatocytes (PHH) were cultured in hepatocytes culture medium (HCM), which was prepared by adding chemicals from HCM SingleQuots Kit into hepatocytes basal medium. LX2 cells and HeLa cells were cultured in high glucose (4.5 g/l) DMEM GlutaMAX medium containing 10% heat-inactivated FBS, 1% non-essential amino acid solution (NEAA), 1% penicillin/streptomycin, and 0.2% 2-mercaptoethanol. For in vitro mRNA transfection, HNF4A mRNA or ZsGreen mRNA were complexed with lipofectamine MessengerMAX following manufacturer's instructions. Briefly, cells were washed with PBS, and the medium was replaced with Gibco Opti-medium before transfection. 1 μg, 0.5 μg, and 0.25 μg mRNA were used to transfect 6-well, 12-well, and 24-well plates, respectively. mRNA/lipofectamine MessengerMAX complex was prepared by keeping a 1:3 ratio.

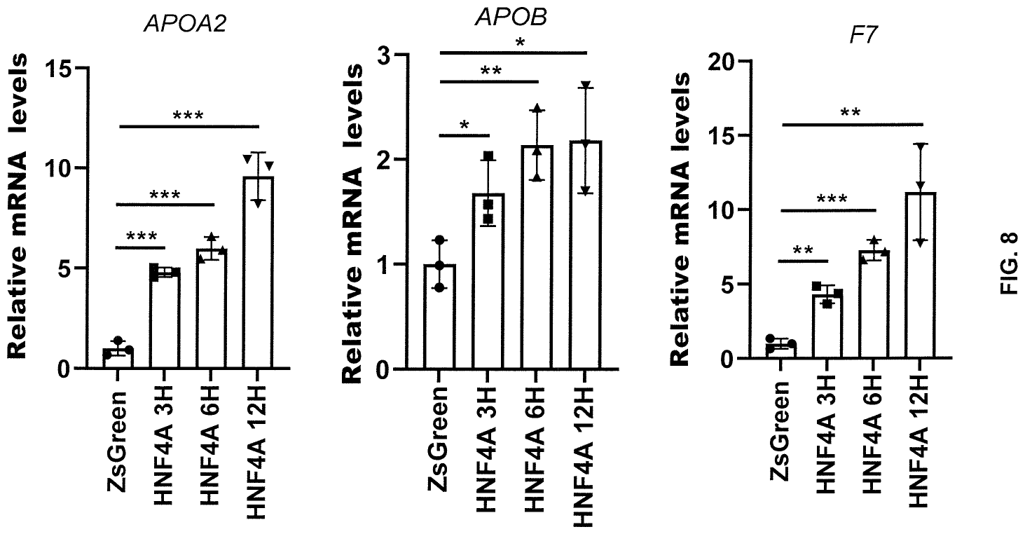

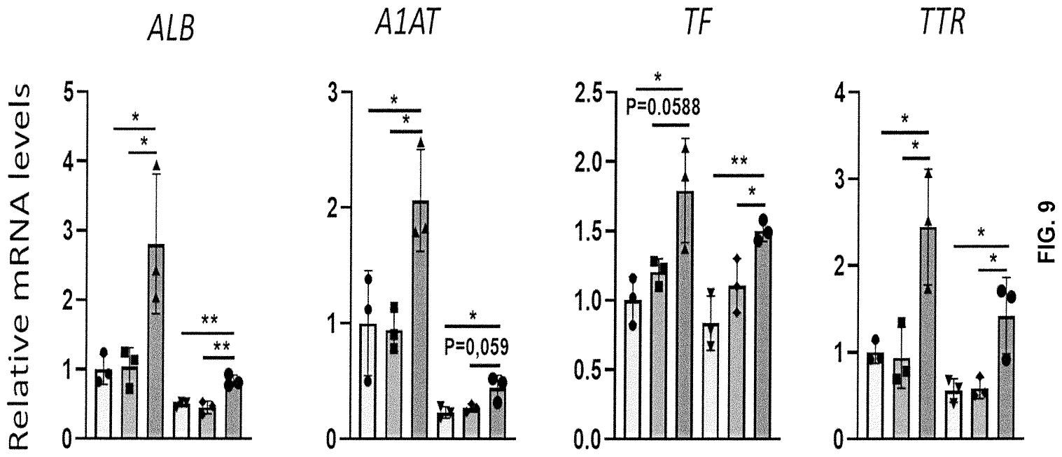

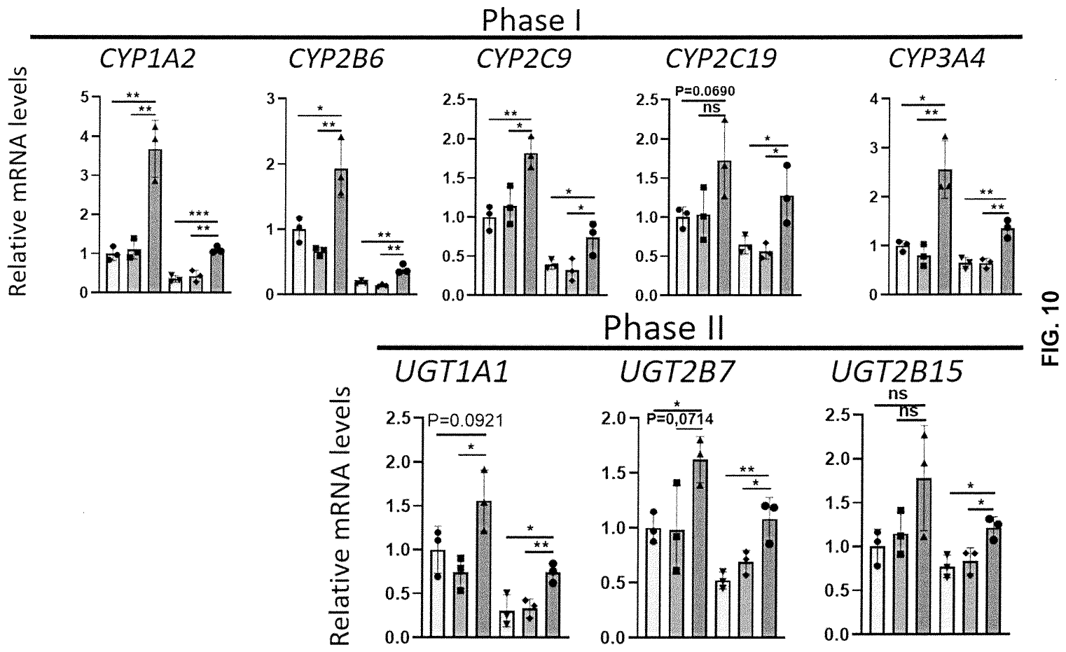

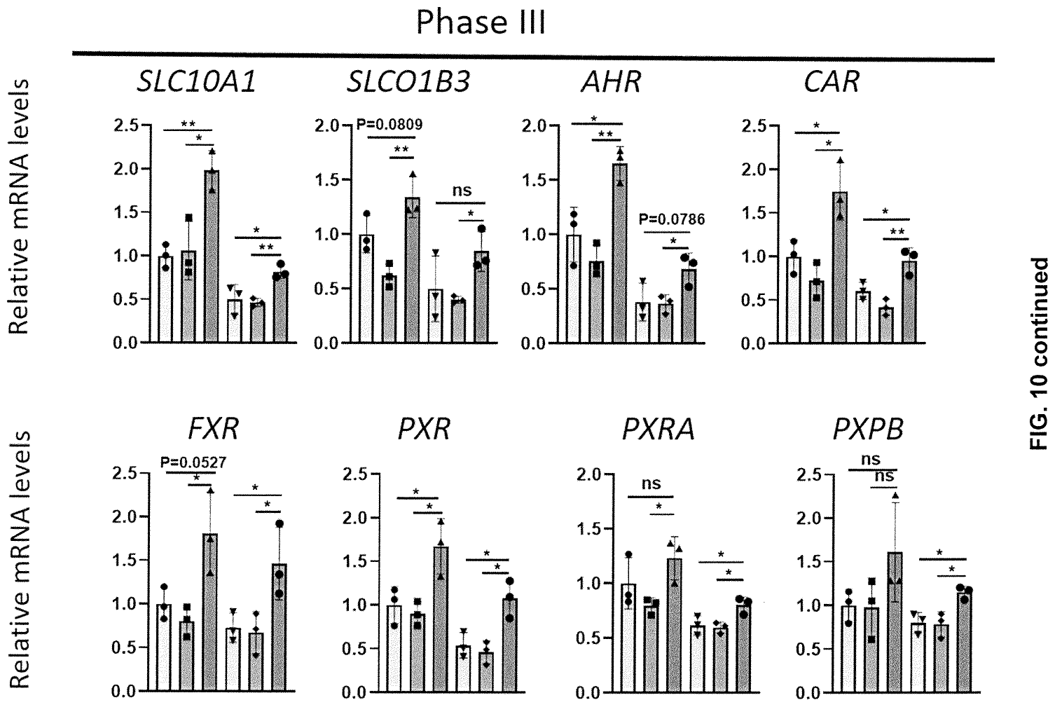

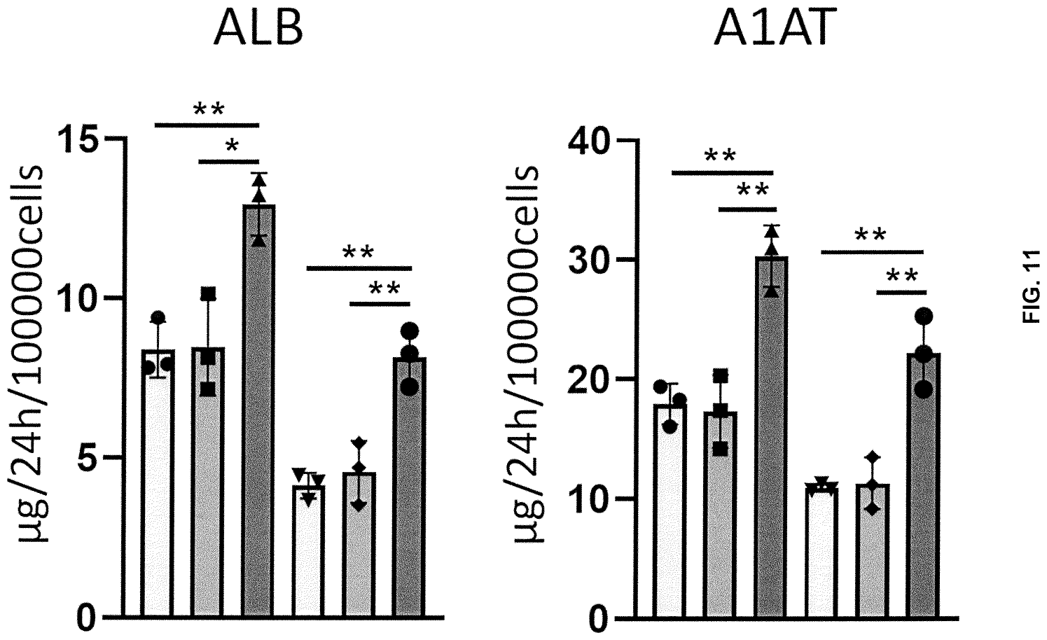

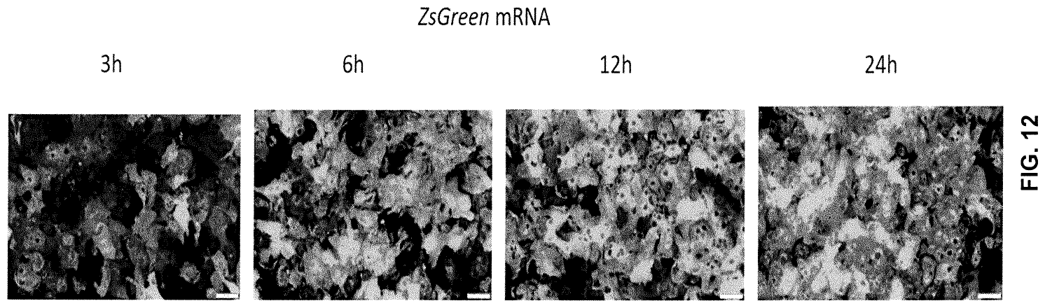

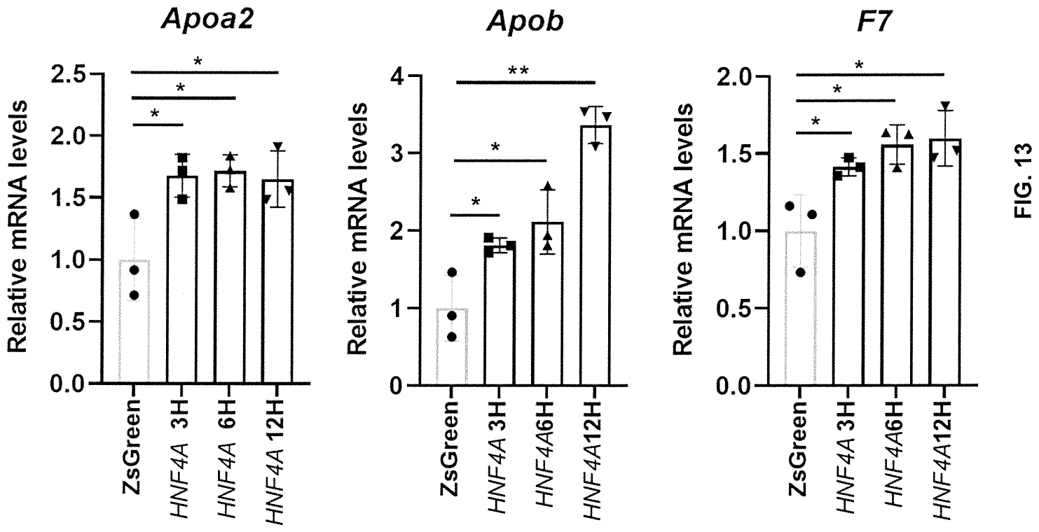

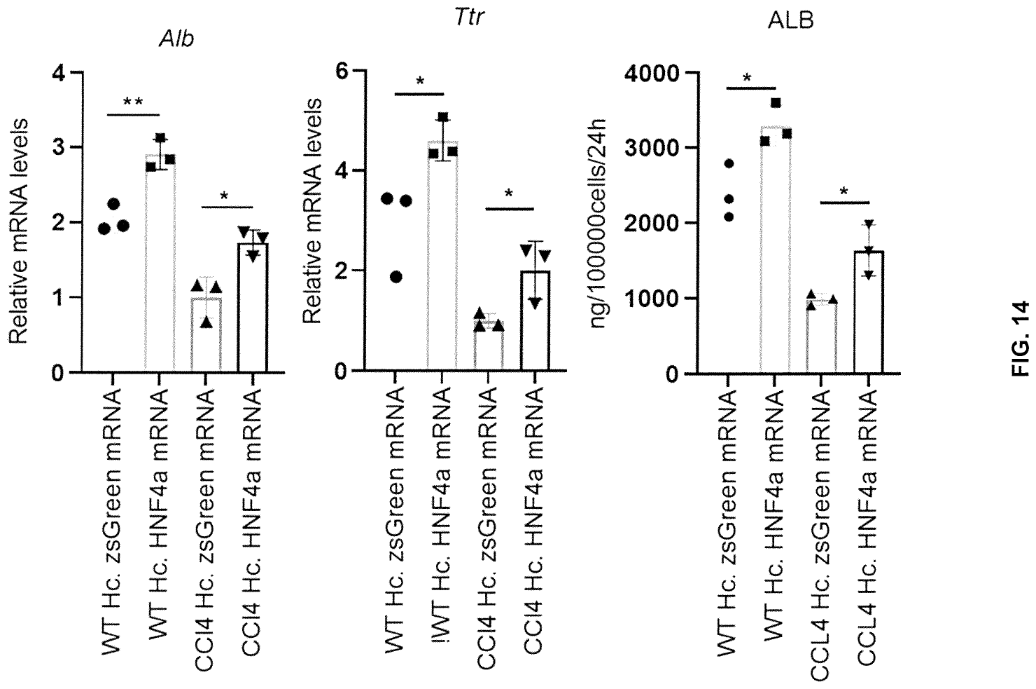

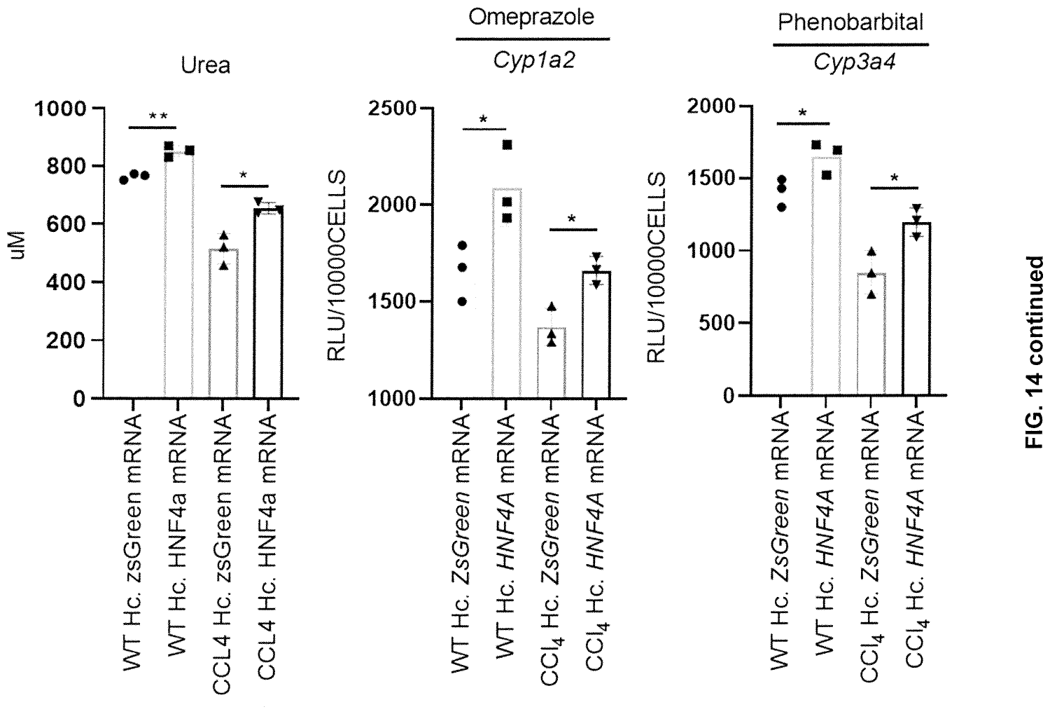

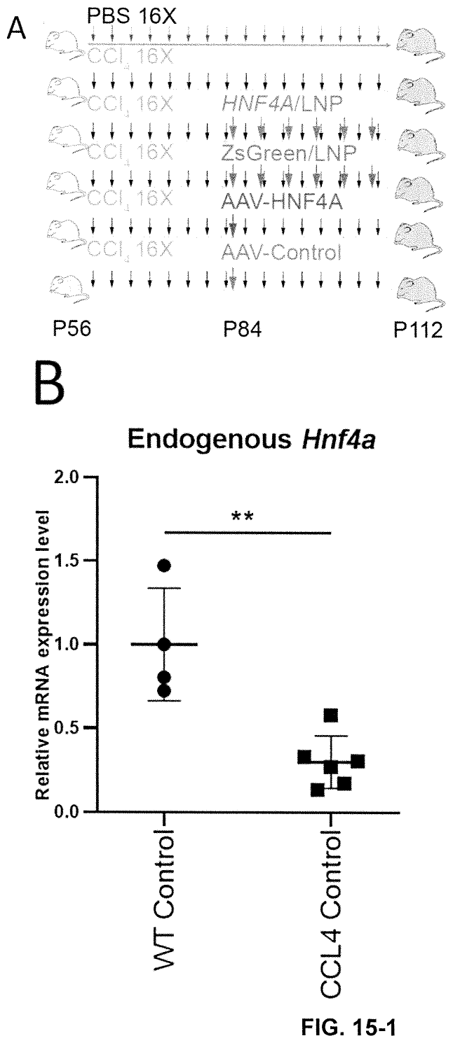

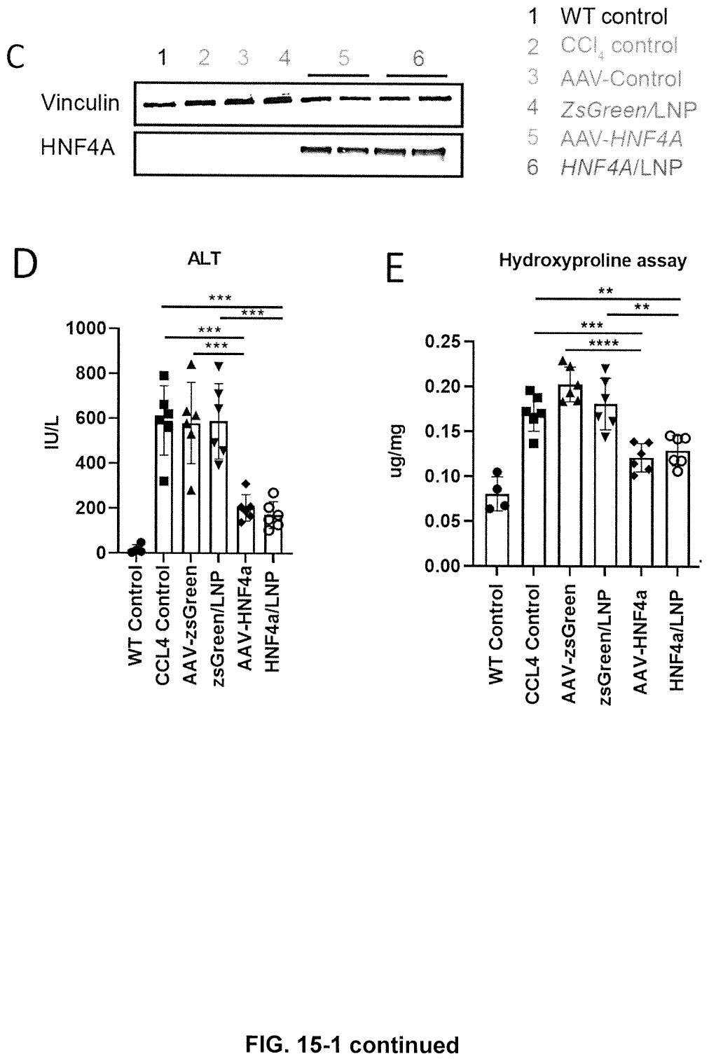

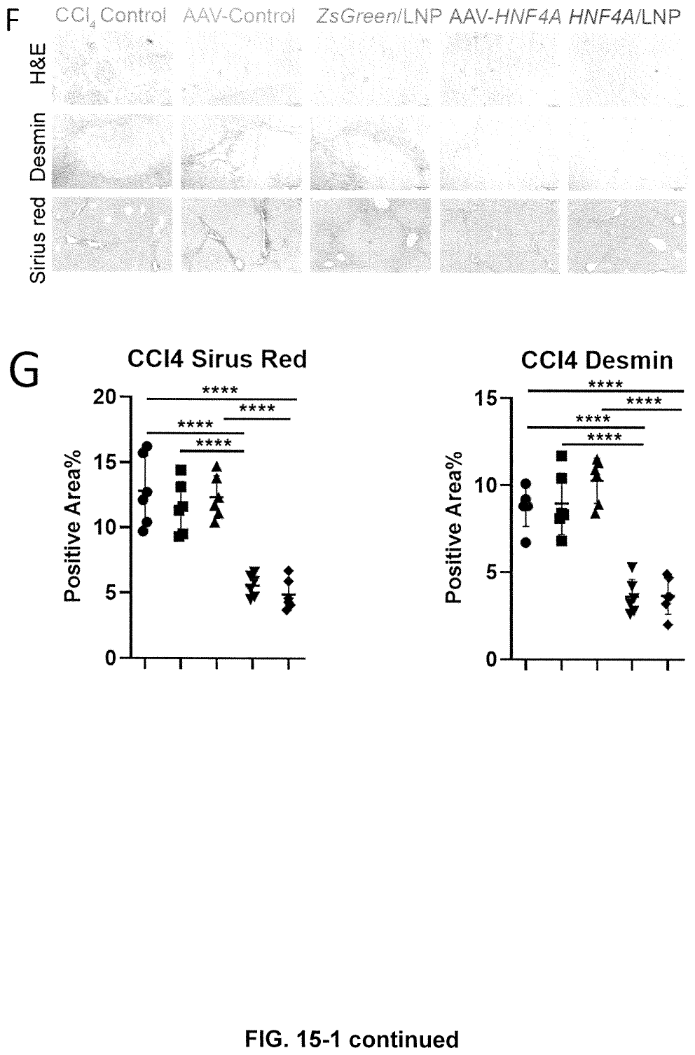

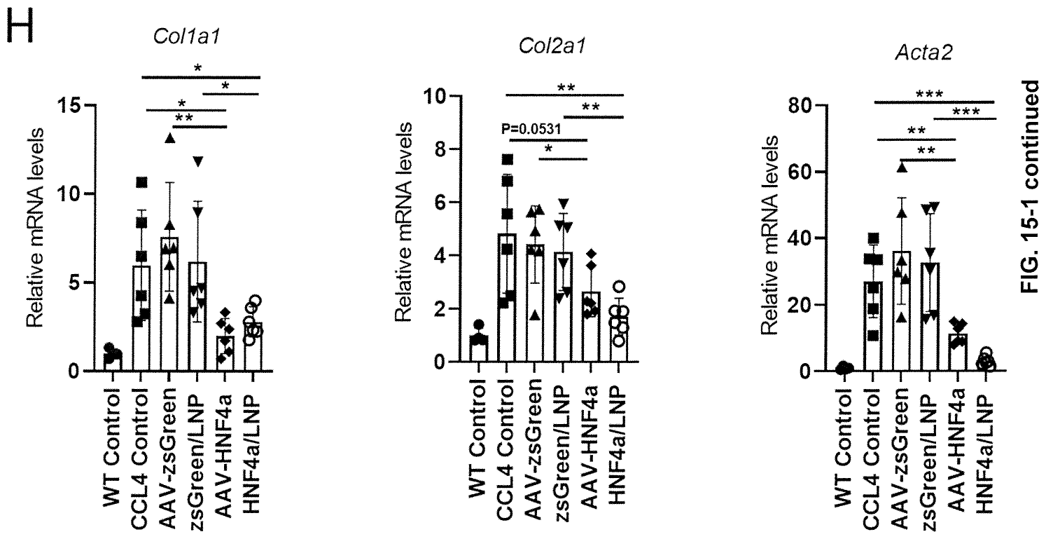

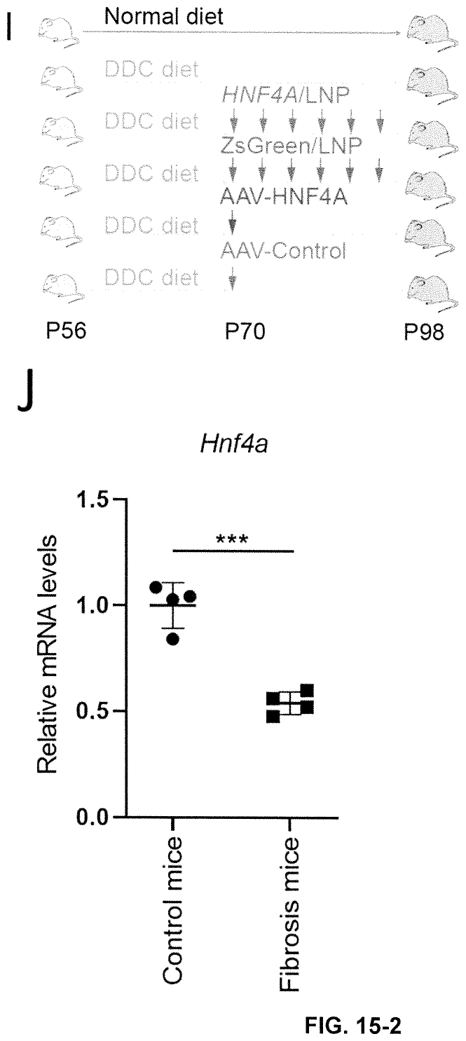

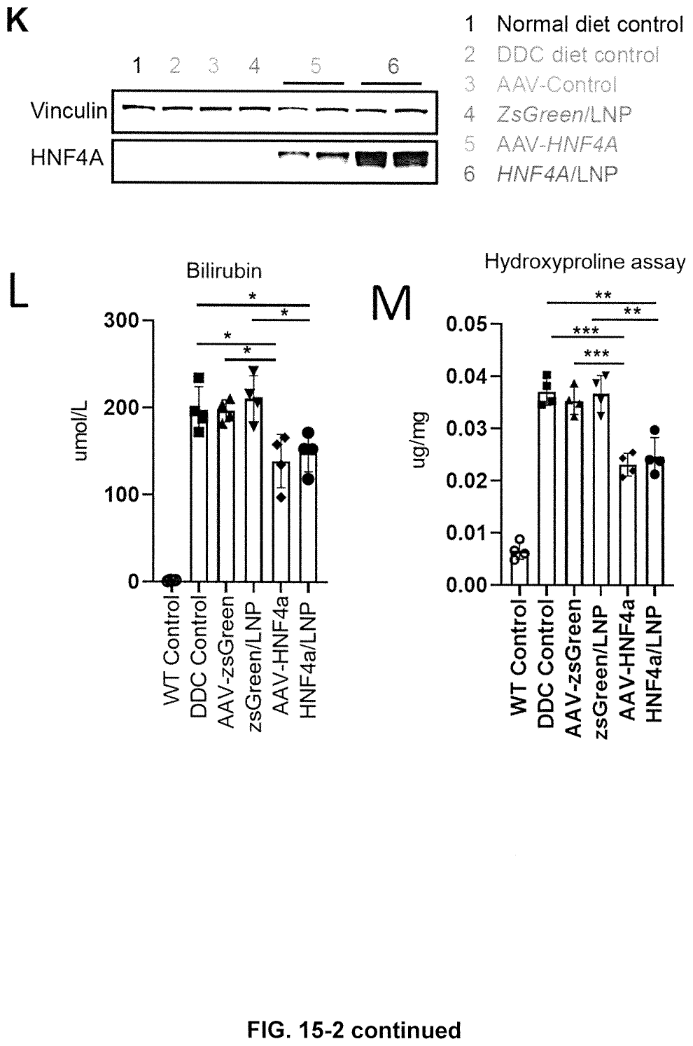

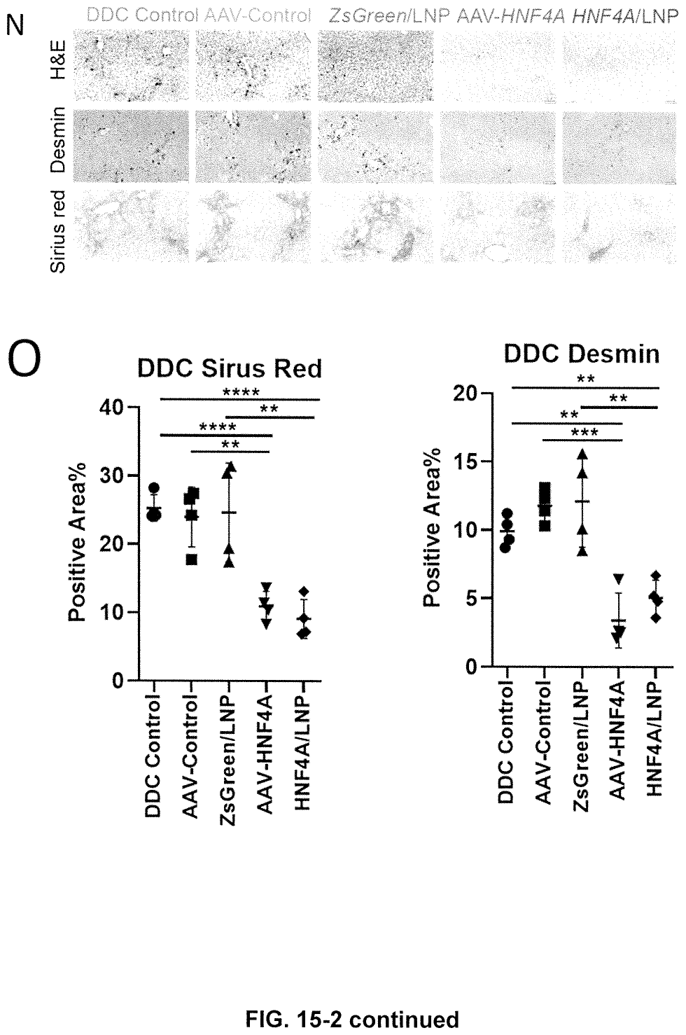

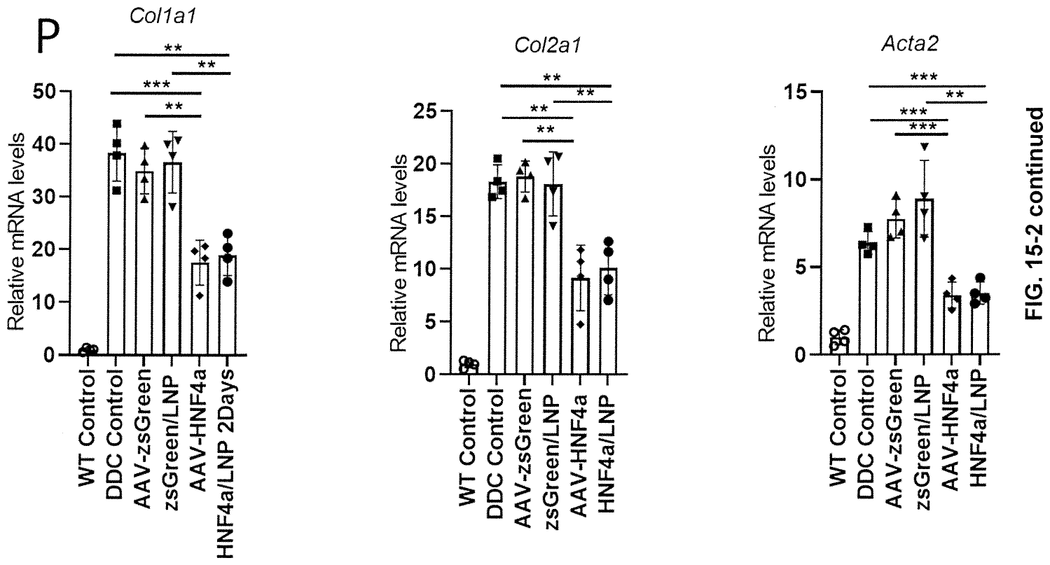

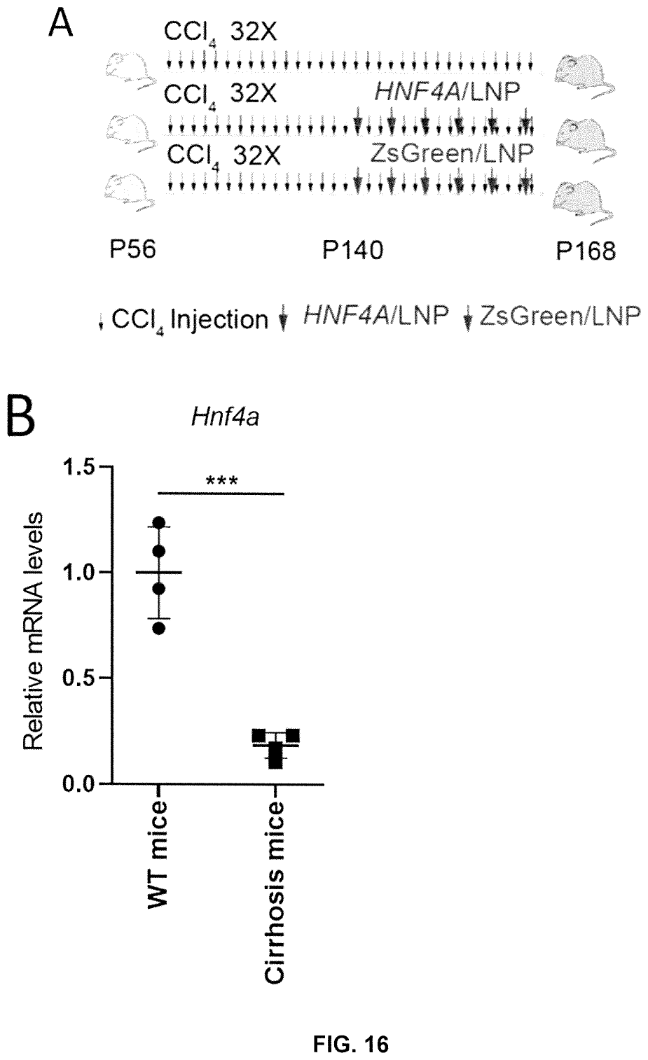

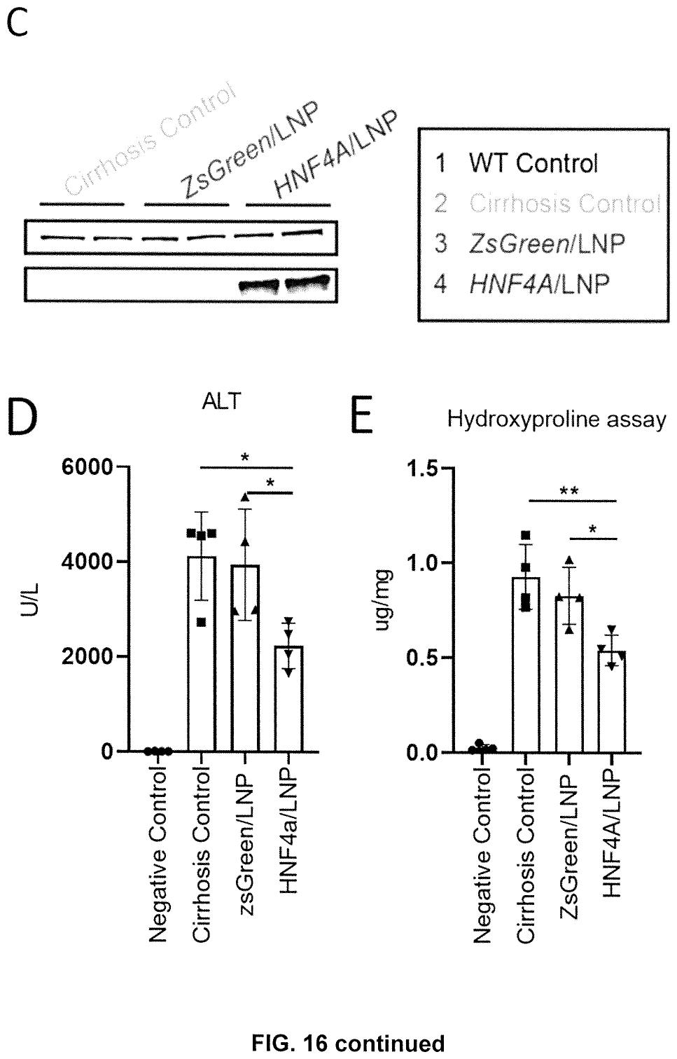

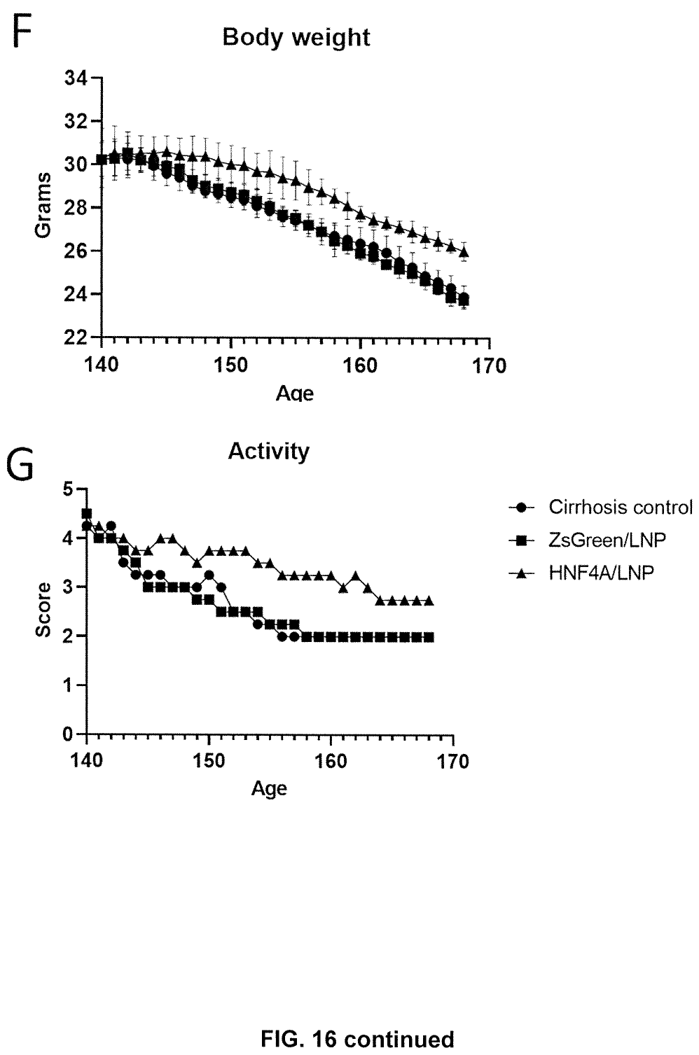

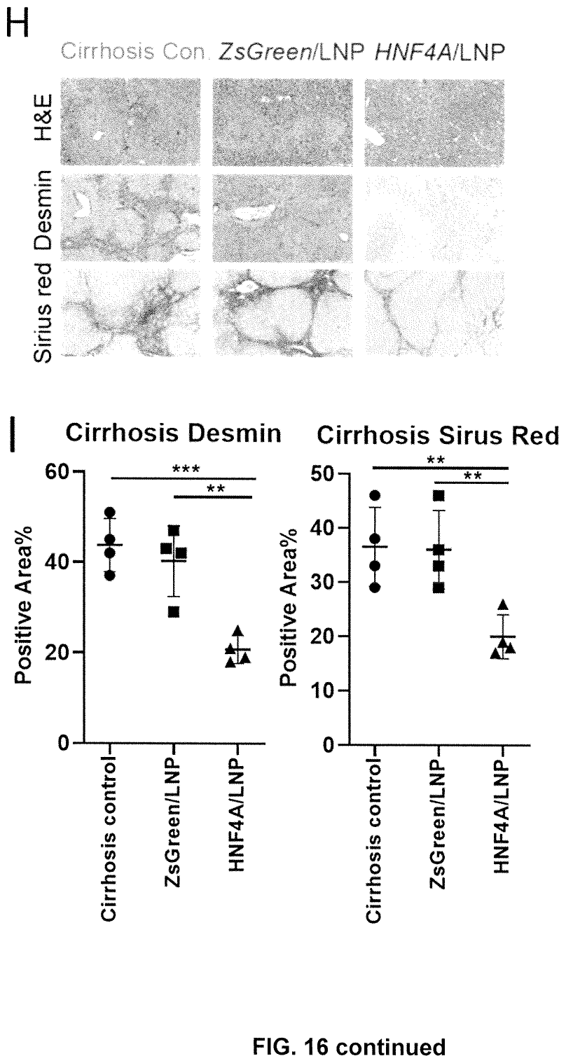

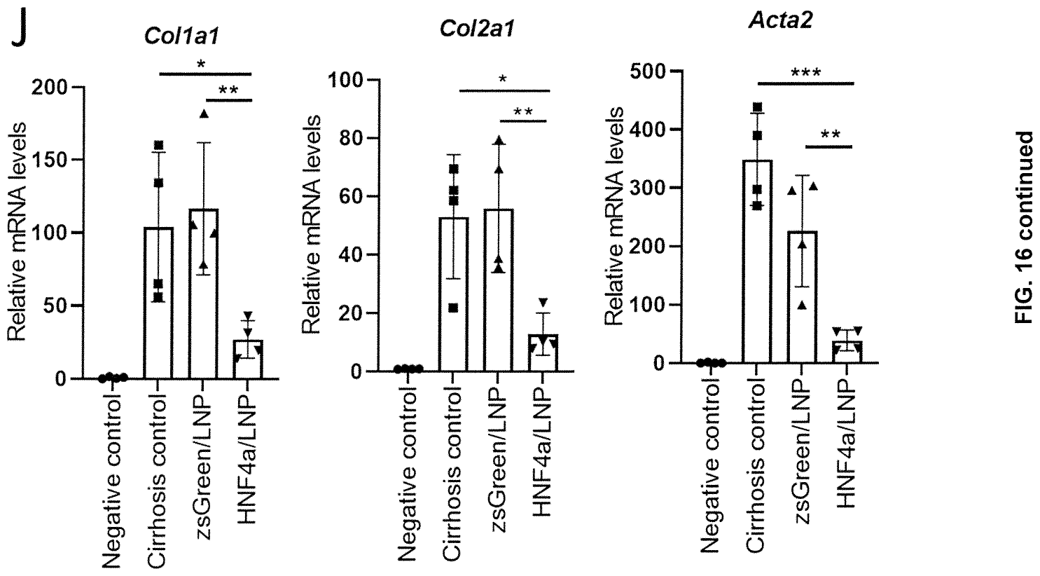

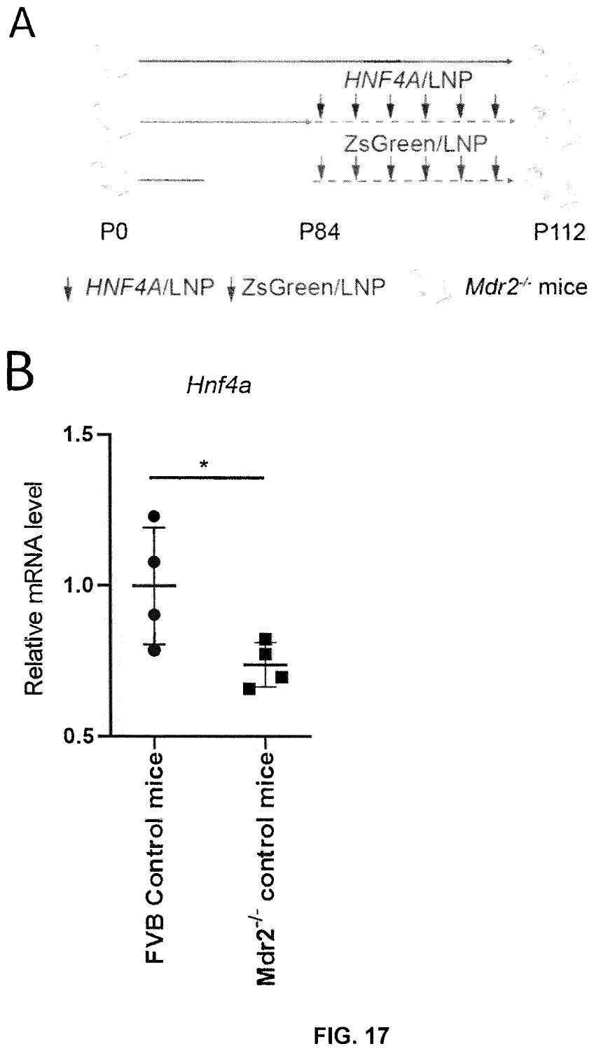

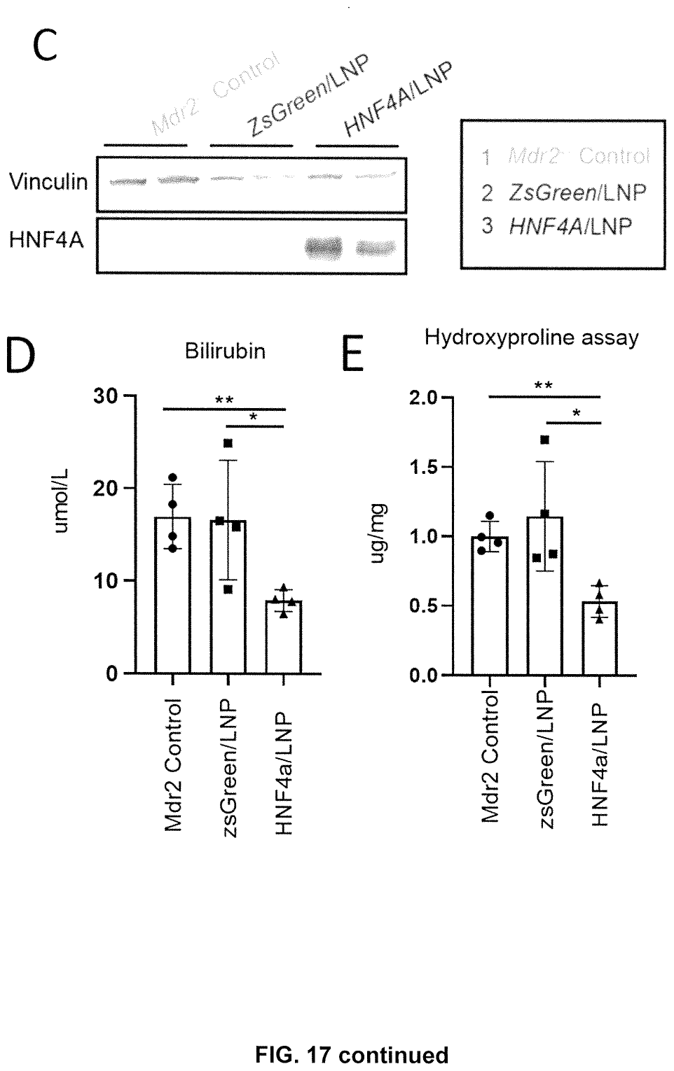

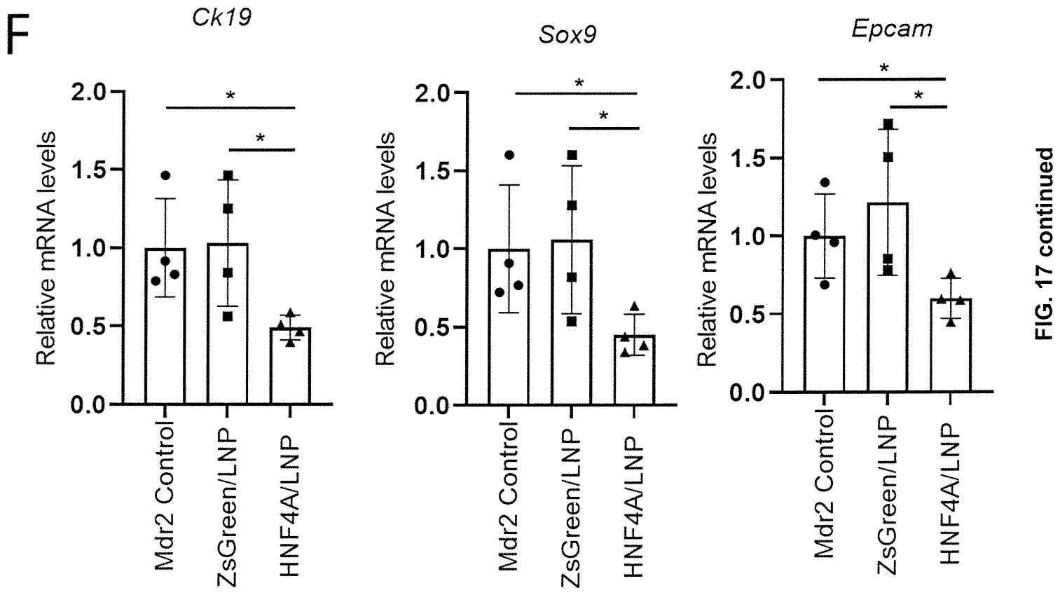

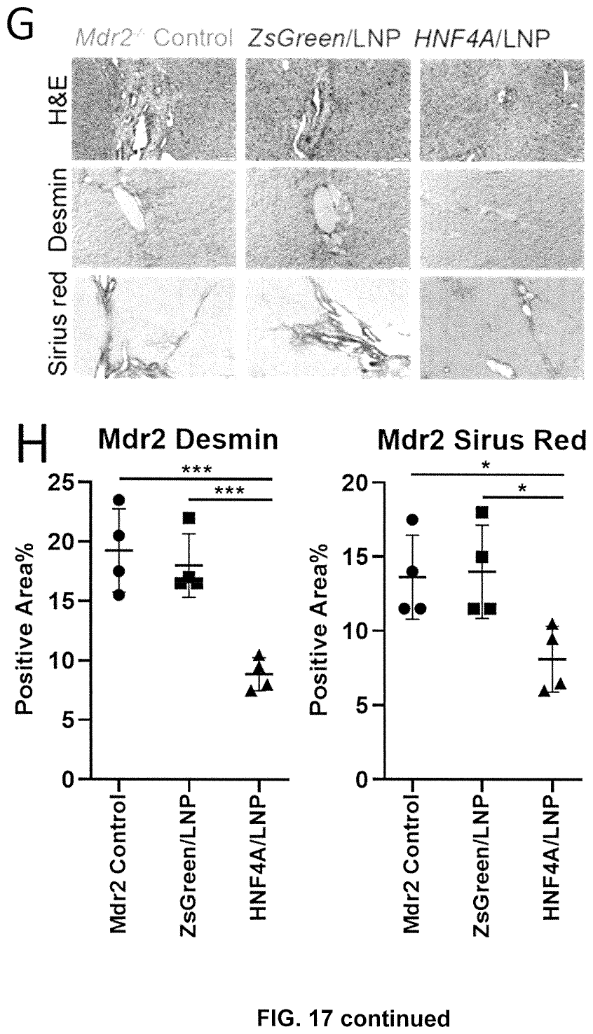

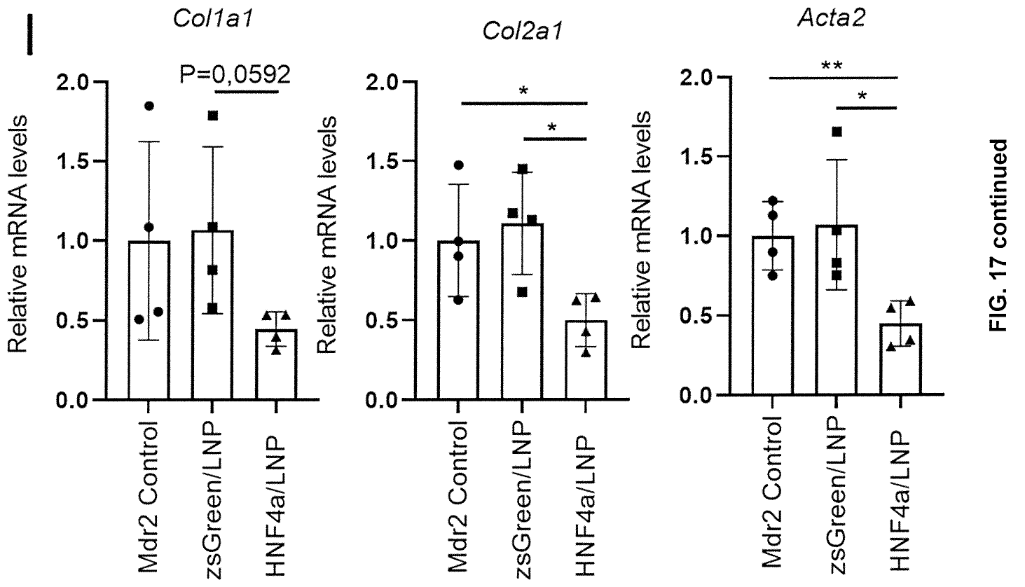

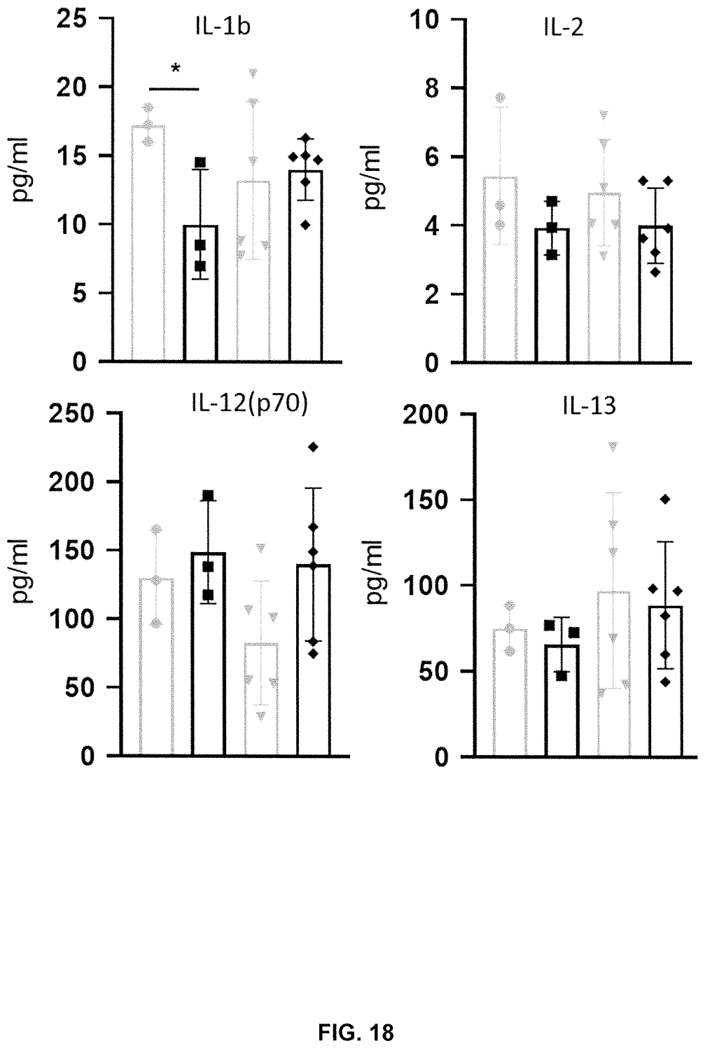

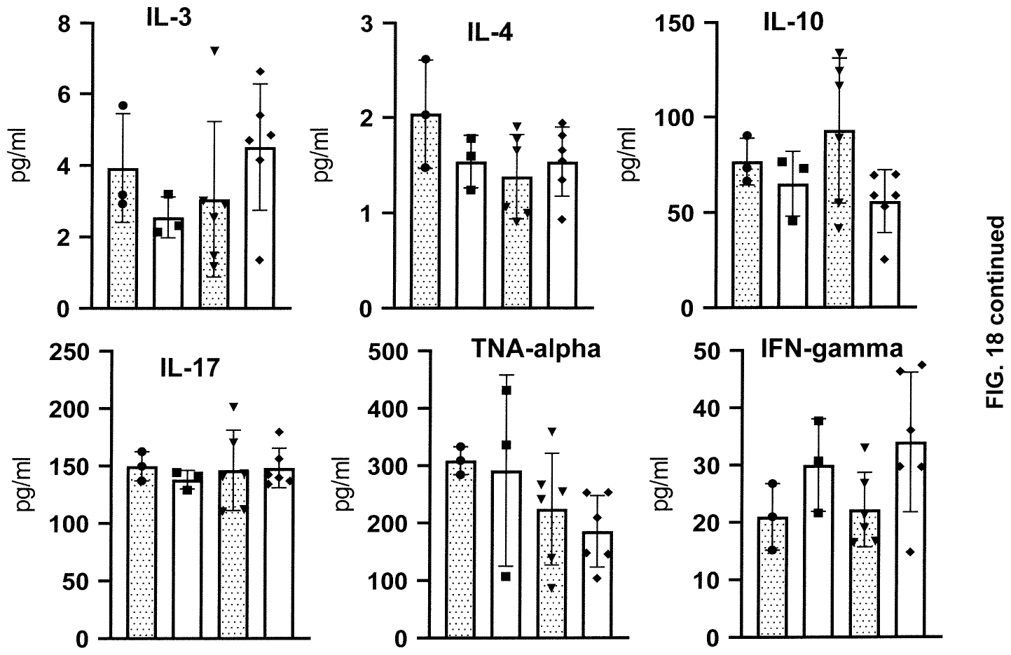

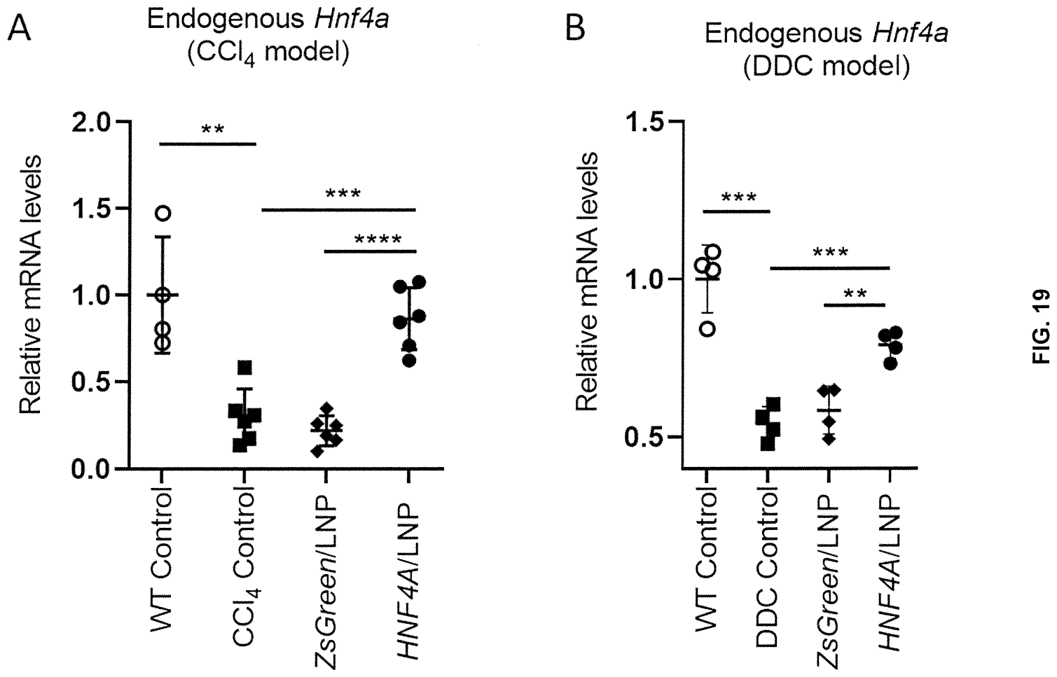

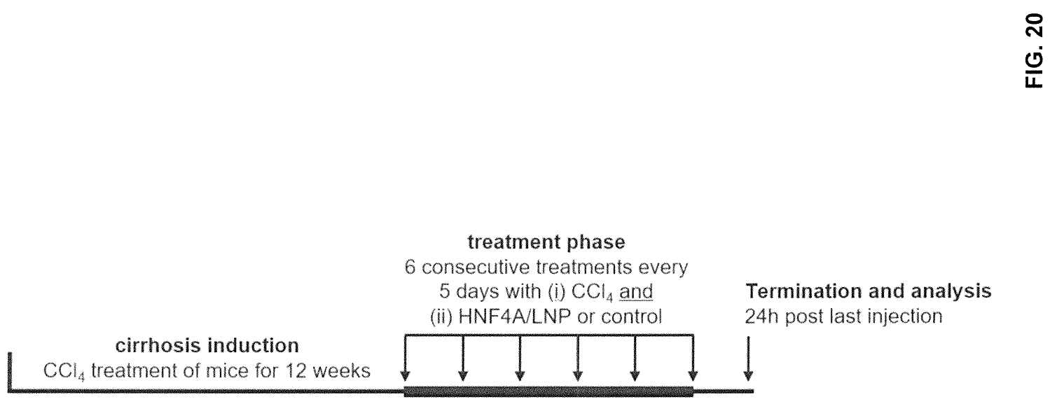

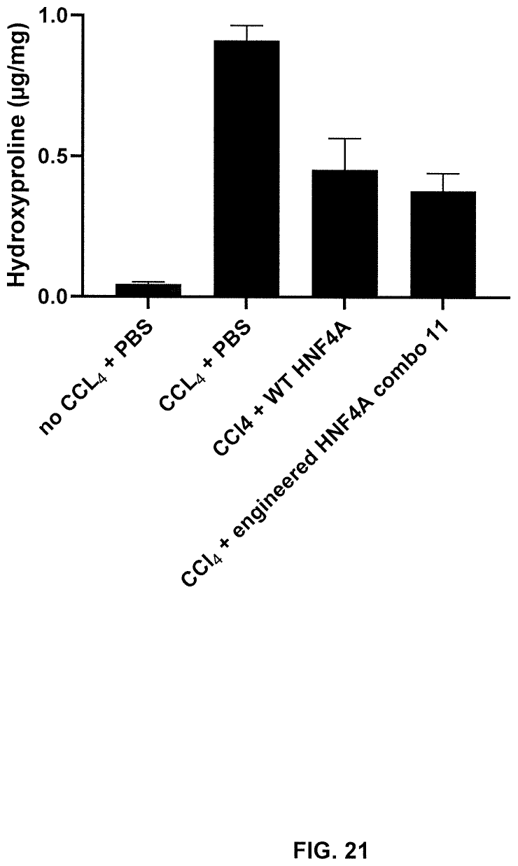

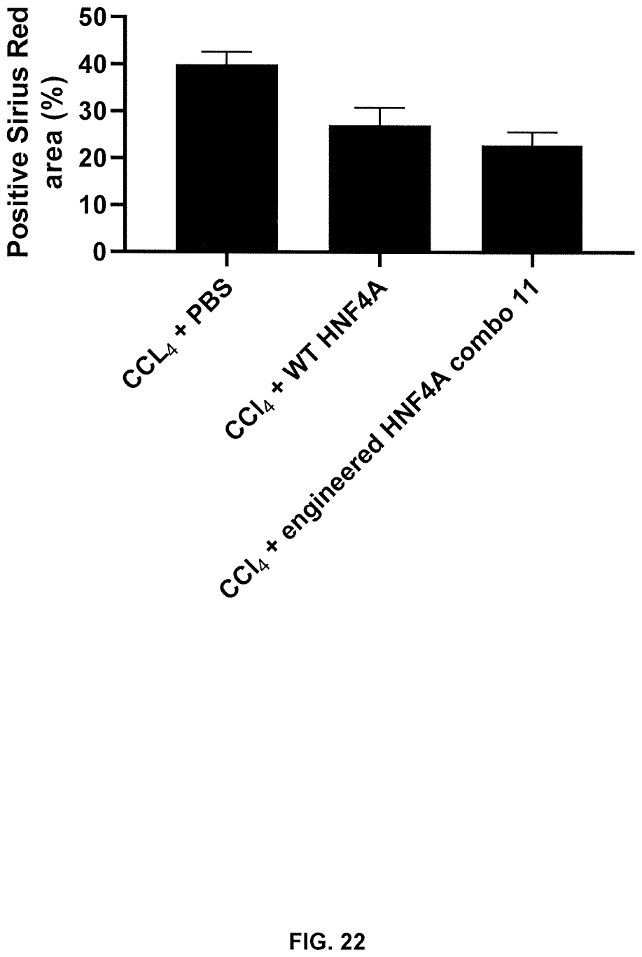

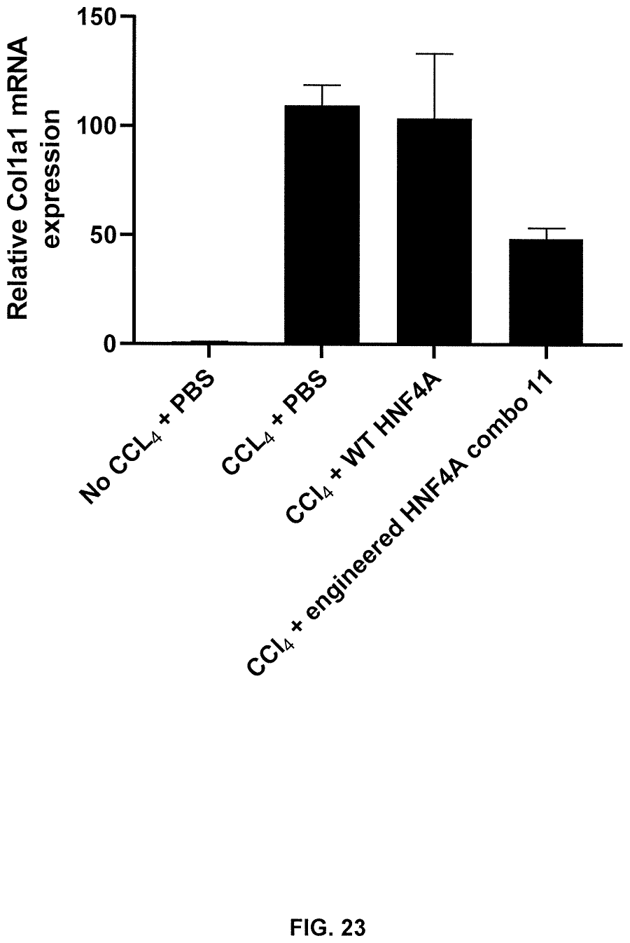

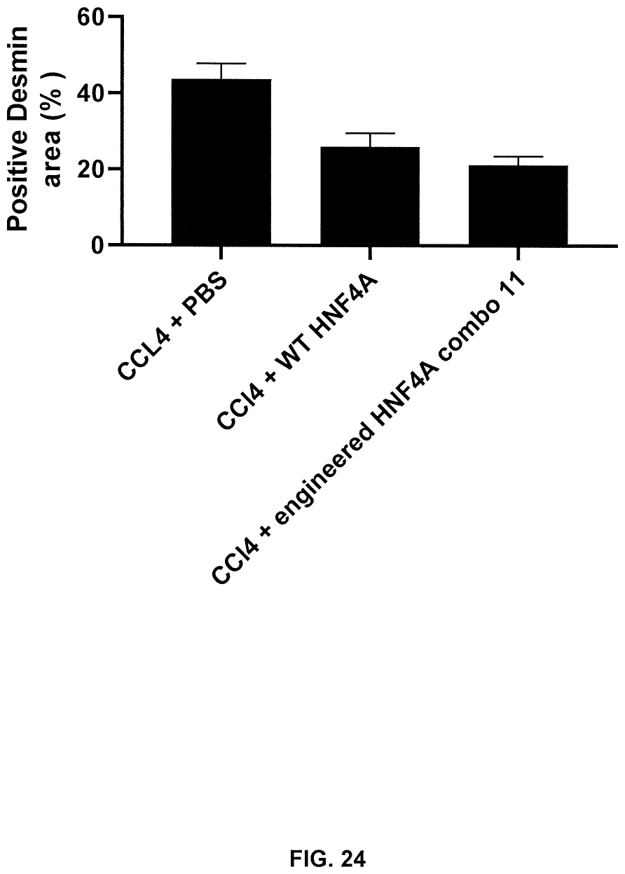

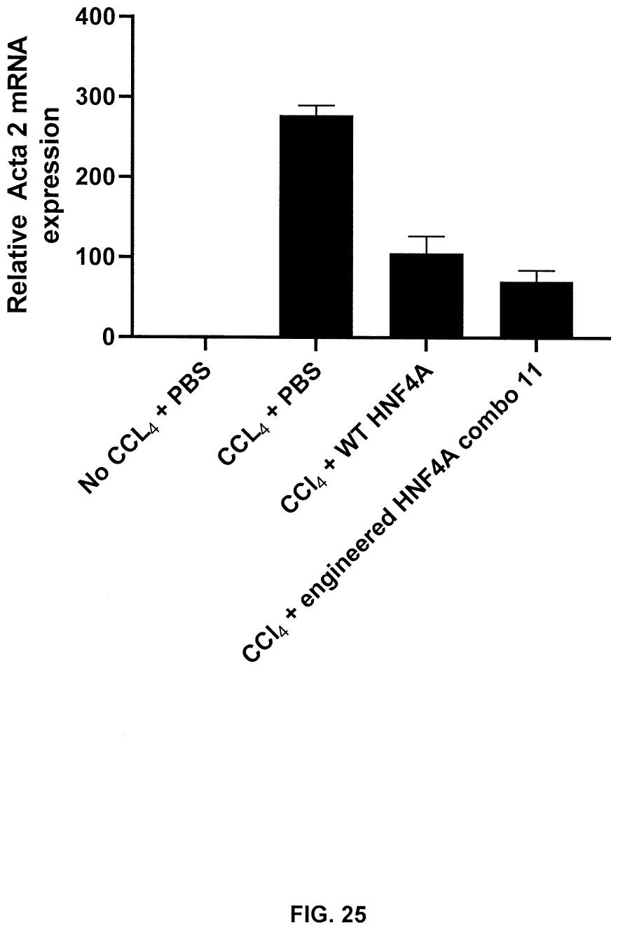

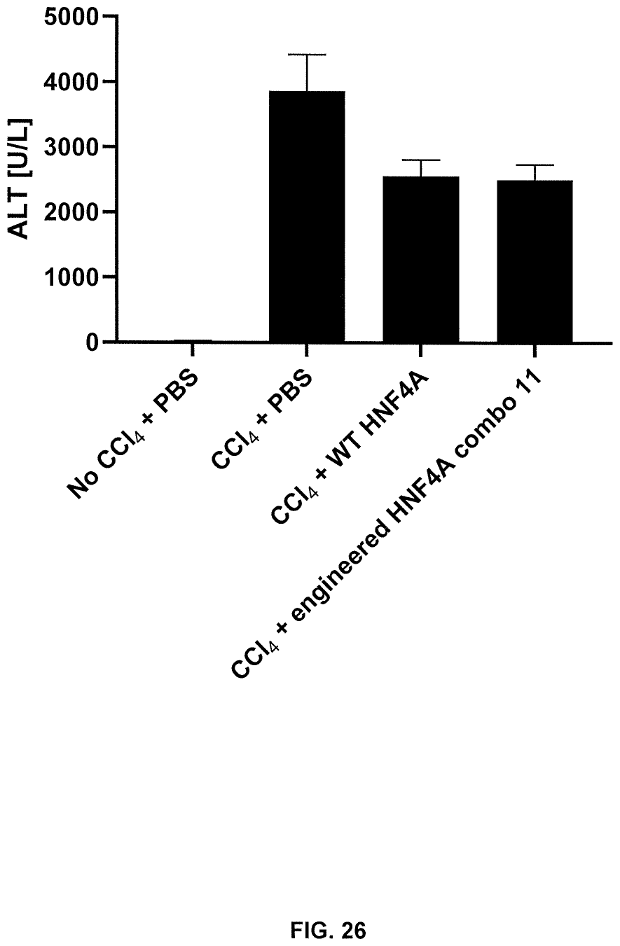

Thereby, it was confirmed, that codon optimized mRNA was functionally active as indicated by significant upregulation of HNF4A-target genes APOA2, APOB and F7 (results see FIG. 8).

Transfection of PHH with HNF4A mRNA further restored impaired functions in PHH from fibrotic livers, as qPCR analyses showed significantly increased or respectively upregulated expression of hepatocyte markers albumin (ALB), alpha-1 antitrypsin (A1A7), transferrin (TF) and transthyretin (TTR) (see FIG. 9) and of various enzymes involved in phase I and phase II drug-metabolism and phase III drug transporters (see FIG. 10). Similar results were achieved upon HNF4A WT mRNA transfection of hepatocytes from non-fibrotic livers (data not shown).

Also a significant increase in secreted protein levels of ALB and A1AT in PHH transfected with HNF4A mRNA further showed that HNF4A WT mRNA in fibrotic PHH was able to revive function of said PHH (FIG. 11).

### Example 4—In Vitro WT HNF4A Expression Leads to Improved Function of Fibrotic and Non-Fibrotic Hepatocytes (PMH)

Analogously to the previous working example, it was tested whether HNF4A mRNA delivery (SEQ ID NO:5715 encoding WT HNF4A protein) restores the function of fibrotic primary mouse hepatocytes (PMH), which showed reduced levels of endogenous Hnf4a mRNA due to induction of acute liver injury through bi-weekly carbon tetrachloride injections for 8 to 16 weeks (details for this procedure, which is applicable also for other working examples, is shown herein below).

In detail, for establishing the liver fibrosis or cirrhosis mouse model, 8-12 weeks old male BALB/c mice were injected via intraperitoneal (i.p.) route with 4 μl/g 10% CCl₄/olive oil, twice a week for 8 weeks (fibrosis) and 16 weeks (cirrhosis), or fed with 3,5-diethoxycarbonyl-1,4-dihydrocollidine (DDC, cholestasis-induced fibrosis) consistently for six weeks according to routine procedures known in the art. Bleeds were performed via sub-mandibular route or via tail nick on scheduled days. Approximately 100 μl of whole blood was collected in serum separator tubes and serum was prepared and stored at −20° C.

As DNASTAR MegAlign showed 95.8% sequence homology between human HNF4A and mouse Hnf4a (comparison not shown), therefore DNA binding domains of both exhibit similar molecular function due to high homology. To determine whether HNF4A mRNA (SEQ ID NO:5715) inhibits liver cirrhosis in mice, firstly ZsGreen mRNA was used as control in PMH to verify the potential to transfect relevant cells in mice, leading to expression of the encoded mRNA. Transfected cells showed robust protein ZsGreen expression (see immunofluorescence images of FIG. 12).

As apparent, HNF4A mRNA transfection induced expression of target genes in PMH (see FIG. 13) and led to restoration of functions in fibrotic PMH (see FIG. 14); control experiments were performed with PMH isolated from WT mice without CCl₄ treatment.

Results: As apparent from FIG. 13 to FIG. 14, detailed in vitro analyses confirmed human HNF4A mRNA delivered into PMH improved functions, as was observed in PHH.

### Example 5—In Vivo Verification of Liver Fibrosis Inhibition in Mouse Model Upon HNF4A mRNA Delivery Encoding HNF4A WT Protein

In the present working example, in vivo targeted delivery of LNP-encapsulated HNF4A-mRNA (SEQ ID NO:5715, encoding HNF4A WT protein) into hepatocytes of fibrotic livers in mice was tested for therapeutic effects.

In detail for testing whether systemic administration of LNP-formulated mRNA encoding HNF4A WT protein inhibits liver fibrosis, the above described liver fibrosis mouse model with toxin- (repeated i.p. CCl₄ injection until termination, 16×) (FIG. 15-1 A-H) or cholestasis-induced fibrosis (induced via 3,5-diethoxycarbonyl-1,4-dihydrocollidine (DDC)-containing diet, continued diet until termination) (FIG. 15-2 I-P) were used. Efficacy of HNF4A/LNP was further compared with recombinant adeno-associated virus (AAV) vector serotype 8 (AAV8), by injecting fibrotic mice with 1×10¹¹ AAV8 particles, encoding optimized human HNF4A under transthyretin promoter.

Accordingly, LNP-formulated mRNA encoding HNF4A WT protein was injected into the animals via intravenous route (i.v.) starting on day 84 post CCl₄/DDC administration initiation. Treatment interval (6 repetitive injections) for HNF4A or ZsGreen mRNA was 5 days while a single injection was chosen for AAV control. A further control mice group received PBS buffer only. Final harvest was performed on day 112, i.e. all animals were weighed, blood was extracted by cardiac bleed for serum preparation, and the animals were euthanized followed by gross necropsy. Subsequently, the liver of each animal was harvested and prepared for further experimentation as described above (Hnf4a qPCRs, Western blots showing HNF4A protein expression, Liver function tests for ALT and bilirubin showing potentially reduced injury upon HNF4A/LNP administration. hydroxyproline assay, preparing of immunohistochemical images of H&E, desmin, and Sirius red stainings; all performed according to standard procedures which are routine for the skilled artisan, exemplary shown in working example 1B).

Results: HNF4A mRNA levels significantly decreased in fibrotic livers from both models (CCl₄ shown in FIG. 15-1 B, DDC-induced shown in FIG. 15-2 J). HNF4A/LNP or ZsGreen/LNP control were injected iv. in fibrotic mice at 2 mg/kg per injection (FIG. 15-1 A, FIG. 15-2 I). HNF4A protein expression was confirmed in HNF4A/LNP-injected mice livers (FIG. 15-1 C, FIG. 15-2 K) and was absent in control mice, since the HNF4A antibody was only specific to human but not mouse HNF4A. HNF4A/LNP-injected mice showed significantly reduced alanine aminotransferase (ALT) (FIG. 15-1 D) and bilirubin (FIG. 15-2 L) indicating improved liver function, and significantly reduced levels of collagen, suggesting decreased fibrosis in both CCl₄ (FIG. 15E) and DDC (FIG. 15-2 M) models. Histological analyses, desmin and Sirius red staining further confirmed reduced fibrosis from CCl₄ (FIG. 15-1 F-G) and DDC groups (FIG. 15-2 N-O). Additionally, qPCR showed significantly decreased expression of fibrogenic marker genes, Col1a1, Col2a1 and Acta2 (FIG. 15-1 H, FIG. 15-2 P).

Further, liver function test, hydroxyproline assay, histological analyses, desmin and Sirius red staining, and qPCR analyses of fibrogenic genes showed attenuation of liver fibrosis by HNF4A/LNP is comparable to AAV-mediated HNF4A delivery in CCl₄-induced (FIG. 15-1 D-H) or DDC-induced fibrosis (FIG. 15-2 L-P).

Accordingly, it could be successfully shown, that LNP-formulated mRNA encoding HNF4A WT protein inhibited toxin- and cholestasis-induced liver fibrosis in a several relevant in vivo models.

### Example 6—In Vivo Verification of Liver Cirrhosis Inhibition in Mouse Model Upon HNF4A mRNA Delivery Encoding HNF4A WT Protein

To determine, whether HNF4A mRNA encoding HNF4A WT protein inhibits liver cirrhosis in mice injected with CCl₄ twice weekly for 16 weeks (FIG. 16A), LNP-formulated mRNA encoding HNF4A WT protein (SEQ ID NO:5715) or LNP-formulated ZsGreen (SEQ ID NO:5714) were administered i.v. starting from 12 weeks, once every 5 days.

Consequently, HNF4A protein expression was measured by Western blot analyses (FIG. 16C), injury was assessed through ALT-measurement, and hydroxyproline content, body weight changes, activity score, histology, desmin, Sirius red staining and qPCR analyses for fibrogenic genes were further performed.

For experimental results see FIG. 16.

Results: as apparent from FIG. 16, cirrhotic mice showed loss of Hnf4a (FIG. 16B). HNF4A/LNP delivery led to HNF4A protein expression, as determined by Western blot analyses (FIG. 16C). Injury was evaluated by measurement of ALT, hydroxyproline content, body weight changes, activity score, histology, desmin, Sirius red staining and qPCR analyses for fibrogenic genes. All these analyses provided evidence that HNF4A/LNP inhibited liver cirrhosis in mice (FIG. 16D-J).

Thus, delivery of human HNF4A mRNA inhibited liver injury in a mouse model with features of cirrhosis.

### Example 7—In Vivo Verification of Suppression of Cholestasis and Fibrosis Upon HNF4A mRNA Delivery Encoding HNF4A WT Protein

In this working example, it was addressed whether HNF4A mRNA encoding HNF4A WT protein is able to suppress injury in multidrug resistance gene 2 knockout mice (Mdr2^{-/-}), a surrogate mouse model of progressive familial intrahepatic cholestasis. 12-week old Mdr2^{-/-} mice were injected with 2 mg/kg HNF4A/LNP (SEQ ID NO:5715) or ZsGreen/LNP (SEQ ID NO:5714), once every 5 days, before they were sacrificed at 16 weeks of age (FIG. 17A). For this evaluation, expression of Hnf4a mRNA in Mdr2^{-/-} mice was compared to age-matched Friend Virus B (FVB, Jackson Laboratory) background control mice (FIG. 17B). HNF4A protein expression, levels of bilirubin, hydroxyproline content, expression of Ck19, Sox9 and Epcam, injury by histology, desmin and Sirius red staining, and expression of fibrogenic genes, Col1a1, Col2a1 and Acta2 were measured (FIG. 17D-I).

For experimental results see FIG. 17.

Results: As apparent from FIG. 17, lower expression of Hnf4a mRNA in Mdr2^{-/-} mice was observed compared to age-matched FVB background control mice (FIG. 17B). Further, HNF4A/LNP-injected mice showed HNF4A protein expression (FIG. 17C), significantly decreased levels of bilirubin, hydroxyproline content, reduced expression of Ck19, Sox9 and Epcam, reduced injury by histology, desmin and Sirius red staining, and decreased expression of fibrogenic genes, Col1a1, Col2a1 and Acta2 (FIG. 17D-I).

Thus, delivery of LNP-formulated mRNA encoding HNF4A WT protein suppressed cholestasis and fibrosis in Mdr21-mice.

### Example 8—In Vivo Tolerability of Repeated Administration of LNP-Formulated HNF4A mRNA Encoding HNF4A WT Protein

In this working example, it was tested, whether repeated HNF4A mRNA encoding HNF4A WT protein LNP-administration was well tolerated in CCl₄-induced fibrotic mice. For this, the sera from mice with single or multiple injections with ZsGreen/LNP (SEQ ID NO:5714) or HNF4A/LNP (codon optimized HNF4A mRNA encoding WT HNF4A (SEQ ID NO:5715)) were subjected to cytokine analyses (n=3 mice for single ZsGreen/LNP or HNF4A/LNP injection group, n=6 mice for multiple ZsGreen/LNP or HNF4A/LNP injection group). Serum was collected at 6 hours after a single time mRNA/LNP injection or last injection during multiple mRNA/LNP injection. 2 mg/kg dose was used for both single and multiple injections experiments (for results, see FIG. 18).

Results: most of the cytokines between single and multiple injections either with ZsGreen/LNP or HNF4A/LNP did not differ significantly. Thus, repeated administration of HNF4A mRNA was found to be well tolerated as for mRNA-doses of up to 2 mg/kg, no significant immune response was induced as apparent from IL-1b, IL-4, IL-12, IL-13, TNF-α, and IFN-γ cytokine measurements.

### Example 9—Restoration of Endogenous Hnf4a Expression Levels after Repeated Delivery of Exogenous HNF4A mRNA Encoding HNF4A WT Protein In Vivo

To test whether transient delivery of HNF4A/LNP restores endogenous Hnf4a expression, qPCR analyses were performed with mice after 8 weeks of CCl₄ treatment or respectively DDC diet. Mice consequently received HNF4A mRNA treatment as described above (codon optimized HNF4A mRNA encoding WT HNF4A (SEQ ID NO:5715), n=6 for each group except WT control mice (n=4)). For results, see FIG. 19A/B.

Results: it was shown by qPCR analyses, that endogenous Hnf4a was downregulated after 8 weeks of CCl₄ treatment or in DDC model mice, however endogenous HNF4A expression increased significantly in mice injected with multiple doses of HNF4A/LNP, indicating a long-lasting phenotypic change in hepatocytes.

Consequently, it could be shown that the endogenous Hnf4a expression level was restored after repeated delivery of exogenous HNF4A mRNA.

### Example 10—Suppression and Reversal of Liver Cirrhosis in an In Vivo Disease Mouse Model Upon Administration of mRNA Encoding Engineered HNF4A Protein Variants

To determine, whether mRNA encoding engineered HNF4A protein variant combo 11 inhibits, reverses and/or attenuates liver cirrhosis in vivo, LNP-formulated mRNA encoding WT HNF4A protein and LNP-formulated mRNA encoding engineered HNF4A protein variant combo 11 were administered to a cirrhotic disease mice model. PBS buffer injection served as a control, as indicated in below Table Ex-4.

In detail, consistent with previous studies, for induction of cirrhosis in mice, mice were injected with CCl₄ every five days for 12 weeks. During treatment phase, the resulting cirrhotic mice were injected i.v. 6 times every 5 days with 0.3 mg/kg of mRNA according to Table Ex-4. In parallel to treatment injections during treatment phase, injection of CCl₄ was continued every 5 days to keep up the fibrosis/cirrhosis trigger. 24 hours after the last i.v. injection, mice were sacrificed and prepared for analysis. A schematic experimental overview is shown in FIG. 20.

TABLE Ex-4
in vivo verification of liver cirrhosis inhibition in mouse model upon
HNF4A mRNA delivery, either encoding WT HNF4A protein (SEQ ID NO: 100)
or encoding engineered HNF4A protein variant combo 11 (SEQ ID NO: 2947).
mRNA was formulated as described above in state of the art LNPs.

| | | | | | Dose, |
|---|---|---|---|---|---|
| | | | | | injection |
| Group | | | SEQ ID NO | No. of | route and |
| No. | Group | HNF4A protein version | (PRT/mRNA) | animals | schedule |
| 1 | no CCl₄ + PBS | N/A | N/A | 3 | N/A |
| | (control 1) | | | | |
| 2 | CCl4 + PBS | N/A | N/A | 3 | N/A |
| | (control 2) | | | | |
| 3 | CCl₄ + WT HNF4A | WT HNF4A | 100/2907 | 4 | 0.3 mg/kg |
| | | | | | 6 i.v. |
| 4 | CCl₄ + engineered | combo 11 (S87A K106R | 140/2947 | 4 | injections |
| | HNF4A protein | K108R K126R K127R S142A | | | every 5 days |
| | variant combo 11 | S143A S148A T166A S167A | | | |
| | | S313A S378A T429A T432A | | | |
| | | S436A K458R S461E) | | | |

In a subsequent detailed analysis phase,
Hydroxyproline assay;
Sirius red stainings;
Col1a1 qPCRs;
Desmin stainings;
Acta2 qPCRs; and
ALT measurements;

were performed, to determine, whether administration of mRNA encoding engineered HNF4A protein variants, can inhibit, reverse and/or attenuate liver cirrhosis in an in vivo model. All analyses were performed according to standard procedures which are routine for the skilled artisan, exemplary shown in working example 1B.

Results: it was shown by
Hydroxyproline assay (for details see FIG. 21);
Sirius red staining and quantification (for details see FIG. 22);
Col1a1 qPCR (for details see FIG. 23);
Desmin staining and quantification (for details see FIG. 24);
Acta2 qPCR (for details see FIG. 25); and
ALT (alanine aminotransferase) measurements (for details see FIG. 26);

analyses, that engineered HNF4A protein variant combo 11 was effective at doses of even 0.3 mg/kg for treating and/or suppressing liver cirrhosis. As apparent from FIG. 21 to FIG. 26, mRNA encoding engineered HNF4A protein variant combo 11 was more potent than mRNA encoding WT HNF4A following statistical analysis (p value comparison), especially regarding effects on hydroxyproline, sirus red staining, desmin staining, and Acta2 RNA levels. As apparent from FIG. 23, the detected Col1a1 RNA levels were comparable for the CCl₄ treated control and mRNA encoding WT HNF4A at 0.3 mg/kg, indicating that the mRNA encoding WT HNF4A was less efficient at this low dose and behaving as loss-of-function. In contrast, mRNA encoding combo 11 still decreased Col1a1 RNA levels at 0.3 mg/kg, confirming the conclusion that mRNA encoding engineered HNF4A protein variant combo 11 was more potent than mRNA encoding WT HNF4A. In conclusion, it could be verified in an in vivo model, that liver cirrhosis can be inhibited, reversed and/or attenuated upon administration of mRNA encoding engineered HNF4A protein variants, especially engineered HNF4A protein variant combo 11 (S87A K106R K108R K126R K127R S142A S143A S148A T166A S167A S313A S378A T429A T432A S436A K458R S461E).

### Example 11 [Prophetic]—In Vivo Verification of Liver Cirrhosis Inhibition in Mouse Model Upon HNF4A mRNA Delivery Encoding Engineered HNF4A Protein Variant S461E

To determine, whether mRNA encoding further engineered HNF4A protein variants inhibit and/or cure liver cirrhosis in vivo, LNP-formulated mRNA encoding WT HNF4A protein and LNP-formulated mRNA encoding engineered HNF4A protein variants are administered to a cirrhotic disease mice model. Buffer serves as a control as indicated in below Table Ex-5.

In detail, similar to previous procedures, for induction of cirrhosis in mice, mice are injected with CCl₄ every five days for 12 weeks. During treatment phase, the resulting cirrhotic mice are injected i.v. 6 times every 5 days with 0.1 mg/kg or 0.3 mg/kg of mRNA according to Table Ex-5. In parallel to treatment injections during treatment phase, also CCl₄ is injected every 5 days to further keep up the fibrosis/cirrhosis trigger. 24 hours after the last i.v. injection, mice are sacrificed and prepared for further analysis. A schematic experimental overview is shown in FIG. 20.

TABLE Ex-5
in vivo verification of liver cirrhosis inhibition in mouse model upon HNF4A mRNA delivery,
either encoding WT HNF4A protein (SEQ ID NO: 100) or encoding engineered HNF4A protein
variants. mRNA is formulated as described above in state of the art LNPs.

| | | | | | Dose, |
|---|---|---|---|---|---|
| | | | | | injection |
| Group | | | SEQ ID NO | No. of | route and |
| No. | Group | HNF4A protein version | (PRT/mRNA) | animals | schedule |
| 1 | no CCl₄ + PBS | N/A | N/A | 3 | N/A |
| | (control 1) | | | | |
| 2 | CCl4 + PBS | N/A | N/A | 3 | N/A |
| | (control 2) | | | | |
| 3 | CCl₄ + WT HNF4A | WT HNF4A | 100/2907 | 4 | 0.3 mg/kg |
| 4 | CCl₄ + engineered | S461E | 129/2936 | 4 | 6 i.v. |
| | HNF4A protein | | | | injections |
| | variant S461E | | | | every 5 days |
| 5 | CCl₄ + engineered | S461E | 129/5720 | 4 | |
| | HNF4A protein | | | | |
| | variant S461E | | | | |
| 6 | CCl₄ + engineered | S461E | 129/5723 | 4 | |
| | HNF4A protein | | | | |
| | variant S461E | | | | |
| 7 | CCl₄ + engineered | S461E | 129/5726 | 4 | |
| | HNF4A protein | | | | |
| | variant S461E | | | | |
| 8 | CCl₄ + WT HNF4A | WT HNF4A | 100/2907 | 4 | 0.1 mg/kg |
| 9 | CCl₄ + engineered | S461E | 129/2936 | 4 | 6 i.v. |
| | HNF4A protein | | | | injections |
| | variant S461E | | | | every 5 days |
| 10 | CCl₄ + engineered | S461E | 129/5720 | 4 | |
| | HNF4A protein | | | | |
| | variant S461E | | | | |
| 11 | CCl₄ + engineered | S461E | 129/5723 | 4 | |
| | HNF4A protein | | | | |
| | variant S461E | | | | |
| 12 | CCl₄ + engineered | S461E | 129/5726 | 4 | |
| | HNF4A protein | | | | |
| | variant S461E | | | | |

In a subsequent detailed analysis phase,
hydroxyproline assays;
Sirius red stainings;
Col1a1 qPCRs;
Desmin stainings;
Acta2 qPCRs; and
ALT measurements;

are performed, to find out, whether administration of mRNA encoding engineered HNF4A protein variants, can inhibit and/or cure liver cirrhosis in an in vivo model. All analyses are performed according to standard procedures which are routine for the skilled artisan, exemplary shown in working example 1B.

Results: it is shown by
hydroxyproline assay;
Sirius red staining;
Col1a1 qPCR;
Desmin staining;
Acta2 qPCR; and
ALT;

measurement and analyses, that engineered HNF4A protein variants are effective for treating and/or suppressing cirrhosis.

1. An isolated mRNA encoding
(i) an engineered hepatocyte nuclear factor 4 alpha (HNF4A) protein variant comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A comprising a S87A mutation and/or a S461E mutation, more preferably an engineered HNF4A comprising a S461E mutation; or
(ii) wild type hepatocyte nuclear factor 4 alpha (HNF4A);
for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer, more preferably treating, preventing, attenuating or inhibiting liver fibrosis or liver cirrhosis.

2. The mRNA of claim 1(i) for use according to claim 1, wherein said mRNA comprises an open reading frame (ORF) encoding an engineered HNF4A comprising one or more amino acid substitution, deletion, and/or insertion mutation leading to an increased HNF4A transcriptional activity, DNA binding capacity, stability, longer-lasting HNF4A half-life and/or therapeutic effect as compared to the unmodified human wild type HNF4A protein according to SEQ ID NO:100,
preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140,
more preferably an engineered HNF4A comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%,
or 100% sequence identity to any one of SEQ ID NO:101-246, most preferably to SEQ ID NO:140 having the biological activity of a HNF4A protein;
or a fragment or variant of said sequences having the biological activity of a HNF4A protein.

3. The mRNA according to any one of claim 1 to claim 2, wherein said mRNA preferably has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any single SEQ ID NO-element of SEQ ID NO:250-297, 299-346, 348-395, 397-444, 446-493, 495-542, 544-591, 593-640, 642-689, 1579-1626, 1628-1675, 1677-1724, 1726-1773, 1775-1822, 1824-1871, 1873-1920, 1922-1969, 1971-2018, 2908-2955, 2957-3004, 3006-3053, 3055-3102, 3104-3151, 3153-3200, 3202-3249, 3251-3298, 3300-3347, 4237-4284, 4286-4333, 4335-4382, 4384-4431, 4433-4480, 4482-4529, 4531-4578, 4580-4627, 4629-4676, 5720, 5721, 5723, 5724, 5726, 5727, 5729, 5730, 5732, 5733, 5735, and 5736, or a fragment or variant of said sequences, wherein the encoded protein has the biological activity of a HNF4A protein.

4. The mRNA according to any one of claim 2 to claim 3, wherein said engineered HNF4A comprises at least one substitution or substitution set at one or more positions selected from the group consisting of R2V, KSV, K179R, K180R, K234R, K300R, K307R, K309R, K447R, K470R, K458R, K106R, K108R, K126R, K127R, S313A, S313E, S142A, S143A, S142E, S143E, T166A, T166E, S148A, S148E, S183A, S183E, S461A, S461E, S167A, S167E, S378A, T429A, T432A, S436A, S378E, T429E, T432E, S436E, S87A, S87E, S95A, S99A, S138A, and T139A and/or combinations thereof, preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140 and wherein the amino acid positions of said amino acid sequence are numbered with reference to the human wild-type HNF4A protein (SEQ ID NO:100), more preferably an mRNA selected from the group consisting of SEQ ID NO:2947, SEQ ID NO:5721, SEQ ID NO:5724 and SEQ ID NO:5727.

5. The mRNA of claim 1(ii) or claim 2 to claim 4 for use according to claim 1, wherein said mRNA comprises an open reading frame (ORF) encoding an unmodified human wild type hepatocyte nuclear factor 4 alpha (HNF4A) according to SEQ ID NO:100, preferably wherein said mRNA has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:247, 248, 249, 298, 347, 396, 445, 494, 543, 592, 641, 1576, 1577, 1578, 1627, 1676, 1725, 1774, 1823, 1872, 1921, 1970, 2905, 2906, 2907, 2956, 3005, 3054, 3103, 3152, 3201, 3250, 3299, 4234, 4235, 4236, 4285, 4334, 4383, 4432, 4481, 4530, 4579, 4628, 5719, 5722, 5725, 5728, 5731, and 5734 or a fragment or variant of said sequences, wherein the encoded protein has the biological activity of a HNF4A protein, optionally wherein the mRNA further comprises an UTR combination selected from the group consisting of (i) a 5′-UTR derived from a mouse solute carrier family 7 (cationic amino acid transporter, y+ system) (SLC7A3) and a 3′-UTR derived from PSMB3; (ii) a 5′-UTR derived from mouse ribosomal protein L31 (RPL31) and a 3′-UTR derived from a human ribosomal protein S9 (RPS9); (iii) a 5′-UTR derived from ubiquilin 2 (Ubqln2) and a 3′-UTR derived from Guanine nucleotide-binding protein G(s) subunit alpha isoforms short (Gnas); and (iv) a 5′-UTR derived from a hydroxysteroid (17-beta) dehydrogenase 4 gene (HSD17B4) and a 3′-UTR derived from a proteasome subunit beta type-3 (PSMB3) UTR.

6. The mRNA according to any one of claim 1 to claim 5, wherein the
(i) G/C content of the HNF4A coding sequence in said mRNA is increased compared to the coding sequence of the corresponding wild type HNF4A coding sequence of SEQ ID NO:247 or 248;
(ii) C content of the HNF4A coding sequence in said mRNA is increased compared to the coding sequence of the corresponding wild type HNF4A coding sequence of SEQ ID NO:247 or 248; and/or wherein
(iii) at least one codon of the HNF4A coding sequence in said mRNA is adapted to human codon usage, wherein the codon adaptation index (CAI) is preferably increased or maximised in the corresponding HNF4A coding sequence compared to the coding sequence of the corresponding wild type HNF4A coding sequence of SEQ ID NO:247 or 248.

7. The mRNA according to any one of claim 1 to claim 6, wherein the mRNA comprises a 5′-cap structure, a poly(A) sequence comprising at least 70 A nucleotides, preferably about 100 A nucleotides, a poly(C) sequence, preferably comprising 10 to 200, 10 to 100, 20 to 70, 20 to 60 or 10 to 40 cytosine nucleotides, and/or at least one histone stem-loop, preferably, wherein the mRNA comprises a 3′-terminal A nucleotide.

8. The mRNA according to any one of claim 1 to claim 7, wherein the mRNA comprises, preferably in 5′ to 3′ direction, the following elements:
a) a 5′-cap1 structure;
b) a 5′-UTR element comprising a nucleic acid sequence, preferably derived from a 5′-UTR of a HSD17B4 gene, comprising the nucleic acid sequence according to SEQ ID NO:1 or 2, or a homolog, a fragment or a variant thereof;
c) at least one coding sequence as defined in any one of claim 1 to claim 10;
d) a 3′-UTR element comprising a nucleic acid sequence, preferably derived from a 3′-UTR of a PSMB3 gene, comprising the nucleic acid sequence according to SEQ ID NO:33 or 34, or a homolog, a fragment or a variant thereof;
e) a poly(A) sequence comprising about 100 adenosine nucleotides, preferably, wherein the mRNA comprises a 3′-terminal A nucleotide;
f) an optional poly(C) tail, preferably comprising 10 to 40 cytosine nucleotides; and/or
g) an optional histone stem-loop, preferably comprising the nucleic acid sequence according to SEQ ID NO:63 or 64.

9. The mRNA according to any one of claim 1 to claim 8, wherein the open reading frame does not comprise any chemically modified uracil or cytosine nucleotides.

10. The mRNA according to any one of claim 1 to claim 8, wherein the mRNA is chemically modified, preferably wherein the mRNA comprises pseudouridine (psi-uridine), N1-methylpseudouridine (N1MPU), 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosme, 7-deazaguanosine, 8-oxoadenosine, 8-oxoguanosine, O(6)-methylguanine, and/or 2-thiocytidine, more preferably wherein all uridine bases of the mRNA are fully chemically modified, even more preferably wherein all uridine bases of the mRNA are pseudouridine or N1-methylpseudouridine (N1MPU) bases, most preferably wherein all uridine bases of the mRNA are N1-methylpseudouridine (N1MPU) bases.

11. A lipid nanoparticle (LNP) comprising the mRNA according to any one of claim 1 to claim 10, wherein the LNP comprises an ionizable or cationic lipid, a phospholipid, a structural lipid, and a polymer conjugated lipid.

12. The LNP according to claim 11, wherein the lipids comprised in the LNP have a molar ratio of about 20-60% cationic or ionizable lipid, about 5-25% non-cationic lipid, about 25-55% sterol and about 0.5-15% polymer conjugated lipid.

13. The LNP according to anyone of claim 11 to claim 12, wherein the LNP does not comprise polyethylene glycol (PEG) or a PEG-modified lipid.

14. A pharmaceutical composition, comprising the mRNA according to any one of claim 1 to claim 10 or the LNP according to any one of claim 11 to claim 13.

15. A kit, preferably kit of parts, comprising at least one mRNA according to any one of claim 1 to claim 10, the LNP according to any one of claim 11 to claim 13, or the pharmaceutical composition according to claim 14, and optionally a liquid vehicle for solubilising and optionally technical instructions with information on the administration and dosage of the pharmaceutical composition.

16. A method of treating, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) or liver cancer, comprising administering to a human subject in need the mRNA according to any one of claim 1 to claim 10, the LNP according to any one of claim 11 to claim 13, the pharmaceutical composition according to claim 14, or the kit or kit of parts according to claim 15, wherein the administration results in treatment, prevention, attenuation, inhibition, or prophylaxis of the disease.

17. The method according to claim 16, wherein administration of the mRNA to a human subject in need results in
(i) improved hepatocyte metabolic activity; and/or
(ii) revived function of hepatocytes; and/or
(iii) increased mRNA levels of hepatocyte markers such as albumin (ALB), alpha-1 antitrypsin (A1AT), transferrin (TF) and/or transthyretin (TTR) in the liver or hepatocytes; and/or
(iv) reduced expression of fibrogenic marker genes Col1a1, Col2a1 Ck19, Sox9, Epcam, and/or Acta2 in the liver or hepatocytes; and/or
(v) decreased levels of bilirubin, hydroxyproline content in hepatocytes in the liver or hepatocytes; and/or
(vi) reduced liver injury as measured by histology, desmin or Sirius red staining;
(vii) increased expression of transporters of drug metabolism in the liver or hepatocytes;
(viii) increased serum paraoxonase and arylesterase 1 (PON1) expression or activity;
(ix) increased endogenous HNF4A or endogenous HNF1A levels or induction of the endogenous HNF1A-HNF4A transcriptional feedback loop; and/or
(x) function and fitness of hepatocytes;
as when compared to a non-treated human subject in need.

18. The method according to any one of claim 16 to claim 17, wherein the mRNA according to any one of claim 1 to claim 9, or the LNP according to any one of claim 11 to claim 13, or the pharmaceutical composition according to claim 14 or the kit or kit of parts according to claim 15 is administered to the subject by subcutaneous, intramuscular or intravenous administration, preferably intravenous administration.

19. The method according to any one of claim 16 to claim 18, wherein the mRNA comprises a 5′- or 3′-untranslated region (UTR) comprising at least one microRNA-binding site, preferably not being a microRNA-122 (miR-122) binding site, more preferably being miR-16, miR-21, miR-24, miR-27, miR-30c, miR-132, miR-133, miR-149, miR-192, miR-194, miR-204, miR-206, miR-208, or miR-223, most preferably being miRNA-148a, miRNA-101, miRNA-192 or miRNA-194, miR-126, miR-142-3p, or miR-142-5p.

20. The method according to any one of claim 16 to claim 19, wherein the method of treating the liver disease or liver disorder, preferably liver fibrosis, liver cirrhosis, hepatocellular carcinoma (HCC), non-alcoholic fatty liver disease (NAFLD), non-alcoholic steatohepatitis (NASH) or liver cancer, involves a single administration of the mRNA, the LNP, the pharmaceutical composition or the kit or kit of parts.

21. The method according to any one of claim 16 to claim 20, wherein the mRNA, the LNP, the pharmaceutical composition or the kit or kit of parts is administered
(a) once, preferably more than once, more preferably wherein administration is repeated for a period of at least 1 month, at least 2 months, at least 3 months, at least 4 months, at least 5 months, at least 6 months, at least one year, or lifelong; or
(b) about once a day, about once a week, about twice a week, about three times a week, about four times a week, about six or seven times a week, about once every two weeks, about once every three weeks, about once a month, about twice a month, about three times a month, or about four times a month.

22. An isolated mRNA according to any one of claim 1 to claim 10, or LNP according to any one of claim 11 to claim 13 or pharmaceutical composition according to claim 14 or kit or kit of parts according to claim 15, for use as a medicament.

23. An engineered HNF4A protein variant, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A protein variant comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138 or SEQ ID NO:140, more preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

24. The engineered HNF4A protein variant according to claim 23, for use as a medicament.

25. An isolated mRNA comprising an open reading frame (ORF) encoding an engineered HNF4A protein variant, comprising one or more amino acid exchange(s), leading to an increased HNF4A transcriptional activity, DNA binding capacity, stability, longer-lasting HNF4A half-life and/or therapeutic effect as compared to the unmodified human wild type HNF4A protein according to SEQ ID NO:100, preferably an engineered HNF4A protein variant comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:101-246, preferably to SEQ ID NO:138, SEQ ID NO:186 or SEQ ID NO:140, more preferably an engineered HNF4A comprising (i) a S87A mutation, (ii) a S461E mutation, (iii) a S87A and a S461E mutation, (iv) S87A K106R K108R K126R K127R, preferably SEQ ID NO:138, (v) S87A K106R K108R K126R K127R S142A S143A S148A T166A S167A S313A S378A T429A T432A S436A K458R, preferably SEQ ID NO:186 or (vi) S87A K106R K108R K126R K127R S142A S143A S148A T166A S167A S313A S378A T429A T432A S436A K458R S461E mutations, preferably SEQ ID NO:140.

26. An isolated mRNA having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NO:250, 299, 348, 397, 446, 495, 544, 593, 642, 1579, 1628, 1677, 1726, 1775, 1824, 1873, 1922, 1971, 2908, 2957, 3006, 3055, 3104, 3153, 3202, 3251, 3300, 4237, 4286, 4335, 4384, 4433, 4482, 4531, 4580, 4629, 251, 300, 349, 398, 447, 496, 545, 594, 643, 1580, 1629, 1678, 1727, 1776, 1825, 1874, 1923, 1972, 2909, 2958, 3007, 3056, 3105, 3154, 3203, 3252, 3301, 4238, 4287, 4336, 4385, 4434, 4483, 4532, 4581, 4630, 252, 301, 350, 399, 448, 497, 546, 595, 644, 1581, 1630, 1679, 1728, 1777, 1826, 1875, 1924, 1973, 2910, 2959, 3008, 3057, 3106, 3155, 3204, 3253, 3302, 4239, 4288, 4337, 4386, 4435, 4484, 4533, 4582, 4631, 253, 302, 351, 400, 449, 498, 547, 596, 645, 1582, 1631, 1680, 1729, 1778, 1827, 1876, 1925, 1974, 2911, 2960, 3009, 3058, 3107, 3156, 3205, 3254, 3303, 4240, 4289, 4338, 4387, 4436, 4485, 4534, 4583, 4632, 254, 303, 352, 401, 450, 499, 548, 597, 646, 1583, 1632, 1681, 1730, 1779, 1828, 1877, 1926, 1975, 2912, 2961, 3010, 3059, 3108, 3157, 3206, 3255, 3304, 4241, 4290, 4339, 4388, 4437, 4486, 4535, 4584, 4633, 255, 304, 353, 402, 451, 500, 549, 598, 647, 1584, 1633, 1682, 1731, 1780, 1829, 1878, 1927, 1976, 2913, 2962, 3011, 3060, 3109, 3158, 3207, 3256, 3305, 4242, 4291, 4340, 4389, 4438, 4487, 4536, 4585, 4634, 256, 305, 354, 403, 452, 501, 550, 599, 648, 1585, 1634, 1683, 1732, 1781, 1830, 1879, 1928, 1977, 2914, 2963, 3012, 3061, 3110, 3159, 3208, 3257, 3306, 4243, 4292, 4341, 4390, 4439, 4488, 4537, 4586, 4635, 257, 306, 355, 404, 453, 502, 551, 600, 649, 1586, 1635, 1684, 1733, 1782, 1831, 1880, 1929, 1978, 2915, 2964, 3013, 3062, 3111, 3160, 3209, 3258, 3307, 4244, 4293, 4342, 4391, 4440, 4489, 4538, 4587, 4636, 258, 307, 356, 405, 454, 503, 552, 601, 650, 1587, 1636, 1685, 1734, 1783, 1832, 1881, 1930, 1979, 2916, 2965, 3014, 3063, 3112, 3161, 3210, 3259, 3308, 4245, 4294, 4343, 4392, 4441, 4490, 4539, 4588, 4637, 259, 308, 357, 406, 455, 504, 553, 602, 651, 1588, 1637, 1686, 1735, 1784, 1833, 1882, 1931, 1980, 2917, 2966, 3015, 3064, 3113, 3162, 3211, 3260, 3309, 4246, 4295, 4344, 4393, 4442, 4491, 4540, 4589, 4638, 260, 309, 358, 407, 456, 505, 554, 603, 652, 1589, 1638, 1687, 1736, 1785, 1834, 1883, 1932, 1981, 2918, 2967, 3016, 3065, 3114, 3163, 3212, 3261, 3310, 4247, 4296, 4345, 4394, 4443, 4492, 4541, 4590, 4639, 261, 310, 359, 408, 457, 506, 555, 604, 653, 1590, 1639, 1688, 1737, 1786, 1835, 1884, 1933, 1982, 2919, 2968, 3017, 3066, 3115, 3164, 3213, 3262, 3311, 4248, 4297, 4346, 4395, 4444, 4493, 4542, 4591, 4640, 262, 311, 360, 409, 458, 507, 556, 605, 654, 1591, 1640, 1689, 1738, 1787, 1836, 1885, 1934, 1983, 2920, 2969, 3018, 3067, 3116, 3165, 3214, 3263, 3312, 4249, 4298, 4347, 4396, 4445, 4494, 4543, 4592, 4641, 263, 312, 361, 410, 459, 508, 557, 606, 655, 1592, 1641, 1690, 1739, 1788, 1837, 1886, 1935, 1984, 2921, 2970, 3019, 3068, 3117, 3166, 3215, 3264, 3313, 4250, 4299, 4348, 4397, 4446, 4495, 4544, 4593, 4642, 264, 313, 362, 411, 460, 509, 558, 607, 656, 1593, 1642, 1691, 1740, 1789, 1838, 1887, 1936, 1985, 2922, 2971, 3020, 3069, 3118, 3167, 3216, 3265, 3314, 4251, 4300, 4349, 4398, 4447, 4496, 4545, 4594, 4643, 265, 314, 363, 412, 461, 510, 559, 608, 657, 1594, 1643, 1692, 1741, 1790, 1839, 1888, 1937, 1986, 2923, 2972, 3021, 3070, 3119, 3168, 3217, 3266, 3315, 4252, 4301, 4350, 4399, 4448, 4497, 4546, 4595, 4644, 266, 315, 364, 413, 462, 511, 560, 609, 658, 1595, 1644, 1693, 1742, 1791, 1840, 1889, 1938, 1987, 2924, 2973, 3022, 3071, 3120, 3169, 3218, 3267, 3316, 4253, 4302, 4351, 4400, 4449, 4498, 4547, 4596, 4645, 267, 316, 365, 414, 463, 512, 561, 610, 659, 1596, 1645, 1694, 1743, 1792, 1841, 1890, 1939, 1988, 2925, 2974, 3023, 3072, 3121, 3170, 3219, 3268, 3317, 4254, 4303, 4352, 4401, 4450, 4499, 4548, 4597, 4646, 268, 317, 366, 415, 464, 513, 562, 611, 660, 1597, 1646, 1695, 1744, 1793, 1842, 1891, 1940, 1989, 2926, 2975, 3024, 3073, 3122, 3171, 3220, 3269, 3318, 4255, 4304, 4353, 4402, 4451, 4500, 4549, 4598, 4647, 269, 318, 367, 416, 465, 514, 563, 612, 661, 1598, 1647, 1696, 1745, 1794, 1843, 1892, 1941, 1990, 2927, 2976, 3025, 3074, 3123, 3172, 3221, 3270, 3319, 4256, 4305, 4354, 4403, 4452, 4501, 4550, 4599, 4648, 270, 319, 368, 417, 466, 515, 564, 613, 662, 1599, 1648, 1697, 1746, 1795, 1844, 1893, 1942, 1991, 2928, 2977, 3026, 3075, 3124, 3173, 3222, 3271, 3320, 4257, 4306, 4355, 4404, 4453, 4502, 4551, 4600, 4649, 271, 320, 369, 418, 467, 516, 565, 614, 663, 1600, 1649, 1698, 1747, 1796, 1845, 1894, 1943, 1992, 2929, 2978, 3027, 3076, 3125, 3174, 3223, 3272, 3321, 4258, 4307, 4356, 4405, 4454, 4503, 4552, 4601, 4650, 272, 321, 370, 419, 468, 517, 566, 615, 664, 1601, 1650, 1699, 1748, 1797, 1846, 1895, 1944, 1993, 2930, 2979, 3028, 3077, 3126, 3175, 3224, 3273, 3322, 4259, 4308, 4357, 4406, 4455, 4504, 4553, 4602, 4651, 273, 322, 371, 420, 469, 518, 567, 616, 665, 1602, 1651, 1700, 1749, 1798, 1847, 1896, 1945, 1994, 2931, 2980, 3029, 3078, 3127, 3176, 3225, 3274, 3323, 4260, 4309, 4358, 4407, 4456, 4505, 4554, 4603, 4652, 274, 323, 372, 421, 470, 519, 568, 617, 666, 1603, 1652, 1701, 1750, 1799, 1848, 1897, 1946, 1995, 2932, 2981, 3030, 3079, 3128, 3177, 3226, 3275, 3324, 4261, 4310, 4359, 4408, 4457, 4506, 4555, 4604, 4653, 275, 324, 373, 422, 471, 520, 569, 618, 667, 1604, 1653, 1702, 1751, 1800, 1849, 1898, 1947, 1996, 2933, 2982, 3031, 3080, 3129, 3178, 3227, 3276, 3325, 4262, 4311, 4360, 4409, 4458, 4507, 4556, 4605, 4654, 276, 325, 374, 423, 472, 521, 570, 619, 668, 1605, 1654, 1703, 1752, 1801, 1850, 1899, 1948, 1997, 2934, 2983, 3032, 3081, 3130, 3179, 3228, 3277, 3326, 4263, 4312, 4361, 4410, 4459, 4508, 4557, 4606, 4655, 277, 326, 375, 424, 473, 522, 571, 620, 669, 1606, 1655, 1704, 1753, 1802, 1851, 1900, 1949, 1998, 2935, 2984, 3033, 3082, 3131, 3180, 3229, 3278, 3327, 4264, 4313, 4362, 4411, 4460, 4509, 4558, 4607, 4656, 278, 327, 376, 425, 474, 523, 572, 621, 670, 1607, 1656, 1705, 1754, 1803, 1852, 1901, 1950, 1999, 2936, 2985, 3034, 3083, 3132, 3181, 3230, 3279, 3328, 4265, 4314, 4363, 4412, 4461, 4510, 4559, 4608, 4657, 279, 328, 377, 426, 475, 524, 573, 622, 671, 1608, 1657, 1706, 1755, 1804, 1853, 1902, 1951, 2000, 2937, 2986, 3035, 3084, 3133, 3182, 3231, 3280, 3329, 4266, 4315, 4364, 4413, 4462, 4511, 4560, 4609, 4658, 280, 329, 378, 427, 476, 525, 574, 623, 672, 1609, 1658, 1707, 1756, 1805, 1854, 1903, 1952, 2001, 2938, 2987, 3036, 3085, 3134, 3183, 3232, 3281, 3330, 4267, 4316, 4365, 4414, 4463, 4512, 4561, 4610, 4659, 281, 330, 379, 428, 477, 526, 575, 624, 673, 1610, 1659, 1708, 1757, 1806, 1855, 1904, 1953, 2002, 2939, 2988, 3037, 3086, 3135, 3184, 3233, 3282, 3331, 4268, 4317, 4366, 4415, 4464, 4513, 4562, 4611, 4660, 282, 331, 380, 429, 478, 527, 576, 625, 674, 1611, 1660, 1709, 1758, 1807, 1856, 1905, 1954, 2003, 2940, 2989, 3038, 3087, 3136, 3185, 3234, 3283, 3332, 4269, 4318, 4367, 4416, 4465, 4514, 4563, 4612, 4661, 283, 332, 381, 430, 479, 528, 577, 626, 675, 1612, 1661, 1710, 1759, 1808, 1857, 1906, 1955, 2004, 2941, 2990, 3039, 3088, 3137, 3186, 3235, 3284, 3333, 4270, 4319, 4368, 4417, 4466, 4515, 4564, 4613, 4662, 284, 333, 382, 431, 480, 529, 578, 627, 676, 1613, 1662, 1711, 1760, 1809, 1858, 1907, 1956, 2005, 2942, 2991, 3040, 3089, 3138, 3187, 3236, 3285, 3334, 4271, 4320, 4369, 4418, 4467, 4516, 4565, 4614, 4663, 285, 334, 383, 432, 481, 530, 579, 628, 677, 1614, 1663, 1712, 1761, 1810, 1859, 1908, 1957, 2006, 2943, 2992, 3041, 3090, 3139, 3188, 3237, 3286, 3335, 4272, 4321, 4370, 4419, 4468, 4517, 4566, 4615, 4664, 286, 335, 384, 433, 482, 531, 580, 629, 678, 1615, 1664, 1713, 1762, 1811, 1860, 1909, 1958, 2007, 2944, 2993, 3042, 3091, 3140, 3189, 3238, 3287, 3336, 4273, 4322, 4371, 4420, 4469, 4518, 4567, 4616, 4665, 287, 336, 385, 434, 483, 532, 581, 630, 679, 1616, 1665, 1714, 1763, 1812, 1861, 1910, 1959, 2008, 2945, 2994, 3043, 3092, 3141, 3190, 3239, 3288, 3337, 4274, 4323, 4372, 4421, 4470, 4519, 4568, 4617, 4666, 288, 337, 386, 435, 484, 533, 582, 631, 680, 1617, 1666, 1715, 1764, 1813, 1862, 1911, 1960, 2009, 2946, 2995, 3044, 3093, 3142, 3191, 3240, 3289, 3338, 4275, 4324, 4373, 4422, 4471, 4520, 4569, 4618, 4667, 289, 338, 387, 436, 485, 534, 583, 632, 681, 1618, 1667, 1716, 1765, 1814, 1863, 1912, 1961, 2010, 2947, 2996, 3045, 3094, 3143, 3192, 3241, 3290, 3339, 4276, 4325, 4374, 4423, 4472, 4521, 4570, 4619, 4668, 290, 339, 388, 437, 486, 535, 584, 633, 682, 1619, 1668, 1717, 1766, 1815, 1864, 1913, 1962, 2011, 2948, 2997, 3046, 3095, 3144, 3193, 3242, 3291, 3340, 4277, 4326, 4375, 4424, 4473, 4522, 4571, 4620, 4669, 291, 340, 389, 438, 487, 536, 585, 634, 683, 1620, 1669, 1718, 1767, 1816, 1865, 1914, 1963, 2012, 2949, 2998, 3047, 3096, 3145, 3194, 3243, 3292, 3341, 4278, 4327, 4376, 4425, 4474, 4523, 4572, 4621, 4670, 292, 341, 390, 439, 488, 537, 586, 635, 684, 1621, 1670, 1719, 1768, 1817, 1866, 1915, 1964, 2013, 2950, 2999, 3048, 3097, 3146, 3195, 3244, 3293, 3342, 4279, 4328, 4377, 4426, 4475, 4524, 4573, 4622, 4671, 293, 342, 391, 440, 489, 538, 587, 636, 685, 1622, 1671, 1720, 1769, 1818, 1867, 1916, 1965, 2014, 2951, 3000, 3049, 3098, 3147, 3196, 3245, 3294, 3343, 4280, 4329, 4378, 4427, 4476, 4525, 4574, 4623, 4672, 294, 343, 392, 441, 490, 539, 588, 637, 686, 1623, 1672, 1721, 1770, 1819, 1868, 1917, 1966, 2015, 2952, 3001, 3050, 3099, 3148, 3197, 3246, 3295, 3344, 4281, 4330, 4379, 4428, 4477, 4526, 4575, 4624, 4673, 295, 344, 393, 442, 491, 540, 589, 638, 687, 1624, 1673, 1722, 1771, 1820, 1869, 1918, 1967, 2016, 2953, 3002, 3051, 3100, 3149, 3198, 3247, 3296, 3345, 4282, 4331, 4380, 4429, 4478, 4527, 4576, 4625, 4674, 296, 345, 394, 443, 492, 541, 590, 639, 688, 1625, 1674, 1723, 1772, 1821, 1870, 1919, 1968, 2017, 2954, 3003, 3052, 3101, 3150, 3199, 3248, 3297, 3346, 4283, 4332, 4381, 4430, 4479, 4528, 4577, 4626, 4675, 297, 346, 395, 444, 493, 542, 591, 640, 689, 1626, 1675, 1724, 1773, 1822, 1871, 1920, 1969, 2018, 2955, 3004, 3053, 3102, 3151, 3200, 3249, 3298, 3347, 4284, 4333, 4382, 4431, 4480, 4529, 4578, 4627, 4676, 5720, 5721, 5723, 5724, 5726, 5727, 5729, 5730, 5732, 5733, 5735, and 5736.

27. An isolated mRNA according to any one of claim 1 to claim 10 and claim 25 to claim 26, LNP according to any one of claim 11 to claim 13, pharmaceutical composition according to claim 14, or kit or kit of parts according to claim 15, for use in treating, reversing, preventing, attenuating or inhibiting a liver disease, preferably selected from the group consisting of liver fibrosis, liver cirrhosis, hepatocellular carcinoma (HCC), non-alcoholic fatty liver disease (NAFLD), non-alcoholic steatohepatitis (NASH) and liver cancer in a human subject in need, comprising administering to a human subject in need the wherein the administration results in treatment, prevention, attenuation, inhibition, or prophylaxis of the liver fibrosis, liver cirrhosis, hepatocellular carcinoma (HCC), non-alcoholic fatty liver disease (NAFLD), non-alcoholic steatohepatitis (NASH) or liver cancer.

28. An isolated nucleic acid construct comprising a nucleic acid sequence encoding the mRNA according to claim 8, preferably an isolated nucleic acid construct having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the sequences selected from the group consisting of SEQ ID NO:1576-5615, 5683-5712, and 5716-5736 or SEQ ID NO:2906, 2956, 3005, 3250, 3299, 5728, 5731, 5734, 5563, 5564, 5565, 5566, 5567, 5568, 5569, 5570, 5571, 5572, 5573, 5574, 5575, 5576, 5577, 5578, 5579, 5580, 5581, 5582, 5583, 5584, 5585, 5586, 5587, 5588, 5589, 5590, 5591, 5592, 5593, 5594, 5595, 5596, 5597, 5598, 5599, 5600, 5601, 5602, 5603, 5604, 5605, 5606, 5607, 5608, 5609, 5610, 5611, 5612, 5613, 5614, and 5615 or to any one of the sequences as disclosed in Table C2 “Constructs of the invention”.

29. A fusion protein comprising the engineered HNF4A protein variant of any one of claim 23 or claim 24 or an mRNA encoding a fusion protein comprising the engineered HNF4A protein variant of any one of claim 23 or claim 24.

30. A vector comprising the isolated mRNA according to any one of claim 1 to claim 10.

31. A host cell carrying the vector of claim 30.
